SEQUENCE LISTING (113 sequences)

SEQ ID NO: 1 - protein, 240 aa, Artificial Sequence
  Synthetic Polypeptide
MEESVVRPSVFVVDGQTDIPFTRLGRSHRRQSCSVARVGLGLLLLLMGAGLAVQGWFLLQLHWRLGEMVTRLPDGPAGSWEQLIQERRSHEVNPAAHLTGANSSLTGSGGPLLWETQLGLAFLRGLSYHDGALVVTKAGYYYIYSKVQLGGVGCPLGLASTITHGLYKRTPRYPEELELLVSQQSPCGRATSSSRVWWDSSFLGGVVHLEAGEKVVVRVLDERLVRLRDGTRSYFGAFMV

SEQ ID NO: 2 - protein, 182 aa, Artificial Sequence
  Synthetic Polypeptide
LQLHWRLGEMVTRLPDGPAGSWEQLIQERRSHEVNPAAHLTGANSSLTGSGGPLLWETQLGLAFLRGLSYHDGALVVTKAGYYYIYSKVQLGGVGCPLGLASTITHGLYKRTPRYPEELELLVSQQSPCGRATSSSRVWWDSSFLGGVVHLEAGEKVVVRVLDERLVRLRDGTRSYFGAFMV

SEQ ID NO: 3 - protein, 167 aa, Artificial Sequence
  Synthetic Polypeptide
MQIPQAPWPVVWAVLQLGWRPGWFLDSPDRPWNPPTFSPALLVVTEGDNATFTCSFSNTSESFVLNWYRMSPSNQTDKLAAFPEDRSQPGQDCRFRVTQLPNGRDFHMSVVRARRNDSGTYLCGAISLAPKAQIKESLRAELRVTERRAEVPTAHPSPSPRPAGQFQ

SEQ ID NO: 4 - protein, 147 aa, Artificial Sequence
  Synthetic Polypeptide
PGWFLDSPDRPWNPPTFSPALLVVTEGDNATFTCSFSNTSESFVLNWYRMSPSNQTDKLAAFPEDRSQPGQDCRFRVTQLPNGRDFHMSVVRARRNDSGTYLCGAISLAPKAQIKESLRAELRVTERRAEVPTAHPSPSPRPAGQFQ

SEQ ID NO: 5 - protein, 559 aa, Artificial Sequence
  Synthetic Polypeptide
LDSPDRPWNPPTFSPALLVVTEGDNATFTCSFSNTSESFVLNWYRMSPSNQTDKLAAFPEDRSQPGQDCRFRVTQLPNGRDFHMSVVRARRNDSGTYLCGAISLAPKAQIKESLRAELRVTERRAEVPTAHPSPSPRPAGQFQSKYGPPCPPCPAPEFLGGPSVFLFPPKPKDQLMISRTPEVTCVVVDVSQEDPEVQFNWYVDGVEVHNAKTKPREEQFNSTYRVVSVLTVLHQDWLSGKEYKCKVSSKGLPSSIEKTISNATGQPREPQVYTLPPSQEEMTKNQVSLTCLVKGFYPSDIAVEWESNGQPENNYKTTPPVLDSDGSFFLYSRLTVDKSRWQEGNVFSCSVLHEALHNHYTQKSLSLSLGKIEGRMDLQLHWRLGEMVTRLPDGPAGSWEQLIQERRSHEVNPAAHLTGANSSLTGSGGPLLWETQLGLAFLRGLSYHDGALVVTKAGYYYIYSKVQLGGVGCPLGLASTITHGLYKRTPRYPEELELLVSQQSPCGRATSSSRVWWDSSFLGGVVHLEAGEKVVVRVLDERLVRLRDGTRSYFGAFMV

SEQ ID NO: 6 - protein, 373 aa, Artificial Sequence
  Synthetic Polypeptide
MEPAGPAPGRLGPLLCLLLAASCAWSGVAGEEELQVIQPDKSVLVAAGETATLRCTATSLIPVGPIQWFRGAGPGRELIYNQKEGHFPRVTTVSDLTKRNNMDFSIRIGNITPADAGTYYCVKFRKGSPDDVEFKSGAGTELSVRAKPSAPVVSGPAARATPQHTVSFTCESHGFSPRDITLKWFKNGNELSDFQTNVDPVGESVSYSIHSTAKVVLTREDVHSQVICEVAHVTLQGDPLRGTANLSETIRVPPTLEVTQQPVRAENQVNVTCQVRKFYPQRLQLTWLENGNVSRTETASTVTENKDGTYNWMSWLLVNVSAHRDDVKLTCQVEHDGQPAVSKSHDLKVSAHPKEQGSNTAAENTGSNERNIY

SEQ ID NO: 7 - protein, 343 aa, Artificial Sequence
  Synthetic Polypeptide
EEELQVIQPDKSVLVAAGETATLRCTATSLIPVGPIQWFRGAGPGRELIYNQKEGHFPRVTTVSDLTKRNNMDFSIRIGNITPADAGTYYCVKFRKGSPDDVEFKSGAGTELSVRAKPSAPVVSGPAARATPQHTVSFTCESHGFSPRDITLKWFKNGNELSDFQTNVDPVGESVSYSIHSTAKVVLTREDVHSQVICEVAHVTLQGDPLRGTANLSETIRVPPTLEVTQQPVRAENQVNVTCQVRKFYPQRLQLTWLENGNVSRTETASTVTENKDGTYNWMSWLLVNVSAHRDDVKLTCQVEHDGQPAVSKSHDLKVSAHPKEQGSNTAAENTGSNERNIY

SEQ ID NO: 8 - protein, 759 aa, Artificial Sequence
  Synthetic Polypeptide
EEELQVIQPDKSVLVAAGETATLRCTATSLIPVGPIQWFRGAGPGRELIYNQKEGHFPRVTTVSDLTKRNNMDFSIRIGNITPADAGTYYCVKFRKGSPDDVEFKSGAGTELSVRAKPSAPVVSGPAARATPQHTVSFTCESHGFSPRDITLKWFKNGNELSDFQTNVDPVGESVSYSIHSTAKVVLTREDVHSQVICEVAHVTLQGDPLRGTANLSETIRVPPTLEVTQQPVRAENQVNVTCQVRKFYPQRLQLTWLENGNVSRTETASTVTENKDGTYNWMSWLLVNVSAHRDDVKLTCQVEHDGQPAVSKSHDLKVSAHPKEQGSNTAAENTGSNERNIYSKYGPPCPPCPAPEFLGGPSVFLFPPKPKDQLMISRTPEVTCVVVDVSQEDPEVQFNWYVDGVEVHNAKTKPREEQFNSTYRVVSVLTVLHQDWLSGKEYKCKVSSKGLPSSIEKTISNATGQPREPQVYTLPPSQEEMTKNQVSLTCLVKGFYPSDIAVEWESNGQPENNYKTTPPVLDSDGSFFLYSRLTVDKSRWQEGNVFSCSVLHEALHNHYTQKSLSLSLGKIEGRMDLQLHWRLGEMVTRLPDGPAGSWEQLIQERRSHEVNPAAHLTGANSSLTGSGGPLLWETQLGLAFLRGLSYHDGALVVTKAGYYYIYSKVQLGGVGCPLGLASTITHGLYKRTPRYPEELELLVSQQSPCGRATSSSRVWWDSSFLGGVVHLEAGEKVVVRVLDERLVRLRDGTRSYFGAFMV

SEQ ID NO: 9 - protein, 141 aa, Artificial Sequence
  Synthetic Polypeptide
MRWCLLLIWAQGLRQAPLASGMMTGTIETTGNISAEKGGSIILQCHLSSTTAQVTQVNWEQQDQLLAICNADLGWHISPSFKDRVAPGPGLGLTLQSLTVNDTGEYFCIYHTYPDGTYTGRIFLEVLESSVAEHGARFQIP

SEQ ID NO: 10 - protein, 120 aa, Artificial Sequence
  Synthetic Polypeptide
MMTGTIETTGNISAEKGGSIILQCHLSSTTAQVTQVNWEQQDQLLAICNADLGWHISPSFKDRVAPGPGLGLTLQSLTVNDTGEYFCIYHTYPDGTYTGRIFLEVLESSVAEHGARFQIP

SEQ ID NO: 11 - protein, 536 aa, Artificial Sequence
  Synthetic Polypeptide
MMTGTIETTGNISAEKGGSIILQCHLSSTTAQVTQVNWEQQDQLLAICNADLGWHISPSFKDRVAPGPGLGLTLQSLTVNDTGEYFCIYHTYPDGTYTGRIFLEVLESSVAEHGARFQIPSKYGPPCPPCPAPEFLGGPSVFLFPPKPKDQLMISRTPEVTCVVVDVSQEDPEVQFNWYVDGVEVHNAKTKPREEQFNSTYRVVSVLTVLHQDWLSGKEYKCKVSSKGLPSSIEKTISNATGQPREPQVYTLPPSQEEMTKNQVSLTCLVKGFYPSDIAVEWESNGQPENNYKTTPPVLDSDGSFFLYSRLTVDKSRWQEGNVFSCSVLHEALHNHYTQKSLSLSLGKIEGRMDLQLHWRLGEMVTRLPDGPAGSWEQLIQERRSHEVNPAAHLTGANSSLTGSGGPLLWETQLGLAFLRGLSYHDGALVVTKAGYYYIYSKVQLGGVGCPLGLASTITHGLYKRTPRYPEELELLVSQQSPCGRATSSSRVWWDSSFLGGVVHLEAGEKVVVRVLDERLVRLRDGTRSYFGAFMV

SEQ ID NO: 12 - protein, 254 aa, Artificial Sequence
  Synthetic Polypeptide
MEYASDASLDPEAPWPPAPRARACRVLPWALVAGLLLLLLLAAACAVFLACPWAVSGARASPGSAASPRLREGPELSPDDPAGLLDLRQGMFAQLVAQNVLLIDGPLSWYSDPGLAGVSLTGGLSYKEDTKELVVAKAGVYYVFFQLELRRVVAGEGSGSVSLALHLQPLRSAAGAAALALTVDLPPASSEARNSAFGFQGRLLHLSAGQRLGVHLHTEARARHAWQLTQGATVLGLFRVTPEIPAGLPSPRSE

SEQ ID NO: 13 - protein, 205 aa, Artificial Sequence
  Synthetic Polypeptide
ACPWAVSGARASPGSAASPRLREGPELSPDDPAGLLDLRQGMFAQLVAQNVLLIDGPLSWYSDPGLAGVSLTGGLSYKEDTKELVVAKAGVYYVFFQLELRRVVAGEGSGSVSLALHLQPLRSAAGAAALALTVDLPPASSEARNSAFGFQGRLLHLSAGQRLGVHLHTEARARHAWQLTQGATVLGLFRVTPEIPAGLPSPRSE

SEQ ID NO: 14 - protein, 559 aa, Artificial Sequence
  Synthetic Polypeptide
MMTGTIETTGNISAEKGGSIILQCHLSSTTAQVTQVNWEQQDQLLAICNADLGWHISPSFKDRVAPGPGLGLTLQSLTVNDTGEYFCIYHTYPDGTYTGRIFLEVLESSVAEHGARFQIPSKYGPPCPPCPAPEFLGGPSVFLFPPKPKDQLMISRTPEVTCVVVDVSQEDPEVQFNWYVDGVEVHNAKTKPREEQFNSTYRVVSVLTVLHQDWLSGKEYKCKVSSKGLPSSIEKTISNATGQPREPQVYTLPPSQEEMTKNQVSLTCLVKGFYPSDIAVEWESNGQPENNYKTTPPVLDSDGSFFLYSRLTVDKSRWQEGNVFSCSVLHEALHNHYTQKSLSLSLGKIEGRMDACPWAVSGARASPGSAASPRLREGPELSPDDPAGLLDLRQGMFAQLVAQNVLLIDGPLSWYSDPGLAGVSLTGGLSYKEDTKELVVAKAGVYYVFFQLELRRVVAGEGSGSVSLALHLQPLRSAAGAAALALTVDLPPASSEARNSAFGFQGRLLHLSAGQRLGVHLHTEARARHAWQLTQGATVLGLFRVTPEIPAGLPSPRSE

SEQ ID NO: 15 - protein, 199 aa, Artificial Sequence
  Synthetic Polypeptide
MTLHPSPITCEFLFSTALISPKMCLSHLENMPLSHSRTQGAQRSSWKLWLFCSIVMLLFLCSFSWLIFIFLQLETAKEPCMAKFGPLPSKWQMASSEPPCVNKVSDWKLEILQNGLYLIYGQVAPNANYNDVAPFEVRLYKNKDMIQTLTNKSKIQNVGGTYELHVGDTIDLIFNSEHQVLKNNTYWGIILLANPQFIS

SEQ ID NO: 16 - protein, 128 aa, Artificial Sequence
  Synthetic Polypeptide
QLETAKEPCMAKFGPLPSKWQMASSEPPCVNKVSDWKLEILQNGLYLIYGQVAPNANYNDVAPFEVRLYKNKDMIQTLTNKSKIQNVGGTYELHVGDTIDLIFNSEHQVLKNNTYWGIILLANPQFIS

SEQ ID NO: 17 - protein, 482 aa, Artificial Sequence
  Synthetic Polypeptide
MMTGTIETTGNISAEKGGSIILQCHLSSTTAQVTQVNWEQQDQLLAICNADLGWHISPSFKDRVAPGPGLGLTLQSLTVNDTGEYFCIYHTYPDGTYTGRIFLEVLESSVAEHGARFQIPSKYGPPCPPCPAPEFLGGPSVFLFPPKPKDQLMISRTPEVTCVVVDVSQEDPEVQFNWYVDGVEVHNAKTKPREEQFNSTYRVVSVLTVLHQDWLSGKEYKCKVSSKGLPSSIEKTISNATGQPREPQVYTLPPSQEEMTKNQVSLTCLVKGFYPSDIAVEWESNGQPENNYKTTPPVLDSDGSFFLYSRLTVDKSRWQEGNVFSCSVLHEALHNHYTQKSLSLSLGKIEGRMDQLETAKEPCMAKFGPLPSKWQMASSEPPCVNKVSDWKLEILQNGLYLIYGQVAPNANYNDVAPFEVRLYKNKDMIQTLTDKSKIQNVGGTYELHVGDTIDLIFNSEHQVLKNNTYWGIILLANPQFIS

SEQ ID NO: 18 - protein, 251 aa, Artificial Sequence
  Synthetic Polypeptide
MAEDLGLSFGETASVEMLPEHGSCRPKARSSSARWALTCCLVLLPFLAGLTTYLLVSQLRAQGEACVQFQALKGQEFAPSHQQVYAPLRADGDKPRAHLTVVRQTPTQHFKNQFPALHWEHELGLAFTKNRMNYTNKFLLIPESGDYFIYSQVTFRGMTSECSEIRQAGRPNKPDSITVVITKVTDSYPEPTQLLMGTKSVCEVGSNWFQPIYLGAMFSLQEGDKLMVNVSDISLVDYTKEDKTFFGAFLL

SEQ ID NO: 19 - protein, 192 aa, Artificial Sequence
  Synthetic Polypeptide
RAQGEACVQFQALKGQEFAPSHQQVYAPLRADGDKPRAHLTVVRQTPTQHFKNQFPALHWEHELGLAFTKNRMNYTNKFLLIPESGDYFIYSQVTFRGMTSECSEIRQAGRPNKPDSITVVITKVTDSYPEPTQLLMGTKSVCEVGSNWFQPIYLGAMFSLQEGDKLMVNVSDISLVDYTKEDKTFFGAFLL

SEQ ID NO: 20 - protein, 549 aa, Artificial Sequence
  Synthetic Polypeptide
MMTGTIETTGNISAEKGGSIILQCHLSSTTAQVTQVNWEQQDQLLAICNADLGWHISPSFKDRVAPGPGLGLTLQSLTVNDTGEYFCIYHTYPDGTYTGRIFLEVLESSVAEHGARFQIPSKYGPPCPPCPAPEFLGGPSVFLFPPKPKDQLMISRTPEVTCVVVDVSQEDPEVQFNWYVDGVEVHNAKTKPREEQFNSTYRVVSVLTVLHQDWLSGKEYKCKVSSKGLPSSIEKTISNATGQPREPQVYTLPPSQEEMTKNQVSLTCLVKGFYPSDIAVEWESNGQPENNYKTTPPVLDSDGSFFLYSRLTVDKSRWQEGNVFSCSVLHEALHNHYTQKSLSLSLGKIEGRMDSQLRAQGEACVQFQALKGQEFAPSHQQVYAPLRADGDKPRAHLTVVRQTPTQHFKNQFPALHWEHELGLAFTKNRMNYTNKFLLIPESGDYFIYSQVTFRGMTSECSEIRQAGRPNKPDSITVVITKVTDSYPEPTQLLMGTKSVCEVGSNWFQPIYLGAMFSLQEGDKLMVNVSDISLVDYTKEDKTFFGAFLL

SEQ ID NO: 21 - protein, 183 aa, Artificial Sequence
  Synthetic Polypeptide
MERVQPLEENVGNAARPRFERNKLLLVASVIQGLGLLLCFTYICLHFSALQVSHRYPRIQSIKVQFTEYKKEKGFILTSQKEDEIMKVQNNSVIINCDGFYLISLKGYFSQEVNISLHYQKDEEPLFQLKKVRSVNSLMVASLTYKDKVYLNVTTDNTSLDDFHVNGGELILIHQNPGEFCVL

SEQ ID NO: 22 - protein, 133 aa, Artificial Sequence
  Synthetic Polypeptide
QVSHRYPRIQSIKVQFTEYKKEKGFILTSQKEDEIMKVQNNSVIINCDGFYLISLKGYFSQEVNISLHYQKDEEPLFQLKKVRSVNSLMVASLTYKDKVYLNVTTDNTSLDDFHVNGGELILIHQNPGEFCVL

SEQ ID NO: 23 - protein, 487 aa, Artificial Sequence
  Synthetic Polypeptide
MMTGTIETTGNISAEKGGSIILQCHLSSTTAQVTQVNWEQQDQLLAICNADLGWHISPSFKDRVAPGPGLGLTLQSLTVNDTGEYFCIYHTYPDGTYTGRIFLEVLESSVAEHGARFQIPSKYGPPCPPCPAPEFLGGPSVFLFPPKPKDQLMISRTPEVTCVVVDVSQEDPEVQFNWYVDGVEVHNAKTKPREEQFNSTYRVVSVLTVLHQDWLSGKEYKCKVSSKGLPSSIEKTISNATGQPREPQVYTLPPSQEEMTKNQVSLTCLVKGFYPSDIAVEWESNGQPENNYKTTPPVLDSDGSFFLYSRLTVDKSRWQEGNVFSCSVLHEALHNHYTQKSLSLSLGKIEGRMDQVSHRYPRIQSIKVQFTEYKKEKGFILTSQKEDEIMKVQNNSVIINCDGFYLISLKGYFSQEVNISLHYQKDEEPLFQLKKVRSVNSLMVASLTYKDKVYLNVTTDNTSLDDFHVNGGELILIHQNPGEFCVL

SEQ ID NO: 24 - protein, 150 aa, Artificial Sequence
  Synthetic Polypeptide
MKTLPAMLGTGKLFWVFFLIPYLDIWNIHGKESCDVQLYIKRQSEHSILAGDPFELECPVKYCANRPHVTWCKLNGTTCVKLEDRQTSWKEEKNISFFILHFEPVLPNDNGSYRCSANFQSNLIESHSTTLYVTDVKSASERPSKDEMAS

SEQ ID NO: 25 - protein, 127 aa, Artificial Sequence
  Synthetic Polypeptide
KESCDVQLYIKRQSEHSILAGDPFELECPVKYCANRPHVTWCKLNGTTCVKLEDRQTSWKEEKNISFFILHFEPVLPNDNGSYRCSANFQSNLIESHSTTLYVTDVKSASERPSKDEMASRPWLLYR

SEQ ID NO: 26 - protein, 234 aa, Artificial Sequence
  Synthetic Polypeptide
MDPGLQQALNGMAPPGDTAMHVPAGSVASHLGTTSRSYFYLTTATLALCLVFTVATIMVLVVQRTDSIPNSPDNVPLKGGNCSEDLLCILKRAPFKKSWAYLQVAKHLNKTKLSWNKDGILHGVRYQDGNLVIQFPGLYFIICQLQFLVQCPNNSVDLKLELLINKHIKKQALVTVCESGMQTKHVYQNLSQFLLDYLQVNTTISVNVDTFQYIDTSTFPLENVLSIFLYSNSD

SEQ ID NO: 27 - protein, 172 aa, Artificial Sequence
  Synthetic Polypeptide
QRTDSIPNSPDNVPLKGGNCSEDLLCILKRAPFKKSWAYLQVAKHLNKTKLSWNKDGILHGVRYQDGNLVIQFPGLYFIICQLQFLVQCPNNSVDLKLELLINKHIKKQALVTVCESGMQTKHVYQNLSQFLLDYLQVNTTISVNVDTFQYIDTSTFPLENVLSIFLYSNSD

SEQ ID NO: 28 - protein, 261 aa, Artificial Sequence
  Synthetic Polypeptide
MIETYNQTSPRSAATGLPISMKIFMYLLTVFLITQMIGSALFAVYLHRRLDKIEDERNLHEDFVFMKTIQRCNTGERSLSLLNCEEIKSQFEGFVKDIMLNKEETKKENSFEMQKGDQNPQIAAHVISEASSKTTSVLQWAEKGYYTMSNNLVTLENGKQLTVKRQGLYYIYAQVTFCSNREASSQAPFIASLCLKSPGRFERILLRAANTHSSAKPCGQQSIHLGGVFELQPGASVFVNVTDPSQVSHGTGFTSFGLLKL

SEQ ID NO: 29 - protein, 215 aa, Artificial Sequence
  Synthetic Polypeptide
HRRLDKIEDERNLHEDFVFMKTIQRCNTGERSLSLLNCEEIKSQFEGFVKDIMLNKEETKKENSFEMQKGDQNPQIAAHVISEASSKTTSVLQWAEKGYYTMSNNLVTLENGKQLTVKRQGLYYIYAQVTFCSNREASSQAPFIASLCLKSPGRFERILLRAANTHSSAKPCGQQSIHLGGVFELQPGASVFVNVTDPSQVSHGTGFTSFGLLKL

SEQ ID NO: 30 - protein, 281 aa, Artificial Sequence
  Synthetic Polypeptide
MQQPFNYPYPQIYWVDSSASSPWAPPGTVLPCPTSVPRRPGQRRPPPPPPPPPLPPPPPPPPLPPLPLPPLKKRGNHSTGLCLLVMFFMVLVALVGLGLGMFQLFHLQKELAELRESTSQMHTASSLEKQIGHPSPPPEKKELRKVAHLTGKSNSRSMPLEWEDTYGIVLLSGVKYKKGGLVINETGLYFVYSKVYFRGQSCNNLPLSHKVYMRNSKYPQDLVMMEGKMMSYCTTGQMWARSSYLGAVFNLTSADHLYVNVSELSLVNFEESQTFFGLYKL

SEQ ID NO: 31 - protein, 179 aa, Artificial Sequence
  Synthetic Polypeptide
QLFHLQKELAELRESTSQMHTASSLEKQIGHPSPPPEKKELRKVAHLTGKSNSRSMPLEWEDTYGIVLLSGVKYKKGGLVINETGLYFVYSKVYFRGQSCNNLPLSHKVYMRNSKYPQDLVMMEGKMMSYCTTGQMWARSSYLGAVFNLTSADHLYVNVSELSLVNFEESQTFFGLYKL

SEQ ID NO: 32-35 - skipped (4 entries)

SEQ ID NO: 36 - protein, 202 aa, Artificial Sequence
  Synthetic Polypeptide
MFSHLPFDCVLLLLLLLLTRSSEVEYRAEVGQNAYLPCFYTPAAPGNLVPVCWGKGACPVFECGNVVLRTDERDVNYWTSRYWLNGDFRKGDVSLTIENVTLADSGIYCCRIQIPGIMNDEKFNLKLVIKPAKVTPAPTRQRDFTAAFPRMLTTRGHGPAETQTLGSLPDINLTQISTLANELRDSRLANDLRDSGATIRIG

SEQ ID NO: 37 - protein, 181 aa, Artificial Sequence
  Synthetic Polypeptide
SEVEYRAEVGQNAYLPCFYTPAAPGNLVPVCWGKGACPVFECGNVVLRTDERDVNYWTSRYWLNGDFRKGDVSLTIENVTLADSGIYCCRIQIPGIMNDEKFNLKLVIKPAKVTPAPTRQRDFTAAFPRMLTTRGHGPAETQTLGSLPDINLTQISTLANELRDSRLANDLRDSGATIRIG

SEQ ID NO: 38 - protein, 150 aa, Artificial Sequence
  Synthetic Polypeptide
MGSPGMVLGLLVQIWALQEASSLSVQQGPNLLQVRQGSQATLVCQVDQATAWERLRVKWTKDGAILCQPYITNGSLSLGVCGPQGRLSWQAPSHLTLQLDPVSLNHSGAYVCWAAVEIPELEEAEGNITRLFVDPDDPTQNRNRIASFPG

SEQ ID NO: 39 - protein, 128 aa, Artificial Sequence
  Synthetic Polypeptide
LSVQQGPNLLQVRQGSQATLVCQVDQATAWERLRVKWTKDGAILCQPYITNGSLSLGVCGPQGRLSWQAPSHLTLQLDPVSLNHSGAYVCWAAVEIPELEEAEGNITRLFVDPDDPTQNRNRIASFPG

SEQ ID NO: 40 - protein, 281 aa, Artificial Sequence [sequence omitted]

SEQ ID NO: 41 - protein, 243 aa, Artificial Sequence
  Synthetic Polypeptide
TNELKQMQDKYSKSGIACFLKEDDSYWDPNDEESMNSPCWQVKWQLRQLVRKMILRTSEETISTVQEKQQNISPLVRERGPQRVAAHITGTRGRSNTLSSPNSKNEKALGRKINSWESSRSGHSFLSNLHLRNGELVIHEKGFYYIYSQTYFRFQEEIKENTKNDKQMVQYIYKYTSYPDPILLMKSARNSCWSKDAEYGLYSIYQGGIFELKENDRIFVSVTNEHLIDMDHEASFFGAFLVG

SEQ ID NO: 42 - protein, 543 aa, Artificial Sequence
  Synthetic Polypeptide
KESCDVQLYIKRQSEHSILAGDPFELECPVKYCANRPHVTWCKLNGTTCVKLEDRQTSWKEEKNISFFILHFEPVLPNDNGSYRCSANFQSNLIESHSTTLYVTDVKSASERPSKDEMASRPWLLYRSKYGPPCPPCPAPEFLGGPSVFLFPPKPKDQLMISRTPEVTCVVVDVSQEDPEVQFNWYVDGVEVHNAKTKPREEQFNSTYRVVSVLTVLHQDWLSGKEYKCKVSSKGLPSSIEKTISNATGQPREPQVYTLPPSQEEMTKNQVSLTCLVKGFYPSDIAVEWESNGQPENNYKTTPPVLDSDGSFFLYSRLTVDKSRWQEGNVFSCSVLHEALHNHYTQKSLSLSLGKIEGRMDLQLHWRLGEMVTRLPDGPAGSWEQLIQERRSHEVNPAAHLTGANSSLTGSGGPLLWETQLGLAFLRGLSYHDGALVVTKAGYYYIYSKVQLGGVGCPLGLASTITHGLYKRTPRYPEELELLVSQQSPCGRATSSSRVWWDSSFLGGVVHLEAGEKVVVRVLDERLVRLRDGTRSYFGAFMV

SEQ ID NO: 43 - protein, 838 aa, Artificial Sequence
  Synthetic Polypeptide
VPVVWAQEGAPAQLPCSPTIPLQDLSLLRRAGVTWQHQPDSGPPAAAPGHPLAPGPHPAAPSSWGPRPRRYTVLSVGPGGLRSGRLPLQPRVQLDERGRQRGDFSLWLRPARRADAGEYRAAVHLRDRALSCRLRLRLGQASMTASPPGSLRASDWVILNCSFSRPDRPASVHWFRNRGQGRVPVRESPHHHLAESFLFLPQVSPMDSGPWGCILTYRDGFNVSIMYNLTVLGLEPPTPLTVYAGAGSRVGLPCRLPAGVGTRSFLTAKWTPPGGGPDLLVTGDNGDFTLRLEDVSQAQAGTYTCHIHLQEQQLNATVTLAIITVTPKSFGSPGSLGKLLCEVTPVSGQERFVWSSLDTPSQRSFSGPWLEAQEAQLLSQPWQCQLYQGERLLGAAVYFTELSSPGAQRSGRAPGALPAGHLSKYGPPCPPCPAPEFLGGPSVFLFPPKPKDQLMISRTPEVTCVVVDVSQEDPEVQFNWYVDGVEVHNAKTKPREEQFNSTYRVVSVLTVLHQDWLSGKEYKCKVSSKGLPSSIEKTISNATGQPREPQVYTLPPSQEEMTKNQVSLTCLVKGFYPSDIAVEWESNGQPENNYKTTPPVLDSDGSFFLYSRLTVDKSRWQEGNVFSCSVLHEALHNHYTQKSLSLSLGKIEGRMDLQLHWRLGEMVTRLPDGPAGSWEQLIQERRSHEVNPAAHLTGANSSLTGSGGPLLWETQLGLAFLRGLSYHDGALVVTKAGYYYIYSKVQLGGVGCPLGLASTITHGLYKRTPRYPEELELLVSQQSPCGRATSSSRVWWDSSFLGGVVHLEAGEKVVVRVLDERLVRLRDGTRSYFGAFMV

SEQ ID NO: 44 - protein, 597 aa, Artificial Sequence
  Synthetic Polypeptide
SEVEYRAEVGQNAYLPCFYTPAAPGNLVPVCWGKGACPVFECGNVVLRTDERDVNYWTSRYWLNGDFRKGDVSLTIENVTLADSGIYCCRIQIPGIMNDEKFNLKLVIKPAKVTPAPTRQRDFTAAFPRMLTTRGHGPAETQTLGSLPDINLTQISTLANELRDSRLANDLRDSGATIRIGSKYGPPCPPCPAPEFLGGPSVFLFPPKPKDQLMISRTPEVTCVVVDVSQEDPEVQFNWYVDGVEVHNAKTKPREEQFNSTYRVVSVLTVLHQDWLSGKEYKCKVSSKGLPSSIEKTISNATGQPREPQVYTLPPSQEEMTKNQVSLTCLVKGFYPSDIAVEWESNGQPENNYKTTPPVLDSDGSFFLYSRLTVDKSRWQEGNVFSCSVLHEALHNHYTQKSLSLSLGKIEGRMDLQLHWRLGEMVTRLPDGPAGSWEQLIQERRSHEVNPAAHLTGANSSLTGSGGPLLWETQLGLAFLRGLSYHDGALVVTKAGYYYIYSKVQLGGVGCPLGLASTITHGLYKRTPRYPEELELLVSQQSPCGRATSSSRVWWDSSFLGGVVHLEAGEKVVVRVLDERLVRLRDGTRSYFGAFMV

SEQ ID NO: 45 - protein, 569 aa, Artificial Sequence
  Synthetic Polypeptide
MMTGTIETTGNISAEKGGSIILQCHLSSTTAQVTQVNWEQQDQLLAICNADLGWHISPSFKDRVAPGPGLGLTLQSLTVNDTGEYFCIYHTYPDGTYTGRIFLEVLESSVAEHGARFQIPSKYGPPCPPCPAPEFLGGPSVFLFPPKPKDQLMISRTPEVTCVVVDVSQEDPEVQFNWYVDGVEVHNAKTKPREEQFNSTYRVVSVLTVLHQDWLSGKEYKCKVSSKGLPSSIEKTISNATGQPREPQVYTLPPSQEEMTKNQVSLTCLVKGFYPSDIAVEWESNGQPENNYKTTPPVLDSDGSFFLYSRLTVDKSRWQEGNVFSCSVLHEALHNHYTQKSLSLSLGKIEGRMDHRRLDKIEDERNLHEDFVFMKTIQRCNTGERSLSLLNCEEIKSQFEGFVKDIMLNKEETKKENSFEMQKGDQNPQIAAHVISEASSKTTSVLQWAEKGYYTMSNNLVTLENGKQLTVKRQGLYYIYAQVTFCSNREASSQAPFIASLCLKSPGRFERILLRAANTHSSAKPCGQQSIHLGGVFELQPGASVFVNVTDPSQVSHGTGFTSFGLLKL

SEQ ID NO: 46 - protein, 217 aa, Artificial Sequence
  Synthetic Polypeptide
APEFLGGPSVFLFPPKPKDTLMISRTPEVTCVVVDVSQEDPEVQFNWYVDGVEVHNAKTKPREEQFNSTYRVVSVLTVLHQDWLSGKEYKCKVSSKGLPSSIEKTISNATGQPREPQVYTLPPSQEEMTKNQVSLTCLVKGFYPSDIAVEWESNGQPENNYKTTPPVLDSDGSFFLYSRLTVDKSSWQEGNVFSCSVMHEALHNHYTQKSLSLSLGK

SEQ ID NO: 47 - protein, 217 aa, Artificial Sequence
  Synthetic Polypeptide
APEFLGGPSVFLFPPKPKDQLMISRTPEVTCVVVDVSQEDPEVQFNWYVDGVEVHNAKTKPREEQFNSTYRVVSVLTTPHSDWLSGKEYKCKVSSKGLPSSIEKTISNATGQPREPQVYTLPPSQEEMTKNQVSLTCLVKGFYPSDIAVEWESNGQPENNYKTTPPVLDSDGSFFLYSRLTVDKSSWQEGNVFSCSVLHEALHNHYTQKSLSLSLGK

SEQ ID NO: 48 - protein, 217 aa, Artificial Sequence
  Synthetic Polypeptide
APEFLGGPSVFLFPPKPKDQLMISRTPEVTCVVVDVSQEDPEVQFNWYVDGVEVHNAKTKPREEQFNSTYRVVSVLTVLHQDWLSGKEYKCKVSSKGLPSSIEKTISNATGQPREPQVYTLPPSQEEMTKNQVSLTCLVKGFYPSDIAVEWESNGQPENNYKTTPPVLDSDGSFFLYSRLTVDKSRWQEGNVFSCSVLHEALHNHYTQKSLSLSLGK

SEQ ID NO: 49 - protein, 11 aa, Artificial Sequence
  Synthetic Polypeptide
SKYGPPCPSCP

SEQ ID NO: 50 - protein, 11 aa, Artificial Sequence
  Synthetic Polypeptide
SKYGPPCPPCP

SEQ ID NO: 51 - protein, 6 aa, Artificial Sequence
  Synthetic Polypeptide
SKYGPP

SEQ ID NO: 52 - protein, 6 aa, Artificial Sequence
  Synthetic Polypeptide
IEGRMD

SEQ ID NO: 53 - protein, 9 aa, Artificial Sequence
  Synthetic Polypeptide
GGGVPRDCG

SEQ ID NO: 54 - protein, 15 aa, Artificial Sequence
  Synthetic Polypeptide
IEGRMDGGGGAGGGG

SEQ ID NO: 55 - protein, 8 aa, Artificial Sequence
  Synthetic Polypeptide
GGGSGGGS

SEQ ID NO: 56 - protein, 12 aa, Artificial Sequence
  Synthetic Polypeptide
GGGSGGGGSGGG

SEQ ID NO: 57 - protein, 14 aa, Artificial Sequence
  Synthetic Polypeptide
EGKSSGSGSESKST

SEQ ID NO: 58 - protein, 4 aa, Artificial Sequence
  Synthetic Polypeptide
GGSG

SEQ ID NO: 59 - protein, 12 aa, Artificial Sequence
  Synthetic Polypeptide
GGSGGGSGGGSG

SEQ ID NO: 60 - protein, 15 aa, Artificial Sequence
  Synthetic Polypeptide
EAAAKEAAAKEAAAK

SEQ ID NO: 61 - protein, 20 aa, Artificial Sequence
  Synthetic Polypeptide
EAAAREAAAREAAAREAAAR

SEQ ID NO: 62 - protein, 17 aa, Artificial Sequence
  Synthetic Polypeptide
GGGGSGGGGSGGGGSAS

SEQ ID NO: 63 - protein, 9 aa, Artificial Sequence
  Synthetic Polypeptide
GGGGAGGGG

SEQ ID NO: 64 - skipped

SEQ ID NO: 65 - protein, 6 aa, Artificial Sequence
  Synthetic Polypeptide
GSGSGS

SEQ ID NO: 66 - protein, 10 aa, Artificial Sequence
  Synthetic Polypeptide
GSGSGSGSGS

SEQ ID NO: 67 - protein, 7 aa, Artificial Sequence
  Synthetic Polypeptide
GGGGSAS

SEQ ID NO: 68 - protein, 20 aa, Artificial Sequence
  Synthetic Polypeptide
APAPAPAPAPAPAPAPAPAP

SEQ ID NO: 69 - protein, 4 aa, Artificial Sequence
  Synthetic Polypeptide
CPPC

SEQ ID NO: 70 - protein, 5 aa, Artificial Sequence
  Synthetic Polypeptide
GGGGS

SEQ ID NO: 71 - protein, 10 aa, Artificial Sequence
  Synthetic Polypeptide
GGGGSGGGGS

SEQ ID NO: 72 - protein, 15 aa, Artificial Sequence
  Synthetic Polypeptide
GGGGSGGGGSGGGGS

SEQ ID NO: 73 - protein, 20 aa, Artificial Sequence
  Synthetic Polypeptide
GGGGSGGGGSGGGGSGGGGS

SEQ ID NO: 74 - protein, 25 aa, Artificial Sequence
  Synthetic Polypeptide
GGGGSGGGGSGGGGSGGGGSGGGGS

SEQ ID NO: 75 - protein, 30 aa, Artificial Sequence
  Synthetic Polypeptide
GGGGSGGGGSGGGGSGGGGSGGGGSGGGGS

SEQ ID NO: 76 - protein, 35 aa, Artificial Sequence
  Synthetic Polypeptide
GGGGSGGGGSGGGGSGGGGSGGGGSGGGGSGGGGS

SEQ ID NO: 77 - protein, 40 aa, Artificial Sequence
  Synthetic Polypeptide
GGGGSGGGGSGGGGSGGGGSGGGGSGGGGSGGGGSGGGGS

SEQ ID NO: 78 - protein, 16 aa, Artificial Sequence
  Synthetic Polypeptide
GGSGGSGGGGSGGGGS

SEQ ID NO: 79 - protein, 8 aa, Artificial Sequence
  Synthetic Polypeptide
GGGGGGGG

SEQ ID NO: 80 - protein, 6 aa, Artificial Sequence
  Synthetic Polypeptide
GGGGGG

SEQ ID NO: 81 - protein, 5 aa, Artificial Sequence
  Synthetic Polypeptide
EAAAK

SEQ ID NO: 82 - protein, 10 aa, Artificial Sequence
  Synthetic Polypeptide
EAAAKEAAAK

SEQ ID NO: 83 - protein, 15 aa, Artificial Sequence
  Synthetic Polypeptide
EAAAKEAAAKEAAAK

SEQ ID NO: 84 - protein, 12 aa, Artificial Sequence
  Synthetic Polypeptide
AEAAAKEAAAKA

SEQ ID NO: 85 - protein, 17 aa, Artificial Sequence
  Synthetic Polypeptide
AEAAAKEAAAKEAAAKA

SEQ ID NO: 86 - protein, 22 aa, Artificial Sequence
  Synthetic Polypeptide
AEAAAKEAAAKEAAAKEAAAKA

SEQ ID NO: 87 - protein, 27 aa, Artificial Sequence
  Synthetic Polypeptide
AEAAAKEAAAKEAAAKEAAAKEAAAKA

SEQ ID NO: 88 - protein, 46 aa, Artificial Sequence
  Synthetic Polypeptide
AEAAAKEAAAKEAAAKEAAAKALEAEAAAKEAAAKEAAAKEAAAKA

SEQ ID NO: 89 - protein, 5 aa, Artificial Sequence
  Synthetic Polypeptide
PAPAP

SEQ ID NO: 90 - protein, 18 aa, Artificial Sequence
  Synthetic Polypeptide
KESGSVSSEQLAQFRSLD

SEQ ID NO: 91 - protein, 12 aa, Artificial Sequence
  Synthetic Polypeptide
GSAGSAAGSGEF

SEQ ID NO: 92 - protein, 5 aa, Artificial Sequence
  Synthetic Polypeptide
GGGSE

SEQ ID NO: 93 - protein, 5 aa, Artificial Sequence
  Synthetic Polypeptide
GSESG

SEQ ID NO: 94 - protein, 5 aa, Artificial Sequence
  Synthetic Polypeptide
GSEGS

SEQ ID NO: 95 - protein, 35 aa, Artificial Sequence
  Synthetic Polypeptide
GEGGSGEGSSGEGSSSEGGGSEGGGSEGGGSEGGS

SEQ ID NO: 96 - protein, 234 aa, Artificial Sequence
  Synthetic Polypeptide
SKYGPPCPSCPAPEFLGGPSVFLFPPKPKDTLMISRTPEVTCVVVDVSQEDPEVQFNWYVDGVEVHNAKTKPREEQFNSTYRVVSVLTVLHQDWLSGKEYKCKVSSKGLPSSIEKTISNATGQPREPQVYTLPPSQEEMTKNQVSLTCLVKGFYPSDIAVEWESNGQPENNYKTTPPVLDSDGSFFLYSRLTVDKSSWQEGNVFSCSVMHEALHNHYTQKSLSLSLGKIEGRMD

SEQ ID NO: 97 - protein, 234 aa, Artificial Sequence
  Synthetic Polypeptide
SKYGPPCPSCPAPEFLGGPSVFLFPPKPKDQLMISRTPEVTCVVVDVSQEDPEVQFNWYVDGVEVHNAKTKPREEQFNSTYRVVSVLTTPHSDWLSGKEYKCKVSSKGLPSSIEKTISNATGQPREPQVYTLPPSQEEMTKNQVSLTCLVKGFYPSDIAVEWESNGQPENNYKTTPPVLDSDGSFFLYSRLTVDKSSWQEGNVFSCSVLHEALHNHYTQKSLSLSLGKIEGRMD

SEQ ID NO: 98 - protein, 234 aa, Artificial Sequence
  Synthetic Polypeptide
SKYGPPCPSCPAPEFLGGPSVFLFPPKPKDQLMISRTPEVTCVVVDVSQEDPEVQFNWYVDGVEVHNAKTKPREEQFNSTYRVVSVLTVLHQDWLSGKEYKCKVSSKGLPSSIEKTISNATGQPREPQVYTLPPSQEEMTKNQVSLTCLVKGFYPSDIAVEWESNGQPENNYKTTPPVLDSDGSFFLYSRLTVDKSRWQEGNVFSCSVLHEALHNHYTQKSLSLSLGKIEGRMD

SEQ ID NO: 99 - protein, 234 aa, Artificial Sequence
  Synthetic Polypeptide
SKYGPPCPPCPAPEFLGGPSVFLFPPKPKDTLMISRTPEVTCVVVDVSQEDPEVQFNWYVDGVEVHNAKTKPREEQFNSTYRVVSVLTVLHQDWLSGKEYKCKVSSKGLPSSIEKTISNATGQPREPQVYTLPPSQEEMTKNQVSLTCLVKGFYPSDIAVEWESNGQPENNYKTTPPVLDSDGSFFLYSRLTVDKSSWQEGNVFSCSVMHEALHNHYTQKSLSLSLGKIEGRMD

SEQ ID NO: 100 - protein, 234 aa, Artificial Sequence
  Synthetic Polypeptide
SKYGPPCPPCPAPEFLGGPSVFLFPPKPKDQLMISRTPEVTCVVVDVSQEDPEVQFNWYVDGVEVHNAKTKPREEQFNSTYRVVSVLTTPHSDWLSGKEYKCKVSSKGLPSSIEKTISNATGQPREPQVYTLPPSQEEMTKNQVSLTCLVKGFYPSDIAVEWESNGQPENNYKTTPPVLDSDGSFFLYSRLTVDKSSWQEGNVFSCSVLHEALHNHYTQKSLSLSLGKIEGRMD

SEQ ID NO: 101 - protein, 234 aa, Artificial Sequence
  Synthetic Polypeptide
SKYGPPCPPCPAPEFLGGPSVFLFPPKPKDQLMISRTPEVTCVVVDVSQEDPEVQFNWYVDGVEVHNAKTKPREEQFNSTYRVVSVLTVLHQDWLSGKEYKCKVSSKGLPSSIEKTISNATGQPREPQVYTLPPSQEEMTKNQVSLTCLVKGFYPSDIAVEWESNGQPENNYKTTPPVLDSDGSFFLYSRLTVDKSRWQEGNVFSCSVLHEALHNHYTQKSLSLSLGKIEGRMD

SEQ ID NO: 102 - protein, 254 aa, Artificial Sequence; identical to SEQ ID NO: 12
  Synthetic Polypeptide

SEQ ID NO: 103 - protein, 782 aa, Artificial Sequence
  Synthetic Polypeptide
EEELQVIQPDKSVLVAAGETATLRCTATSLIPVGPIQWFRGAGPGRELIYNQKEGHFPRVTTVSDLTKRNNMDFSIRIGNITPADAGTYYCVKFRKGSPDDVEFKSGAGTELSVRAKPSAPVVSGPAARATPQHTVSFTCESHGFSPRDITLKWFKNGNELSDFQTNVDPVGESVSYSIHSTAKVVLTREDVHSQVICEVAHVTLQGDPLRGTANLSETIRVPPTLEVTQQPVRAENQVNVTCQVRKFYPQRLQLTWLENGNVSRTETASTVTENKDGTYNWMSWLLVNVSAHRDDVKLTCQVEHDGQPAVSKSHDLKVSAHPKEQGSNTAAENTGSNERNIYSKYGPPCPPCPAPEFLGGPSVFLFPPKPKDQLMISRTPEVTCVVVDVSQEDPEVQFNWYVDGVEVHNAKTKPREEQFNSTYRVVSVLTVLHQDWLSGKEYKCKVSSKGLPSSIEKTISNATGQPREPQVYTLPPSQEEMTKNQVSLTCLVKGFYPSDIAVEWESNGQPENNYKTTPPVLDSDGSFFLYSRLTVDKSRWQEGNVFSCSVLHEALHNHYTQKSLSLSLGKIEGRMDACPWAVSGARASPGSAASPRLREGPELSPDDPAGLLDLRQGMFAQLVAQNVLLIDGPLSWYSDPGLAGVSLTGGLSYKEDTKELVVAKAGVYYVFFQLELRRVVAGEGSGSVSLALHLQPLRSAAGAAALALTVDLPPASSEARNSAFGFQGRLLHLSAGQRLGVHLHTEARARHAWQLTQGATVLGLFRVTPEIPAGLPSPRSE

SEQ ID NO: 104 - protein, 23 aa, Artificial Sequence
  Synthetic Polypeptide
MAAVGPRTGPGTGAEALALAAEL

SEQ ID NO: 105 - protein, 13 aa, Artificial Sequence
  Synthetic Polypeptide
AATRAPPFPLPCP

SEQ ID NO: 106 - protein, 16 aa, Artificial Sequence
  Synthetic Polypeptide
HGSQAAAARAAAARCG

SEQ ID NO: 107 - protein, 30 aa, Artificial Sequence
  Synthetic Polypeptide
NVSLKTFVLKGMLKKFKEDLRGELEKEEKV

SEQ ID NO: 108 - protein, 781 aa, Artificial Sequence
  Synthetic Polypeptide
KELKVTQPEKSVSVAAGDSTVLNCTLTSLLPVGPIKWYRGVGQSRLLIYSFTGEHFPRVTNVSDATKRNNMDFSIRISNVTPEDAGTYYCVKFQKGPSEPDTEIQSGGGTEVYVLAKPSPPEVSGPADRGIPDQKVNFTCKSHGFSPRNITLKWFKDGQELHHLETTVNPSGKNVSYNISSTVRVVLNSMDVHSKVICEVAHITLDRSPLRGIANLSNFIRVSPTVKVTQQSPTSMNQVNLTCRAERFYPEDLQLIWLENGNVSRNDTPKNLTKNTDGTYNYTSLFLVNSSAHREDVVFTCQVKHDQQPAITRNHTVLGLAHSSDQGSMQTFPGNNATHNWNVPRDCGCKPCICTVPEVSSVFIFPPKPKDVLTITLTPKVTCVVVDISKDDPEVQFSWFVDDVEVHTAQTQPREEQFNSTFRSVSELPIMHQDWLNGKEFKCRVNSAAFPAPIEKTISKTKGRPKAPQVYTIPPPKEQMAKDKVSLTCMITDFFPEDITVEWQWNGQPAENYKNTQPIMDTDGSYFVYSKLNVQKSNWEAGNTFTCSVLHEGLHNHHTEKSLSHSPGIIEGRMDRTEPRPALTITTSPNLGTRENNADQVTPVSHIGCPNTTQQGSPVFAKLLAKNQASLCNTTLNWHSQDGAGSSYLSQGLRYEEDKKELVVDSPGLYYVFLELKLSPTFTNTGHKVQGWVSLVLQAKPQVDDFDNLALTVELFPCSMENKLVDRSWSQLLLLKAGHRLSVGLRAYLHGAQDAYRDWELSYPNTTSFGLFLVKPDNPWE

SEQ ID NO: 109 - protein, 540 aa, Artificial Sequence
  Synthetic Polypeptide
MMTGTIETTGNISAEKGGSIILQCHLSSTTAQVTQVNWEQQDQLLAICNADLGWHISPSFKDRVAPGPGLGLTLQSLTVNDTGEYFCIYHTYPDGTYTGRIFLEVLESSVAEHGARFQIPEPKSCDKTHTCPPCPAPELLGGPSVFLFPPKPKDTLMISRTPEVTCVVVDVSHEDPEVKFNWYVDGVEVHNAKTKPREEQYASTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGKIEGRMDLQLHWRLGEMVTRLPDGPAGSWEQLIQERRSHEVNPAAHLTGANSSLTGSGGPLLWETQLGLAFLRGLSYHDGALVVTKAGYYYIYSKVQLGGVGCPLGLASTITHGLYKRTPRYPEELELLVSQQSPCGRATSSSRVWWDSSFLGGVVHLEAGEKVVVRVLDERLVRLRDGTRSYFGAFMV

SEQ ID NO: 110 - protein, 540 aa, Artificial Sequence
  Synthetic Polypeptide
MMTGTIETTGNISAEKGGSIILQCHLSSTTAQVTQVNWEQQDQLLAICNADLGWHISPSFKDRVAPGPGLGLTLQSLTVNDTGEYFCIYHTYPDGTYTGRIFLEVLESSVAEHGARFQIPEPKSCDKTHTCPPCPAPEAAGGPSVFLFPPKPKDTLMISRTPEVTCVVVDVSHEDPEVKFNWYVDGVEVHNAKTKPREEQYASTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGKIEGRMDLQLHWRLGEMVTRLPDGPAGSWEQLIQERRSHEVNPAAHLTGANSSLTGSGGPLLWETQLGLAFLRGLSYHDGALVVTKAGYYYIYSKVQLGGVGCPLGLASTITHGLYKRTPRYPEELELLVSQQSPCGRATSSSRVWWDSSFLGGVVHLEAGEKVVVRVLDERLVRLRDGTRSYFGAFMV

SEQ ID NO: 111 - protein, 534 aa, Artificial Sequence
  Synthetic Polypeptide
TIDTKRNISAEEGGSVILQCHFSSDTAEVTQVDWKQQDQLLAIYSVDLGWHVASVFSDRVVPGPSLGLTFQSLTMNDTGEYFCTYHTYPGGIYKGRIFLKVQESSDDRNGLAQFQTAPLGVPRDCGCKPCICTVPEVSSVFIFPPKPKDVLTITLTPKVTCVVVDISKDDPEVQFSWFVDDVEVHTAQTQPREEQFNSTFRSVSELPIMHQDWLNGKEFKCRVNSAAFPAPIEKTISKTKGRPKAPQVYTIPPPKEQMAKDKVSLTCMITDFFPEDITVEWQWNGQPAENYKNTQPIMDTDGSYFVYSKLNVQKSNWEAGNTFTCSVLHEGLHNHHTEKSLSHSPGKIEGRMDLHQRLGDIVAHLPDGGKGSWEKLIQDQRSHQANPAAHLTGANASLIGIGGPLLWETRLGLAFLRGLTYHDGALVTMEPGYYYVYSKVQLSGVGCPQGLANGLPITHGLYKRTSRYPKELELLVSRRSPCGRANSSRVWWDSSFLGGVVHLEAGEEVVVRVPGNRLVRPRDGTRSYFGAFMV

SEQ ID NO: 112 - protein, 232 aa, Artificial Sequence
  Synthetic Polypeptide
EPKSCDKTHTCPPCPAPELLGGPSVFLFPPKPKDTLMISRTPEVTCVVVDVSHEDPEVKFNWYVDGVEVHNAKTKPREEQYASTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 113 - protein, 232 aa, Artificial Sequence
  Synthetic Polypeptide
EPKSCDKTHTCPPCPAPEAAGGPSVFLFPPKPKDTLMISRTPEVTCVVVDVSHEDPEVKFNWYVDGVEVHNAKTKPREEQYASTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

(19) United States
(12) Patent Application Publication
(10) Pub. No.: US 2024/0016892 A1
(43) Pub. Date: Jan. 18, 2024

(54) METHODS OF TREATING CANCER USING TIGIT-AND LIGHT-BASED CHIMERIC PROTEINS

(71) Applicant: Shattuck Labs, Inc., Austin, TX (US)

(72) Inventors: Taylor SCHREIBER, Austin, TX (US); George FROMM, Austin, TX (US); Suresh DE SILVA, Austin, TX (US)

(21) Appl. No.: 18/265,048

(22) PCT Filed: Dec. 3, 2021
(86) PCT No.: PCT/US21/61841

§ 371 Date: Jun. 2, 2023

Related U.S. Application Data
(60) Provisional application No. 63/276,066, filed on Nov. 5, 2021.
(60) Provisional application No. 63/215,735, filed on Jun. 28, 2021.
(60) Provisional application No. 63/173,090, filed on Apr. 9, 2021.
(60) Provisional application No. 63/121,083, filed on Dec. 3, 2020.

(51) Int. Cl. A61K 38/17; A61K 38/19; A61K 49/00; A61P 35/00; G01N 33/574; C12Q 1/6886

(52) U.S. Cl. CPC A61K 38/1774; A61K 38/191; A61K 49/0004; A61P 35/00; G01N 33/57484; C12Q 1/6886; C07K 2319/30; C12Q 2600/106; A61B 10/0045

(57) ABSTRACT

The present disclosure relates, inter alia, to compositions and methods, including chimeric proteins that find use in the treatment of disease, and to detection and treatment of drug resistant cancer using chimeric proteins.

### PRIORITY

This application claims the benefit of, and priority to, U.S. Provisional Application Nos. 63/121,083, filed Dec. 3, 2020; 63/173,090, filed Apr. 9, 2021; 63/215,735, filed Jun. 28, 2021; and 63/276,066, filed Nov. 5, 2021, the contents of each of which are hereby incorporated by reference in its entirety.

### FIELD OF THE DISCLOSURE

The present disclosure relates, inter alia, to compositions and methods, including chimeric proteins that find use in the treatment of disease, and to detection and treatment of drug resistant cancer using chimeric proteins.

### DESCRIPTION OF THE TEXT FILE SUBMITTED ELECTRONICALLY

The contents of the text file submitted electronically herewith are incorporated herein by reference in their entirety: a computer readable format copy of the Sequence Listing (filename: SHK-038PC2_ST25; date created: Dec. 1, 2021; size 177,048 bytes).

### BACKGROUND

The field of cancer immunotherapy has grown tremendously over the past several years. This has been largely driven by the clinical efficacy of antibodies targeting the family of checkpoint molecules (e.g., CTLA-4 and PD-1/L1) in many tumor types. However, despite this success, clinical response to these agents as monotherapy occurs in a minority of patients (10-45% in various solid tumors), and these therapies are hindered by side effects.

Discovery of the proper dose and regimen of such agents is crucial to efficacious treatment of cancers. Developing novel treatment strategies, including dosing and regimens, remains a formidable task given the complexity of the human immune system, the high cost, and the potential for toxicity which may result from such interventions.

In addition, drug resistance remains one of the biggest challenges in cancer therapy, including immunotherapy. It is also common that a patient with advanced cancer receives a drug that helps shrink their tumors, but then weeks or months later the cancer comes back and the drug no longer works. Thus, drug resistance, either existing before treatment (intrinsic or primary resistance) or developed after therapy (acquired resistance), is responsible for most relapses of cancer, one of the major causes of death. Therefore, better understanding the mechanisms of drug resistance is required to provide guidance to future cancer treatment. Unfortunately, few effective therapeutic options are available for some patients having cancers that are resistant to the anti-checkpoint therapies. Thus, new therapies for patients suffering from drug resistant cancer and methods for selecting appropriate drugs for patients having drug-resistant cancer are required for improving outcomes in cancer patients.

### SUMMARY

In various aspects, the present technology provides for compositions and methods that are useful for cancer immunotherapy. In addition, the present disclosure provides, in part, methods for selecting patients for cancer treatment, and methods for cancer treatment, based on, for instance, based on gene expression profiles of anti-PD-1 resistant cancers.

In one aspect, the present disclosure relates to a method for treating a cancer in a subject in need thereof the method comprising a step of administering to the subject an effective amount of a chimeric protein having a general structure of: N terminus—(a)—(b)—(c)—C terminus, wherein: (a) is a first domain comprising an extracellular domain of human T cell immunoreceptor with Ig and ITIM domains (TIGIT), (b) is a linker adjoining the first and second domains, wherein the linker comprises a hinge-CH2-CH3 Fc domain, and (c) is a second domain comprising an extracellular domain of human LIGHT (lymphotoxin-like, exhibits inducible expression and competes with HSV glycoprotein D for herpesvirus entry mediator (HVEM), a receptor expressed by T lymphocytes). In embodiments, the dose of the chimeric protein administered is between about 0.0001 mg/kg and about 50.0 mg/kg, optionally selected from about 1 mg/kg, about 3 mg/kg, about 6 mg/kg, or about 10 mg/kg, about 12 mg/kg, about 15 mg/kg, about 18 mg/kg, about 20 mg/kg, about 22 mg/kg, about 25 mg/kg, about 27 mg/kg, about 30 mg/kg, about 33 mg/kg, about 35 mg/kg, about 37 mg/kg, about mg/kg, about 42 mg/kg, about 45 mg/kg, about 48 mg/kg, and about 50 mg/kg. in embodiments, the subject is a human, optionally an adult human.

In one aspect, the present disclosure relates to a method for inducing lymphocyte expansion in a subject in need thereof, the method comprising a step of administering to the subject an effective amount of a chimeric protein having a general structure of: N terminus—(a)—(b)—(c)—C terminus, wherein: (a) is a first domain comprising an extracellular domain of human T cell immunoreceptor with Ig and ITIM domains (TIGIT), (b) is a linker adjoining the first and second domains, wherein the linker comprises a hinge-CH2-CH3 Fc domain, and (c) is a second domain comprising an extracellular domain of human LIGHT (lymphotoxin-like, exhibits inducible expression and competes with HSV glycoprotein D for herpesvirus entry mediator (HVEM), a receptor expressed by T lymphocytes).

In one aspect, the present disclosure relates to a method for inducing lymphocyte margination in a subject in need thereof, the method comprising a step of administering to the subject an effective amount of a chimeric protein having a general structure of: N terminus—(a)—(b)—(c)—C terminus, wherein: (a) is a first domain comprising an extracellular domain of human T cell immunoreceptor with Ig and ITIM domains (TIGIT), (b) is a linker adjoining the first and second domains, wherein the linker comprises a hinge-CH2-CH3 Fc domain, and (c) is a second domain comprising an extracellular domain of human LIGHT (lymphotoxin-like, exhibits inducible expression and competes with HSV glycoprotein D for herpesvirus entry mediator (HVEM), a receptor expressed by T lymphocytes).

In one aspect, the present disclosure relates to a method of evaluating the efficacy of a cancer treatment in a subject in need thereof, the method comprising the steps of: (i) administering a dose of a chimeric protein having a general structure of: N terminus—(a)—(b)—(c)—C terminus, wherein: (a) is a first domain comprising an extracellular domain of human T cell immunoreceptor with Ig and ITIM domains (TIGIT), (b) is a linker adjoining the first and second domains, wherein the linker comprises a hinge-CH2-CH3 Fc domain, and (c) is a second domain comprising an extracellular domain of human LIGHT (lymphotoxin-like, exhibits inducible expression and competes with HSV glycoprotein D for herpesvirus entry mediator (HVEM), a receptor expressed by T lymphocytes). wherein the dose is from about 0.03 mg/kg to about 50 mg/kg; (ii) obtaining a biological sample from the subject; (iii) performing an assay on the biological sample to determine level and/or activity of a cytokine selected from IL-2, IL-10, IP-10 (CXCL10), MCP-1, MIP-1β (CCL4) TARC (CCL17), IFNγ, IL-8, IL-12, and SDF1a (CXCL12); and (iv) continuing dosing if the subject has an increase in the level and/or activity of at least one cytokine selected from IL-2, IL-10, IP-10 (CXCL10), MCP-1, MIP-1β (CCL4) TARC (CCL17), IFNγ, IL-8, IL-12, and SDF1a (CXCL12).

In one aspect, the present disclosure relates to a method of selecting a subject for treatment with a therapy for cancer, the method comprising the steps of: (i) administering a dose of a chimeric protein having a general structure of: N terminus—(a)—(b)—(c)—C terminus, wherein: (a) is a first domain comprising an extracellular domain of human T cell immunoreceptor with Ig and ITIM domains (TIGIT), (b) is a linker adjoining the first and second domains, wherein the linker comprises a hinge-CH2-CH3 Fc domain, and (c) is a second domain comprising an extracellular domain of human LIGHT (lymphotoxin-like, exhibits inducible expression and competes with HSV glycoprotein D for herpesvirus entry mediator (HVEM), a receptor expressed by T lymphocytes). wherein the dose is from about 0.03 mg/kg to about 50 mg/kg; (ii) obtaining a biological sample from the subject; (iii) performing an assay on the biological sample to determine level and/or activity of a cytokine selected from IL-2, IL-10, IP-10 (CXCL10), MCP-1, MIP-1β (CCL4) TARC (CCL17), IFNγ, IL-8, IL-12, and SDF1a (CXCL12); and (iv) selecting the subject for treatment with the therapy for cancer if the subject has an increase in the level and/or activity of at least one cytokine selected from IL-2, IL-10, IP-10 (CXCL10), MCP-1, MIP-1β(CCL4) TARC (CCL17), IFNγ, IL-8, IL-12, and SDF1a (CXCL12).

In one aspect, the present disclosure relates to a method of determining a cancer treatment for a patient, the method comprising: (i) obtaining a biological sample from a subject; (ii) evaluating the sample for the upregulation of one or more genes associated with a gene ontology (GO) pathway selected from positive regulation of cell cycle process, regulation of G1/S transition, regulation of cell division, regulation of cell proliferation, positive regulation of IκB kinase/NFκB signaling, type I IFN signaling pathway, cellular response to IFNγ, positive regulation of IFNα production, positive regulation of defense response, positive regulation of IFNβ production, regulation of inflammatory response, regulation of innate immune response, negative regulation of antigen processing/presentation, and antigen processing/presentation of endogenous peptides via MHC class I; and/or downregulation of one or more genes associated with a gene ontology (GO) pathway selected from phospholipid efflux, negative regulation of fibrinolysis, chylomicron assembly, plasma membrane repair, SRP-dependent co-translational protein targeting to membrane, ribosomal small subunit assembly, phospholipid efflux, regulation of translation, mitochondrial respiratory chain complex I, mitochondrial translational elongation, DNA-dependent DNA replication, and ATP biosynthetic process; and (iii) selecting the cancer therapy based on the evaluation of step (b), wherein the cancer therapy comprises a chimeric protein of a general structure of: N terminus—(a)—(b)—(c)—C terminus, wherein: (A) (a) is a first domain comprising an extracellular domain of a Type I transmembrane protein, the transmembrane protein being TIGIT, (b) is a linker comprising at least one cysteine residue capable of forming a disulfide bond, and (c) is a second domain comprising an extracellular domain of Type II transmembrane protein, the transmembrane protein being LIGHT, wherein the linker connects the first domain and the second domain and optionally comprises one or more joining linkers, such joining linkers being selected from SEQ ID NOs: 49-95; or (B) (a) is a first domain comprising an extracellular domain of a Type I transmembrane protein, the transmembrane protein being SIRPα, (b) is a linker comprising at least one cysteine residue capable of forming a disulfide bond, and (c) is a second domain comprising an extracellular domain of Type II transmembrane protein, the transmembrane protein being 4-1BBL, wherein the linker connects the first domain and the second domain and optionally comprises one or more joining linkers, such joining linkers being selected from SEQ ID NOs: 49-95.

In one aspect, the present disclosure relates to a method for selecting a patient for a cancer treatment, the method comprising: (i) obtaining a biological sample from a subject; (ii) evaluating the sample for the upregulation of one or more genes associated with a gene ontology (GO) pathway selected from positive regulation of cell cycle process, regulation of G1/S transition, regulation of cell division, regulation of cell proliferation, positive regulation of IκB kinase/NFκB signaling, type I IFN signaling pathway, cellular response to IFNγ, positive regulation of IFNα production, positive regulation of defense response, positive regulation of IFNβ production, regulation of inflammatory response, regulation of innate immune response, negative regulation of antigen processing/presentation, and antigen processing/presentation of endogenous peptides via MHC class I; and/or downregulation of one or more genes associated with a gene ontology (GO) pathway selected from phospholipid efflux, negative regulation of fibrinolysis, chylomicron assembly, plasma membrane repair, SRP-dependent co-translational protein targeting to membrane, ribosomal small subunit assembly, phospholipid efflux, regulation of translation, mitochondrial respiratory chain complex I, mitochondrial translational elongation, DNA-dependent DNA replication, and ATP biosynthetic process; and (iii) selecting a cancer therapy wherein the cancer therapy comprises a chimeric protein of a general structure of: N terminus—(a)—(b)—(c)—C terminus, wherein (A) (a) is a first domain comprising an extracellular domain of a Type I transmembrane protein, the transmembrane protein being TIGIT, (b) is a linker comprising at least one cysteine residue capable of forming a disulfide bond, and (c) is a second domain comprising an extracellular domain of Type II transmembrane protein, the transmembrane protein being LIGHT, wherein the linker connects the first domain and the second domain and optionally comprises one or more joining linkers, such joining linkers being selected from SEQ ID NOs: 49-95; or (B) (a) is a first domain comprising an extracellular domain of a Type I transmembrane protein, the transmembrane protein being SIRPα, (b) is a linker comprising at least one cysteine residue capable of forming a disulfide bond, and (c) is a second domain comprising an extracellular domain of Type II transmembrane protein, the transmembrane protein being 4-1BBL, wherein the linker connects the first domain and the second domain and optionally comprises one or more joining linkers, such joining linkers being selected from SEQ ID NOs: 49-95.

In one aspect, the present disclosure relates to a method of treating cancer, the method comprising: (i) obtaining a biological sample from a subject; (ii) evaluating the sample for the upregulation of one or more genes associated with a gene ontology (GO) pathway selected from positive regulation of cell cycle process, regulation of G1/S transition, regulation of cell division, regulation of cell proliferation, positive regulation of IκB kinase/NFκB signaling, type I IFN signaling pathway, cellular response to IFNγ, positive regulation of IFNα production, positive regulation of defense response, positive regulation of IFNβ production, regulation of inflammatory response, regulation of innate immune response, negative regulation of antigen processing/presentation, and antigen processing/presentation of endogenous peptides via MHC class I; and/or downregulation of one or more genes associated with a gene ontology (GO) pathway selected from phospholipid efflux, negative regulation of fibrinolysis, chylomicron assembly, plasma membrane repair, SRP-dependent co-translational protein targeting to membrane, ribosomal small subunit assembly, phospholipid efflux, regulation of translation, mitochondrial respiratory chain complex I, mitochondrial translational elongation, DNA-dependent DNA replication, and ATP biosynthetic process; and (iii) selecting the cancer therapy wherein the cancer therapy comprises a chimeric protein of a general structure of: N terminus—(a)—(b)—(c)—C terminus, wherein: (A) (a) is a first domain comprising an extracellular domain of a Type I transmembrane protein, the transmembrane protein being TIGIT, (b) is a linker comprising at least one cysteine residue capable of forming a disulfide bond, and (c) is a second domain comprising an extracellular domain of Type II transmembrane protein, the transmembrane protein being LIGHT, wherein the linker connects the first domain and the second domain and optionally comprises one or more joining linkers, such joining linkers being selected from SEQ ID NOs: 49-95; or (B) (a) is a first domain comprising an extracellular domain of a Type I transmembrane protein, the transmembrane protein being SIRPα, (b) is a linker comprising at least one cysteine residue capable of forming a disulfide bond, and (c) is a second domain comprising an extracellular domain of Type II transmembrane protein, the transmembrane protein being 4-1BBL, wherein the linker connects the first domain and the second domain and optionally comprises one or more joining linkers, such joining linkers being selected from SEQ ID NOs: 49-95.

In embodiments, a lack of upregulation of one or more genes associated with cellular response to IFNγ compared to a prior biological sample from the subject indicates a response to the cancer therapy with an ability to inhibit function and/or activity of PD-1, PD-L1 and/or PD-L2. In embodiments, a lack of upregulation of one or more genes associated with cellular response to IFNγ compared to a patient that is known to be sensitive to anti-PD-1 therapy indicates a response to the cancer therapy with an ability to inhibit function and/or activity of PD-1, PD-L1 and/or PD-L2. In embodiments, a lack of upregulation of one or more genes associated with cellular response to IFNγ compared to a standard indicates a response to the cancer therapy with an ability to inhibit function and/or activity of PD-1, PD-L1 and/or PD-L2.

In embodiments, an upregulation of one or more genes associated with cellular response to IFNγ compared to a prior biological sample from the subject indicates resistance, a lack of response, or recalcitrance to the cancer therapy with an ability to inhibit function and/or activity of PD-1, PD-L1 and/or PD-L2. In embodiments, an upregulation of one or more genes associated with cellular response to IFNγ compared to a patient that is known to be sensitive to anti-PD-1 therapy indicates resistance, a lack of response, or recalcitrance to the cancer therapy with an ability to inhibit function and/or activity of PD-1, PD-L1 and/or PD-L2. In embodiments, an upregulation of one or more genes associated with cellular response to IFNγ compared to a standard indicates resistance, a lack of response, or recalcitrance to the cancer therapy with an ability to inhibit function and/or activity of PD-1, PD-L1 and/or PD-L2.

In embodiments, an upregulation of one or more genes associated with type I IFN signaling pathway compared to a prior biological sample from the subject indicates resistance, a lack of response, or recalcitrance to the cancer therapy with an ability to inhibit function and/or activity of PD-1, PD-L1 and/or PD-L2. In embodiments, an upregulation of one or more genes associated with type I IFN signaling pathway compared to a patient that is known to be sensitive to anti-PD-1 therapy indicates resistance, a lack of response, or recalcitrance to the cancer therapy with an ability to inhibit function and/or activity of PD-1, PD-L1 and/or PD-L2. In embodiments, an upregulation of one or more genes associated with type I IFN signaling pathway compared to a standard indicates resistance, a lack of response, or recalcitrance to the cancer therapy with an ability to inhibit function and/or activity of PD-1, PD-L1 and/or PD-L2.

In embodiments, a lack of upregulation of one or more genes associated with type I IFN signaling pathway compared to a prior biological sample from the subject indicates a response to the cancer therapy with an ability to inhibit function and/or activity of PD-1, PD-L1 and/or PD-L2. In embodiments, a lack of upregulation of one or more genes associated with type I IFN signaling pathway compared to a patient that is known to be sensitive to anti-PD-1 therapy indicates a response to the cancer therapy with an ability to inhibit function and/or activity of PD-1, PD-L1 and/or PD-L2. In embodiments, a lack of upregulation of one or more genes associated with type I IFN signaling pathway compared to a standard indicates a response to the cancer therapy with an ability to inhibit function and/or activity of PD-1, PD-L1 and/or PD-L2.

In embodiments, the biological sample is a fresh tissue sample, frozen tumor tissue specimen, cultured cells, circulating tumor cells, or a formalin-fixed paraffin-embedded tumor tissue specimen, In embodiments, the biological sample is a biopsy sample. In embodiments, the biological sample comprises a body fluid selected from blood, plasma, serum, lacrimal fluid, tears, bone marrow, blood, blood cells, ascites, tissue or fine needle biopsy sample, cell-containing body fluid, free floating nucleic acids, sputum, saliva, urine, cerebrospinal fluid, peritoneal fluid, pleural fluid, feces, lymph, gynecological fluid, skin swab, vaginal swab, oral swab, nasal swab, washing or lavage such as a ductal lavage or broncheoalveolar lavage, aspirate, scraping, bone marrow specimen, tissue biopsy specimen, surgical specimen, feces, other body fluids, secretions, and/or excretions, and/or cells therefrom.

In embodiments, the biological sample comprises at least one tumor cell.

In embodiments, the evaluating is performed by DNA sequencing, RNA sequencing, immunohistochemical staining, western blotting, in cell western, immunofluorescent staining, ELISA, and fluorescent activating cell sorting (FAGS) or a combination thereof.

In embodiments, the evaluating is performed by contacting the sample with an agent that specifically binds to one or more proteins encoded by one or more genes associated with a gene ontology (GO) pathways identified herein.

In embodiments, the evaluating is performed by contacting the sample with an agent that specifically binds to one or more of nucleic acids of one or more genes associated with a gene ontology (GO) pathway identified herein.

In embodiments, the evaluating informs classifying the patient into a high or low risk group. In embodiments, the high risk classification comprises a high level of tumor cells having resistance to the cancer therapy with an ability to inhibit function and/or activity of PD-1, PD-1.1 and/or PD-L2. In embodiments, the low risk classification comprises a low level of tumor cells having resistance to the cancer therapy with an ability to inhibit function and/or activity of PD-1, PD-L1 and/or PD-L2.

In embodiments, the upregulation is in comparison to a healthy tissue. In embodiments, the upregulation is in comparison to another biological sample from patient that is known to be sensitive to anti-PD-1 therapy. In embodiments, the upregulation is in comparison to a prior biological sample obtained from the subject. In embodiments, the downregulation is in comparison to a healthy tissue. In embodiments, the downregulation is in comparison to another biological sample from patient that is known to be sensitive to anti-PD-1 therapy. In embodiments, the downregulation is in comparison to a prior biological sample obtained from the subject.

In one aspect, the present disclosure relates to a method for treating cancer in a subject in need thereof comprising administering an effective amount of a pharmaceutical composition to a subject in need thereof, the pharmaceutical composition comprising a chimeric protein comprising: N terminus—(a)—(b)—(c)—C terminus, wherein: (A) (a) is a first domain comprising an extracellular domain of a Type I transmembrane protein, the transmembrane protein being TIGIT, (b) is a linker comprising at least one cysteine residue capable of forming a disulfide bond, and (c) is a second domain comprising an extracellular domain of Type II transmembrane protein, the transmembrane protein being LIGHT, wherein the linker connects the first domain and the second domain and optionally comprises one or more joining linkers, such joining linkers being selected from SEQ ID NOs: 49-95; or (B) (a) is a first domain comprising an extracellular domain of a Type I transmembrane protein, the transmembrane protein being SIRPα, (b) is a linker comprising at least one cysteine residue capable of forming a disulfide bond, and (c) is a second domain comprising an extracellular domain of Type II transmembrane protein, the transmembrane protein being 4-1BBL, wherein the linker connects the first domain and the second domain and optionally comprises one or more joining linkers, such joining linkers being selected from SEQ ID NOs: 49-95, wherein the cancer is or is believed to be resistant to an anti-checkpoint agent having an ability to inhibit function and/or activity of PD-1, PD-L1 and/or PD-L2.

In embodiments, the linker is a polypeptide selected from a flexible amino acid sequence, an IgG hinge region, or an antibody sequence. In embodiments, the linker comprises hinge-CH2-CH3 Fc domain derived from IgG1 or IgG4. In embodiments, the hinge-CH2-CH3 Fc domain is derived from human IgG1. In embodiments, the hinge-CH2-CH3 Fc domain is derived from human IgG4. In embodiments, the linker comprises an amino acid sequence that is at least 95%, or at least 96%, or at least 97%, or at least 98%, or at least 99% identical to the amino acid sequence of SEQ ID NO: 46, SEQ ID NO: 47, SEQ ID NO: 112, or SEQ ID NO: 113. In embodiments, the linker comprises two or more joining linkers each joining linker independently selected from SEQ ID NOs: 49-95; wherein one joining linker is N terminal to the hinge-CH2-CH3 Fc domain and another joining linker is C terminal to the hinge-CH2-CH3 Fc domain.

In embodiments, the first domain comprises an amino acid sequence that is at least 95%, or at least 96%, or at least 97%, or at least 98%, or at least 99% identical to the amino acid sequence of SEQ ID NO: 10. In embodiments, the first domain comprises an amino acid sequence that is at least 95%, or at least 96%, or at least 97%, or at least 98%, or at least 99% identical to the amino acid sequence of SEQ ID NO: 2. In embodiments, chimeric protein comprises an amino acid sequence that is at least 95%, or at least 96%, or at least 97%, or at least 98%, or at least 99% identical to the amino acid sequence of SEQ ID NO: 11. In embodiments, the chimeric protein is a recombinant fusion protein.

In one aspect, the present disclosure relates to a method of determining a cancer treatment for a patient, the method comprising: (I) obtaining a biological sample from a subject; (II) evaluating the biological sample for the expression of: (i) a gene selected from CD274, B2M, STAT1, STAT2, TRIM7, IRF1, TAP1, TAP2, CASP1, IRF, LTBR, PVR, GASTA3, LRG1, SPRY2, ARG1, TRIM8, TRIM2, MAPK81P1, TRIM6, and KRT1; and/or (ii) a gene selected from RPL41, RPS15, RPS8, TRIM7 and LRG1; and (III) selecting the cancer therapy based on the evaluation of step (II), wherein the cancer therapy comprises a chimeric protein of a general structure of: N terminus—(a)—(b)—(c)—C terminus, wherein: (A) (a) is a first domain comprising an extracellular domain of a Type I transmembrane protein, the transmembrane protein being TIGIT, (b) is a linker comprising at least one cysteine residue capable of forming a disulfide bond, and (c) is a second domain comprising an extracellular domain of Type II transmembrane protein, the transmembrane protein being LIGHT, wherein the linker connects the first domain and the second domain and optionally comprises one or more joining linkers, such joining linkers being selected from SEQ ID NOs: 49-95; or (B) (a) is a first domain comprising an extracellular domain of a Type I transmembrane protein, the transmembrane protein being SIRPα, (b) is a linker comprising at least one cysteine residue capable of forming a disulfide bond, and (c) is a second domain comprising an extracellular domain of Type II transmembrane protein, the transmembrane protein being 4-1BBL, wherein the linker connects the first domain and the second domain and optionally comprises one or more joining linkers, such joining linkers being selected from SEQ ID NOs: 49-95.

In one aspect, the present disclosure relates to a method for selecting a patient for a cancer treatment, the method comprising: (I) obtaining a biological sample from a subject; (II) evaluating the biological sample for the expression of: (i) a gene selected from CD274, B2M, STAT1, STAT2, TRIM7, IRF1, TAP1, TAP2, CASP1, IRF, LTBR, PVR, GASTA3, LRG1, SPRY2, ARG1, TRIM8, TRIM2, MAPK81P1, TRIM6, and KRT1; and/or (ii) a gene selected from RPL41, RPS15, RPS8, TRIM7 and LRG1; and (III) selecting the cancer therapy based on the evaluation of step (II), wherein the cancer therapy comprises a chimeric protein of a general structure of: N terminus—(a)—(b)—(c)—C terminus, wherein: (A) (a) is a first domain comprising an extracellular domain of a Type I transmembrane protein, the transmembrane protein being TIGIT, (b) is a linker comprising at least one cysteine residue capable of forming a disulfide bond, and (c) is a second domain comprising an extracellular domain of Type II transmembrane protein, the transmembrane protein being LIGHT, wherein the linker connects the first domain and the second domain and optionally comprises one or more joining linkers, such joining linkers being selected from SEQ ID NOs: 49-95; or (B) (a) is a first domain comprising an extracellular domain of a Type I transmembrane protein, the transmembrane protein being SIRPα, (b) is a linker comprising at least one cysteine residue capable of forming a disulfide bond, and (c) is a second domain comprising an extracellular domain of Type II transmembrane protein, the transmembrane protein being 4-1BBL, wherein the linker connects the first domain and the second domain and optionally comprises one or more joining linkers, such joining linkers being selected from SEQ ID NOs: 49-95.

In one aspect, the present disclosure relates to a method of treating cancer, the method comprising: (I) obtaining a biological sample from a subject; (II) evaluating the biological sample for the expression of: (i) a gene selected from CD274, B2M, STAT1, STAT2, TRIM7, IRF1, TAP1, TAP2, CASP1, IRF, LTBR, PVR, GASTA3, LRG1, SPRY2, ARG1, TRIM8, TRIM2, MAPK81P1, TRIM6, and KRT1; and/or (ii) a gene selected from RPL41, RPS15, RPS8, TRIM7 and LRG1; and (III) wherein the cancer therapy comprises a chimeric protein of a general structure of: N terminus—(a)—(b)—(c)—C terminus, wherein: (A) (a) is a first domain comprising an extracellular domain of a Type I transmembrane protein, the transmembrane protein being TIGIT, (b) is a linker comprising at least one cysteine residue capable of forming a disulfide bond, and (c) is a second domain comprising an extracellular domain of Type II transmembrane protein, the transmembrane protein being LIGHT, wherein the linker connects the first domain and the second domain and optionally comprises one or more joining linkers, such joining linkers being selected from SEQ ID NOs: 49-95; or (B) (a) is a first domain comprising an extracellular domain of a Type I transmembrane protein, the transmembrane protein being SIRPα, (b) is a linker comprising at least one cysteine residue capable of forming a disulfide bond, and (c) is a second domain comprising an extracellular domain of Type II transmembrane protein, the transmembrane protein being 4-1BBL, wherein the linker connects the first domain and the second domain and optionally comprises one or more joining linkers, such joining linkers being selected from SEQ ID NOs: 49-95; and (VI) optionally administering the cancer therapy with an ability to inhibit function and/or activity of PD-1, PD-L1 and/or PD-L2. In one aspect, the present disclosure relates to a method of determining a cancer treatment for a patient, the method comprising: (I) obtaining a biological sample from a subject; (II) evaluating the biological sample for the activation of a pathway selected from Mapk8ip1, Trim7, Elk1, Lrg1, Arg1, Rap1, and Ras; and (III) selecting the cancer therapy based on the evaluation of step (II), wherein the cancer therapy comprises a chimeric protein of a general structure of: N terminus—(a)—(b)—(c)—C terminus, wherein: (A) (a) is a first domain comprising an extracellular domain of a Type I transmembrane protein, the transmembrane protein being TIGIT, (b) is a linker comprising at least one cysteine residue capable of forming a disulfide bond, and (c) is a second domain comprising an extracellular domain of Type II transmembrane protein, the transmembrane protein being LIGHT, wherein the linker connects the first domain and the second domain and optionally comprises one or more joining linkers, such joining linkers being selected from SEQ ID NOs: 49-95; or (B) (a) is a first domain comprising an extracellular domain of a Type I transmembrane protein, the transmembrane protein being SIRPα, (b) is a linker comprising at least one cysteine residue capable of forming a disulfide bond, and (c) is a second domain comprising an extracellular domain of Type II transmembrane protein, the transmembrane protein being 4-1BBL, wherein the linker connects the first domain and the second domain and optionally comprises one or more joining linkers, such joining linkers being selected from SEQ ID NOs: 49-95.

In one aspect, the present disclosure relates to a method for selecting a patient for a cancer treatment, the method comprising: (I) obtaining a biological sample from a subject; (II) evaluating the biological sample for the activation of a pathway selected from Mapk8ip1, Trim7, Elk1, Lrg1, Arg1, Rap1, and Ras; and (III) selecting the cancer therapy based on the evaluation of step (II), wherein the cancer therapy comprises a chimeric protein of a general structure of: N terminus—(a)—(b)—(c)—C terminus, wherein: (A) (a) is a first domain comprising an extracellular domain of a Type I transmembrane protein, the transmembrane protein being TIGIT, (b) is a linker comprising at least one cysteine residue capable of forming a disulfide bond, and (c) is a second domain comprising an extracellular domain of Type II transmembrane protein, the transmembrane protein being LIGHT, wherein the linker connects the first domain and the second domain and optionally comprises one or more joining linkers, such joining linkers being selected from SEQ ID NOs: 49-95; or (B) (a) is a first domain comprising an extracellular domain of a Type I transmembrane protein, the transmembrane protein being SIRPα, (b) is a linker comprising at least one cysteine residue capable of forming a disulfide bond, and (c) is a second domain comprising an extracellular domain of Type II transmembrane protein, the transmembrane protein being 4-1BBL, wherein the linker connects the first domain and the second domain and optionally comprises one or more joining linkers, such joining linkers being selected from SEQ ID NOs: 49-95.

In one aspect, the present disclosure relates to a method of treating cancer, the method comprising: (I) obtaining a biological sample from a subject; (II) evaluating the biological sample for the activation of a pathway selected from Mapk8ip1, Trim7, Elk1, Lrg1, Arg1, Rap1, and Ras; and (II) wherein the cancer therapy comprises a chimeric protein of a general structure of: N terminus—(a)—(b)—(c)—C terminus, wherein: (A) (a) is a first domain comprising an extracellular domain of a Type I transmembrane protein, the transmembrane protein being TIGIT, (b) is a linker comprising at least one cysteine residue capable of forming a disulfide bond, and (c) is a second domain comprising an extracellular domain of Type II transmembrane protein, the transmembrane protein being LIGHT, wherein the linker connects the first domain and the second domain and optionally comprises one or more joining linkers, such joining linkers being selected from SEQ ID NOs: 49-95; or (B) (a) is a first domain comprising an extracellular domain of a Type I transmembrane protein, the transmembrane protein being SIRPα, (b) is a linker comprising at least one cysteine residue capable of forming a disulfide bond, and (c) is a second domain comprising an extracellular domain of Type II transmembrane protein, the transmembrane protein being 4-1BBL, wherein the linker connects the first domain and the second domain and optionally comprises one or more joining linkers, such joining linkers being selected from SEQ ID NOs: 49-95; and (IV) optionally administering the cancer therapy with an ability to inhibit function and/or activity of PD-1, PD-L1 and/or PD-L2.

In embodiments, the anti-checkpoint agent an antibody selected from nivolumab (OPDIVO), pembrolizumab (KEYTRUDA), pidilizumab (CT-011, CURE TECH), MK-3475 (MERCK), BMS 936559, MPDL328OA (ROCHE), Cemiplimab (LIBTAYO), Atezolizumab (TECENTRIQ), Avelumab (BAVENCIO), and Durvalumab (imfinzi).

In embodiments, the method further comprises administration of an anti-checkpoint agent. In embodiments, the anti-checkpoint agent an antibody selected from nivolumab (OPDIVO), pembrolizumab (KEYTRUDA), pidilizumab (CT-011, CURE TECH), MK-3475 (MERCK), BMS 936559, MPDL328OA (ROCHE), Cemiplimab (LIBTAYO), Atezolizumab (TECENTRIQ), Avelumab (BAVENCIO), and Durvalumab (imfinzi). In embodiments, the pharmaceutical composition comprising the chimeric protein and the anti-checkpoint agent are administered simultaneously or contemporaneously. In embodiments, the pharmaceutical composition comprising the chimeric protein is administered after the anti-checkpoint agent is administered. In embodiments, the pharmaceutical composition comprising the chimeric protein is administered before the anti-checkpoint agent is administered.

Any aspects disclosed herein may be combined with any other aspects.

### BRIEF DESCRIPTION OF THE FIGURES

The patent or application file contains at least one drawing executed in color. Copies of this patent or patent application publication with color drawing(s) will be provided by the Office upon request and payment of the necessary fee.

FIG. 1A and FIG. 1B illustrate the generation of anti-PD-1-resistant CT26 tumors. FIG. 1A shows a schematic representation of the method used to generation of anti-PD-1-resistant CT26 tumors. Briefly, BALB/C mice were inoculated with 500,000 murine colon carcinoma CT26 cells, and when average tumor volume reached mm³ (indicating day 0), mice were treated with an anti-PD-1 (clone RMP1-14; BioXcell) antibody. Tumors were excised and surviving tumor cells were dissociated and cultures in vitro. These cells were called “1st round,” “1st generation,” or “F1 generation” cells. 1st generation cells were inoculated in BALB/C mice, and following another treatment course of anti-PD-1 “2nd round,” “2nd generation,” or “F2 generation” cells were isolated. Following two more rounds, (a total of 4 rounds) of anti-PD-1 selection, “4th round,” “4th generation,” or “F4 generation” cells were isolated. FIG. 1B shows a graph comparing the efficacy of an anti-PD-1 antibody (100 μg anti-PD-1 clone RMP1-14; BioXcell) in BALB/C mice harboring CT26 parental cells and PD-1 resistant cells. BALB/C mice were inoculated with CT26 parental cells and PD-1 resistant 4th generation cells in rear flanks. When the starting tumor volume (STV) reached 80-100 mm³, mice were randomly divided in the following two treatment groups: (1) vehicle (PBS), and (2) anti-PD-1 antibody. Mice were given a series of intraperitoneal injections of vehicle or 100 μg anti-PD-1 (clone RMP1-14; BioXcell) on days 0, 3, and 6. Tumor volumes were measured on indicated days.

FIG. 2A to FIG. 2D show the transcriptomic profiling of anti-PD-1 resistant cell lines using RNA-seq. FIG. 2A (Top Panel) shows the Principal Component Analysis (PCA) that spatially separated the samples based on transcriptome expression. FIG. 2A (Bottom Panel) shows the Differentially Expressed Genes (DEGs) that were determined between the groups (parent vs. 2nd generation, parent vs. 4th generation, 2nd generation vs. 4th generation), and plotted in the heatmap. Hierarchical clustering was performed to rank order genes on each row, which separated genes into 2 major clusters in each comparison, where a subset of gene expression was lower (blue) in one dataset and higher (red) in the other. FIG. 2B shows the up- or down-regulated genes in each dataset. The genes were input into PANTHER to identify Gene Ontologies (GO) associated with each gene set. Gene sets are shown with associated p-values. FIG. 2C (Top Panel) shows a Venn diagram of overlap in gene expression between all datasets. FIG. 2C (Bottom Panel) shows the transcripts per million (TPM; normalized expression data) at select genes, demonstrating higher baseline expression of genes associated with PD-L1, antigen processing/presentation, protein translation, ER trafficking; in some datasets over others. FIG. 2D shows the transcripts per million (TPM; normalized expression data) of select genes, demonstrating higher baseline expression of genes associated with PD-L1, antigen processing/presentation, protein translation, ER trafficking; in some datasets over others.

FIG. 3 illustrates that there is a discordance in gene expression and cell surface protein expression in PD-L1/2 MHC Class I and B2M. Parental and 4th generation anti-PD-1 resistant cells were harvested from culture and analyzed by flow cytometry for surface expression of PD-L1, PD-L2, MHC Class I, and β2 microglobulin (B2M). Gates were drawn as shown, and shown above each plot is the percentage of cells in each gate, and to the right of each percentage, the MFI (mean fluorescent intensity) of each marker.

FIG. 4A and FIG. 4B show the transcriptomic profiling of anti-PD-1 resistant cell lines. FIG. 4A shows the in comparison of 2nd- and 4th generation anti-PD-1 resistance cell lines with B16.F10 is a murine melanoma tumor, which served as a model of anti-PD-1 “primary resistance,” as these tumors are not responsive to anti-PD-1 therapy. Cells were cultured for 24 hours in IFNγ to assess in vitro responsiveness. This mimics how tumor cells respond in vivo, as immune cell infiltrate and secrete effector cytokines like IFNγ. FIG. 4A (Left Panel) shows the DEGs that were identified between untreated and IFNγ treated parental CT26. Of these, 338 genes had usable data from the other datasets; and those values are shown in the other columns. Log 2 fold-change was plotted in the heatmap and genes are hierarchical clustered based on parental CT26. Genes separated into 3 major clusters. FIG. 4A (Right Panel) shows the GO pathways associated with the DEGs. Associated genes were input into PANTHER to identify pathways associated with the dysregulated genes. FIG. 4B shows the transcripts per million (TPM; normalized expression data) at select genes, demonstrating that although CT26 anti-PD-1 resistant cells have baseline hyperactivation of type I and type II interferons, when those cells are challenged with IFNγ, those cells downregulate these genes.

FIG. 5A to FIG. 5D illustrate the paradoxical dysregulation of certain genes associated with the acquired resistance to anti-PD-1. The genes encoding CD274 (FIG. 5A) and B2m (FIG. 5B), which are overexpressed in the 4th generation anti-PD-1 resistant cells, are overexpressed in wild type CT26 cells but are repressed in the 4th generation anti-PD-1 resistant cells in the presence of IFNγ. On the other hand, the genes encoding Trim7 (FIG. 5C) and Lrg1 (FIG. 5D), which are repressed in the 4th generation anti-PD-1 resistant cells, are repressed in wild type CT26 cells but are but are overexpressed in the 4th generation anti-PD-1 resistant cells in response to IFNγ. Second generation anti-PD-1 resistant cells show an intermediate phenotype.

FIG. 6A and FIG. 6F show the identification of driver genes involved in acquired resistance to anti-PD-1 and the functional pathways affected by the driver genes. FIG. 6A, Panels 1˜4 show the methodology used for the identification of the driver genes. FIG. 6A (Panel 1) shows that 1,999 genes are downregulated and 3607 genes are upregulated the 4th generation anti-PD-1 resistant cells in the presence of IFNγ compared to the CT26 cells. FIG. 6A (Panel 2) shows that 1,060 genes that are downregulated the 4th generation anti-PD-1 resistant cells but are upregulated the CT26 cells. FIG. 6A (Panel 3) shows that 688 genes that are downregulated the 4th generation anti-PD-1 resistant cells compared to the CT26 cells as revealed by sorting according to responsiveness to IFNγ. FIG. 6A (Panel 4) shows that 70 genes that upregulated in vivo in the 4th generation anti-PD-1 resistant cells compared to the CT26 cells. FIG. 6B shows the gene ontology (GO) pathways associated with the genes identified using the methodology of FIG. 6A. FIG. 6C shows the functional pathways affected by the TRIM family of proteins. FIG. 6D shows the functional pathways in which Elk1 and c-Jun play a role. FIG. 6E shows the functional connections between Lrg1, B2m and Arg1 with other genes. FIG. 6F shows the levels of expression of Elk1 in tumors and surrounding normal tissue in the Cancer Genome Atlas (TCGA) cancer genomics program.

FIG. 7A to FIG. 7D show that Stat1 (FIG. 7A), Stat2 (FIG. 7B), Irf1 (FIG. 7C) and Tap1 (FIG. 7D) genes, which are overexpressed in response to IFNγ, are overexpressed in the 4th generation anti-PD-1 resistant cells, and are repressed in the 4th generation anti-PD-1 resistant cells in the presence of IFNγ. Second generation anti-PD-1 resistant cells show an intermediate phenotype.

FIG. 8A and FIG. 8D show the pathway analysis of the differentially expressed genes, which led to the identification of the Ras and Rap1 signaling pathways. FIG. 8A shows the identification of pathways using the WEB-based GEne SeT AnaLysis Toolkit (WebGestalt) using the top 1,000 genes from FIG. 6A (Panel 2). FIG. 8B shows a Volcano plot of the data presented in FIG. 8A. FIG. 8C shows the RAS signaling pathway. The convergence with Raf/Mek/Erk signaling is shown using ovals. FIG. 8D shows the RAP1 signaling pathway. The convergence with Raf/Mek/Erk signaling is shown using ovals.

FIG. 9A to FIG. 9C show that Ccl5 (RANTES) (FIG. 9A), Cxcl10 (IP-10) (FIG. 9B), and Ifnb1 (FIG. 9C) genes, which are overexpressed in response to IFNγ, are overexpressed in the 4th generation anti-PD-1 resistant cells, and are repressed in the 4th generation anti-PD-1 resistant cells in the presence of IFNγ. Second generation anti-PD-1 resistant cells show an intermediate phenotype.

FIG. 10A to FIG. 10H show the construction and characterization of the TIGIT-Fc-LIGHT chimeric protein. FIG. 10A shows a molecular model of the TIGIT-Fc-LIGHT chimeric protein, which exists as a hexamer having two functional sets of LIGHT trimers based on the PDB (protein data bank) structures, with dimerization of the central IgG4 Fc domain and trimerization of the TNF-ligand domain. FIG. 10B are Western blots showing characterization of the TIGIT-Fc-LIGHT chimeric protein. The Western blots demonstrate the chimeric protein's native state and tendency to form a multimer. Untreated samples (i.e., without a reducing agent or a deglycosylation agent, yet boiled) of the TIGIT-Fc-LIGHT chimeric protein, were loaded into the lane marked as NR of each of the blots. Samples in the lane marked as R were treated with a reducing agent, p-mercaptoethanol, and were boiled. Samples in the lane marked as DG were treated with a deglycosylation agent, the reducing agent, and were boiled. The lane marked as L included the protein size ladder. Each individual domain of the chimeric protein was probed using an anti-TIGIT antibody (left blot), an anti-Fc antibody (center blot), or an anti-LIGHT antibody (right blot). FIG. 10C shows the Meso Scale Discovery (MSD) ELISA assays demonstrating simultaneous binding of the TIGIT-Fc-LIGHT chimeric protein to checkpoint targets (PVR) and immune co-stimulatory receptors found on myeloid, CD8+ T, and NK cells (LTβR and HVEM). FIG. 10D shows the cell based binding assays developed to assess cell surface receptor binding (using CHOK1 cells engineered to express hPVR, hHVEM, and also A375 cells that express human LTβR). FIG. 10E shows the activation of downstream signaling in Jurkat effector cells by the TIGIT-Fc-LIGHT chimeric protein. NFκB/NIK reporter cells were incubated with a recombinant Fc-LIGHT control or the TIGIT-Fc-LIGHT chimeric protein (18 nM each), and signaling activity was assessed through luciferase detection.

FIG. 10F shows that the TIGIT-Fc-LIGHT chimeric protein (or the murine surrogate, both at 10 nM) activates T cells through IL-2 induction, as assessed in human PBMC or mouse splenocytes co-cultured with the super-antigen SEB for 3 days. FIG. 10G shows the Jurkat effector cells—part of a commercial 2 cell reporter system—express TIGIT, DNAM-1, and HVEM as shown using flow cytometry. FIG. 10H shows the activation of Jurkat effector and CHO/hPVR reporter cells. These cells were co-cultured with (all at 150 nM) an IgG4 control, a DNAM-1 blocking antibody, the TIGIT-Fc-LIGHT chimeric protein, or the TIGIT-Fc-LIGHT chimeric protein pre-incubated with a LIGHT blocking antibody for 6 hours, and then luciferase signaling activity was assessed using a luminometer.

FIG. 11 shows the tumor growth inhibition of CT26 (left panel), CT26 anti-PD-1 resistant cell (middle panel), or B16.F10 (right panel) allografts compared to vehicle-alone treated mice in mice treated as indicated. The dotted line shows the amount of tumor growth inhibition caused an anti-PD-1 antibody (clone 10F.9G2).

FIG. 12A to FIG. 12J show the in vivo efficacy of the TIGIT-Fc-LIGHT chimeric protein against cancer. FIG. 12A shows the individual animal tumor growth curves, the average day in which each group reached tumor burden, and the number of mice that completely rejected the tumor in response to treatment. FIG. 12B shows Kaplan-Meier curves showing the overall survival of mice harboring allografts of CT26 colorectal cancer tumors, anti-PD-1 resistant CT26 tumors or B16.F10 tumors over the course of the experiment. FIG. 12C shows the individual animal tumor growth curves of mice harboring a CT26 wild type tumors and treated with 200 μg of the murine TIGIT-Fc-LIGHT chimeric protein (mTIGIT-Fc-LIGHT), or 100 μg of anti-LTβR, anti-TIGIT, Fc-LIGHT, anti-PD-1, anti-PD-L1, or the indicated combinations. Also shown are the average day in which the entire group reached tumor burden (also indicated by dotted line), and the number of mice that completely rejected tumors/the total group size analyzed. FIG. 12D shows the Kaplan-Meier survival plot through day 36 of the time-course from FIG. 12C. FIG. 12E shows the individual animal tumor growth curves of mice harboring a B16.F10 tumors and treated with 200 μg of mTIGIT-Fc-LIGHT, or 100 μg of anti-TIGIT, Fc-LIGHT, anti-PD-1, anti-PD-L1, or, or the indicated combinations. Also shown are the average day in which the entire group reached tumor burden (also indicated by dotted line), and the number of mice that completely rejected tumors/the total group size analyzed. FIG. 12F shows the Kaplan-Meier survival plot through day 36 of the time-course from FIG. 12E. FIG. 12G shows the proportion of antigen-specific CD8+ T cells (AH1+) and NK cells infiltrating CT26 colorectal cancer tumors or anti-PD-1 resistant CT26 tumors (CT26/AR) 7 days after tumor inoculation (following treatment on days 0, 3, 6). FIG. 12H shows the impact of CD4+ T, CD8+ T, or NK cell depletion on the in vivo activity of the TIGIT-Fc-LIGHT chimeric protein in the B16.F10 model. When B16.F10 tumors reached an average starting tumor volume (STV) of 112.57 mm³ indicating day 0. Mice were treated with a course of CD4, CD8, or NK depleting antibodies on days −1, 1, and 7. Mice were given 3 IP injections on days 0, 3, and 6; each consisting of 200 mg of mTIGIT-Fc-LIGHT and the change in tumor volumes from day 0 to day 11 was plotted. FIG. 12I shows the individual animal tumor growth curves of mice harboring an anti-PD-1 resistant CT26 tumors and treated with 200 μg of mTIGIT-Fc-LIGHT, or 100 μg of anti-TIGIT, Fc-LIGHT, anti-PD-1, anti-PD-L1, or the indicated combinations. Also shown are the average day in which the entire group reached tumor burden (also indicated by dotted line), and the number of mice that completely rejected tumors/the total group size analyzed. FIG. 12J shows the Kaplan-Meier survival plot through day 36 of the time-course from FIG. 12I. FIG. 12K shows the induction of total CD8+ cells in mice treated with the indicated doses of the TIGIT-Fc-LIGHT chimeric protein. FIG. 12L shows the induction of total CD4+DNAM1+ cells in mice treated with the indicated doses of the TIGIT-Fc-LIGHT chimeric protein. FIG. 12M shows the induction of total CD4+ cells in mice treated with the indicated doses of the TIGIT-Fc-LIGHT chimeric protein. FIG. 12N shows the induction of total CD11 b+ cells in mice treated with the indicated doses of the TIGIT-Fc-LIGHT chimeric protein. FIG. 12O shows the induction of activated CD11b+CD80+ cells in mice treated with the indicated doses of the TIGIT-Fc-LIGHT chimeric protein. FIG. 12P shows the induction of activated CD11b+CD86+ cells in mice treated with the indicated doses of the TIGIT-Fc-LIGHT chimeric protein.

FIG. 13A to FIG. 13G show the expression of immune co-stimulatory receptor across various peripheral blood cell subtypes and within human and mouse TILs. FIG. 13A shows a heatmap depicting the relative expression of 53 immune co-stimulatory genes across TOGA cancers, ranked ordered based on high- to low-mean expression across all tumor types. FIG. 13B shows the normalized expression level of TNFRSF14, CD226, and LTBR across all TOGA tumor types is plotted on the same scale, to depict relative mRNA expression of these target genes in relation to each other. FIG. 13C shows the shows the relative level of HVEM and DNAM-1 in naïve CD8+ T cells (Tn) and T stem cell memory CD8+ T cells (Tscm). Human naïve CD8+ T cells isolated from healthy donor PBMC using magnetic separation, were cultured in AIMV media with CD3/CD28 beads, human recombinant IL-2, and the GSK3r3 inhibitor TWS119 for 9 days. Following this time-course, cells were isolated and assessed by flow cytometry according to a previously identified antibody panel that characterizes naïve CD8+ T cells (Tn) and T stem cell memory CD8+ T cells (Tscm). The relative level of HVEM and DNAM-1 are depicted and quantitated across both Tscm and Tn cells. FIG. 13D shows the Uniform Manifold Approximation and Projection (UMAP) spatial organization in Seurat identified clusters (top) and SingleR immune cell subtypes according to ImmuneExp (bottom). Human PBMC cultured in AIMV media for 2 days were isolated and subjected to single-cell RNA sequencing (scRNA-seq). UMAP spatial organization was assessed using Seurat identified clusters (top) and SingleR to plot immune cell subtypes according to ImmuneExp (bottom). FIG. 13E shows a heatmap depicting the relative expression of 53 immune co-stimulatory genes across Seurat identified clusters. Genes are ranked ordered based on high to low-mean expression across all clusters. The intensity of expression is shown based on the minimum and maximum values in each column. Three genes of interest, TNFRSF14 (HVEM), CD226 (DNAM-1), and LTBR (LTβR) are highlighted. FIG. 13F shows the normalized expression level of TNFRSF14, CD226, and LTBR across Seurat identified clusters is plotted on the same scale, to depict relative mRNA expression of these target genes in relation to each other. FIG. 13G shows the relative cell surface expression of HVEM and DNAM-1 in tumor infiltrating lymphocytes (TILs) Murine CT26 wild-type colorectal (CT26/WT) tumors, CT26 tumors engineered to develop CPI acquired resistance (CT26/AR), or B16.F10 melanoma tumors, were inoculated into their respective recipient mice. Tumors were allowed to establish, and when they reached mm³ (˜10-14 days after the initial inoculation), tumors were excised, dissociated, and the resulting tumor infiltrating lymphocytes (TIL) were assessed by flow cytometry. Relative cell surface expression of HVEM and DNAM-1 was assessed in NK cells (gated on NKP46+ cells in CT26 tumors and NK1.1 in B16.F10 tumors) and CD8+ T cells (gated on CD3+CD8+ cells).

FIG. 14A to FIG. 14H show that the TIGIT-Fc-LIGHT chimeric protein directly activates myeloid, T, and NK cells regardless of Fc composition. Human donor PBMC were either untreated (UT) or cultured in AIMV media with IgG1 or IgG4 variants of the TIGIT-Fc-LIGHT chimeric protein; referred to as the TIGIT-Fc(IgG1)-LIGHT chimeric protein or G1 and the TIGIT-Fc(IgG4)-LIGHT chimeric protein or G4. FIG. 14A shows the phase contrast images of PBMC cultured for 7 days+/− the TIGIT-Fc(IgG1)-LIGHT or TIGIT-Fc(IgG4)-LIGHT chimeric protein, depicting adherence of cells to the plate and the formation of a spindle-like myeloid morphology. FIG. 14B shows the proliferation/confluency of PBMC cultures were incubated on the Incucyte time-lapse imager and proliferation/confluency was assessed over a 6 day time-course in the presence or absence of 150 nM of TIGIT-Fc(IgG4)-LIGHT. 20× images were taken, in duplicate for each of 3 donors across 4 fields of view. FIG. 14C shows the cytokines induced by the TIGIT-Fc-LIGHT chimeric protein as assessed in the culture supernatant using Meso Scale Discovery (MSD) ELISA assays. After 2 days in culture, supernatant was removed and a series of cytokines were assessed using a MSD multiplex cytokine panel. FIG. 14D shows the number of differentially expressed genes (DEGs) as assessed by single-cell RNA sequencing. On day 2, untreated (UT), TIGIT-Fc(IgG1)-LIGHT-treated (G1), and TIGIT-Fc(IgG4)-LIGHT-treated (G4) PBMC single cells were isolated and subjected to single-cell RNA sequencing. Differentially Expressed Genes (DEGs) were identified between the TIGIT-Fc(IgG1)-LIGHT and TIGIT-Fc(IgG4)-LIGHT chimeric proteins and the untreated group across each of the 16 Seurat clusters identified in FIG. 13D and FIG. 13E. FIG. 14E shows the heatmaps depicting the relative differences in expression at DEGs identified across all 16 Seurat clusters. Genes of interest are labeled at clusters corresponding to myeloid, NK, and CD8+ T cell populations. FIG. 14F shows the Uniform Manifold Approximation and Projection (UMAP) spatial distribution of untreated, TIGIT-Fc(IgG1)-LIGHT, and TIGIT-Fc(IgG4)-LIGHT datasets. The populations of cells that correspond to myeloid, NK, and CD8+ T cells (as identified using ImmuneExp annotations), are highlighted and the percentage of cells falling in those gates across treatment groups are shown. FIG. 14G shows the pathways associated with the TIGIT-Fc-LIGHT chimeric protein induced differentially expressed genes (DEGs) as identified by protein analysis through evolutionary relationships (PANTHER). The number and directionality of the DEGs are shown. Gene ontology analysis (using PANTHER) was performed on the lists of up- and down-regulated genes, and significantly enriched pathways (FDR p-value<0.05) are shown. FIG. 14H shows the fold change in myeloid cells isolated from human PBMC from mice treated with the TIGIT-Fc(IgG1)-LIGHT and TIGIT-Fc(IgG4)-LIGHT chimeric proteins in comparison with untreated mice.

FIG. 15 shows the tumor volumes of CT26 or anti-PD-1 resistant CT26 (CT26/AR) tumor allografts in mice treated with vehicle only, an anti-TIGIT antibody (clone 1G9), an anti-PD-1 antibody (clone RPM1-14), a combination of the anti-TIGIT and the anti-PD-1 antibodies, the TIGIT-Fc-LIGHT chimeric protein, and a combination of the TIGIT-Fc-LIGHT chimeric protein and the anti-PD-1 antibody.

FIG. 16 shows a cartoon showing the structure of an illustrative human SIRPα-Fc-4-1BBL chimeric protein (top panel), and western blots showing characterization of the human SIRPα-Fc-4-1BBL chimeric protein (bottom panels. The western blots demonstrate the chimeric protein's native state and tendency to form a multimer. Molecular weight markers were loaded in the first lane of each blot. Untreated samples (i.e., without a reducing agent or a deglycosylation agent, yet boiled) of the human SIRPα-Fc-4-1BBL chimeric protein, were loaded into the second lane of each of the blots. Samples that were treated with a reducing agent, β-mercaptoethanol, and boiled were loaded into the third lane of each of the blots. Samples in the fourth lane of each of the blots were treated with a deglycosylation agent, the reducing agent, and were boiled. Each individual domain of the chimeric protein was probed using an anti-human SIRPα antibody (left blot), an anti-Fc (H+L) antibody (center blot), or an anti-4-1BBL antibody (right blot).

FIG. 17A to FIG. 17C show the binding analysis of the human SIRPα-Fc-4-1BBL chimeric protein to human CD47, human 4-1BB as measured using the Meso Scale Discovery (MSD) platform. FIG. 17A shows the binding of the human SIRPα-Fc-4-1BBL chimeric protein to human CD47-His. FIG. 17B shows the binding of the human SIRPα-Fc-4-1BBL chimeric protein to human 4-1BB-His. FIG. 17C shows the contemporaneous binding of the human SIRPα-Fc-4-1BBL chimeric protein to human 4-1BB-His and CD47-His.

FIG. 18A to FIG. 18C show the binding by the human SIRPα-Fc-4-1BBL chimeric protein to cells expressing 4-1BB and activation of the cells. FIG. 18A shows the binding of the human SIRPα-Fc-4-1BBL chimeric protein to HT1080 cells overexpressing 4-1BB as assayed by flow cytometry. FIG. 18B shows the quantitation of shows the binding of the human SIRPα-Fc-4-1BBL chimeric protein to HT1080 cells overexpressing 4-1BB as assayed by flow cytometry. FIG. 18C shows the IL-8 secretion in response to the 4-1BB/4-1BBL signaling induced by the human SIRPα-Fc-4-1BBL chimeric protein.

FIG. 19 shows a cartoon showing the structure of an illustrative mouse SIRPα-Fc-4-1BBL chimeric protein (top panel), and western blots showing characterization of the mouse SIRPα-Fc-4-1BBL chimeric protein (bottom panels). The western blots demonstrate the chimeric protein's native state and tendency to form a multimer. Molecular weight markers were loaded in the first lane of each blot. Untreated samples (i.e., without a reducing agent or a deglycosylation agent, yet boiled) of the mouse SIRPα-Fc-4-1BBL chimeric protein, were loaded into the second lane of each of the blots. Samples that were treated with a reducing agent, β-mercaptoethanol, and boiled were loaded into the third lane of each of the blots. Samples in the fourth lane of each of the blots were treated with a deglycosylation agent, the reducing agent, and were boiled. Each individual domain of the chimeric protein was probed using an anti-mouse SIRPα antibody (left blot), an anti-Fc (H+L) antibody (center blot), or an anti-4-1BBL antibody (right blot).

FIG. 20A to FIG. 20D show the binding analysis of the mouse SIRPα-Fc-4-1BBL chimeric protein to mouse CD47, mouse 4-1BB as measured using the Meso Scale Discovery (MSD) platform. FIG. 20A shows the binding of the mouse SIRPα-Fc-4-1BBL chimeric protein to anti-mouse Fc antibody. FIG. 20B shows the binding of the mouse SIRPα-Fc-4-1BBL chimeric protein to mouse 4-1BB-His. FIG. 20C shows the binding of the mouse SIRPα-Fc-4-1BBL chimeric protein to mouse CD47-His. FIG. 20D shows the contemporaneous binding of the mouse SIRPα-Fc-4-1BBL chimeric protein to mouse 4-1BB-His and CD47-His.

FIG. 21A to FIG. 21C show the comparison of anti-tumor activity of the SIRPα-Fc-4-1BBL chimeric protein and anti-PD-1 antibody in CT26 allograft model and 4th generation anti-PD-1 resistant cells. FIG. 21A shows a line graph of average tumor growth as a function of time. FIG. 21B shows a bar graph showing tumor volumes on day 17. FIG. 21C shows a Kaplan-Meier survival curve.

FIG. 22A to FIG. 22H show the pharmacodynamic (PD) activity of the TIGIT-Fc-LIGHT chimeric protein in cynomolgus macaque. FIG. 22A shows a dose dependent decrease or margination of lymphocytes. FIG. 22B shows the decrease or margination of CD3+ T cells. FIG. 22C shows the principal component analysis (PCA) distribution of animals based on the 2-hour post-dose cytokine signature. FIG. 22D shows the 2-hour post-dose cytokine signature of pro-inflammatory cytokines in a PCA vector plot. FIG. 22E shows the cytokine response as assessed using Meso Scale Discovery (MSD) assays. FIG. 22F shows the fold induction of IL-2. FIG. 22G shows the fold induction of IP-10. FIG. 22H shows the levels of CXCL-10 during and after first, second and third doses.

FIG. 23A to FIG. 23C show the immune cell phenotype analysis using mRNA and cell surface expression. FIG. 23A shows the analysis of previously published Affymetrix microarray data on isolated T stem cell memory (Tscm), T central memory (Tcm), T effector memory (Tem), and naïve T cells (Tn). The data was analyzed for the expression of TNFRSF14 (HVEM), CD226 (DNAM-1), and TIGIT. FIG. 23B shows the Uniform Manifold Approximation and Projection (UMAP) spatial organization in two additional immune gene set annotations. HPCA (Human Primary Cell Atlas) and Novershtern (Physical Module Networks), were used to assess the UMAP spatial distribution of the human PBMC scRNA-seq data presented in FIG. 13D. FIG. 23C shows the results of the flow cytometry analysis that was used to assess the cell surface expression of TIGIT, DNAM-1, and HVEM on murine T cells stimulated for 2 days with anti-mouse CD3/CD28 beads and IL-2, or murine NK cells (both isolated from the spleens of healthy animals). T cells were pre-gated on CD3 and NK cells on NKP46.

FIG. 24A to FIG. 24G show the characterization of the murine surrogate and functional activity of the mouse and human TIGIT-Fc-LIGHT chimeric protein molecules. FIG. 24A shows the characterization of the murine TIGIT-Fc-LIGHT chimeric protein surrogate (also referred to as mTIGIT-Fc-LIGHT), as assessed by Western blot using antibodies that probed each domain (TIGIT, Fc, and LIGHT), under non-reduced, reduced, and reduced+deglycosylated conditions. FIG. 24B shows the binding of the human TIGIT-Fc-LIGHT chimeric protein to recombinant human Nectin-4 was assessed using MSD. FIG. 24C (left panel) shows the levels of DcR3 in blood serum samples collected from human healthy donor and cancer patient. FIG. 24C (right panel) shows the binding of the binding of the human TIGIT-Fc-LIGHT chimeric protein to HVEM in the presence of soluble DcR3. To assess whether serum soluble DcR3 could interfere with the TIGIT-Fc-LIGHT chimeric protein binding to HVEM, the dual potency assay was performed with the TIGIT-Fc-LIGHT chimeric protein preincubated for 20 minutes on ice in each of the serum samples independently. The dual potency assay was then run using the MSD platform. FIG. 24D shows the binding of the mouse TIGIT-Fc-LIGHT chimeric protein (mTIGIT-Fc-LIGHT) to recombinant targets was assessed by ELISA. Binding to mouse LTβR expressing CHO-K1 cells was assessed using flow cytometry. FIG. 24E shows the binding of mTIGIT-Fc-LIGHT to CT26/WT, CT26/AR, and B16.F10 tumor cells as assessed using flow cytometry. FIG. 24F shows the ability of mTIGIT-Fc-LIGHT to enhance the killing of CT26 tumor cells by NK cells or CD8+ T cells (stimulated for 2 days with anti-mouse CD3/CD28 beads) as assessed using the Incuctye platform, where cleaved caspase 3/7 fluorescence was assessed over time. FIG. 24G shows the induction of gene expression of CXCL8, and CCL2 by the TIGIT Fc-LIGHT chimeric protein. Human A375 cells were incubated with an anti-LTβR agonist antibody or the TIGIT-Fc-LIGHT chimeric protein for 3 hours. RNA was harvested, reverse transcribed, and gene expression of ACTB, GAPDH, CXCL8, and CCL2 was assessed using qPCR.

FIG. 25A to FIG. 25G show the TIGIT-Fc-LIGHT chimeric protein (TIGIT-Fc(IgG1)-LIGHT, or TIGIT-Fc(IgG4)-LIGHT) activity in stimulating HVEM+ and LTβR+ immune cells. FIG. 25A shows the assessment of the TIGIT-Fc(IgG1)-LIGHT chimeric protein molecule by Western blot using antibodies that probed each domain (TIGIT, Fc, and LIGHT), under non-reduced, reduced, and reduced+deglycosylated conditions. FIG. 25B shows the results of an AIMV proliferation assay as performed with human PBMC+/−TIGIT-Fc(IgG1)-LIGHT, or TIGIT-Fc(IgG4)-LIGHT (150 nM each). Cell morphology was assessed at 2 days (left) in addition to the proliferative capacity of the culture using the Promega MTS proliferation assay (right). FIG. 25C shows the MSD receptor binding assays performed to demonstrate binding to target receptors using the TIGIT-Fc(IgG1)-LIGHT chimeric protein. The IgG1 molecule was also shown to engage effector Fc gamma receptors (drug concentration at 10 μg/mL), and also the FIG. 25D shows the cytokine analysis using AIMV PBMC culture on day 2 from the same donor PBMCs presented in FIG. 14A to FIG. 14C. Cytokines were assessed using a MSD multiplex where PBMC were treated with an Fc effector competent TIGIT antibody+/−anti-PD-1 (pembrolizumab)(All at 150 nM). The untreated bars are identical to how they are presented in FIG. 14C. FIG. 25E shows the UMAP depictions of the spatial expression of TIGIT, LTBR, TNFRSF14, CD226, PVR, PVRL2, PVRL3, and NECTIN4 from the human PBMC scRNA-seq data sets generated from untreated (UT), TIGIT-Fc(IgG1)-LIGHT, and TIGIT-Fc(IgG4)-LIGHT treatment groups. FIG. 25F shows the normalized expression from genes of interest isolated from the scRNA-seq dataset. FIG. 25G shows the assessment of differentially expressed genes between the TIGIT-Fc(IgG1)-LIGHT and TIGIT-Fc(IgG4)-LIGHT treatment groups, indicating, inter alia, the datasets are near identical (fold change >2-fold or <−2-fold, and adjusted p-values <0.05).

FIG. 26A to FIG. 26H show the anti-tumor activity of the TIGIT-Fc-LIGHT chimeric protein. FIG. 26A shows the Kaplan-Meier survival curves from additional CT26/WT treatment groups presented in FIG. 12C. FIG. 26B shows the anti-tumor activity of a commercial mouse anti-LTβR antibody. FIG. 26C shows the results of an assessment of memory immune response to secondary tumors. Mice that were available 29 days following the initial treatment, were inoculated with a second CT26 tumor on the opposing flank, with no subsequent re-treatment. The growth of secondary tumors was assessed over time as an indication of whether a memory immune response was generated in treated animals. The vehicle treated group consists of new mice that were inoculated with CT26 tumors as a reference for tumor growth. FIG. 26D shows the double positive effector memory T cells (DEPC). In re-challenged animals, peripheral blood was collected on day 39, and double positive effector memory T cells (DEPC) were assessed using flow cytometry. FIG. 26E shows the analysis of tumor infiltrating lymphocytes (TILs) in dissociated tumors. In a cohort of treated animals, tumors were isolated 9 days after the initial treatment, then dissociated, and the resulting cells were analyzed by flow cytometry. FIG. 26F shows the analysis of cytokines in dissociated tumors. The supernatant from dissociated tumors was assessed for cytokine expression using Luminex multiplex arrays. FIG. 26G shows the Kaplan-Meier survival curves from additional B16.F10 treatment groups presented in FIG. 12F. FIG. 26H shows the flow cytometry analysis of CD4/CD8 T cell or NK cell depletion in the peripheral blood on day 7, after 3 IP doses of depleting antibodies on days −1, 1, and day 7 (before peripheral blood collection).

FIG. 27A to FIG. 27D show the additional activity and anti-tumor efficacy data from the CT26/AR model. FIG. 27A shows the Kaplan-Meier survival curves from additional CT26/AR treatment groups presented in FIG. 12J. FIG. 27B shows the amino acid alignment of the cytoplasmic portions of CD226 and HVEM, using Clustal Omega multiple sequence alignment. Residues that have previously been implicated in PD-1 regulation of DNAM-1 are underlined. FIG. 27C shows the TIL analysis by flow cytometry showing the percentage of antigen-specific CD8+ T cells (CD8+AH1 tetramer-F) out of total CD3+ mononuclear cells (MNC). FIG. 27D shows the TIL analysis by flow cytometry showing the percentage of NK cells (NKP46+) out of total CD3-MNC.

FIG. 28A to FIG. 28C show the pharmacodynamic activity of the TIGIT-Fc-LIGHT chimeric protein in non-human primate toxicology studies. FIG. 28A shows the flow cytometry phenotyping of example HVEM expression on peripheral blood isolated from an animal pre-dose 1, and gated on CD45+CD3+CD8+ T cells. FIG. 28B shows the maximum post-dose cytokine response plotted across all dose groups, for each individual animal. Shading was used to highlight the dose response. FIG. 28C shows the mouse serum cytokine analysis performed on blood serum collected 9 days following treatment with the mTIGIT-Fc-LIGHT chimeric protein.

### DETAILED DESCRIPTION

The current disclosure is based, in part, on the discovery of upregulation or downregulation of certain functions that are associated with resistance to anti-PD-1 therapy (acquired or primary resistance), and that that the TIGIT-Fc-LIGHT chimeric protein or the SIRPα-Fc-4-1BBL chimeric protein is effective in cancers having acquired or primary resistance to anti-PD-1 therapy. Surprisingly, the combination of the TIGIT-Fc-LIGHT chimeric protein and an agent that blocks the PD-1/PD-L1 axis is effective against cancers acquired or primary resistance to anti-PD-1 therapy. Thus, these results establish therapy for cancers having acquired or primary resistance to anti-PD-1 therapy.

The current disclosure is based, in part, on the discovery that does upto 40 mg/kg of the TIGIT-Fc-LIGHT chimeric protein are safely tolerated in non-huma primates (NHP), and that treatment of NHP with the TIGIT-Fc-LIGHT chimeric protein induced lymphocyte expansion, lymphocyte margination and a specific post-dose cytokine signature, with no evidence of the cytokine release syndrome.

In embodiments, these results establish biomarkers associated with acquired and primary resistance to anti-PD-1 therapy. Therefore, based on these biomarkers, in embodiments disclosed herein a patient may be selected for treatment with an anti-PD-1 therapy based on evaluating the sample for the presence, absence, or level of genes associated with one or more gene ontology (GO) pathways disclosed herein from a biological sample from the patient. For example, in embodiments, the observed upregulation or downregulation of one or more genes associated with in various GO functions disclosed herein may be used to diagnose resistance to anti-PD-1 therapy (acquired or primary resistance).

Single-cell RNA sequencing of tumors, which have developed acquired resistance to PD-1 inhibitory antibodies, disclosed herein demonstrated the progressive acquisition of a transcriptionally hyperactive phenotype. Specifically, a larger number of transcripts were upregulated than downregulated in PD-1 acquired resistant tumors than the parental PD-1 antibody sensitive tumor. This observation suggests, without wishing to be bound by theory, that acquired resistance is an active process, in which tumor cells, which upregulate genes in certain pathways, acquire a survival advantage as compared to those cells that do not upregulate, or which downregulate, overall transcriptional activity. Accordingly, in embodiments, the tumors that have acquired resistance to checkpoint inhibitors, including PD-1 or PD-L1 blocking agents, may have increased sensitivity to a chimeric protein of a general structure of: N terminus—(a)—(b)—(c)—C terminus, wherein: (A) (a) is a first domain comprising an extracellular domain of a Type I transmembrane protein, the transmembrane protein being TIGIT, (b) is a linker comprising at least one cysteine residue capable of forming a disulfide bond, and (c) is a second domain comprising an extracellular domain of Type II transmembrane protein, the transmembrane protein being LIGHT, wherein the linker connects the first domain and the second domain and optionally comprises one or more joining linkers, such joining linkers being selected from SEQ ID NOs: 49-95; or (B) (a) is a first domain comprising an extracellular domain of a Type I transmembrane protein, the transmembrane protein being SIRPα, (b) is a linker comprising at least one cysteine residue capable of forming a disulfide bond, and (c) is a second domain comprising an extracellular domain of Type II transmembrane protein, the transmembrane protein being 4-1BBL, wherein the linker connects the first domain and the second domain and optionally comprises one or more joining linkers, such joining linkers being selected from SEQ ID NOs: 49-95.

In addition, the data presented herein demonstrate that while acquired resistant tumors are characterized by a transcriptionally hyperactive phenotype, many of the transcripts that are upregulated are not accompanied by an increase in corresponding protein expression. For example, the genes associated with CD274 (PD-L1), beta 2 macroglobulin, and other transcripts associated with interferon sensitivity and antigen presentation are upregulated in acquired resistant tumors, however there is not a corresponding increase in the amount of PD-L1 or beta 2 macroglobulin protein expression in acquired resistant tumors. Together, these findings suggest, inter alia, that acquired resistant tumors are attempting to upregulate many of the key genes that would drive increased sensitivity to an anti-tumor immune response, but that an acquired defect in a post-transcriptional protein or proteins has disrupted the response. For example, the increased PD-L1 mRNA levels would be expected to translate to increased levels of the PD-L1 protein. Accordingly, in embodiments, the modulators of one or more processes including: protein translation (e.g., assembly and/or function of ribosomal complex, adequate expression and/or function of tRNA, adequate synthesis and/or uptake of amino acids, etc.), post-translational modification (e.g., decoration of the translated protein with carbohydrates important for function or protection from degradation), or transport mechanisms (e.g., post-translational peptide processing, signal peptide recognition and cleavage, transport through the ER/Golgi network, etc.) may be helpful as combining defects in post-translational processes to create synthetic lethal phenotypes in cancer cell populations developing resistance to PD-1 or PD-L1 blocking agents. In embodiments, the classes of chemotherapy may be used as neoadjuvant therapy or as adjuvant therapy.

Chimeric Proteins

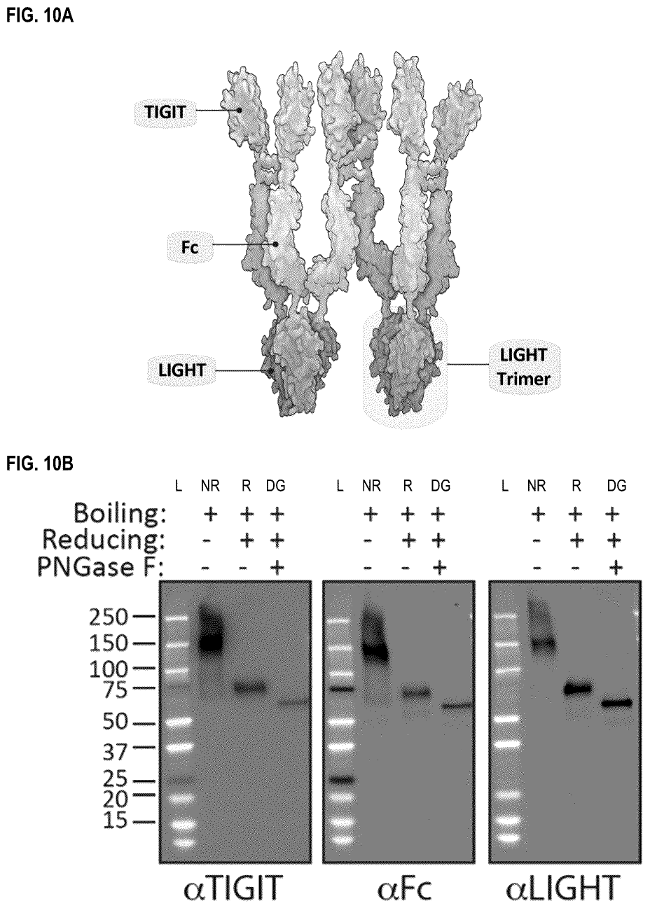

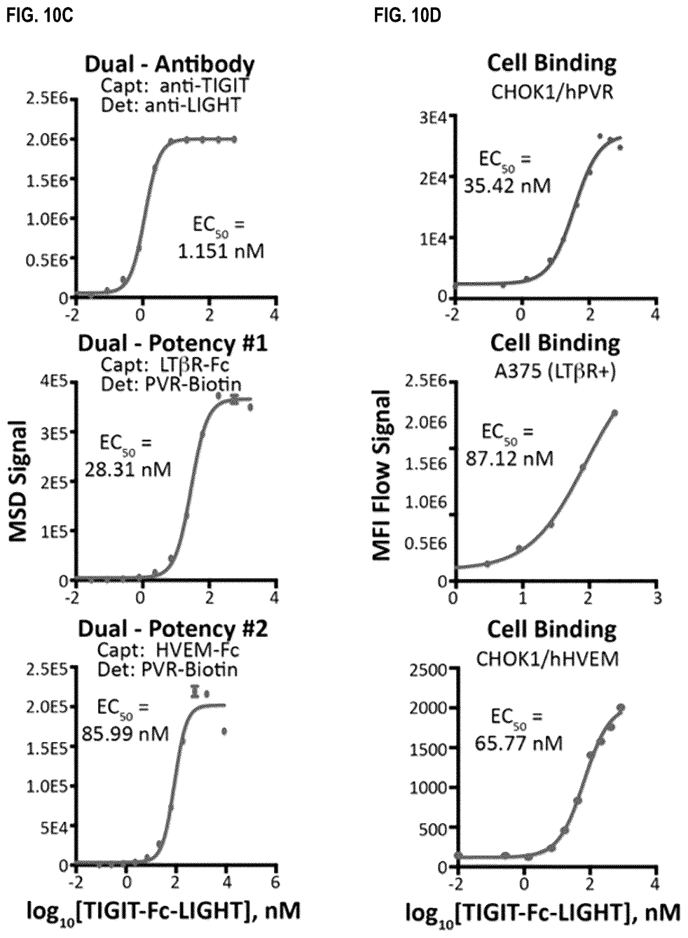

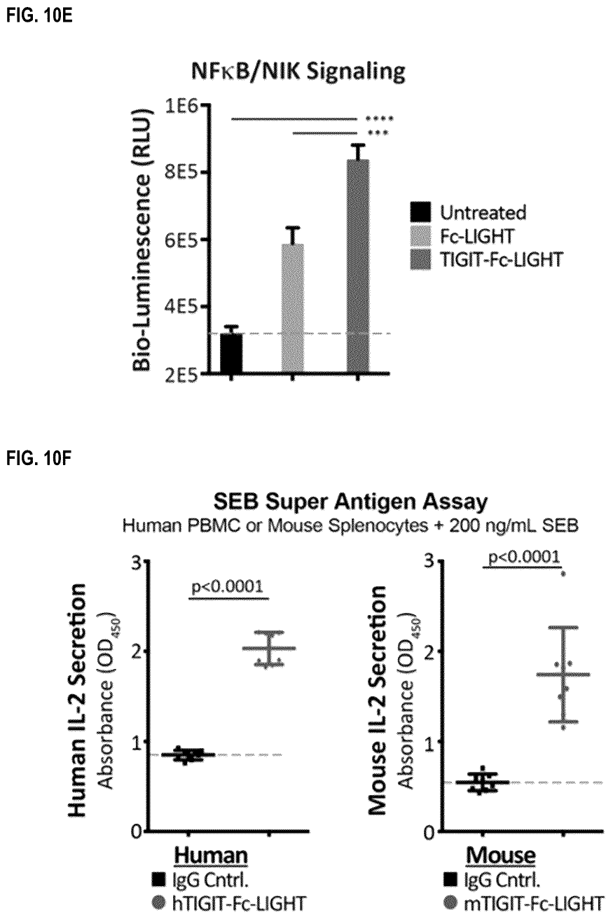

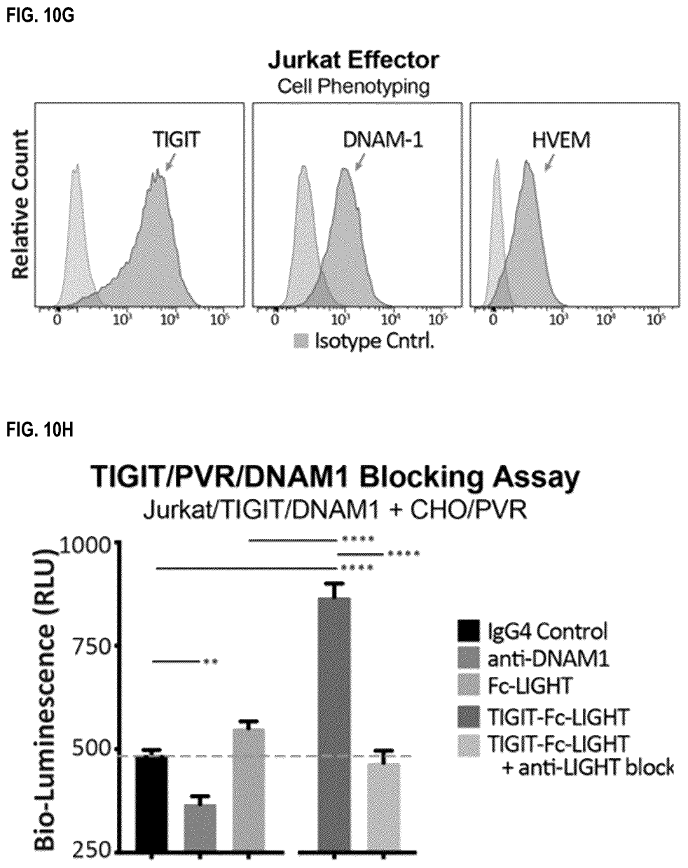

In some aspects, the chimeric protein is of a general structure of: N terminus—(a)—(b)—(c)—C terminus, where (a) is a first domain comprising an extracellular domain of a Type I transmembrane protein, wherein the Type I membrane protein is T cell immunoreceptor with Ig and ITIM domains (TIGIT), (b) is a linker having at least one cysteine residue capable of forming a disulfide bond (including without limitation, hinge-CH2-CH3 Fc domain is derived from human IgG4), and (c) is a second domain comprising an extracellular domain of Type II transmembrane protein being selected from 4-1BBL, GITRL, TL1A, and LIGHT, where the linker connects the first domain and the second domain and optionally comprises one or more joining linkers as described herein, where one of the first and second extracellular domains is an immune inhibitory signal and one of the first and second extracellular domains is an immune stimulatory signal. Exemplary chimeric proteins are disclosed in WO2018157162, the entire contents of which are hereby incorporated by reference. A molecular model of an exemplary chimeric protein, the TIGIT-Fc-LIGHT chimeric protein, which exists as a hexamer having functional sets of LIGHT trimers, is shown in FIG. 10A.

In some aspects, the chimeric protein is of a general structure of: N terminus—(a)—(b)—(c)—C terminus, where (a) is a first domain comprising an extracellular domain of a Type I transmembrane protein, wherein the Type I membrane protein is signal regulatory protein a (SIRPα), (b) is a linker having at least one cysteine residue capable of forming a disulfide bond (including without limitation, hinge-CH2-CH3 Fc domain is derived from human IgG4), and (c) is a second domain comprising an extracellular domain of Type II transmembrane protein being selected from 4-1BBL, CD40L, OX40L, CD30L, and GITRL, where the linker connects the first domain and the second domain and optionally comprises one or more joining linkers as described herein, where one of the first and second extracellular domains is an immune inhibitory signal and one of the first and second extracellular domains is an immune stimulatory signal. Exemplary chimeric proteins are disclosed in WO 2017/059168, WO 2018/157163, WO 2018/157164, WO 2018/157165, WO 2018/157162, WO 2019/246508, WO 2020/047325, WO 2020/047327, WO 2020/047328, WO 2020/047329, WO 2020/047319, WO 2020/047322, WO 2020/146393, WO 2020/176718, WO 2020/232365, the contents of each of which are hereby incorporated by reference in their entireties.

In embodiments, chimeric protein refers to a recombinant fusion protein, e.g., a single polypeptide having the extracellular domains described herein. For example, in embodiments, the chimeric protein is translated as a single unit in a cell. In embodiments, chimeric protein refers to a recombinant protein of multiple polypeptides, e.g., multiple extracellular domains described herein, that are linked to yield a single unit, e.g., in vitro (e.g., with one or more synthetic linkers described herein).

In embodiments, the chimeric protein is chemically synthesized as one polypeptide or each domain may be chemically synthesized separately and then combined. In embodiments, a portion of the chimeric protein is translated and a portion is chemically synthesized.

In embodiments, an extracellular domain refers to a portion of a transmembrane protein which is capable of interacting with the extracellular environment. In embodiments, an extracellular domain refers to a portion of a transmembrane protein which is sufficient to bind to a ligand or receptor and effective transmit a signal to a cell. In embodiments, an extracellular domain is the entire amino acid sequence of a transmembrane protein which is external of a cell or the cell membrane. In embodiments, an extracellular domain is the that portion of an amino acid sequence of a transmembrane protein which is external of a cell or the cell membrane and is needed for signal transduction and/or ligand binding as may be assayed using methods know in the art (e.g., in vitro ligand binding and/or cellular activation assays).

In embodiments, an immune inhibitory signal refers to a signal that diminishes or eliminates an immune response. For example, in the context of oncology, such signals may diminish or eliminate antitumor immunity. Under normal physiological conditions, inhibitory signals are useful in the maintenance of self-tolerance (e.g., prevention of autoimmunity) and also to protect tissues from damage when the immune system is responding to pathogenic infection. For instance, without limitation, immune inhibitory signal may be identified by detecting an increase in cellular proliferation, cytokine production, cell killing activity or phagocytic activity when such an inhibitory signal is blocked.

In embodiments, an immune stimulatory signal refers to a signal that enhances an immune response. For example, in the context of oncology, such signals may enhance antitumor immunity. For instance, without limitation, immune stimulatory signal may be identified by directly stimulating proliferation, cytokine production, killing activity or phagocytic activity of leukocytes. Specific examples include direct stimulation of TNF superfamily receptors such as OX40, LTβR, 4-1BB or TNFRSF25 using either receptor agonist antibodies or using chimeric proteins encoding the ligands for such receptors (OX40L, LIGHT, 4-1BBL, TL1A, respectively). Stimulation from any one of these receptors may directly stimulate the proliferation and cytokine production of individual T cell subsets. Another example includes direct stimulation of an immune inhibitory cell with through a receptor that inhibits the activity of such an immune suppressor cell. This would include, for example, stimulation of CD4+FoxP3+ regulatory T cells with a GITR agonist antibody or GITRL containing chimeric protein, which would reduce the ability of those regulatory T cells to suppress the proliferation of conventional CD4+ or CD8+ T cells. In another example, this would include stimulation of CD40 on the surface of an antigen presenting cell using a CD40 agonist antibody or a chimeric protein containing CD40L, causing activation of antigen presenting cells including enhanced ability of those cells to present antigen in the context of appropriate native costimulatory molecules, including those in the B7 or TNF superfamily. In another example, this would include stimulation of LTBR on the surface of a lymphoid or stromal cell using a LIGHT containing chimeric protein, causing activation of the lymphoid cell and/or production of pro-inflammatory cytokines or chemokines to further stimulate an immune response, optionally within a tumor.

Membrane proteins typically consist of an extracellular domain, one or a series of transmembrane domains, and an intracellular domain. Without wishing to be bound by theory, the extracellular domain of a membrane protein is responsible for interacting with a soluble or membrane bound receptor or ligand. Without wishing to be bound by theory, the trans-membrane domain(s) are responsible for localizing a protein to the plasma membrane. Without wishing to be bound by theory, the intracellular domain of a membrane protein is responsible for coordinating interactions with cellular signaling molecules to coordinate intracellular responses with the extracellular environment (or visa-versa). There are two types of single-pass membrane proteins, those with an extracellular amino terminus and intracellular carboxy terminus (Type I) and those with an extracellular carboxy terminus and intracellular amino terminus (Type II). Both Type I and Type II membrane proteins can be either receptors or ligands. For Type I membrane proteins, the amino terminus of the protein faces outside the cell, and therefore contains the functional domains that are responsible for interacting with other binding partners (either ligands or receptors) in the extracellular environment. For Type II membrane proteins, the carboxy terminus of the protein faces outside the cell, and therefore contains the functional domains that are responsible for interacting with other binding partners (either ligands or receptors) in the extracellular environment. Thus, these two types of proteins have opposite orientations to each other.

Because the outward facing domains of Type I and Type II membrane proteins are opposite, it is possible to link the extracellular domains of a Type I and Type II membrane protein such that the ‘outward facing’ domains of the molecules are also in opposing orientation to each other. The resulting construct would therefore consist of the extracellular domain of a Type I membrane protein on the ‘left’ side of the molecule, connected to the extracellular domain of a Type II membrane protein on the ‘right’ side of the molecule using a linker sequence. This construct could be produced by cloning of these three fragments (the extracellular domain of a Type I protein, followed by a linker sequence, followed by the extracellular domain of a Type II protein) into a vector (plasmid, viral or other) wherein the amino terminus of the complete sequence corresponded to the ‘left’ side of the molecule containing the Type I protein and the carboxy terminus of the complete sequence corresponded to the ‘right’ side of the molecule containing the Type II protein. Accordingly, in embodiments, the present chimeric proteins are engineered as such.

In embodiments, the extracellular domain may be used to produce a soluble protein to competitively inhibit signaling by that receptors ligand. In embodiments, the extracellular domain may be used to provide artificial signaling.

In embodiments, the extracellular domain of a Type I transmembrane protein is an immune inhibitory signal. In embodiments, the extracellular domain of a Type II transmembrane protein is an immune stimulatory signal.

In embodiments, the present chimeric proteins comprise an extracellular domain of a Type I transmembrane protein, or a functional fragment thereof. In embodiments, the present chimeric proteins comprise an extracellular domain of a Type II transmembrane protein, or a functional fragment thereof. In embodiments, the present chimeric proteins comprise an extracellular domain of a Type I transmembrane protein, or a functional fragment thereof, and an extracellular domain of a Type II transmembrane protein, or a functional fragment thereof.

The activation of regulatory T cells is critically influenced by costimulatory and coinhibitory signals. Two major families of costimulatory molecules include the B7 and the tumor necrosis factor (TNF) families. These molecules bind to receptors on T cells belonging to the CD28 or TNF receptor families, respectively. Many well-defined coinhibitors and their receptors belong to the B7 and CD28 families.

In embodiments, the present chimeric proteins may be engineered to target one or more molecules involved in immune inhibition, including for example TIGIT.

In embodiments, the chimeric protein of the present disclosure comprises an extracellular domain of an immune inhibitory agent, including for example TIGIT.

In embodiments, the present chimeric proteins may be engineered to target one or more molecules involved in immune inhibition, including for example SIRPα.

In embodiments, the chimeric protein of the present disclosure comprises an extracellular domain of an immune inhibitory agent, including for example SIRPα.

In embodiments, the chimeric protein of the present disclosure comprises an extracellular domain of a Type I membrane protein which has immune inhibitory properties. In embodiments, the chimeric protein is engineered to disrupt, block, reduce, and/or inhibit the transmission of an immune inhibitory signal.

In embodiments, the chimeric protein of the present disclosure comprises an extracellular domain of an immune stimulatory signal is LIGHT (CD258).

In embodiments, the chimeric protein simulates binding of an inhibitory signal ligand to its cognate receptor (e.g., TIGIT to CD155/PVR, Nectin-2, Nectin-3 and/or Nectin-4) but inhibits the inhibitory signal transmission to an immune cell (e.g., a T cell, macrophage or other leukocyte).

In embodiments, the chimeric protein simulates binding of an inhibitory signal ligand to its cognate receptor (e.g., SIRPα to CD47) but inhibits the inhibitory signal transmission to an immune cell (e.g., an eat me sigal for macrophage, a B cell, or other phagocyte or antigen presenting cell).

In embodiments, the chimeric protein comprises an immune inhibitory receptor extracellular domain and an immune stimulatory ligand extracellular domain which can, without limitation, deliver an immune stimulation to a T cell while masking a tumor cell's immune inhibitory signals. In embodiments, the chimeric protein delivers a signal that has the net result of T cell activation.

In embodiments, the chimeric protein comprises an immune inhibitory signal which is an ECD of a receptor of an immune inhibitory signal and this acts on a tumor cell that bears a cognate ligand of the immune inhibitory signal. In embodiments, the chimeric protein comprises an immune stimulatory signal which is an ECD of a ligand of an immune stimulatory signal and this acts on a T cell that bears a cognate receptor of the immune stimulatory signal. In embodiments, the chimeric protein comprises both (i) an immune inhibitory signal which is a receptor of an immune inhibitory signal and this acts on a tumor cell that bears a cognate ligand of the immune inhibitory signal and (ii) an immune stimulatory signal which is a ligand of an immune stimulatory signal and this acts on a T cell that bears a cognate receptor of the immune stimulatory signal.

In embodiments, the chimeric protein of the present disclosure comprises an extracellular domain of one or more of the immune-modulating agents described in Mahoney, Nature Reviews Drug Discovery 2015:14; 561-585, the entire contents of which are hereby incorporated by reference.

In embodiments, a chimeric protein is capable of binding murine ligand(s)/receptor(s).

In embodiments, a chimeric protein is capable of binding human ligand(s)/receptor(s)

In embodiments, the chimeric protein of the present disclosure comprises an extracellular domain of a Type II membrane protein which has immune stimulatory properties. In embodiments, the chimeric protein is engineered to enhance, increase, and/or stimulate the transmission of an immune stimulatory signal.

For instance, in embodiments, the extracellular domain of a Type I transmembrane protein is from TIGIT.

TIGIT is a poliovirus receptor (PVR)—like protein, an immunoreceptor expressed on T cells that contains immunoglobulin and immunoreceptor tyrosine-based inhibitory motif (ITIM) domains. As such, TIGIT acts as an inhibitory immune checkpoint on both T cells and natural killer (NK) cells, providing an opportunity to target both the adaptive and innate arms of the immune system.

TIGIT is expressed on NK cells and subsets of activated, memory and regulatory T cells, and particularly on follicular helper T cells within secondary lymphoid organs CD155/PVR is up-regulated on endothelial cells by IFN-gamma and is highly expressed on immature thymocytes, lymph node dendritic cells, and tumor cells of epithelial and neuronal origin. In embodiments, the present chimeric proteins (e.g., comprising the TIGIT ECD) modulate any of the cells described immediately above (e.g., in the context of an immune synapse).

TIGIT binds CD155/PVR, Nectin-2, Nectin-3 and Nectin-4. In embodiments, the present chimeric proteins (e.g., comprising the TIGIT ECD) modulate the binding of TIGIT to CD155/PVR (e.g., reduce or disrupt the binding or signal transmission). In embodiments, the present chimeric proteins (e.g., comprising the TIGIT ECD) modulate the binding of TIGIT to Nectin-2 (e.g., reduce or disrupt the binding or signal transmission). In embodiments, the present chimeric proteins (e.g., comprising the TIGIT ECD) modulate the binding of TIGIT to Nectin-3 (e.g., reduce or disrupt the binding or signal transmission). In embodiments, the present chimeric proteins (e.g., comprising the TIGIT ECD) modulate the binding of TIGIT to Nectin-4 (e.g., reduce or disrupt the binding or signal transmission).

In embodiments, the chimeric protein is of a general structure of: N terminus—(a)—(b)—(c)—C terminus, where (a) is a first domain comprising an extracellular domain of a Type I transmembrane protein, the transmembrane protein being TIGIT, (b) is a linker comprising at least one cysteine residue capable of forming a disulfide bond (including without limitation, hinge-CH2-CH3 Fc domain is derived from human IgG4), and (c) is a second domain comprising an extracellular domain of Type II transmembrane protein, the transmembrane protein being selected from 4-1BBL, GITRL, TL1A, and LIGHT, where the linker connects the first domain and the second domain and optionally comprises one or more joining linkers as described herein.

In embodiments, the chimeric protein comprises the extracellular domain of the immune inhibitory agent TIGIT and is paired with an immune stimulatory agent as follows: TIGIT/OX-40L; TIGIT/4-1BBL, TIGIT/LIGHT; TIGIT/GITRL; TIGIT/CD70; TIGIT/CD30L; TIGIT/CD40L; TIGIT/CD137L; TIGIT/TL1A; and TIGIT/OX40L. In embodiments the chimeric protein is the TIGIT-Fc-4-1BBL, TIGIT-Fc-GITRL, TIGIT-Fc-LIGHT, TIGIT-Fc-OX40L, or TIGIT-Fc-TL1A chimeric protein, in which the Fc represents a linker that comprises at least a portion of an Fc domain of an antibody and which comprises at least one cysteine residue capable of forming a disulfide bond.

For instance, in embodiments, the extracellular domain of a Type II transmembrane protein is from LIGHT.

LIGHT (HVEM-L, TNFSF14, or CD258), an entity homologous to lymphotoxins, with inducible nature, and able to compete with herpes simplex virus glycoprotein D for herpes virus entry mediator (HVEM)/tumor necrosis factor (TNF)-related 2 is a member of the TNF superfamily. It is a 29-kDa Type II transmembrane protein, is expressed as a homotrimer on activated T cells as well as DCs, and has three receptors, namely, HVEM, LT-8 receptor (LTβR, TNFRSF3) and decoy receptor 3 (DcR3). Without wishing to be bound by theory, three receptors with distinct cellular expression patterns have been known to interact with LIGHT: HVEM (TNFRSF14, CD270) detected on activated DCs, T and B cells, NK cells, monocytes, and endothelial cells; LTβR found on follicular DCs and stromal cells and binds LIGHT; and the soluble entity decoy receptor 3 (DcR3) detected on diverse cancer cells such as multiple myeloma and diffuse large B-cell lymphoma. In embodiments, the present chimeric proteins can disrupt or decrease the interaction of LIGHT with one or more of these three receptors.

LIGHT binds LTBR, and potentially HVEM as well as DcR3. In embodiments, the present chimeric proteins (e.g., comprising the LIGHT ECD) modulate the binding of LIGHT to LTBR (e.g., increase or promote the binding or signal transmission). LTBR is expressed by visceral, lymphoid, and other stroma, epithelia and myeloid cells, but not lymphocytes. In embodiments, the present chimeric proteins (e.g., comprising the LIGHT ECD) modulate one or more of visceral, lymphoid, and other stroma, epithelia and myeloid cells. In embodiments, the present chimeric proteins (e.g., comprising the LIGHT ECD) modulate the binding of LIGHT to HVEM (e.g., increase or promote the binding or signal transmission). In embodiments, the present chimeric proteins (e.g., comprising the LIGHT ECD) modulate the binding of LIGHT to DcR3 (e.g., increase or promote the binding or signal transmission).

In embodiments, the portion of 4-1BBL is a portion of the extracellular domain of 4-1BBL. In embodiments, the present chimeric protein further comprises a domain, e.g., the extracellular domain, of the immune stimulatory molecule 4-1BB ligand (4-1BBL). 4-1BBL is a type II transmembrane protein belonging to the Tumor Necrosis Factor (TNF) superfamily.

In embodiments, the second domain is a portion of 4-1BBL. In embodiments, the second domain comprises substantially all the extracellular domain of 4-1BBL. In embodiments, the second domain is capable of binding 4-1BB (also known as cluster of differentiation 137 (CD137) or tumor necrosis factor ligand superfamily member 9 (TNFSF9)). In embodiments, the binding to 4-1BB increases or activates an immune stimulatory signal. In embodiments, the binding to 4-1BB costimulates CD4 and/or CD8 T-cells. 4-1BBL is also known as cluster of differentiation 137 ligand (CD137L). Thus, throughout this disclosure, 4-1BBL and CD137L are synonymous, when referenced alone and/or when referenced in context of a chimeric protein, thus, for example, SIRPα-Fc-4-1BBL is the same chimeric protein as SIRPα-Fc-CD137L.

4-1BB ligand (4-1BBL) binds to the 4-1BB receptor on activated T Lymphocytes and antigen-presenting cells (APC). 4-1BB signaling is believed to follow an immune synapse, formed by 4-1BB+ lymphocytes and 4-1BBL+ antigen-presenting cells. For example, 4-1BBL binding induces B cell proliferation and immunoglobulin production. T cells are the major 4-1BB-expressing cells and may engage 4-1BBL on macrophages and or APCs for their activation. CD8+ T cells release IL-13 as well as IFN-γ through 4-1BB signaling.

In embodiments, the present chimeric protein comprises a domain, e.g., the extracellular domain, of human 4-1BBL. The human 4-1BBL comprises the following amino acid sequence:

MEYASDASLDPEAPWPPAPRARACRVLPWALVAGLLLLLLLAAACAVFLACPWAVSGARASPGSAASPRL REGPELSPDDPAGLLDLRQGMFAQLVAQNVLLIDGPLSWYSDPGLAGVSLTGGLSYKEDTKELVVAKAGV YYVFFQLELRRVVAGEGSGSVSLALHLQPLRSAAGAAALALTVDLPPASSEARNSAFGFQGRLLHLSAGQR LGVHLHTEARARHAWQLTQGATVLGLFRVTPEIPAGLPSPRSE (SEQ ID NO: 102)

The amino acid sequence of extracellular domain human 4-1BBL (amino acids 50-254 of SEQ ID NO: 102) is the following:

ACPWAVSGARASPGSAASPRLREGPELSPDDPAGLLDLRQGMFAQLVAQNVLLIDGPLSWYSDPGLAGV SLTGGLSYKEDTKELVVAKAGVYYVFFQLELRRVVAGEGSGSVSLALHLQPLRSAAGAAALALTVDLPPAS SEARNSAFGFQGRLLHLSAGQRLGVHLHTEARARHAWQLTQGATVLGLFRVTPEIPAGLPSPRSE (SEQ ID NO: 13)

In embodiments, the present chimeric protein comprises the extracellular domain of human 4-1BBL which has the amino acid sequence of SEQ ID NO: 13. In embodiments, the present chimeric proteins may comprise the extracellular domain of 4-1BBL as described herein, or a variant or functional fragment thereof. For instance, the chimeric protein may comprise a sequence of the extracellular domain of 4-1BBL as provided above, or a variant or functional fragment thereof having at least about 60%, or at least about 61%, or at least about 62%, or at least about 63%, or at least about 64%, or at least about 65%, or at least about 66%, or at least about 67%, or at least about 68%, or at least about 69%, or at least about 70%, or at least about 71%, or at least about 72%, or at least about 73%, or at least about 74%, or at least about 75%, or at least about 76%, or at least about 77%, or at least about 78%, or at least about 79%, or at least about 80%, or at least about 81%, or at least about 82%, or at least about 83%, or at least about 84%, or at least about 85%, or at least about 86%, or at least about 87%, or at least about 88%, or at least about 89%, or at least about 90%, or at least about 91%, or at least about 92%, or at least about 93%, or at least about 94%, or at least about 95%, or at least about 96%, or at least about 97%, or at least about 98%, or at least about 99%) sequence identity with the amino acid sequence of the extracellular domain of 4-1BBL as described herein.

4-1BBL derivatives can be constructed from available structural data, including that described by Won et al., “The structure of the trimer of human 4-1BB ligand is unique among members of the tumor necrosis factor superfamily.” J. Biol. Chem. 285: 9202-9210 (2010); Gilbreth et al., “Crystal structure of the human 4-1BB/4-1BBL complex.” J Biol Chem 293: 9880-9891 (2018); and Bitra et al., “Crystal structures of the human 4-1BB receptor bound to its ligand 4-1BBL reveal covalent receptor dimerization as a potential signaling amplifier.” J Biol Chem 293: 9958-9969 (2018).

In embodiments, the present chimeric proteins may comprise a variant extracellular domain of 4-1BBL in which the signal peptide (e.g., as provided in SEQ ID NO: 59) is replaced with an alternative signal peptide. In embodiments, the present chimeric protein may comprise a variant extracellular domain of 4-1BBL which is expressed from a cDNA that has been codon-optimized for expression in protein producing cells such as Chinese Hamster Ovary (CHO) or HEK cells.

In embodiments, the extracellular domain of 4-1BBL refers to a portion of protein which is capable of interacting with the extracellular environment. In embodiments, the extracellular domain of 4-1BBL is the entire amino acid sequence of the protein which is external of a cell or the cell membrane. In embodiments, the extracellular domain of 4-1BBL is a portion of an amino acid sequence of the protein which is external of a cell or the cell membrane and is needed for signal transduction and/or ligand binding as may be assayed using methods know in the art.

In embodiments, the extracellular domain of 4-1BBL refers to a portion of the protein which is capable for binding to the 4-1BB receptor. Like other TNF superfamily members, membrane-bound 4-1BBL exists as a homotrimer. 4-1BBL binds to 4-1BB, a member of the TNF receptor superfamily that is expressed predominantly on antigen presenting cells.

In embodiments, the chimeric protein of the invention binds to human 4-1BB with a K_{D} of less than about 1 μM, about 900 nM, about 800 nM, about 700 nM, about 600 nM, about 550 nM, about 530 nM, about 500 nM, about 400 nM, about 300 nM, about 200 nM, about 100 nM, about 90 nM, about 80 nM, about 70 nM, about 60 nM, about 55 nM, about 50 nM, about 45 nM, about 40 nM, about 35 nM, about 30 nM, about 25 nM, about 20 nM, about 15 nM, about 10 nM, or about 5 nM, or about 1 nM (as measured, for example, by surface plasmon resonance or biolayer interferometry). In embodiments, the chimeric protein binds to human 4-1BB with a K_{D} of less than about 1 nM, about 900 pM, about 800 pM, about 700 pM, about 600 pM, about 500 pM, about 400 pM, about 300 pM, about 200 pM, about 100 pM, about 90 pM, about 80 pM, about 70 pM, about 60 pM about 55 pM about 50 pM about 45 pM, about 40 pM, about 35 pM, about 30 pM, about 25 pM, about 20 pM, about 15 pM, or about 10 pM, or about 1 pM (as measured, for example, by surface plasmon resonance or biolayer interferometry). In embodiments, the chimeric protein binds to human 4-1BB with a KD of from about 300 pM to about 700 pM.

In embodiments, a chimeric protein of the present invention comprises: (1) a first domain comprising the amino acid sequence of SEQ ID NO: 58, (b) a second domain comprises the amino acid sequence of SEQ ID NO: 13, and (c) a linker comprises an amino acid sequence that is at least 95% identical to SEQ ID NO: 46, SEQ ID NO: 47, SEQ ID NO: 112 or SEQ ID NO: 113.

For instance, in embodiments, the extracellular domain of a Type I transmembrane protein is from signal regulatory protein a (SIRPα).

Signal regulatory protein a (SIRPα) is an inhibitory receptor of a broadly expressed transmembrane protein CD47 (also called the “don't eat me” signal). SIRPα is a regulatory membrane glycoprotein from SIRP family expressed mainly by macrophages and other myeloid cells.

The interaction of SIRPα and CD47 leads to activation of tyrosine phosphatases that inhibit myosin accumulation at the submembrane assembly site of the phagocytic synapse, resulting in the blockade of phagocytosis. Therefore, CD47 acts as a “don't eat me signal” for healthy self-cells; accordingly, loss of CD47 leads to phagocytosis of aged or damaged cells. Taking advantage of this anti-phagocytic signal provided by CD47, many types of tumors overexpress this protein, thereby avoiding phagocytosis by macrophages and aiding in the survival of cancer cells. SIRPα binds CD47. In embodiments, the present chimeric proteins (e.g., comprising the SIRPα ECD) modulate the binding of SIRPα to CD47 (e.g., reduce or disrupt the binding or signal transmission).

In embodiments, the present chimeric proteins (e.g., comprising the SIRPα ECD) modulate any of the cells described immediately above (e.g., in the context of an immune synapse).

In embodiments, a chimeric protein used in methods of the present invention comprises the extracellular domain of human SIRPα(CD172a) which comprises the following amino acid sequence:

(SEQ ID NO: 7)
EEELQVIQPDKSVLVAAGETATLRCTATSLIPVGPIQWFRGAGPGRELIY
NQKEGHFPRVTTVSDLTKRNNMDFSIRIGNITPADAGTYYCVKFRKGSPD
DVEFKSGAGTELSVRAKPSAPVVSGPAARATPQHTVSFTCESHGFSPRDI
TLKWFKNGNELSDFQTNVDPVGESVSYSIHSTAKVVLTREDVHSQVICEV
AHVTLQGDPLRGTANLSETIRVPPTLEVTQQPVRAENQVNVTCQVRKFYP
QRLQLTWLENGNVSRTETASTVTENKDGTYNWMSWLLVNVSAHRDDVKLT
CQVEHDGQPAVSKSHDLKVSAHPKEQGSNTAAENTGSNERNIY.

In embodiments, a chimeric protein used in methods of the present invention comprises a variant of the extracellular domain of SIRPα(CD172a). As examples, the variant may have at least about 60%, or at least about 61%, or at least about 62%, or at least about 63%, or at least about 64%, or at least about 65%, or at least about 66%, or at least about 67%, or at least about 68%, or at least about 69%, or at least about 70%, or at least about 71%, or at least about 72%, or at least about 73%, or at least about 74%, or at least about 75%, or at least about 76%, or at least about 77%, or at least about 78%, or at least about 79%, or at least about 80%, or at least about 81%, or at least about 82%, or at least about 83%, or at least about 84%, or at least about 85%, or at least about 86%, or at least about 87%, or at least about 88%, or at least about 89%, or at least about 90%, or at least about 91%, or at least about 92%, or at least about 93%, or at least about 94%, or at least about 95%, or at least about 96%, or at least about 97%, or at least about 98%, or at least about 99% sequence identity with SEQ ID NO: 7.

In embodiments, the variant of the extracellular domain of SIRPα(CD172a) has at least about 95% sequence identity with SEQ ID NO: 7.

One of ordinary skill may select variants of the known amino acid sequence of SIRPα(CD172a) by consulting the literature, e.g., LEE, et al., “Novel Structural Determinants of SIRPα that Mediate Binding of CD47,” The Journal of Immunology, 179, 7741-7750, 2007 and HATHERLEY, et al., “The Structure of the Macrophage Signal Regulatory Protein a (SIRPα) Inhibitory Receptor Reveals a Binding Face Reminiscent of That Used by T Cell Receptors,” The Journal Of Biological Chemistry, Vol. 282, No. 19, pp. 14567-14575, 2007, each of which is incorporated by reference in its entirety.

In embodiments, a heterologous chimeric protein comprises a first domain which comprises substantially the entire extracellular domain of SIRPα (CD172a), and/or the second domain which comprises substantially the entire extracellular domain of 4-1BBL. In embodiments, the first domain which comprises substantially the entire extracellular domain of SIRPα (CD172a). In embodiments, the second domain which comprises substantially the entire extracellular domain of 4-1BBL.

In embodiments, the chimeric protein is of a general structure of: N terminus—(a)—(b)—(c)—C terminus, where (a) is a first domain comprising an extracellular domain of a Type I transmembrane protein, the transmembrane protein being SIRPα, (b) is a linker comprising at least one cysteine residue capable of forming a disulfide bond (including without limitation, hinge-CH2-CH3 Fc domain is derived from human IgG4), and (c) is a second domain comprising an extracellular domain of Type II transmembrane protein, the transmembrane protein being selected from 4-1BBL, GITRL, TL1A, and LIGHT, where the linker connects the first domain and the second domain and optionally comprises one or more joining linkers as described herein.

In embodiments, the chimeric protein comprises the extracellular domain of the immune inhibitory agent SIRPα and is paired with an immune stimulatory agent as follows: CD172a(SIRPα)/OX-40L; CD172a(SIRPα)/4-1BBL, CD172a(SIRPα)/LIGHT; CD172a(SIRPα)/GITRL; CD172a(SIRPα)/CD70; CD172a(SIRPα)/CD30L; CD172a(SIRPα)/CD40L; CD172a(SIRPα)/CD137L; CD172a(SIRPα)/TL1A; and CD172a(SIRPα)/OX40L. In embodiments the chimeric protein is SIRPα-Fc-4-1BBL, SIRPα-Fc-GITRL, SIRPα-Fc-LIGHT, SIRPα-Fc-OX40L, or SIRPα-Fc-TL1A, in which the Fc represents a linker that comprises at least a portion of an Fc domain of an antibody and which comprises at least one cysteine residue capable of forming a disulfide bond.

In embodiments, the chimeric protein is of a general structure of: N terminus—(a)—(b)—(c)—C terminus, where (a) is a first domain comprising an extracellular domain of a Type I transmembrane protein, the transmembrane protein being selected from PD-1, CD172a(SIRPα), and TIGIT, (b) is a linker comprising at least one cysteine residue capable of forming a disulfide bond (including without limitation, hinge-CH2-CH3 Fc domain is derived from human IgG4), and (c) is a second domain comprising an extracellular domain of Type II transmembrane protein, the transmembrane protein being LIGHT, where the linker connects the first domain and the second domain and optionally comprises one or more joining linkers as described herein.

In embodiments, the chimeric protein is of a general structure of: N terminus—(a)—(b)—(c)—C terminus, where (a) is a first domain comprising an extracellular domain of a Type I transmembrane protein, the transmembrane protein being selected from CD172a(SIRPα), (b) is a linker comprising at least one cysteine residue capable of forming a disulfide bond (including without limitation, hinge-CH2-CH3 Fc domain is derived from human IgG4), and (c) is a second domain comprising an extracellular domain of Type II transmembrane protein, the transmembrane protein being 4-1BBL, where the linker connects the first domain and the second domain and optionally comprises one or more joining linkers as described herein.

In embodiments, the chimeric protein comprises the extracellular domain of the immune stimulatory agent LIGHT and is paired with an immune inhibitory agent as follows: PD-1/LIGHT, CD172a(SIRPα)/LIGHT, and TIGIT/LIGHT. In embodiments the chimeric protein is the PD-1-Fc-LIGHT, CD172a(SIRPα)-Fc-LIGHT, and TIGIT-Fc-LIGHT chimeric protein, in which the Fc represents a linker that comprises at least a portion of an Fc domain of an antibody and which comprises at least one cysteine residue capable of forming a disulfide bond.

In embodiments, the chimeric protein comprises the extracellular domain of the immune stimulatory agent LIGHT and is paired with an immune inhibitory agent as follows: CD172a(SIRPα)/OX-40L; CD172a(SIRPα)/4-1BBL, CD172a(SIRPα)/LIGHT; CD172a(SIRPα)/GITRL; CD172a(SIRPα)/CD70; CD172a(SIRPα)/CD30L; CD172a(SIRPα)/CD40L; CD172a(SIRPα)/CD137L; CD172a(SIRPα)/TL1A; and CD172a(SIRPα)/OX40L. In embodiments the chimeric protein is CD172a(SIRPα)-Fc-4-1BBL, CD172a(SIRPα)-Fc-CD40L, CD172a(SIRPα)/-Fc-LIGHT, and CD172a(SIRPα)-Fc-CD30L, in which the Fc represents a linker that comprises at least a portion of an Fc domain of an antibody and which comprises at least one cysteine residue capable of forming a disulfide bond.

In an embodiment, the chimeric protein comprises the extracellular domain of the immune inhibitory agent and is paired with the immune stimulatory agent. In embodiments, the chimeric protein binds to a cognate receptor or ligand with a K_{D} of about 1 nM to about 5 nM, for example, about 1 nM, about 1.5 nM, about 2 nM, about 2.5 nM, about 3 nM, about 3.5 nM, about 4 nM, about 4.5 nM, or about 5 nM. In embodiments, the chimeric protein binds to a cognate receptor or ligand with a K_{D} of about 5 nM to about 15 nM, for example, about 5 nM, about 5.5 nM, about 6 nM, about 6.5 nM, about 7 nM, about 7.5 nM, about 8 nM, about 8.5 nM, about 9 nM, about 9.5 nM, about 10 nM, about 10.5 nM, about 11 nM, about 11.5 nM, about 12 nM, about 12.5 nM, about 13 nM, about 13.5 nM, about 14 nM, about 14.5 nM, or about 15 nM.

In embodiments, the chimeric protein exhibits enhanced stability and protein half-life. In embodiments, the chimeric protein binds to FcRn with high affinity. In embodiments, the chimeric protein may bind to FcRn with a K_{D} of about 1 nM to about 80 nM. For example, the chimeric protein may bind to FcRn with a K_{D} of about 1 nM, about 2 nM, about 3 nM, about 4 nM, about 5 nM, about 6 nM, about 7 nM, about 8 nM, about 9 nM, about 10 nM, about 15 nM, about 20 nM, about 25 nM, about 30 nM, about 35 nM, about 40 nM, about 45 nM, about 50 nM, about 55 nM, about 60 nM, about 65 nM, about 70 nM, about 71 nM, about 72 nM, about 73 nM, about 74 nM, about 75 nM, about 76 nM, about 77 nM, about 78 nM, about 79 nM, or about 80 nM. In an embodiment, the chimeric protein may bind to FcRn with a K_{D} of about 9 nM. In embodiments, the chimeric protein does not substantially bind to other Fc receptors (i.e. other than FcRn) with effector function.

In embodiments, there is provided a method of treating a cancer and/or inflammatory disease (e.g., any one of those described elsewhere herein) by administering to a subject one or more of a CD172a(SIRPα)-Fc-LIGHT, PD-1-Fc-LIGHT, CD172a(SIRPα)-Fc-LIGHT, and TIGIT-Fc-LIGHT chimeric protein, in which the Fc represents a linker that comprises at least a portion of an Fc domain of an antibody and which comprises at least one cysteine residue capable of forming a disulfide bond. In embodiments, the method generates a memory response which may, e.g., be capable of preventing relapse. In embodiments, the method includes a sustained therapeutic effect of the one or more of a PD-1-Fc-LIGHT, CD172a(SIRPα)-Fc-LIGHT, and TIGIT-Fc-LIGHT chimeric protein, e.g., due to binding of the extracellular domain components to their respective binding partners with slow off rates (K_{d} or K_{off}) to optionally provide sustained negative signal masking effect and/or a longer positive signal effect, e.g., to allow an effector cell to be adequately stimulated for an anti-tumor effect. In embodiments, there is provided a method of treating a cancer and/or inflammatory disease (e.g., any one of those described elsewhere herein) by administering to a subject one or more of a CD172a(SIRPα)-Fc-4-1BBL, CD172a(SIRPα)-Fc-CD40L, CD172a(SIRPα)/-Fc-LIGHT, and CD172a(SIRPα)-Fc-CD30L chimeric protein, in which the Fc represents a linker that comprises at least a portion of an Fc domain of an antibody and which comprises at least one cysteine residue capable of forming a disulfide bond.

In embodiments, there is provided a method of treating a cancer or an inflammatory disease (e.g., any one of those described elsewhere herein) by administering to a subject one or more of a In embodiments, there is provided a method of treating a cancer and/or inflammatory disease (e.g., any one of those described elsewhere herein) by administering to a subject one or more of a CD172a(SIRPα)-Fc-4-1BBL, CD172a(SIRPα)-Fc-CD40L, CD172a(SIRPα)/-Fc-LIGHT, and CD172a(SIRPα)-Fc-CD30L chimeric protein, in which the Fc represents a linker that comprises at least a portion of an Fc domain of an antibody and which comprises at least one cysteine residue capable of forming a disulfide bond. chimeric protein, in which the Fc represents a linker that comprises at least a portion of an Fc domain of an antibody and which comprises at least one cysteine residue capable of forming a disulfide bond. In embodiments, the method generates a memory response which may, e.g., be capable of preventing relapse. In embodiments, the method includes a sustained therapeutic effect of the one or more of a TIGIT-Fc-4-1BBL, TIGIT-Fc-GITRL, TIGIT-Fc-TL1A, and TIGIT-Fc-LIGHT chimeric protein, e.g., due to binding of the extracellular domain components to their respective binding partners with slow off rates (K_{d} or K_{off}) to optionally provide sustained negative signal masking effect and/or a longer positive signal effect, e.g., to allow an effector cell to be adequately stimulated for an anti-tumor effect.

In embodiments, there is provided a method of treating a cancer or an inflammatory disease (e.g., any one of those described elsewhere herein) by administering to a subject one or more of a In embodiments, there is provided a method of treating a cancer and/or inflammatory disease (e.g., any one of those described elsewhere herein) by administering to a subject one or more of a CD172a(SIRPα)-Fc-4-1BBL, CD172a(SIRPα)-Fc-CD40L, CD172a(SIRPα)/-Fc-LIGHT, and CD172a(SIRPα)-Fc-CD30L chimeric protein, in which the Fc represents a linker that comprises at least a portion of an Fc domain of an antibody and which comprises at least one cysteine residue capable of forming a disulfide bond. chimeric protein, in which the Fc represents a linker that comprises at least a portion of an Fc domain of an antibody and which comprises at least one cysteine residue capable of forming a disulfide bond. In embodiments, the method generates a memory response which may, e.g., be capable of preventing relapse. In embodiments, the method includes a sustained therapeutic effect of the one or more of a In embodiments, there is provided a method of treating a cancer and/or inflammatory disease (e.g., any one of those described elsewhere herein) by administering to a subject one or more of a CD172a(SIRPα)-Fc-4-1BBL, CD172a(SIRPα)-Fc-CD40L, CD172a(SIRPα)/-Fc-LIGHT, and CD172a(SIRPα)-Fc-CD30L chimeric protein, in which the Fc represents a linker that comprises at least a portion of an Fc domain of an antibody and which comprises at least one cysteine residue capable of forming a disulfide bond, e.g., due to binding of the extracellular domain components to their respective binding partners with slow off rates (K_{d} or K_{off}) to optionally provide sustained negative signal masking effect and/or a longer positive signal effect, e.g., to allow an effector cell to be adequately stimulated for an anti-tumor effect.

In embodiments, the present chimeric proteins may comprises variants of the extracellular domains described herein, for instance, a sequence having at least about 60%, or at least about 61%, or at least about 62%, or at least about 63%, or at least about 64%, or at least about 65%, or at least about 66%, or at least about 67%, or at least about 68%, or at least about 69%, or at least about 70%, or at least about 71%, or at least about 72%, or at least about 73%, or at least about 74%, or at least about 75%, or at least about 76%, or at least about 77%, or at least about 78%, or at least about 79%, or at least about 80%, or at least about 81%, or at least about 82%, or at least about 83%, or at least about 84%, or at least about 85%, or at least about 86%, or at least about 87%, or at least about 88%, or at least about 89%, or at least about 90%, or at least about 91%, or at least about 92%, or at least about 93%, or at least about 94%, or at least about 95%, or at least about 96%, or at least about 97%, or at least about 98%, or at least about 99%) sequence identity with the known amino acid or nucleic acid sequence of any of the disclosed extracellular domains, e.g., human extracellular domains, e.g., one or more of SEQ ID NOs: 2, 4, 7, 10, 13, 16, 19, 22, 25, 27, 29, 31, 37, 41 or 103.

In embodiments, the chimeric protein of the present disclosure comprises an extracellular domain of LIGHT (SEQ ID NO: 2).

In embodiments, the chimeric protein of the present disclosure comprises an extracellular domain of PD-1 (SEQ ID NO: 4).

In embodiments, the chimeric protein of the present disclosure comprises an extracellular domain of TIGIT (SEQ ID NO: 10).

In embodiments, the chimeric protein of the present disclosure comprises an extracellular domain of CD172a(SIRPα) (SEQ ID NO: 7).

In embodiments, the chimeric protein of the present disclosure comprises an extracellular domain of 4-1BBL (SEQ ID NO: 13).

In embodiments, the chimeric protein of the present disclosure comprises an extracellular domain of CD172a(SIRPα) (SEQ ID NO: 7) and the extracellular domain of 4-1BBL (SEQ ID NO: 13).

In embodiments, the chimeric protein of the present disclosure comprises an extracellular domain of LIGHT (SEQ ID NO: 2) and the extracellular domain of PD-1 (SEQ ID NO: 4).

In embodiments, the chimeric protein of the present disclosure comprises an extracellular domain of LIGHT (SEQ ID NO: 2) and the extracellular domain of TIGIT (SEQ ID NO: 10).

In embodiments, the chimeric protein of the present disclosure comprises an extracellular domain of LIGHT (SEQ ID NO: 2) and the extracellular domain of CD172a(SIRPα) (SEQ ID NO: 7).

In embodiments, the chimeric protein of the present disclosure comprises the hinge-CH2-CH3 domain from a human IgG4 antibody sequence (SEQ ID NO: 46, 47, or 48).

In embodiments, the chimeric protein of the present disclosure comprises the hinge-CH2-CH3 domain from a human IgG4 antibody sequence (SEQ ID NO: 46, 47, or 48) and this sequence is flanked by at least one joining linker selected from SKYGPPCPSCP (SEQ ID NO: 49), SKYGPPCPPCP (SEQ ID NO: 50), IEGRMD SEQ ID NO: 52 (optionally SKYGPPCPSCP (SEQ ID NO: 49) or SKYGPPCPPCP (SEQ ID NO: 50) is N terminal and one of IEGRMD SEQ ID NO: 52 is C terminal).

In embodiments, the chimeric protein of the present disclosure comprises the hinge-CH2-CH3 domain from a human IgG1 antibody sequence (SEQ ID NO: 112, 113).

In embodiments, the chimeric protein of the present disclosure comprises the hinge-CH2-CH3 domain from a human IgG1 antibody sequence (SEQ ID NO: 112, 113) and this sequence is flanked by at least one joining linker selected from SKYGPPCPSCP (SEQ ID NO: 49), SKYGPPCPPCP (SEQ ID NO: 50), IEGRMD SEQ ID NO: 52 (optionally SKYGPPCPSCP (SEQ ID NO: 49) or SKYGPPCPPCP (SEQ ID NO: 50) is N terminal and one of IEGRMD SEQ ID NO: 52 is C terminal).

In embodiments, a chimeric protein comprises a modular linker.

In embodiments, the chimeric protein of the present disclosure comprises an extracellular domain of LIGHT and the extracellular domain of PD-1, using the hinge-CH2-CH3 domain from a human IgG4 antibody sequence as a linker (this PD-1-Fc-LIGHT chimera is SEQ ID NO: 5).

In embodiments, the chimeric protein of the present disclosure comprises an extracellular domain of LIGHT and the extracellular domain of TIGIT, using the hinge-CH2-CH3 domain from a human IgG4 antibody sequence as a linker (this TIGIT-Fc-LIGHT chimera is SEQ ID NO: 11).

In embodiments, the chimeric protein of the present disclosure comprises an extracellular domain of TIGIT and the extracellular domain of LIGHT, using the hinge-CH2-CH3 domain from a human IgG4 antibody sequence as a linker. In embodiments, the human TIGIT-Fc-LIGHT chimera has the following sequence (the extracellular domain (ECD) of human TIGIT is indicated by underline, a variant IgG4 CH2-CH3-Fc domain is shown in an italic font, joining linkers are shown in a boldface font, and the extracellular domain (ECD) of human LIGHT is indicated by an underlined, italic font):

(SEQ ID NO: 11)
MMTGTIETTGNISAEKGGSIILQCHLSSTTAQVTQVNWEQQDQLLAICNA
DLGWHISPSFKDRVAPGPGLGLTLQSLTVNDTGEYFCIYHTYPDGTYTGR
IFLEVLESSVAEHGARFQIPSKYGPPCPPCPAPEFLGGPSVFLFPPKPKD
QLMISRTPEVTCVVVDVSQEDPEVQFNWYVDGVEVHNAKTKPREEQFNST
YRVVSVLTVLHQDWLSGKEYKCKVSSKGLPSSIEKTISNATGQPREPQVY
TLPPSQEEMTKNQVSLTCLVKGFYPSDIAVEWESNGQPENNYKTTPPVLD
SDGSFFLYSRLTVDKSRWQEGNVFSCSVLHEALHNHYTQKSLSLSLGKIE
GRMDLQLHWRLGEMVTRLPDGPAGSWEQLIQERRSHEVNPAAHLTGANSS
LTGSGGPLLWETQLGLAFLRGLSYHDGALVVTKAGYYYIYSKVQLGGVGC
PLGLASTITHGLYKRTPRYPEELELLVSQQSPCGRATSSSRVWWDSSFLG
GVVHLEAGEKVVVRVLDERLVRLRDGTRSYFGAFMV.

In embodiments, the chimeric protein of the present disclosure comprises an extracellular domain of TIGIT and the extracellular domain of LIGHT, using the hinge-CH2-CH3 domain from a human IgG1 antibody sequence as a linker. this protein is also referred to herein as the TIGIT-Fc(IgG1)-LIGHT chimeric protein. In embodiments, the human TIGIT-Fc(IgG1)-LIGHT chimera has the following sequence (the extracellular domain (ECD) of human TIGIT is indicated by underline, a CH2-CH3-Fc domain derived from IgG1 is shown in an italic font, a joining linker is shown in a boldface font, and the extracellular domain (ECD) of human LIGHT is indicated by an underlined, italic font):

(SEQ ID NO: 109)
MMTGTIETTGNISAEKGGSIILQCHLSSTTAQVTQVNWEQQDQLLAICNA
DLGWHISPSFKDRVAPGPGLGLTLQSLTVNDTGEYFCIYHTYPDGTYTGR
IFLEVLESSVAEHGARFQIPEPKSCDKTHTCPPCPAPELLGGPSVFLFPP
KPKDTLMISRTPEVTCVVVDVSHEDPEVKFNWYVDGVEVHNAKTKPREEQ
YASTYRVVSVLTVLHQDWLNGKEYKCKVSNKALPAPIEKTISKAKGQPRE
PQVYTLPPSRDELTKNQVSLTCLVKGFYPSDIAVEWESNGQPENNYKTTP
PVLDSDGSFFLYSKLTVDKSRWQQGNVFSCSVMHEALHNHYTQKSLSLSP
GKIEGRMDLQLHWRLGEMVTRLPDGPAGSWEQLIQERRSHEVNPAAHLTG
ANSSLTGSGGPLLWETQLGLAFLRGLSYHDGALVVTKAGYYYIYSKVQLG
GVGCPLGLASTITHGLYKRTPRYPEELELLVSQQSPCGRATSSSRVWWDS
SFLGGVVHLEAGEKVVVRVLDERLVRLRDGTRSYFGAFMV

In embodiments, the chimeric protein of the present disclosure comprises an extracellular domain of TIGIT and the extracellular domain of LIGHT, using the hinge-CH2-CH3 domain from a human IgG1 antibody sequence as a linker. In embodiments, the human TIGIT-Fc-LIGHT chimera has the following sequence (the extracellular domain (ECD) of human TIGIT is indicated by underline, a variant IgG1 CH2-CH3-Fc domain (IgG1-LALA) is shown in an italic font, with a couple of mutations indicated in bold font, a joining linker is shown in a boldface font, and the extracellular domain (ECD) of human LIGHT is indicated by an underlined, italic font):

(SEQ ID NO: 110)
MMTGTIETTGNISAEKGGSIILQCHLSSTTAQVTQVNWEQQDQLLAICNA
DLGWHISPSFKDRVAPGPGLGLTLQSLTVNDTGEYFCIYHTYPDGTYTGR
IFLEVLESSVAEHGARFQIPEPKSCDKTHTCPPCPAPE [symbol] GGPSVFLFPP
KPKDTLMISRTPEVTCVVVDVSHEDPEVKFNWYVDGVEVHNAKTKPREEQ
YASTYRVVSVLTVLHQDWLNGKEYKCKVSNKALPAPIEKTISKAKGQPRE
PQVYTLPPSRDELTKNQVSLTCLVKGFYPSDIAVEWESNGQPENNYKTTP
PVLDSDGSFFLYSKLTVDKSRWQQGNVFSCSVMHEALHNHYTQKSLSLSP
GKIEGRMDLQLHWRLGEMVTRLPDGPAGSWEQLIQERRSHEVNPAAHLTG
ANSSLTGSGGPLLWETQLGLAFLRGLSYHDGALVVTKAGYYYIYSKVQLG
GVGCPLGLASTITHGLYKRTPRYPEELELLVSQQSPCGRATSSSRVWWDS
SFLGGVVHLEAGEKVVVRVLDERLVRLRDGTRSYFGAFMV

In embodiments, the chimeric protein of the present disclosure comprises an extracellular domain of mouse TIGIT and the extracellular domain of mouse LIGHT, using the hinge-CH2-CH3 domain from a mouse IgG1 antibody sequence as a linker. In embodiments, the mouse TIGIT-Fc-LIGHT chimera has the following sequence (the extracellular domain (ECD) of mouse TIGIT is indicated by underline, a variant IgG1 CH2-CH3-Fc domain is shown in an italic font, with a couple of mutations indicated in bold font, a joining linker is shown in a boldface font, and the extracellular domain (ECD) of mouse LIGHT is indicated by an underlined, italic font):

(SEQ ID NO: 111)
TIDTKRNISAEEGGSVILQCHFSSDTAEVTQVDWKQQDQLLAIYSVDLGW
HVASVFSDRVVPGPSLGLTFQSLTMNDTGEYFCTYHTYPGGIYKGRIFLK
VQESSDDRNGLAQFQTAPLGVPRDCGCKPCICTVPEVSSVFIFPPKPKDV
LTITLTPKVTCVVVDISKDDPEVQFSWFVDDVEVHTAQTQPREEQFNSTF
RSVSELPIMHQDWLNGKEFKCRVNSAAFPAPIEKTISKTKGRPKAPQVYT
IPPPKEQMAKDKVSLTCMITDFFPEDITVEWQWNGQPAENYKNTQPIMDT
DGSYFVYSKLNVQKSNWEAGNTFTCSVLHEGLHNHHTEKSLSHSPGKIEG
RMDLHQRLGDIVAHLPDGGKGSWEKLIQDQRSHQANPAAHLTGANASLIG
IGGPLLWETRLGLAFLRGLTYHDGALVTMEPGYYYVYSKVQLSGVGCPQG
LANGLPITHGLYKRTSRYPKELELLVSRRSPCGRANSSRVWWDSSFLGGV
VHLEAGEEVVVRVPGNRLVRPRDGTRSYFGAFMV

In embodiments, the present TIGIT-Fc-LIGHT chimeric protein may be variants described herein, for instance, the present chimeric proteins may have a sequence having at least about 60%, or at least about 61%, or at least about 62%, or at least about 63%, or at least about 64%, or at least about 65%, or at least about 66%, or at least about 67%, or at least about 68%, or at least about 69%, or at least about 70%, or at least about 71%, or at least about 72%, or at least about 73%, or at least about 74%, or at least about 75%, or at least about 76%, or at least about 77%, or at least about 78%, or at least about 79%, or at least about 80%, or at least about 81%, or at least about 82%, or at least about 83%, or at least about 84%, or at least about 85%, or at least about 86%, or at least about 87%, or at least about 88%, or at least about 89%, or at least about 90%, or at least about 91%, or at least about 92%, or at least about 93%, or at least about 94%, or at least about 95%, or at least about 96%, or at least about 97%, or at least about 98%, or at least about 99%) sequence identity with the amino acid sequence of one of SEQ ID NO: 11, 109, and 110.

In embodiments, the chimeric protein of the present disclosure comprises an extracellular domain of SIRPα and the extracellular domain of 4-1BBL, using the hinge-CH2-CH3 domain from a human IgG4 antibody sequence as a linker. In embodiments, the human SIRPα-Fc-4-1BBL chimera has the following sequence (the extracellular domain (ECD) of SIRPα is indicated by underline, a variant IgG4 CH2-CH3-Fc domain is shown in an italic font, joining linkers are shown in a boldface font, and the extracellular domain (ECD) of 4-1BBL is indicated by an underlined, italic font):

(SEQ ID NO: 103)
EEELQVIQPDKSVLVAAGETATLRCTATSLIPVGPIQWFRGAGPGRELIY
NQKEGHFPRVTTVSDLTKRNNMDFSIRIGNITPADAGTYYCVKFRKGSPD
DVEFKSGAGTELSVRAKPSAPVVSGPAARATPQHTVSFTCESHGFSPRDI
TLKWFKNGNELSDFQTNVDPVGESVSYSIHSTAKVVLTREDVHSQVICEV
AHVTLQGDPLRGTANLSETIRVPPTLEVTQQPVRAENQVNVTCQVRKFYP
QRLQLTWLENGNVSRTETASTVTENKDGTYNWMSWLLVNVSAHRDDVKLT
CQVEHDGQPAVSKSHDLKVSAHPKEQGSNTAAENTGSNERNIYSKYGPPC
PPCPAPEFLGGPSVFLFPPKPKDQLMISRTPEVTCVVVDVSQEDPEVQFN
WYVDGVEVHNAKTKPREEQFNSTYRVVSVLTVLHQDWLSGKEYKCKVSSK
GLPSSIEKTISNATGQPREPQVYTLPPSQEEMTKNQVSLTCLVKGFYPSD
IAVEWESNGQPENNYKTTPPVLDSDGSFFLYSRLTVDKSRWQEGNVFSCS
VLHEALHNHYTQKSLSLSLGKIEGRMDACPWAVSGARASPGSAASPRLRE
GPELSPDDPAGLLDLRQGMFAQLVAQNVLLIDGPLSWYSDPGLAGVSLTG
GLSYKEDTKELVVAKAGVYYVFFQLELRRVVAGEGSGSVSLALHLQPLRS
AAGAAALALTVDLPPASSEARNSAFGFQGRLLHLSAGQRLGVHLHTEARA
RHAWQLTQGATVLGLFRVTPEIPAGLPSPRSE.

In embodiments, the mouse SIRPα-Fc-4-1BBL chimera has the following sequence (the extracellular domain (ECD) of SIRPα is indicated by underline, a mouse IgG1 CH2-CH3-Fc domain is shown in an italic font, joining linker is shown in a boldface font, and the extracellular domain (ECD) of 4-1BBL is indicated by an underlined, italic font):

(SEQ ID NO: 108)
KELKVTQPEKSVSVAAGDSTVLNCTLTSLLPVGPIKWYRGVGQSRLLIYS
FTGEHFPRVTNVSDATKRNNMDFSIRISNVTPEDAGTYYCVKFQKGPSEP
DTEIQSGGGTEVYVLAKPSPPEVSGPADRGIPDQKVNFTCKSHGFSPRNI
TLKWFKDGQELHHLETTVNPSGKNVSYNISSTVRVVLNSMDVHSKVICEV
AHITLDRSPLRGIANLSNFIRVSPTVKVTQQSPTSMNQVNLTCRAERFYP
EDLQLIWLENGNVSRNDTPKNLTKNTDGTYNYTSLFLVNSSAHREDVVFT
CQVKHDQQPAITRNHTVLGLAHSSDQGSMQTFPGNNATHNWNVPRDCGCK
PCICTVPEVSSVFIFPPKPKDVLTITLTPKVTCVVVDISKDDPEVQFSWF
VDDVEVHTAQTQPREEQFNSTFRSVSELPIMHQDWLNGKEFKCRVNSAAF
PAPIEKTISKTKGRPKAPQVYTIPPPKEQMAKDKVSLTCMITDFFPEDIT
VEWQWNGQPAENYKNTQPIMDTDGSYFVYSKLNVQKSNWEAGNTFTCSVL
HEGLHNHHTEKSLSHSPGIIEGRMDRTEPRPALTITTSPNLGTRENNADQ
VTPVSHIGCPNTTQQGSPVFAKLLAKNQASLCNTTLNWHSQDGAGSSYLS
QGLRYEEDKKELVVDSPGLYYVFLELKLSPTFTNTGHKVQGWVSLVLQAK
PQVDDFDNLALTVELFPCSMENKLVDRSWSQLLLLKAGHRLSVGLRAYLH
GAQDAYRDWELSYPNTTSFGLFLVKPDNPWE

In embodiments, the present SIRPα-Fc-4-1BBL chimeric protein may be variants described herein, for instance, the present chimeric proteins may have a sequence having at least about 60%, or at least about 61%, or at least about 62%, or at least about 63%, or at least about 64%, or at least about 65%, or at least about 66%, or at least about 67%, or at least about 68%, or at least about 69%, or at least about 70%, or at least about 71%, or at least about 72%, or at least about 73%, or at least about 74%, or at least about 75%, or at least about 76%, or at least about 77%, or at least about 78%, or at least about 79%, or at least about 80%, or at least about 81%, or at least about 82%, or at least about 83%, or at least about 84%, or at least about 85%, or at least about 86%, or at least about 87%, or at least about 88%, or at least about 89%, or at least about 90%, or at least about 91%, or at least about 92%, or at least about 93%, or at least about 94%, or at least about 95%, or at least about 96%, or at least about 97%, or at least about 98%, or at least about 99%) sequence identity with the amino acid sequence of SEQ ID NO: 103.

In embodiments, the chimeric protein of the present disclosure comprises an extracellular domain of LIGHT and the extracellular domain of CD172a(SIRPα), using the hinge-CH2-CH3 domain from a human IgG4 antibody sequence as a linker (this CD172a(SIRPα)-Fc-LIGHT chimera is SEQ ID NO: 8).

In embodiments, the chimeric protein of the present disclosure comprises an extracellular domain of TIGIT (SEQ ID NO: 10).

In embodiments, the chimeric protein of the present disclosure comprises an extracellular domain of 4-1BBL (SEQ ID NO: 13).

In embodiments, the chimeric protein of the present disclosure comprises an extracellular domain of GITRL (SEQ ID NO: 16).

In embodiments, the chimeric protein of the present disclosure comprises an extracellular domain of TL1A (SEQ ID NO: 19).

In embodiments, the chimeric protein of the present disclosure comprises an extracellular domain of LIGHT (SEQ ID NO: 2).

In embodiments, the chimeric protein of the present disclosure comprises an extracellular domain of OX40L (SEQ ID NO: 22).

In embodiments, the chimeric protein of the present disclosure comprises an extracellular domain of TIGIT (SEQ ID NO: 10) and the extracellular domain of 4-1BBL (SEQ ID NO: 13).

In embodiments, the chimeric protein of the present disclosure comprises an extracellular domain of TIGIT (SEQ ID NO: 10) and the extracellular domain of GITRL (SEQ ID NO: 16).

In embodiments, the chimeric protein of the present disclosure comprises an extracellular domain of TIGIT (SEQ ID NO: 10) and the extracellular domain of TL1A (SEQ ID NO: 19).

In embodiments, the chimeric protein of the present disclosure comprises an extracellular domain of TIGIT (SEQ ID NO: 10) and the extracellular domain of LIGHT (SEQ ID NO: 2).

In embodiments, the chimeric protein of the present disclosure comprises an extracellular domain of TIGIT

(SEQ ID NO: 10) and the extracellular domain of OX40L (SEQ ID NO: 22).

In embodiments, the chimeric protein of the present disclosure comprises an extracellular domain of CD172a(SIRPα) (SEQ ID NO: 7) and the extracellular domain of 4-1BBL (SEQ ID NO: 13).

In embodiments, the chimeric protein of the present disclosure comprises an extracellular domain of CD172a(SIRPα) (SEQ ID NO: 7) and the extracellular domain of GITRL (SEQ ID NO: 16).

In embodiments, the chimeric protein of the present disclosure comprises an extracellular domain of CD172a(SIRPα) (SEQ ID NO: 7) and the extracellular domain of TL1A (SEQ ID NO: 19).

In embodiments, the chimeric protein of the present disclosure comprises an extracellular domain of CD172a(SIRPα) (SEQ ID NO: 7) and the extracellular domain of LIGHT (SEQ ID NO: 2).

In embodiments, the chimeric protein of the present disclosure comprises an extracellular domain of CD172a(SIRPα) (SEQ ID NO: 7) and the extracellular domain of OX40L (SEQ ID NO: 22).

In embodiments, the chimeric protein of the present disclosure comprises the hinge-CH2-CH3 domain from a human IgG4 antibody sequence (SEQ ID NO: 46, 47, or 48).

In embodiments, the chimeric protein of the present disclosure comprises the hinge-CH2-CH3 domain from a human IgG1 antibody sequence (SEQ ID NO: 112 or 113).

In embodiments, the chimeric protein of the present disclosure comprises the hinge-CH2-CH3 domain from a human IgG4 antibody sequence (SEQ ID NO: 46, 47, or 48) and this sequence is flanked by at least one joining linker selected from SKYGPPCPSCP (SEQ ID NO: 49), SKYGPPCPPCP (SEQ ID NO: 50), IEGRMD SEQ ID NO: 52 (optionally SKYGPPCPSCP (SEQ ID NO: 49) or SKYGPPCPPCP (SEQ ID NO: 50) is N terminal and one of IEGRMD SEQ ID NO: 52 is C terminal).

In embodiments, the chimeric protein of the present disclosure comprises the hinge-CH2-CH3 domain from a human IgG1 antibody sequence (SEQ ID NO: 112 or 113) and this sequence is flanked by at least one joining linker selected from SKYGPPCPSCP (SEQ ID NO: 49), SKYGPPCPPCP (SEQ ID NO: 50), IEGRMD SEQ ID NO: 52 (optionally SKYGPPCPSCP (SEQ ID NO: 49) or SKYGPPCPPCP (SEQ ID NO: 50) is N terminal and one of IEGRMD SEQ ID NO: 52 is C terminal).

In embodiments, the chimeric protein of the present disclosure comprises an extracellular domain of TIGIT and the extracellular domain of 4-1BBL, using the hinge-CH2-CH3 domain from a human IgG4 antibody sequence as a linker (this TIGIT-Fc-4-1BBL chimera is SEQ ID NO: 14).

In embodiments, the chimeric protein of the present disclosure comprises an extracellular domain of TIGIT and the extracellular domain of GITRL, using the hinge-CH2-CH3 domain from a human IgG4 antibody sequence as a linker (this TIGIT-Fc-GITRL chimera is SEQ ID NO: 17).

In embodiments, the chimeric protein of the present disclosure comprises an extracellular domain of TIGIT and the extracellular domain of TL1A, using the hinge-CH2-CH3 domain from a human IgG4 antibody sequence as a linker (this TIGIT-Fc-TL1A chimera is SEQ ID NO: 20).

In embodiments, the chimeric protein of the present disclosure comprises an extracellular domain of TIGIT and the extracellular domain of LIGHT, using the hinge-CH2-CH3 domain from a human IgG4 antibody sequence as a linker (this TIGIT-Fc-LIGHT chimera is SEQ ID NO: 11).

In embodiments, the chimeric protein of the present disclosure comprises an extracellular domain of TIGIT and the extracellular domain of OX40L, using the hinge-CH2-CH3 domain from a human IgG4 antibody sequence as a linker (this TIGIT-Fc-OX40L chimera is SEQ ID NO: 23).

In embodiments, the chimeric protein of the present disclosure comprises an extracellular domain of CD172a(SIRPα) and the extracellular domain of 4-1BBL, using the hinge-CH2-CH3 domain from a human IgG4 antibody sequence as a linker (this CD172a(SIRPα-Fc-4-1BBL chimera is SEQ ID NO: 103).

In embodiments, a chimeric protein can comprise an extracellular domain from a sequence identified herein combined with an extracellular domain from another sequence identified herein. For example, the sequence of a TIGIT-Fc-TL1A chimeric protein could include the extracellular domain of TIGIT as disclosed above in SEQ ID NO: 10 and the extracellular domain of TL1A as disclosed above in SEQ ID NO: 19.

In embodiments, additional chimeric proteins and methods using the additional chimeric proteins (e.g., in treating a cancer and/or treating an inflammatory disease): TIGIT-Fc-4-1BBL, TIGIT-Fc-CD30L, TIGIT-Fc-FasL, TIGIT-Fc-GITRL, TIGIT-Fc-TL1A, and TIGIT-Fc-TRAIL. The amino acid sequence for 4-1BBL, CD30L, FasL, GITRL, TL1A, and TRAIL, respectively, comprises SEQ ID NO: 12, 26, 30, 15, 18, and 40. The amino acid sequence for extracellular domain of 4-1BBL, CD30L, FasL, GITRL, TL1A, and TRAIL, respectively, are SEQ ID NO: 13, 27, 31, 16, 19, and 41.

In embodiments, the present chimeric proteins may be variants described herein, for instance, the present chimeric proteins may have a sequence having at least about 60%, or at least about 61%, or at least about 62%, or at least about 63%, or at least about 64%, or at least about 65%, or at least about 66%, or at least about 67%, or at least about 68%, or at least about 69%, or at least about 70%, or at least about 71%, or at least about 72%, or at least about 73%, or at least about 74%, or at least about 75%, or at least about 76%, or at least about 77%, or at least about 78%, or at least about 79%, or at least about 80%, or at least about 81%, or at least about 82%, or at least about 83%, or at least about 84%, or at least about 85%, or at least about 86%, or at least about 87%, or at least about 88%, or at least about 89%, or at least about 90%, or at least about 91%, or at least about 92%, or at least about 93%, or at least about 94%, or at least about 95%, or at least about 96%, or at least about 97%, or at least about 98%, or at least about 99%) sequence identity with the amino acid sequence of the present chimeric proteins, e.g., one or more of SEQ ID NOs: 5, 8, 11, 14, 17, 20, 23, 42, 43, 44, 45, or 103.

In embodiments, the chimeric protein may comprise an amino acid sequence having one or more amino acid mutations relative to any of the protein sequences described herein. In embodiments, the one or more amino acid mutations may be independently selected from substitutions, insertions, deletions, and truncations.

In embodiments, the amino acid mutations are amino acid substitutions, and may include conservative and/or non-conservative substitutions.

“Conservative substitutions” may be made, for instance, on the basis of similarity in polarity, charge, size, solubility, hydrophobicity, hydrophilicity, and/or the amphipathic nature of the amino acid residues involved. The 20 naturally occurring amino acids can be grouped into the following six standard amino acid groups: (1) hydrophobic: Met, Ala, Val, Leu, Ile; (2) neutral hydrophilic: Cys, Ser, Thr; Asn, Gln; (3) acidic: Asp, Glu; (4) basic: His, Lys, Arg; (5) residues that influence chain orientation: Gly, Pro; and (6) aromatic: Trp, Tyr, Phe.

As used herein, “conservative substitutions” are defined as exchanges of an amino acid by another amino acid listed within the same group of the six standard amino acid groups shown above. For example, the exchange of Asp by Glu retains one negative charge in the so modified polypeptide. In addition, glycine and proline may be substituted for one another based on their ability to disrupt α-helices.

As used herein, “non-conservative substitutions” are defined as exchanges of an amino acid by another amino acid listed in a different group of the six standard amino acid groups (1) to (6) shown above.

In embodiments, the substitutions may also include non-classical amino acids (e.g., selenocysteine, pyrrolysine, N-formylmethionine ρ-alanine, GABA and δ-Aminolevulinic acid, 4-aminobenzoic acid (PABA), D-isomers of the common amino acids, 2,4-diaminobutyric acid, α-amino isobutyric acid, 4-aminobutyric acid, Abu, 2-amino butyric acid, γ-Abu, ε-Ahx, 6-amino hexanoic acid, Aib, 2-amino isobutyric acid, 3-amino propionic acid, ornithine, norleucine, norvaline, hydroxyproline, sarcosme, citrulline, homocitrulline, cysteic acid, t-butylglycine, t-butylalanine, phenylglycine, cyclohexylalanine, β-alanine, fluoro-amino acids, designer amino acids such as β methyl amino acids, C α-methyl amino acids, N α-methyl amino acids, and amino acid analogs in general).

Mutations may also be made to the nucleotide sequences of the chimeric proteins by reference to the genetic code, including taking into account codon degeneracy.

In embodiments, the chimeric protein comprises a linker. In embodiments, the linker comprising at least one cysteine residue capable of forming a disulfide bond. As described elsewhere herein, such at least one cysteine residue capable of forming a disulfide bond is, without wishing to be bound by theory, responsible for maintain a proper multimeric state of the chimeric protein and allowing for efficient production.

In embodiments, the linker may be derived from naturally-occurring multi-domain proteins or are empirical linkers as described, for example, in Chichili et al., (2013), Protein Sci. 22(2):153-167, Chen et al., (2013), Adv Drug Deliv Rev. 65(10):1357-1369, the entire contents of which are hereby incorporated by reference. In embodiments, the linker may be designed using linker designing databases and computer programs such as those described in Chen et al., (2013), Adv Drug Deliv Rev. 65(10):1357-1369 and Crasto et. al., (2000), Protein Eng. 13(5):309-312, the entire contents of which are hereby incorporated by reference.

In embodiments, the linker is a synthetic linker such as PEG.

In embodiments, the linker is a polypeptide. In embodiments, the linker is less than about 500 amino acids long, about 450 amino acids long, about 400 amino acids long, about 350 amino acids long, about 300 amino acids long, about 250 amino acids long, about 200 amino acids long, about 150 amino acids long, or about 100 amino acids long. For example, the linker may be less than about 100, about 95, about 90, about 85, about 80, about 75, about 70, about 65, about 60, about 55, about 50, about 45, about 40, about 35, about about 25, about 20, about 19, about 18, about 17, about 16, about 15, about 14, about 13, about 12, about 11, about 10, about 9, about 8, about 7, about 6, about 5, about 4, about 3, or about 2 amino acids long. In embodiments, the linker is flexible. In another embodiment, the linker is rigid.

In embodiments, the linker is substantially comprised of glycine and serine residues (e.g., about 30%, or about 40%, or about 50%, or about 60%, or about 70%, or about 80%, or about 90%, or about 95%, or about 97%, or about 98%, or about 99%, or about 100% glycines and serines).

In embodiments, the linker is a hinge region of an antibody (e.g., of IgG, IgA, IgD, and IgE, inclusive of subclasses (e.g., IgG1, IgG2, IgG3, and IgG4, and IgA1 and IgA2)). The hinge region, found in IgG, IgA, IgD, and IgE class antibodies, acts as a flexible spacer, allowing the Fab portion to move freely in space. In contrast to the constant regions, the hinge domains are structurally diverse, varying in both sequence and length among immunoglobulin classes and subclasses. For example, the length and flexibility of the hinge region varies among the IgG subclasses. The hinge region of IgG1 encompasses amino acids 216-231 and, because it is freely flexible, the Fab fragments can rotate about their axes of symmetry and move within a sphere centered at the first of two inter-heavy chain disulfide bridges. IgG2 has a shorter hinge than IgG1, with 12 amino acid residues and four disulfide bridges. The hinge region of IgG2 lacks a glycine residue, is relatively short, and contains a rigid poly-proline double helix, stabilized by extra inter-heavy chain disulfide bridges. These properties restrict the flexibility of the IgG2 molecule. IgG3 differs from the other subclasses by its unique extended hinge region (about four times as long as the IgG1 hinge), containing 62 amino acids (including 21 prolines and 11 cysteines), forming an inflexible poly-proline double helix. In IgG3, the Fab fragments are relatively far away from the Fc fragment, giving the molecule a greater flexibility. The elongated hinge in IgG3 is also responsible for its higher molecular weight compared to the other subclasses. The hinge region of IgG4 is shorter than that of IgG1 and its flexibility is intermediate between that of IgG1 and IgG2. The flexibility of the hinge regions reportedly decreases in the order IgG3>IgG1>IgG4>IgG2. In embodiments, the linker may be derived from human IgG4 and contain one or more mutations to enhance dimerization (including S228P) or FcRn binding.

According to crystallographic studies, the immunoglobulin hinge region can be further subdivided functionally into three regions: the upper hinge region, the core region, and the lower hinge region. See Shin et al., 1992 Immunological Reviews 130:87. The upper hinge region includes amino acids from the carboxyl end of CH1 to the first residue in the hinge that restricts motion, generally the first cysteine residue that forms an interchain disulfide bond between the two heavy chains. The length of the upper hinge region correlates with the segmental flexibility of the antibody. The core hinge region contains the inter-heavy chain disulfide bridges, and the lower hinge region joins the amino terminal end of the C_{H2} domain and includes residues in C_{H2}. Id. The core hinge region of wild-type human IgG1 contains the sequence Cys-Pro-Pro-Cys which, when dimerized by disulfide bond formation, results in a cyclic octapeptide believed to act as a pivot, thus conferring flexibility. In embodiments, the present linker comprises, one, or two, or three of the upper hinge region, the core region, and the lower hinge region of any antibody (e.g., of IgG, IgA, IgD, and IgE, inclusive of subclasses (e.g., IgG1, IgG2, IgG3, and IgG4, and IgA1 and IgA2)). The hinge region may also contain one or more glycosylation sites, which include a number of structurally distinct types of sites for carbohydrate attachment. For example, IgA1 contains five glycosylation sites within a 17-amino-acid segment of the hinge region, conferring resistance of the hinge region polypeptide to intestinal proteases, considered an advantageous property for a secretory immunoglobulin. In embodiments, the linker of the present disclosure comprises one or more glycosylation sites.

In embodiments, the linker comprises an Fc domain of an antibody (e.g., of IgG, IgA, IgD, and IgE, inclusive of subclasses (e.g., IgG1, IgG2, IgG3, and IgG4, and IgA1 and IgA2)). In embodiments, the linker comprises a hinge-CH2-CH3 Fc domain derived from a human IgG4 antibody. In embodiments, the linker comprises a hinge-CH2-CH3 Fc domain derived from a human IgG1 antibody. In embodiments, the Fc domain exhibits increased affinity for and enhanced binding to the neonatal Fc receptor (FcRn). In embodiments, the Fc domain includes one or more mutations that increases the affinity and enhances binding to FcRn. Without wishing to be bound by theory, it is believed that increased affinity and enhanced binding to FcRn increases the in vivo half-life of the present chimeric proteins.

In embodiments, the Fc domain linker contains one or more amino acid substitutions at amino acid residue 250, 252, 254, 256, 308, 309, 311, 416, 428, 433 or 434 (in accordance with Kabat numbering, as in as in Kabat, et al., Sequences of Proteins of Immunological Interest, 5th Ed. Public Health Service, National Institutes of Health, Bethesda, Md. (1991) expressly incorporated herein by reference), or equivalents thereof. In an embodiment, the amino acid substitution at amino acid residue 250 is a substitution with glutamine. In an embodiment, the amino acid substitution at amino acid residue 252 is a substitution with tyrosine, phenylalanine, tryptophan or threonine. In an embodiment, the amino acid substitution at amino acid residue 254 is a substitution with threonine. In an embodiment, the amino acid substitution at amino acid residue 256 is a substitution with serine, arginine, glutamine, glutamic acid, aspartic acid, or threonine. In an embodiment, the amino acid substitution at amino acid residue 308 is a substitution with threonine. In an embodiment, the amino acid substitution at amino acid residue 309 is a substitution with proline. In an embodiment, the amino acid substitution at amino acid residue 311 is a substitution with serine. In an embodiment, the amino acid substitution at amino acid residue 385 is a substitution with arginine, aspartic acid, serine, threonine, histidine, lysine, alanine or glycine. In an embodiment, the amino acid substitution at amino acid residue 386 is a substitution with threonine, proline, aspartic acid, serine, lysine, arginine, isoleucine, or methionine. In an embodiment, the amino acid substitution at amino acid residue 387 is a substitution with arginine, proline, histidine, serine, threonine, or alanine. In an embodiment, the amino acid substitution at amino acid residue 389 is a substitution with proline, serine or asparagine. In an embodiment, the amino acid substitution at amino acid residue 416 is a substitution with leucine. In an embodiment, the amino acid substitution at amino acid residue 428 is a substitution with serine. In an embodiment, the amino acid substitution at amino acid residue 433 is a substitution with arginine, serine, isoleucine, proline, or glutamine. In an embodiment, the amino acid substitution at amino acid residue 434 is a substitution with histidine, phenylalanine, or tyrosine.

In embodiments, the Fc domain linker (e.g., comprising an IgG constant region) comprises one or more mutations such as substitutions at amino acid residue 252, 254, 256, 433, 434, or 436 (in accordance with Kabat numbering, as in as in Kabat, et al., Sequences of Proteins of Immunological Interest, 5th Ed. Public Health Service, National Institutes of Health, Bethesda, Md. (1991) expressly incorporated herein by reference). In an embodiment, the IgG constant region includes a triple M252Y/S254T/T256E mutation or YTE mutation. In another embodiment, the IgG constant region includes a triple H433K/N434F/Y436H mutation or KFH mutation. In a further embodiment, the IgG constant region includes an YTE and KFH mutation in combination.

In embodiments, the modified humanized antibodies of the invention comprise an IgG constant region that contains one or more mutations at amino acid residues 250, 253, 307, 310, 380, 416, 428, 433, 434, and 435. Illustrative mutations include T250Q, M428L, T307A, E380A, I253A, H310A, R416S, M428L, H433K, N434A, N434F, N434S, and H435A. In an embodiment, the IgG constant region comprises a M428L/N434S mutation or LS mutation. In another embodiment, the IgG constant region comprises a T250Q/M428L mutation or QL mutation. In another embodiment, the IgG constant region comprises an N434A mutation. In another embodiment, the IgG constant region comprises a T307A/E380A/N434A mutation or AAA mutation. In another embodiment, the IgG constant region comprises an I253A/H310A/H435A mutation or IHH mutation. In another embodiment, the IgG constant region comprises a H433K/N434F mutation. In another embodiment, the IgG constant region comprises a M252Y/S254T/T256E and a H433K/N434F mutation in combination.

Additional illustrative mutations in the IgG constant region are described, for example, in Robbie, et al., Antimicrobial Agents and Chemotherapy (2013), 57(12):6147-6153, Dall'Acqua et al., JBC (2006), 281(33):23514-24, Dall'Acqua et al., Journal of Immunology (2002), 169:5171-80, Ko et al. Nature (2014) 514:642-645, Grevys et al. Journal of Immunology. (2015), 194(11):5497-508, and U.S. Pat. No. 7,083,784, the entire contents of which are hereby incorporated by reference.

In embodiments, the linker comprises the amino acid sequence of SEQ ID NO: 46, or at least 90%, or 93%, or 95%, or 97%, or 98%, or 99% identity thereto. In embodiments, mutations are made to SEQ ID NO: 46 to increase stability and/or half-life. For instance, in embodiments, the linker has the amino acid sequence of SEQ ID NO: 47, or at least 90%, or 93%, or 95%, or 97%, or 98%, or 99% identity thereto. In embodiments, the linker comprises the amino acid sequence of SEQ ID NO: 48, or at least 90%, or 93%, or 95%, or 97%, or 98%, or 99% identity thereto.

In embodiments, the linker comprises the amino acid sequence of SEQ ID NO: 113, or at least 90%, or 93%, or 95%, or 97%, or 98%, or 99% identity thereto. In embodiments, mutations are made to SEQ ID NO: 113 to increase stability and/or half-life. For instance, in embodiments, the linker has the amino acid sequence of SEQ ID NO: 47, or at least 90%, or 93%, or 95%, or 97%, or 98%, or 99% identity thereto. In embodiments, the linker comprises the amino acid sequence of SEQ ID NO: 48, or at least 90%, or 93%, or 95%, or 97%, or 98%, or 99% identity thereto.

Without wishing to be bound by theory, including a linker comprising at least a part of an Fc domain in a chimeric protein, helps avoid formation of insoluble and, likely, non-functional protein concatemers and/or aggregates. This is in part due to the presence of cysteines in the Fc domain which are capable of forming disulfide bonds between chimeric proteins.

An illustrative Fc stabilizing mutant is S228P. Illustrative Fc half-life extending mutants are T250Q, M428L, V308T, L309P, and Q311S and the present linkers may comprise 1, or 2, or 3, or 4, or 5 of these mutants.

Further, one or more joining linkers may be employed to connect an Fc domain in a linker (e.g., one of SEQ ID NO: 46, SEQ ID NO: 47, SEQ ID NO: 48, SEQ ID NO: 112, or SEQ ID NO: 113, or at least 90%, or 93%, or 95%, or 97%, or 98%, or 99% identity thereto) and the extracellular domains. For example, any one of SEQ ID NO: 49, SEQ ID NO: 50, SEQ ID NO: 51, SEQ ID NO: 52, SEQ ID NO: 53, SEQ ID NO: 54, or variants thereof may connect an extracellular domain as described herein and a linker as described herein. Optionally, any one of SEQ ID NO: 49, SEQ ID NO: 50, SEQ ID NO: 51, SEQ ID NO: 52, SEQ ID NO: 53, SEQ ID NO: 54, or variants thereof are displaced between an extracellular domain as described herein and a linker as described herein. Optionally, any one of SEQ ID NOs: 49 to 95, or variants thereof are located between an extracellular domain as described herein and an Fc domain as described herein. In embodiments, a chimeric protein comprises one joining linker preceding an Fc domain and a second joining linker following the Fc domain; thus, a chimeric protein may comprise the following structure:
ECD 1—Joining Linker 1—Fc Domain—Joining Linker 2—ECD 2.

In embodiments, the first and second joining linkers may be different or they may be the same.

The amino acid sequences of illustrative linkers are provided in Table 1 below:

TABLE 1
Illustrative linkers
(Fc domain linkers and joining linkers)

| SEQ ID | |
|---|---|
| NO. | Sequence |
| 46 | APEFLGGPSVFLFPPKPKDTLMISRTPEVTCVVVDVSQED |
| | PEVQFNWYVDGVEVHNAKTKPREEQFNSTYRVVSVLTVLH |
| | QDWLSGKEYKCKVSSKGLPSSIEKTISNATGQPREPQVYT |
| | LPPSQEEMTKNQVSLTCLVKGFYPSDIAVEWESNGQPENN |
| | YKTTPPVLDSDGSFFLYSRLTVDKSSWQEGNVFSCSVMHE |
| | ALHNHYTQKSLSLSLGK |
| 47 | APEFLGGPSVFLFPPKPKDQLMISRTPEVTCVVVDVSQED |
| | PEVQFNWYVDGVEVHNAKTKPREEQFNSTYRVVSVLTTPH |
| | SDWLSGKEYKCKVSSKGLPSSIEKTISNATGQPREPQVYT |
| | LPPSQEEMTKNQVSLTCLVKGFYPSDIAVEWESNGQPENN |
| | YKTTPPVLDSDGSFFLYSRLTVDKSSWQEGNVFSCSVLHE |
| | ALHNHYTQKSLSLSLGK |
| 48 | APEFLGGPSVFLFPPKPKDQLMISRTPEVTCVVVDVSQED |
| | PEVQFNWYVDGVEVHNAKTKPREEQFNSTYRVVSVLTVLH |
| | QDWLSGKEYKCKVSSKGLPSSIEKTISNATGQPREPQVYT |
| | LPPSQEEMTKNQVSLTCLVKGFYPSDIAVEWESNGQPENN |
| | YKTTPPVLDSDGSFFLYSRLTVDKSRWQEGNVFSCSVLHE |
| | ALHNHYTQKSLSLSLGK |
| 112 | EPKSCDKTHTCPPCPAPELLGGPSVFLFPPKPKDTLMISR |
| | TPEVTCVVVDVSHEDPEVKFNWYVDGVEVHNAKTKPREEQ |
| | YASTYRVVSVLTVLHQDWLNGKEYKCKVSNKALPAPIEKT |
| | ISKAKGQPREPQVYTLPPSRDELTKNQVSLTCLVKGFYPS |
| | DIAVEWESNGQPENNYKTTPPVLDSDGSFFLYSKLTVDKS |
| | RWQQGNVFSCSVMHEALHNHYTQKSLSLSPGK |
| 113 | EPKSCDKTHTCPPCPAPEAAGGPSVFLFPPKPKDTLMISR |
| | TPEVTCVVVDVSHEDPEVKFNWYVDGVEVHNAKTKPREEQ |
| | YASTYRVVSVLTVLHQDWLNGKEYKCKVSNKALPAPIEKT |
| | ISKAKGQPREPQVYTLPPSRDELTKNQVSLTCLVKGFYPS |
| | DIAVEWESNGQPENNYKTTPPVLDSDGSFFLYSKLTVDKS |
| | RWQQGNVFSCSVMHEALHNHYTQKSLSLSPGK |
| 49 | SKYGPPCPSCP |
| 50 | SKYGPPCPPCP |
| 51 | SKYGPP |
| 52 | IEGRMD |
| 53 | GGGVPRDCG |
| 54 | IEGRMDGGGGAGGGG |
| 55 | GGGSGGGS |
| 56 | GGGSGGGGSGGG |
| 57 | EGKSSGSGSESKST |
| 58 | GGSG |
| 59 | GGSGGGSGGGSG |
| 60 | EAAAKEAAAKEAAAK |
| 61 | EAAAREAAAREAAAREAAAR |
| 62 | GGGGSGGGGSGGGGSAS |
| 63 | GGGGAGGGG |
| 64 | GS or GGS or LE |
| 65 | GSGSGS |
| 66 | GSGSGSGSGS |
| 67 | GGGGSAS |
| 68 | APAPAPAPAPAPAPAPAPAP |
| 69 | CPPC |
| 70 | GGGGS |
| 71 | GGGGSGGGGS |
| 72 | GGGGSGGGGSGGGGS |
| 73 | GGGGSGGGGSGGGGSGGGGS |
| 74 | GGGGSGGGGSGGGGSGGGGSGGGGS |
| 75 | GGGGSGGGGGGGGSGGGGSGGGGSGGGGS |
| 76 | GGGGSGGGGSGGGGSGGGGSGGGGSGGGGSGGGGS |
| 77 | GGGGSGGGGSGGGGSGGGGSGGGGSGGGGSGGGGSGGGGS |
| 78 | GGSGGSGGGGSGGGGS |
| 79 | GGGGGGGG |
| 80 | GGGGGG |
| 81 | EAAAK |
| 82 | EAAAKEAAAK |
| 83 | EAAAKEAAAKEAAAK |
| 84 | AEAAAKEAAAKA |
| 85 | AEAAAKEAAAKEAAAKA |
| 86 | AEAAAKEAAAKEAAAKEAAAKA |
| 87 | AEAAAKEAAAKEAAAKEAAAKEAAAKA |
| 88 | AEAAAKEAAAKEAAAKEAAAKALEAEAAAKEAAAKEAAAK |
| | EAAAKA |
| 89 | PAPAP |
| 90 | KESGSVSSEQLAQFRSLD |
| 91 | GSAGSAAGSGEF |
| 92 | GGGSE |
| 93 | GSESG |
| 94 | GSEGS |
| 95 | GEGGSGEGSSGEGSSSEGGGSEGGGSEGGGSEGGS |

Additional illustrative joining linkers include, but are not limited to, linkers having the sequence LE, GGGGS (SEQ ID NO: 70), (GGGGS)ₙ (n=1-4) (SEQ ID NO: 70-73), (Gly)₈ (SEQ ID NO: 79), (Gly)₆ (SEQ ID NO: 80), (EAAAK)ₙ (n=1-3) (SEQ ID NO: 81-83), A(EAAAK)ₙA (n=2-5) (SEQ ID NO: 84-87), AEAAAKEAAAKA (SEQ ID NO: 84), A(EAAAK)₄ALEA(EAAAK)₄A (SEQ ID NO: 88), PAPAP (SEQ ID NO: 89), KESGSVSSEQLAQFRSLD (SEQ ID NO: 90), EGKSSGSGSESKST (SEQ ID NO: 57), GSAGSAAGSGEF (SEQ ID NO: 91), and (XP)ₙ, with X designating any amino acid, e.g., Ala, Lys, or Glu.

In embodiments, the joining linker is substantially comprised of glycine and serine residues (e.g., about 30%, or about 40%, or about 50%, or about 60%, or about 70%, or about 80%, or about 90%, or about 95%, or about 97%, or about 98%, or about 99%, or about 100% glycines and serines). For example, in embodiments, the joining linker is (Gly4Ser)ₙ, where n is from about 1 to about 8, e.g., 1, 2, 3, 4, 5, 6, 7, or 8 (SEQ ID NO: to SEQ ID NO: 77, respectively). In embodiments, the joining linker sequence is GGSGGSGGGGSGGGGS (SEQ ID NO: 78). Additional illustrative joining linkers include, but are not limited to, linkers having the sequence LE, (Gly)₈ (SEQ ID NO: 79), (Gly)₆ (SEQ ID NO: 80), (EAAAK)ₙ (n=1-3) (SEQ ID NO: 81-SEQ ID NO: 83), A(EAAAK)ₙA (n=2-5) (SEQ ID NO: 84-SEQ ID NO: 87), A(EAAAK)₄ALEA(EAAAK)₄A (SEQ ID NO: 88), PAPAP (SEQ ID NO: 89), KESGSVSSEQLAQFRSLD (SEQ ID NO: 90), GSAGSAAGSGEF (SEQ ID NO: 91), and (XP)ₙ, with X designating any amino acid, e.g., Ala, Lys, or Glu. In embodiments, the joining linker is GGS.

In embodiments, the joining linker is one or more of GGGSE (SEQ ID NO: 92), GSESG (SEQ ID NO: 93), GSEGS (SEQ ID NO: 94), GEGGSGEGSSGEGSSSEGGGSEGGGSEGGGSEGGS (SEQ ID NO: 95), and a joining linker of randomly placed G, S, and E every 4 amino acid intervals.

In embodiments, a chimeric protein comprises a modular linker.

In embodiments, the linker may be flexible, including without limitation highly flexible. In embodiments, the linker may be rigid, including without limitation a rigid alpha helix.

In embodiments, the linker may be functional. For example, without limitation, the linker may function to improve the folding and/or stability, improve the expression, improve the pharmacokinetics, and/or improve the bioactivity of the present chimeric protein. In another example, the linker may function to target the chimeric protein to a particular cell type or location.

In embodiments, the present chimeric proteins are capable of, and can be used in methods comprising, promoting immune activation (e.g., against tumors). In embodiments, the present chimeric proteins are capable of, and can be used in methods comprising, suppressing immune inhibition (e.g., that allows tumors to survive). In embodiments, the present chimeric proteins provide improved immune activation and/or improved suppression of immune inhibition due to the proximity of signaling that is provided by the chimeric nature of the constructs.

In embodiments, the present chimeric proteins are capable of, or can be used in methods comprising, modulating the amplitude of an immune response, e.g., modulating the level of effector output. In embodiments, e.g., when used for the treatment of cancer, the present chimeric proteins alter the extent of immune stimulation as compared to immune inhibition to increase the amplitude of a T cell response, including, without limitation, stimulating increased levels of cytokine production, proliferation or target killing potential.

In embodiments the present chimeric proteins, in embodiments are capable of, or find use in methods involving, masking an inhibitory ligand on the surface of a tumor cell and replacing that immune inhibitory ligand with an immune stimulatory ligand. Accordingly, the present chimeric proteins, in embodiments are capable of, or find use in methods involving, reducing or eliminating an inhibitory immune signal and/or increasing or activating an immune stimulatory signal. For example, a tumor cell bearing an inhibitory signal (and thus evading an immune response) may be substituted for a positive signal binding on a T cell that can then attack a tumor cell. Accordingly, in embodiments, an inhibitory immune signal is masked by the present constructs and a stimulatory immune signal is activated. Such beneficial properties are enhanced by the single construct approach of the present chimeric proteins. For instance, the signal replacement can be effected nearly simultaneously and the signal replacement is tailored to be local at a site of clinical importance (e.g., the tumor microenvironment). Further embodiments apply the same principle to other chimeric protein constructs, such as, for example, (i) the extracellular domain of TIGIT and (ii) extracellular domain of 4-1BBL; (i) the extracellular domain of TIGIT and (ii) extracellular domain of GITRL; (i) the extracellular domain of TIGIT and (ii) extracellular domain of TL1A; (i) the extracellular domain of TIGIT and (ii) extracellular domain of LIGHT; and (i) the extracellular domain of PD-1 and (ii) extracellular domain of LIGHT; and (i) the extracellular domain of CD172a(SIRPα) and (ii) extracellular domain of LIGHT; and (i) the extracellular domain of TIGIT and (ii) extracellular domain of LIGHT; (i) the extracellular domain of CD172a(SIRPα) and (ii) extracellular domain of 4-1BBL; (i) the extracellular domain of CD172a(SIRPα) and (ii) extracellular domain of GITRL; (i) the extracellular domain of CD172a(SIRPα) and (ii) extracellular domain of TL1A; (i) the extracellular domain of CD172a(SIRPα) and (ii) extracellular domain of LIGHT; and (i) the extracellular domain of PD-1 and (ii) extracellular domain of LIGHT; and (i) the extracellular domain of CD172a(SIRPα) and (ii) extracellular domain of LIGHT; and (i) the extracellular domain of CD172a(SIRPα) and (ii) extracellular domain of LIGHT; among others.

In embodiments, the present chimeric proteins are capable of, or find use in methods comprising, stimulating or enhancing the binding of immune stimulatory receptor/ligand pairs. Illustrative T cell costimulatory receptors and their ligands include OX-40:OX40-L, CD27:CD70, CD30:CD3O-L, CD40:CD4O-L; CD137:CD137-L, HVEM:LIGHT, GITR:GITR-L, TNFRSF25:TL1A, DR5:TRAIL, and BTLA:HVEM. In embodiments, the present chimeric proteins are capable of, or find use in methods comprising, inhibiting or reducing the binding of immune inhibitory receptor/ligand pairs. Illustrative T cell coinhibitory receptors and their ligands include, for example, CTLA-4:CD80/CD86, PD-1:PD-L1/PD-L2, BTLA:HVEM, TIM-3:galectin-9/phosphatidylserine, TIGIT/CD155 or CD112, CD172a(SIRPα)/CD47, B7H3R/B7H3, B7H4R/B7H4, CD244/CD48, TMIGD2/HHLA2, among others.

In embodiments, the present chimeric protein blocks, reduces and/or inhibits PD-1 and PD-L1 or PD-L2 and/or the binding of PD-1 with PD-L1 or PD-L2. In embodiments, the present chimeric protein blocks, reduces and/or inhibits the activity of CTLA-4 and/or the binding of CTLA-4 with one or more of AP2M1, CD80, CD86, SHP-2, and PPP2R5A. In embodiments, the present chimeric protein increases and/or stimulates GITR and/or the binding of GITR with one or more of GITR ligand. In embodiments, the present chimeric protein increases and/or stimulates OX40 and/or the binding of OX40 with one or more of OX40 ligand.

In embodiments, the present chimeric proteins are capable of, or find use in methods involving, enhancing, restoring, promoting and/or stimulating immune modulation. In embodiments, the present chimeric proteins described herein, restore, promote and/or stimulate the activity or activation of one or more immune cells against tumor cells including, but not limited to: T cells, cytotoxic T lymphocytes, T helper cells, natural killer (NK) cells, natural killer T (NKT) cells, anti-tumor macrophages (e.g., M1 macrophages), B cells, and dendritic cells. In embodiments, the present chimeric proteins enhance, restore, promote and/or stimulate the activity and/or activation of T cells, including, by way of a non-limiting example, activating and/or stimulating one or more T-cell intrinsic signals, including a pro-survival signal; an autocrine or paracrine growth signal; a p38 MAPK-, ERK-, STAT-, JAK-, AKT- or PI3K-mediated signal; an anti-apoptotic signal; and/or a signal promoting and/or necessary for one or more of: proinflammatory cytokine production or T cell migration or T cell tumor infiltration.

In embodiments, the present chimeric proteins are capable of, or find use in methods involving, causing an increase of one or more of T cells (including without limitation cytotoxic T lymphocytes, T helper cells, natural killer T (NKT) cells), B cells, natural killer (NK) cells, natural killer T (NKT) cells, dendritic cells, monocytes, and macrophages (e.g., one or more of M1 and M2) into a tumor or the tumor microenvironment. In embodiments, the present chimeric proteins are capable of, or find use in methods involving, inhibiting and/or causing a decrease in recruitment of immunosuppressive cells (e.g., myeloid-derived suppressor cells (MDSCs), regulatory T cells (Tregs), tumor associated neutrophils (TANs), M2 macrophages, and tumor associated macrophages (TAMs)) to the tumor and/or tumor microenvironment (TME). In embodiments, the present therapies may alter the ratio of M1 versus M2 macrophages in the tumor site and/or TME to favor M1 macrophages.

In embodiments, the present chimeric proteins are capable of, and can be used in methods comprising, inhibiting and/or reducing T cell inactivation and/or immune tolerance to a tumor, comprising administering an effective amount of a chimeric protein described herein to a subject. In embodiments, the present chimeric proteins are able to increase the serum levels of various cytokines including, but not limited to, one or more of IFNγ, TNFα, IL-2, IL-4, IL-5, IL-6, IL-9, IL-10, IL-13, IL-17A, IL-17F, and IL-22. In embodiments, the present chimeric proteins are capable of enhancing IL-2, IL-4, IL-5, IL-10, IL-13, IL-17A, IL-22, TNFα or IFNγ in the serum of a treated subject. In embodiments, administration of the present chimeric protein is capable of enhancing TNFα secretion. In a specific embodiment, administration of the present chimeric protein is capable of enhancing superantigen mediated TNFα secretion by leukocytes. Detection of such a cytokine response may provide a method to determine the optimal dosing regimen for the indicated chimeric protein.

In embodiments, the present chimeric proteins inhibit, block and/or reduce cell death of an anti-tumor CD8+ and/or CD4+ T cell; or stimulate, induce, and/or increase cell death of a pro-tumor T cell. T cell exhaustion is a state of T cell dysfunction characterized by progressive loss of proliferative and effector functions, culminating in clonal deletion. Accordingly, a pro-tumor T cell refers to a state of T cell dysfunction that arises during many chronic infections and cancer. This dysfunction is defined by poor proliferative and/or effector functions, sustained expression of inhibitory receptors and a transcriptional state distinct from that of functional effector or memory T cells. Exhaustion prevents optimal control of infection and tumors. In addition, an anti-tumor CD8+ and/or CD4+ T cell refers to T cells that can mount an immune response to a tumor. Illustrative pro-tumor T cells include, but are not limited to, Tregs, CD4+ and/or CD8+ T cells expressing one or more checkpoint inhibitory receptors, Th2 cells and Th17 cells. Checkpoint inhibitory receptors refers to receptors expressed on immune cells that prevent or inhibit uncontrolled immune responses.

In embodiments, the present chimeric proteins are capable of, and can be used in methods comprising, increasing a ratio of effector T cells to regulatory T cells. Illustrative effector T cells include ICOS+ effector T cells; cytotoxic T cells (e.g., αβ TCR, CD3⁺, CD8⁺, CD45RO⁺); CD4⁺ effector T cells (e.g., αβ TCR, CD3⁺, CD4⁺, CCR7⁺, CD62Lhi, IL⁻7R/CD127⁺); CD8⁺ effector T cells (e.g., αβ TCR, CD3⁺, CD8⁺, CCR7⁺, CD62Lhi, IL⁺7R/CD127⁺); effector memory T cells (e.g., CD62Llow, CD44⁺, TCR, CD3⁺, IL⁻7R/CD127⁺, IL-15R⁺, CCR7low); central memory T cells (e.g., CCR7⁺, CD62L⁺, CD27⁺; or CCR7hi, CD44⁺, CD62Lhi, TCR, CD3⁺, IL-7R/CD127⁺, IL-15R⁺); CD62L⁺ effector T cells; CD8⁺ effector memory T cells (TEM) including early effector memory T cells (CD27⁺CD62L⁻) and late effector memory T cells (CD27⁻CD62L⁻) (TemE and TemL, respectively); CD127(⁺)CD25(low/−) effector T cells; CD127(⁻)CD25(⁻) effector T cells; CD8⁺ stem cell memory effector cells (TSCM) (e.g., CD44(low)CD62L(high)CD122(high)sca(⁺)); TH1 effector T-cells (e.g., CXCR3⁺, CXCR6⁺ and CCR5⁺; or αβ TCR, CD3⁺, CD4⁺, IL-12R⁺, IFNγR⁺, CXCR3⁺), TH2 effector T cells (e.g., CCR3⁺, CCR4⁺ and CCR8⁺; or αβ TCR, CD3⁺, CD4⁺, IL-4R⁺, IL-33R⁺, CCR4⁺, IL-17RB⁺, CRTH2⁺); TH9 effector T cells (e.g., αβ TCR, CD3⁺, CD4⁺); TH17 effector T cells (e.g., αβ TCR, CD3⁺, CD4⁺, IL-23R⁺, CCR6⁺, IL-1R⁺); CD4⁺CD45RO⁺CCR7⁺ effector T cells, CD4⁺CD45RO⁺CCR7(−) effector T cells; and effector T cells secreting IL-2, IL-4 and/or IFN-γ. Illustrative regulatory T cells include ICOS⁺ regulatory T cells, CD4⁺CD25⁺FOXP3⁺ regulatory T cells, CD4⁺CD25⁺ regulatory T cells, CD4⁺CD25⁻ regulatory T cells, CD4⁺CD25high regulatory T cells, TIM-3⁺PD-1⁺ regulatory T cells, lymphocyte activation gene-3 (LAG-3)⁺ regulatory T cells, CTLA-4/CD152⁺ regulatory T cells, neuropilin-1 (Nrp-1)⁺ regulatory T cells, CCR4⁺CCR8⁺ regulatory T cells, CD62L (L-selectin)⁺ regulatory T cells, CD45RBlow regulatory T cells, CD1271ow regulatory T cells, LRRC32/GARP⁺ regulatory T cells, CD39⁺ regulatory T cells, GITR⁺ regulatory T cells, LAP⁺ regulatory T cells, 1B11⁺ regulatory T cells, BTLA⁺ regulatory T cells, type 1 regulatory T cells (Tr1 cells),T helper type 3 (Th3) cells, regulatory cell of natural killer T cell phenotype (NKTregs), CD8⁺ regulatory T cells, CD8⁺CD28⁻ regulatory T cells and/or regulatory T-cells secreting IL-10, IL-35, TGF-8, TNF-α, Galectin-1, IFN-γ and/or MCP1.

In embodiments, the chimeric protein generates a memory response which may, e.g., be capable of preventing relapse or protecting the animal from a rechallenge. Thus, an animal treated with the chimeric protein is later able to attack tumor cells and/or prevent development of tumors when rechallenged after an initial treatment with the chimeric protein. Accordingly, a chimeric protein of the present disclosure stimulates both active tumor destruction and also immune recognition of tumor antigens, which are essential in programming a memory response capable of preventing relapse.

In embodiments, the present chimeric proteins are capable of, and can be used in methods comprising, transiently stimulating effector T cells for no longer than about 12 hours, about 24 hours, about 48 hours, about 72 hours or about 96 hours or about 1 week or about 2 weeks. In embodiments, the present chimeric proteins are capable of, and can be used in methods comprising, transiently depleting or inhibiting regulatory T cells for no longer than about 12 hours, about 24 hours, about 48 hours, about 72 hours or about 96 hours or about 1 week or about 2 weeks. In embodiments, the transient stimulation of effector T cells and/or transient depletion or inhibition of regulatory T cells occurs substantially in a patient's bloodstream or in a particular tissue/location including lymphoid tissues such as for example, the bone marrow, lymph-node, spleen, thymus, mucosa-associated lymphoid tissue (MALT), non-lymphoid tissues, or in the tumor microenvironment.

In embodiments, the present chimeric proteins provide advantages including, without limitation, ease of use and ease of production. This is because two distinct immunotherapy agents are combined into a single product which allows for a single manufacturing process instead of two independent manufacturing processes. In addition, administration of a single agent instead of two separate agents allows for easier administration and greater patient compliance. Further, in contrast to, for example, monoclonal antibodies, which are large multimeric proteins containing numerous disulfide bonds and post-translational modifications such as glycosylation, the present chimeric proteins are easier and more cost effective to manufacture.

In embodiments, the present chimeric protein is producible in a mammalian host cell as a secretable and fully functional single polypeptide chain.

In embodiments, the present chimeric protein unexpectedly provides binding of the extracellular domain components to their respective binding partners with slow off rates (K_{d} or K_{off}). In embodiments, this provides an unexpectedly long interaction of the receptor to ligand and vice versa. Such an effect allows for a sustained negative signal masking effect. Further, in embodiments, this delivers a longer positive signal effect, e.g., to allow an effector cell to be adequately stimulated for an anti-tumor effect. For example, the present chimeric protein, e.g., via the long off rate binding allows sufficient signal transmission to provide T cell proliferation and allow for anti-tumor attack. By way of further example, the present chimeric protein, e.g., via the long off rate binding allows sufficient signal transmission to provide release of stimulatory signals, such as, for example, cytokines.

The stable synapse of cells promoted by the present agents (e.g., a tumor cell bearing negative signals and a T cell which could attack the tumor) provides spatial orientation to favor tumor reduction—such as positioning the T cells to attack tumor cells and/or sterically preventing the tumor cell from delivering negative signals, including negative signals beyond those masked by the chimeric protein of the invention.

In embodiments, this provides longer on-target (e.g., intra-tumoral) half-life (tip) as compared to serum tip of the chimeric proteins. Such properties could have the combined advantage of reducing off-target toxicities which may be associated with systemic distribution of the chimeric proteins.

In embodiments, the present agents allow certain immune cells to act, e.g., in an antitumoral manner, by preventing and/or disrupting inhibition of NK cells and/or subsets of activated, memory and/or regulatory T cells, and/or helper T cells by blocking a signal via TIGIT and, optionally, create further immune responses via 4-1BBL-, and/or GITRL-, and/or TL1A-, and/or LIGHT-based stimulatory signaling.

In embodiments, the present agents allow certain immune cells to act, e.g., in an antitumoral manner, by stimulating and/or increasing stimulatory LIGHT-based signaling, e.g., on visceral and/or lymphoid and/or other stroma and/or epithelia and/or myeloid cells and, optionally, create further immune responses via blockade or reduction of PD-1−, and/or CD172a(SIRPα)−, and/or TIGIT-based inhibitory signaling.

Further, in embodiments, the present chimeric proteins provide synergistic therapeutic effects as it allows for improved site-specific interplay of two immunotherapy agents.

In embodiments, the present chimeric proteins provide the potential for reducing off-site and/or systemic toxicity.

In embodiments, the present chimeric proteins provide reduced side-effects, e.g., GI complications, relative to current immunotherapies, e.g., antibodies directed to checkpoint molecules as described herein. Illustrative GI complications include abdominal pain, appetite loss, autoimmune effects, constipation, cramping, dehydration, diarrhea, eating problems, fatigue, flatulence, fluid in the abdomen or ascites, gastrointestinal (GI) dysbiosis, GI mucositis, inflammatory bowel disease, irritable bowel syndrome (IBS-D and IBS-C), nausea, pain, stool or urine changes, ulcerative colitis, vomiting, weight gain from retaining fluid, and/or weakness.

In embodiments, the linker is a polypeptide selected from a flexible amino acid sequence, an IgG hinge region, or an antibody sequence. In embodiments, the linker comprises hinge-CH2-CH3 Fc domain derived from IgG1 or IgG4. In embodiments, the hinge-CH2-CH3 Fc domain is derived from human IgG1. In embodiments, the hinge-CH2-CH3 Fc domain is derived from human IgG4. In embodiments, the linker comprises an amino acid sequence that is at least 95%, or at least 96%, or at least 97%, or at least 98%, or at least 99% identical to the amino acid sequence of SEQ ID NO: 46, SEQ ID NO: 47, SEQ ID NO: 112, or SEQ ID NO: 113. In embodiments, the linker comprises two or more joining linkers each joining linker independently selected from SEQ ID NOs: 49-95; wherein one joining linker is N terminal to the hinge-CH2-CH3 Fc domain and another joining linker is C terminal to the hinge-CH2-CH3 Fc domain.

In embodiments, the first domain comprises an amino acid sequence that is at least 95%, or at least 96%, or at least 97%, or at least 98%, or at least 99% identical to the amino acid sequence of SEQ ID NO: 10. In embodiments, the first domain comprises an amino acid sequence that is at least 95%, or at least 96%, or at least 97%, or at least 98%, or at least 99% identical to the amino acid sequence of SEQ ID NO: 2. In embodiments, chimeric protein comprises an amino acid sequence that is at least 95%, or at least 96%, or at least 97%, or at least 98%, or at least 99% identical to the amino acid sequence of SEQ ID NO: 11. In embodiments, the chimeric protein is a recombinant fusion protein.

Methods of Treatment

In one aspect, the present disclosure relates to a method for treating a cancer in a subject in need thereof the method comprising a step of administering to the subject an effective amount of a chimeric protein having a general structure of: N terminus—(a)—(b)—(c)—C terminus, wherein: (a) is a first domain comprising an extracellular domain of human T cell immunoreceptor with Ig and ITIM domains (TIGIT), (b) is a linker adjoining the first and second domains, wherein the linker comprises a hinge-CH2-CH3 Fc domain, and (c) is a second domain comprising an extracellular domain of human LIGHT (lymphotoxin-like, exhibits inducible expression and competes with HSV glycoprotein D for herpesvirus entry mediator (HVEM), a receptor expressed by T lymphocytes). In embodiments, the dose of the chimeric protein administered is between about 0.0001 mg/kg and about 50.0 mg/kg, optionally selected from about 1 mg/kg, about 3 mg/kg, about 6 mg/kg, or about 10 mg/kg, about 12 mg/kg, about 15 mg/kg, about 18 mg/kg, about 20 mg/kg, about 22 mg/kg, about 25 mg/kg, about 27 mg/kg, about 30 mg/kg, about 33 mg/kg, about 35 mg/kg, about 37 mg/kg, about mg/kg, about 42 mg/kg, about 45 mg/kg, about 48 mg/kg, and about 50 mg/kg. in embodiments, the subject is a human, optionally an adult human.

In embodiments, the chimeric protein is administered at least about one time a week. In embodiments, the chimeric protein is administered at least about one time a month. In embodiments, the chimeric protein is administered at least about two times a month. In embodiments, the chimeric protein is administered at least about three times a month.

In embodiments, the cancer comprises a solid tumor (local and/or metastatic) or a lymphoma. In embodiments, the cancer is an advanced cancer. In embodiments, the cancer is selected from Hodgkin's and non-Hodgkin's lymphoma, B-cell lymphoma (including low grade/follicular non-Hodgkin's lymphoma (NHL); small lymphocytic (SL) NHL; intermediate grade/follicular NHL; intermediate grade diffuse NHL; high grade immunoblastic NHL; high grade lymphoblastic NHL; high grade small non-cleaved cell NHL; bulky disease NHL; mantle cell lymphoma; AIDS-related lymphoma; and Waldenstrom's Macroglobulinemia; chronic lymphocytic leukemia (CLL); acute lymphoblastic leukemia (ALL); Hairy cell leukemia; or chronic myeloblastic leukemia, basal cell carcinoma, biliary tract cancer; bladder cancer; bone cancer; brain and central nervous system cancer; breast cancer; cancer of the peritoneum; cervical cancer; choriocarcinoma; colon and rectum cancer; connective tissue cancer; cancer of the digestive system; endometrial cancer; esophageal cancer; eye cancer; cancer of the head and neck; gastric cancer (including gastrointestinal cancer); glioblastoma; hepatic carcinoma; hepatoma; intra-epithelial neoplasm; kidney or renal cancer; larynx cancer; leukemia; liver cancer; lung cancer (e.g., small-cell lung cancer, non-small cell lung cancer, adenocarcinoma of the lung, and squamous carcinoma of the lung); melanoma; myeloma; neuroblastoma; oral cavity cancer (lip, tongue, mouth, and pharynx); ovarian cancer; pancreatic cancer; prostate cancer; retinoblastoma; rhabdomyosarcoma; rectal cancer; cancer of the respiratory system; salivary gland carcinoma; sarcoma; skin cancer; squamous cell cancer; stomach cancer; testicular cancer; thyroid cancer; uterine or endometrial cancer; cancer of the urinary system; vulval cancer; lymphoma including Hodgkin's and non-Hodgkin's lymphoma, as well as B-cell lymphoma (including low grade/follicular non-Hodgkin's lymphoma (NHL); small lymphocytic (SL) NHL; intermediate grade/follicular NHL; intermediate grade diffuse NHL; high grade immunoblastic NHL; high grade lymphoblastic NHL; high grade small non-cleaved cell NHL; bulky disease NHL; mantle cell lymphoma; AIDS-related lymphoma; and Waldenstrom's Macroglobulinemia; chronic lymphocytic leukemia (CLL); acute lymphoblastic leukemia (ALL); Hairy cell leukemia; chronic myeloblastic leukemia; as well as other carcinomas and sarcomas; and post-transplant lymphoproliferative disorder (PTLD), as well as abnormal vascular proliferation associated with phakomatoses, edema (e.g., that associated with brain tumors), Meigs' syndrome cancer; renal carcinoma; colorectal cancer; and adrenal cancer.

In one aspect, the present disclosure relates to a method for inducing lymphocyte expansion in a subject in need thereof, the method comprising a step of administering to the subject an effective amount of a chimeric protein having a general structure of: N terminus—(a)—(b)—(c)—C terminus, wherein: (a) is a first domain comprising an extracellular domain of human T cell immunoreceptor with Ig and ITIM domains (TIGIT), (b) is a linker adjoining the first and second domains, wherein the linker comprises a hinge-CH2-CH3 Fc domain, and (c) is a second domain comprising an extracellular domain of human LIGHT (lymphotoxin-like, exhibits inducible expression and competes with HSV glycoprotein D for herpesvirus entry mediator (HVEM), a receptor expressed by T lymphocytes).

In one aspect, the present disclosure relates to a method for inducing lymphocyte margination in a subject in need thereof, the method comprising a step of administering to the subject an effective amount of a chimeric protein having a general structure of: N terminus—(a)—(b)—(c)—C terminus, wherein: (a) is a first domain comprising an extracellular domain of human T cell immunoreceptor with Ig and ITIM domains (TIGIT), (b) is a linker adjoining the first and second domains, wherein the linker comprises a hinge-CH2-CH3 Fc domain, and (c) is a second domain comprising an extracellular domain of human LIGHT (lymphotoxin-like, exhibits inducible expression and competes with HSV glycoprotein D for herpesvirus entry mediator (HVEM), a receptor expressed by T lymphocytes).

In embodiments, the subject is dosed with a dosing regimen selected from about every 3 days to about every days, about every week to about every 2 weeks, about every 10 days to about every 3 weeks, about every 2 weeks to about every 4 weeks, about every 3 weeks to about every 5 weeks, about every 4 weeks to about every 6 weeks, about every 5 weeks to about every 7 weeks, about every 6 weeks to about every 8 weeks, and about every 6 weeks to about every 2 months.

In embodiments, the first domain is capable of binding a TIGIT ligand. In embodiments, the first domain comprises substantially all of the extracellular domain of TIGIT. In embodiments, the second domain is capable of binding a LIGHT receptor. In embodiments, the second domain comprises substantially all of the extracellular domain of LIGHT.

In embodiments, the linker is a polypeptide selected from a flexible amino acid sequence, an IgG hinge region, and/or an antibody sequence. In embodiments, the linker comprises hinge-CH2-CH3 Fc domain derived from IgG1 or IgG4. In embodiments, the hinge-CH2-CH3 Fc domain is derived from human IgG1 or human IgG4. In embodiments, the linker comprises an amino acid sequence that is at least 95%, or at least 96%, or at least 97%, or at least 98%, or at least 99% identical to the amino acid sequence of SEQ ID NO: 46, SEQ ID NO: 47, SEQ ID NO: 112, or SEQ ID NO: 113. In embodiments, the linker comprises one or more joining linkers, such joining linkers independently selected from SEQ ID NOs: 49-95. In embodiments, the linker comprises two or more joining linkers each joining linker independently selected from SEQ ID NOs: 49-95; wherein one joining linker is N terminal to the hinge-CH2-CH3 Fc domain and another joining linker is C terminal to the hinge-CH2-CH3 Fc domain.

In embodiments, the first domain comprises an amino acid sequence that is at least 95%, or at least 96%, or at least 97%, or at least 98%, or at least 99% identical to the amino acid sequence of SEQ ID NO: 10. In embodiments, the second domain comprises an amino acid sequence that is at least 95%, or at least 96%, or at least 97%, or at least 98%, or at least 99% identical to the amino acid sequence of SEQ ID NO: 2.

In embodiments, (a) the first domain comprises an amino acid sequence that is at least 95% identical to the amino acid sequence of SEQ ID NO: 10, (b) the second domain comprises the amino acid sequence that is at least 95% identical to the amino acid sequence of SEQ ID NO: 2, and (c) the linker comprises an amino acid sequence that is at least 95% identical to the amino acid sequence of SEQ ID NO: 46, SEQ ID NO: 47, SEQ ID NO: 112, or SEQ ID NO: 113. In embodiments, (a) the first domain comprises the amino acid sequence of SEQ ID NO: 10, (b) the second domain comprises the amino acid sequence of SEQ ID NO: 2, and (c) the linker comprises an amino acid sequence that is at least 95% identical to the amino acid sequence of SEQ ID NO: 46, SEQ ID NO: 47, SEQ ID NO: 112, or SEQ ID NO: 113.

In embodiments, the chimeric protein further comprises at least one joining linker comprising an amino acid sequence selected from SKYGPPCPSCP (SEQ ID NO: 49), SKYGPPCPPCP (SEQ ID NO: 50), IEGRMD (SEQ ID NO: 52). In embodiments, the chimeric protein comprises the joining linker comprising the amino acid sequence of IEGRMD (SEQ ID NO: 52). In embodiments, the amino acid sequence of IEGRMD is located at C-terminal end of the amino acid sequence of SEQ ID NO: 46, SEQ ID NO: 47, SEQ ID NO: 112, or SEQ ID NO: 113.

In embodiments, the chimeric protein comprises an amino acid sequence that is at least 95%, or at least 96%, or at least 97%, or at least 98%, or at least 99% identical to an amino acid sequence selected from SEQ ID NO: 11, SEQ ID NO: 109, and SEQ ID NO: 110. In embodiments, the chimeric protein comprises an amino acid sequence that is at least 98% identical to an amino acid sequence selected from SEQ ID NO: 11, SEQ ID NO: 109, and SEQ ID NO: 110. In embodiments, the chimeric protein comprises an amino acid sequence that is at least 99% identical to an amino acid sequence selected from SEQ ID NO: 11, SEQ ID NO: 109, and SEQ ID NO: 110. In embodiments, the chimeric protein comprises an amino acid sequence that is at least 99.2% identical to an amino acid sequence selected from SEQ ID NO: 11, SEQ ID NO: 109, and SEQ ID NO: 110. In embodiments, the chimeric protein comprises an amino acid sequence that is at least 99.4% identical to an amino acid sequence selected from SEQ ID NO: 11, SEQ ID NO: 109, and SEQ ID NO: 110. In embodiments, the chimeric protein comprises an amino acid sequence that is at least 99.6% identical to an amino acid sequence selected from SEQ ID NO: 11, SEQ ID NO: 109, and SEQ ID NO: 110.

In embodiments, the chimeric protein comprises an amino acid sequence that is at least 99.8% identical to an amino acid sequence selected from SEQ ID NO: 11, SEQ ID NO: 109, and SEQ ID NO: 110. In embodiments, the chimeric protein comprises an amino acid sequence selected from SEQ ID NO: 11, SEQ ID NO: 109, and SEQ ID NO: 110. In embodiments, the chimeric protein comprises an amino acid sequence of SEQ ID NO: 11.

In embodiments, the treatment induces an increase in the level and/or activity of one or more cytokines selected from IL-2, IL-10, IP-10 (CXCL10), MCP-1, MIP-1β (CCL4) TARC (CCL17), IFNγ, IL-8, IL-12, and SDF1a (CXCL12). In embodiments, the treatment does not induce a cytokine release syndrome. In embodiments, the treatment smaller increases in IL-6 compared to other immunotherapy agents such as certain anti-CD40 antibodies or anti-PD-1 agents.

In embodiments, the subject has received, been tolerant to, or is ineligible for standard therapy and/or the cancer has no approved therapy considered to be standard of care. In embodiments, the subject is not receiving a concurrent chemotherapy, immunotherapy, biologic or hormonal therapy.

Methods of Determining a Cancer Treatment for a Patient; Methods of Selecting a Patient for a Cancer Treatment

In one aspect, the present disclosure relates to a method of evaluating the efficacy of a cancer treatment in a subject in need thereof, the method comprising the steps of: (i) administering a dose of a chimeric protein having a general structure of: N terminus—(a)—(b)—(c)—C terminus, wherein: (a) is a first domain comprising an extracellular domain of human T cell immunoreceptor with Ig and ITIM domains (TIGIT), (b) is a linker adjoining the first and second domains, wherein the linker comprises a hinge-CH2-CH3 Fc domain, and (c) is a second domain comprising an extracellular domain of human LIGHT (lymphotoxin-like, exhibits inducible expression and competes with HSV glycoprotein D for herpesvirus entry mediator (HVEM), a receptor expressed by T lymphocytes). wherein the dose is from about 0.03 mg/kg to about 50 mg/kg; (ii) obtaining a biological sample from the subject; (iii) performing an assay on the biological sample to determine level and/or activity of a cytokine selected from IL-2, IL-10, IP-10 (CXCL10), MCP-1, MIP-1β (CCL4) TARC (CCL17), IFNγ, IL-8, IL-12, and SDF1a (CXCL12); and (iv) continuing dosing if the subject has an increase in the level and/or activity of at least one cytokine selected from IL-2, IL-10, IP-10 (CXCL10), MCP-1, MIP-1β (CCL4) TARC (CCL17), IFNγ, IL-8, IL-12, and SDF1a (CXCL12).

In one aspect, the present disclosure relates to a method of selecting a subject for treatment with a therapy for cancer, the method comprising the steps of: (i) administering a dose of a chimeric protein having a general structure of: N terminus—(a)—(b)—(c)—C terminus, wherein: (a) is a first domain comprising an extracellular domain of human T cell immunoreceptor with Ig and ITIM domains (TIGIT), (b) is a linker adjoining the first and second domains, wherein the linker comprises a hinge-CH2-CH3 Fc domain, and (c) is a second domain comprising an extracellular domain of human LIGHT (lymphotoxin-like, exhibits inducible expression and competes with HSV glycoprotein D for herpesvirus entry mediator (HVEM), a receptor expressed by T lymphocytes). wherein the dose is from about 0.03 mg/kg to about 50 mg/kg; (ii) obtaining a biological sample from the subject; (iii) performing an assay on the biological sample to determine level and/or activity of a cytokine selected from IL-2, IL-10, IP-10 (CXCL10), MCP-1, MIP-1β (CCL4) TARC (CCL17), IFNγ, IL-8, IL-12, and SDF1a (CXCL12); and (iv) selecting the subject for treatment with the therapy for cancer if the subject has an increase in the level and/or activity of at least one cytokine selected from IL-2, IL-10, IP-10 (CXCL10), MCP-1, MIP-1β (CCL4) TARC (CCL17), IFNγ, IL-8, IL-12, and SDF1a (CXCL12).

In embodiments, the biological sample is a fresh tissue sample, frozen tumor tissue specimen, cultured cells, circulating tumor cells, or a formalin-fixed paraffin-embedded tumor tissue specimen. In embodiments, the biological sample is a biopsy sample. In embodiments, the biopsy sample is selected from endoscopic biopsy, bone marrow biopsy, endoscopic biopsy (e.g., cystoscopy, bronchoscopy and colonoscopy), needle biopsy (e.g., fine-needle aspiration, core needle biopsy, vacuum-assisted biopsy, X-ray-assisted biopsy, computerized tomography (CT)-assisted biopsy, magnetic resonance imaging (MRI)-assisted biopsy and ultrasound-assisted biopsy), skin biopsy (e.g., shave biopsy, punch biopsy, and incisional biopsy) and surgical biopsy. In embodiments, the biological sample comprises a body fluid selected from blood, plasma, serum, lacrimal fluid, tears, bone marrow, blood, blood cells, ascites, tissue or fine needle biopsy sample, cell-containing body fluid, free floating nucleic acids, sputum, saliva, urine, cerebrospinal fluid, peritoneal fluid, pleural fluid, feces, lymph, gynecological fluid, skin swab, vaginal swab, oral swab, nasal swab, washing or lavage such as a ductal lavage or broncheoalveolar lavage, aspirate, scraping, bone marrow specimen, tissue biopsy specimen, surgical specimen, feces, other body fluids, secretions, and/or excretions, and/or cells therefrom.

In embodiments, the biological sample is obtained by a technique selected from scrapes, swabs, and biopsy. In embodiments, the biological sample is obtained by use of brushes, (cotton) swabs, spatula, rinse/wash fluids, punch biopsy devices, puncture of cavities with needles or surgical instrumentation. In embodiments, the biological sample comprises at least one tumor cell.

In embodiments, the tumor is selected from Hodgkin's and non-Hodgkin's lymphoma, B-cell lymphoma (including low grade/follicular non-Hodgkin's lymphoma (NHL); small lymphocytic (SL) NHL; intermediate grade/follicular NHL; intermediate grade diffuse NHL; high grade immunoblastic NHL; high grade lymphoblastic NHL; high grade small non-cleaved cell NHL; bulky disease NHL; mantle cell lymphoma; AIDS-related lymphoma; and Waldenstrom's Macroglobulinemia; chronic lymphocytic leukemia (CLL); acute lymphoblastic leukemia (ALL); Hairy cell leukemia; or chronic myeloblastic leukemia, basal cell carcinoma, biliary tract cancer; bladder cancer; bone cancer; brain and central nervous system cancer; breast cancer; cancer of the peritoneum; cervical cancer; choriocarcinoma; colon and rectum cancer; connective tissue cancer; cancer of the digestive system; endometrial cancer; esophageal cancer; eye cancer; cancer of the head and neck; gastric cancer (including gastrointestinal cancer); glioblastoma; hepatic carcinoma; hepatoma; intra-epithelial neoplasm; kidney or renal cancer; larynx cancer; leukemia; liver cancer; lung cancer (e.g., small-cell lung cancer, non-small cell lung cancer, adenocarcinoma of the lung, and squamous carcinoma of the lung); melanoma; myeloma; neuroblastoma; oral cavity cancer (lip, tongue, mouth, and pharynx); ovarian cancer; pancreatic cancer; prostate cancer; retinoblastoma; rhabdomyosarcoma; rectal cancer; cancer of the respiratory system; salivary gland carcinoma; sarcoma; skin cancer; squamous cell cancer; stomach cancer; testicular cancer; thyroid cancer; uterine or endometrial cancer; cancer of the urinary system; vulval cancer; lymphoma including Hodgkin's and non-Hodgkin's lymphoma, as well as B-cell lymphoma (including low grade/follicular non-Hodgkin's lymphoma (NHL); small lymphocytic (SL) NHL; intermediate grade/follicular NHL; intermediate grade diffuse NHL; high grade immunoblastic NHL; high grade lymphoblastic NHL; high grade small non-cleaved cell NHL; bulky disease NHL; mantle cell lymphoma; AIDS-related lymphoma; and Waldenstrom's Macroglobulinemia; chronic lymphocytic leukemia (CLL); acute lymphoblastic leukemia (ALL); Hairy cell leukemia; chronic myeloblastic leukemia; as well as other carcinomas and sarcomas; and post-transplant lymphoproliferative disorder (PTLD), as well as abnormal vascular proliferation associated with phakomatoses, edema (e.g., that associated with brain tumors), Meigs' syndrome cancer; renal carcinoma; colorectal cancer; and adrenal cancer.

In embodiments, the assay is performed by DNA sequencing, RNA sequencing, immunohistochemical staining, western blotting, in cell western, immunofluorescent staining, ELISA, and fluorescent activating cell sorting (FACS) or a combination thereof.

In embodiments, the assay is performed by contacting the sample with one or more agents that specifically binds to at least one cytokine selected from IL-2, IL-10, IP-10 (CXCL10), MCP-1, MIP-1β (CCL4) TARC (CCL17), IFNγ, IL-8, IL-12, and SDF1a (CXCL12). In embodiments, the agents that specifically binds to at least one cytokine comprise one or more antibody, antibody-like molecule or binding a fragment thereof.

In embodiments, the assay is performed by contacting the sample with one or more agents that specifically binds to at least one nucleic acid encoding a cytokine selected from IL-2, IL-10, IP-10 (CXCL10), MCP-1, MIP-1β (CCL4) TARC (CCL17), IFNγ, IL-8, IL-12, and SDF1a (CXCL12 In embodiments, the agent that specifically binds to at least one nucleic acid is a nucleic acid primer or probe.

is capable of binding a TIGIT ligand. In embodiments, the first domain comprises substantially all of the extracellular domain of TIGIT. In embodiments, the second domain is capable of binding a LIGHT receptor. In embodiments, the second domain comprises substantially all of the extracellular domain of LIGHT.

In embodiments, the linker is a polypeptide selected from a flexible amino acid sequence, an IgG hinge region, and/or an antibody sequence. In embodiments, the linker comprises hinge-CH2-CH3 Fc domain derived from IgG1 or IgG4. In embodiments, the hinge-CH2-CH3 Fc domain is derived from human IgG1 or human IgG4. In embodiments, the linker comprises an amino acid sequence that is at least 95%, or at least 96%, or at least 97%, or at least 98%, or at least 99% identical to the amino acid sequence of SEQ ID NO: 46, SEQ ID NO: 47, SEQ ID NO: 112, or SEQ ID NO: 113. In embodiments, the linker comprises one or more joining linkers, such joining linkers independently selected from SEQ ID NOs: 49-95. In embodiments, the linker comprises two or more joining linkers each joining linker independently selected from SEQ ID NOs: 49-95; wherein one joining linker is N terminal to the hinge-CH2-CH3 Fc domain and another joining linker is C terminal to the hinge-CH2-CH3 Fc domain.

In embodiments, the first domain comprises an amino acid sequence that is at least 95%, or at least 96%, or at least 97%, or at least 98%, or at least 99% identical to the amino acid sequence of SEQ ID NO: 10. In embodiments, the second domain comprises an amino acid sequence that is at least 95%, or at least 96%, or at least 97%, or at least 98%, or at least 99% identical to the amino acid sequence of SEQ ID NO: 2.

In embodiments, (a) the first domain comprises an amino acid sequence that is at least 95% identical to the amino acid sequence of SEQ ID NO: 10, (b) the second domain comprises the amino acid sequence that is at least 95% identical to the amino acid sequence of SEQ ID NO: 2, and (c) the linker comprises an amino acid sequence that is at least 95% identical to the amino acid sequence of SEQ ID NO: 46, SEQ ID NO: 47, SEQ ID NO: 112, or SEQ ID NO: 113. In embodiments, (a) the first domain comprises the amino acid sequence of SEQ ID NO: 10, (b) the second domain comprises the amino acid sequence of SEQ ID NO: 2, and (c) the linker comprises an amino acid sequence that is at least 95% identical to the amino acid sequence of SEQ ID NO: 46, SEQ ID NO: 47, SEQ ID NO: 112, or SEQ ID NO: 113.

In embodiments, the chimeric protein further comprises at least one joining linker comprising an amino acid sequence selected from SKYGPPCPSCP (SEQ ID NO: 49), SKYGPPCPPCP (SEQ ID NO: 50), IEGRMD (SEQ ID NO: 52). In embodiments, the chimeric protein comprises the joining linker comprising the amino acid sequence of IEGRMD (SEQ ID NO: 52). In embodiments, the amino acid sequence of IEGRMD is located at C-terminal end of the amino acid sequence of SEQ ID NO: 46, SEQ ID NO: 47, SEQ ID NO: 112, or SEQ ID NO: 113.

In embodiments, the chimeric protein comprises an amino acid sequence that is at least 95%, or at least 96%, or at least 97%, or at least 98%, or at least 99% identical to an amino acid sequence selected from SEQ ID NO: 11, SEQ ID NO: 109, and SEQ ID NO: 110. In embodiments, the chimeric protein comprises an amino acid sequence that is at least 98% identical to an amino acid sequence selected from SEQ ID NO: 11, SEQ ID NO: 109, and SEQ ID NO: 110. In embodiments, the chimeric protein comprises an amino acid sequence that is at least 99% identical to an amino acid sequence selected from SEQ ID NO: 11, SEQ ID NO: 109, and SEQ ID NO: 110. In embodiments, the chimeric protein comprises an amino acid sequence that is at least 99.2% identical to an amino acid sequence selected from SEQ ID NO: 11, SEQ ID NO: 109, and SEQ ID NO: 110. In embodiments, the chimeric protein comprises an amino acid sequence that is at least 99.4% identical to an amino acid sequence selected from SEQ ID NO: 11, SEQ ID NO: 109, and SEQ ID NO: 110. In embodiments, the chimeric protein comprises an amino acid sequence that is at least 99.6% identical to an amino acid sequence selected from SEQ ID NO: 11, SEQ ID NO: 109, and SEQ ID NO: 110. In embodiments, the chimeric protein comprises an amino acid sequence that is at least 99.8% identical to an amino acid sequence selected from SEQ ID NO: 11, SEQ ID NO: 109, and SEQ ID NO: 110. In embodiments, the chimeric protein comprises an amino acid sequence selected from SEQ ID NO: 11, SEQ ID NO: 109, and SEQ ID NO: 110. In embodiments, the chimeric protein comprises an amino acid sequence of SEQ ID NO: 11.

In one aspect, the present disclosure relates to a method of determining a cancer treatment for a patient, the method comprising: (a) obtaining a biological sample from a subject; (b) evaluating the sample for the presence, absence, or level of one or more gene associated with a gene ontology (GO) pathway selected from: (i) positive regulation of cell cycle process, regulation of G1/S transition, regulation of cell division, regulation of cell proliferation, positive regulation of IκB kinase/NFκB signaling, type I IFN signaling pathway, cellular response to IFNγ, positive regulation of IFNα production, positive regulation of defense response, positive regulation of IFNβ production, regulation of inflammatory response, regulation of innate immune response, negative regulation of antigen processing and presentation, and antigen processing, presentation of endogenous peptides via MHC class I; and/or (ii) phospholipid efflux, negative regulation of fibrinolysis, chylomicron assembly, plasma membrane repair, SRP-dependent co-translational protein targeting to membrane, ribosomal small subunit assembly, phospholipid efflux, regulation of translation, mitochondrial respiratory chain complex I, mitochondrial translational elongation, DNA-dependent DNA replication, and ATP biosynthetic process; and (c) selecting the cancer therapy with an ability to inhibit function and/or activity of PD-1, PD-L1 and/or PD-L2 based on the evaluation of step (b).

In one aspect, the present disclosure relates to a method for selecting a patient for a cancer treatment, the method comprising: (a) obtaining a biological sample from a subject; (b) evaluating the sample for the presence, absence, or level of one or more genes associated with a gene ontology (GO) pathway selected from: (i) positive regulation of cell cycle process, regulation of G1/S transition, regulation of cell division, regulation of cell proliferation, positive regulation of IκB kinase/NFκB signaling, type I IFN signaling pathway, cellular response to IFNγ, positive regulation of IFNα production, positive regulation of defense response, positive regulation of IFNβ production, regulation of inflammatory response, regulation of innate immune response, negative regulation of antigen processing and presentation, and antigen processing, presentation of endogenous peptides via MHC class I; and/or (ii) phospholipid efflux, negative regulation of fibrinolysis, chylomicron assembly, plasma membrane repair, SRP-dependent co-translational protein targeting to membrane, ribosomal small subunit assembly, phospholipid efflux, regulation of translation, mitochondrial respiratory chain complex I, mitochondrial translational elongation, DNA-dependent DNA replication, and

ATP biosynthetic process; and (c) selecting a cancer therapy with an ability to inhibit function and/or activity of PD-1, PD-L1 and/or PD-L2.

In one aspect, the present disclosure relates to a method of treating cancer, the method comprising: (I) obtaining a biological sample from a subject; (II) evaluating the sample for the presence, absence, or level of one or more genes associated with a gene ontology (GO) pathway selected from: (i) positive regulation of cell cycle process, regulation of G1/S transition, regulation of cell division, regulation of cell proliferation, positive regulation of IκB kinase/NFκB signaling, type I IFN signaling pathway, cellular response to IFNγ, positive regulation of IFNα production, positive regulation of defense response, positive regulation of IFNβ production, regulation of inflammatory response, regulation of innate immune response, negative regulation of antigen processing and presentation, and antigen processing, presentation of endogenous peptides via MHC class I; and/or (ii) phospholipid efflux, negative regulation of fibrinolysis, chylomicron assembly, plasma membrane repair, SRP-dependent co-translational protein targeting to membrane, ribosomal small subunit assembly, phospholipid efflux, regulation of translation, mitochondrial respiratory chain complex I, mitochondrial translational elongation, DNA-dependent DNA replication, and ATP biosynthetic process; and (III) selecting the cancer therapy with a chimeric protein of a general structure of: N terminus—(a)—(b)—(c)— C terminus, wherein: (A) (a) is a first domain comprising an extracellular domain of a Type I transmembrane protein, the transmembrane protein being TIGIT, (b) is a linker comprising at least one cysteine residue capable of forming a disulfide bond, and (c) is a second domain comprising an extracellular domain of Type II transmembrane protein, the transmembrane protein being LIGHT, wherein the linker connects the first domain and the second domain and optionally comprises one or more joining linkers, such joining linkers being selected from SEQ ID NOs: 49-95; or (B) (a) is a first domain comprising an extracellular domain of a Type I transmembrane protein, the transmembrane protein being SIRPα, (b) is a linker comprising at least one cysteine residue capable of forming a disulfide bond, and (c) is a second domain comprising an extracellular domain of Type II transmembrane protein, the transmembrane protein being 4-1BBL, wherein the linker connects the first domain and the second domain and optionally comprises one or more joining linkers, such joining linkers being selected from SEQ ID NOs: 49-95; and (IV) optionally administering the cancer therapy with an ability to inhibit function and/or activity of PD-1, PD-L1 and/or PD-L2.

In non-limiting embodiments, administering the cancer therapy with an ability to inhibit function and/or activity of PD-1. PD-L1 and/or PD-L2 is continued if the upregulation of genes associated with a GO pathway selected from positive regulation of cell cycle process, regulation of G1/S transition, regulation of cell division, regulation of cell proliferation, positive regulation of IκB kinase/NFκB signaling, type I IFN signaling pathway, cellular response to IFNγ, positive regulation of IFNα production, positive regulation of defense response, positive regulation of IFNβ production, regulation of inflammatory response, regulation of innate immune response, negative regulation of antigen processing and presentation, and antigen processing, presentation of endogenous peptides via MHC class I is not observed and/or if the downregulation of genes associated with a GO pathway selected from phospholipid efflux, negative regulation of fibrinolysis, chylomicron assembly, plasma membrane repair, SRP-dependent co-translational protein targeting to membrane, ribosomal small subunit assembly, phospholipid efflux, regulation of translation, mitochondrial respiratory chain complex I, mitochondrial translational elongation, DNA-dependent DNA replication, and ATP biosynthetic process is not observed.

In non-limiting embodiments, administering the cancer therapy with an ability to inhibit function and/or activity of PD-1. PD-L1 and/or PD-L2 is continued if the upregulation of genes associated with a GO pathway selected from positive regulation of cell cycle process, regulation of G1/S transition, regulation of cell division, regulation of cell proliferation, positive regulation of IκB kinase/NFκB signaling, type I IFN signaling pathway, cellular response to IFNγ, positive regulation of IFNα production, positive regulation of defense response, positive regulation of IFN production, regulation of inflammatory response, regulation of innate immune response, negative regulation of antigen processing and presentation, and antigen processing, presentation of endogenous peptides via MHC class I is observed and/or if the downregulation of genes associated with a GO pathway selected from phospholipid efflux, negative regulation of fibrinolysis, chylomicron assembly, plasma membrane repair, SRP-dependent co-translational protein targeting to membrane, ribosomal small subunit assembly, phospholipid efflux, regulation of translation, mitochondrial respiratory chain complex I, mitochondrial translational elongation, DNA-dependent DNA replication, and ATP biosynthetic process is observed, wherein the supplementation of administration of cancer therapy with a chimeric protein of a general structure of: N terminus—(a)—(b)—(c)—C terminus, wherein: (A) (a) is a first domain comprising an extracellular domain of a Type I transmembrane protein, the transmembrane protein being TIGIT, (b) is a linker comprising at least one cysteine residue capable of forming a disulfide bond, and (c) is a second domain comprising an extracellular domain of Type II transmembrane protein, the transmembrane protein being LIGHT, wherein the linker connects the first domain and the second domain and optionally comprises one or more joining linkers, such joining linkers being selected from SEQ ID NOs: 49-95; or (B) (a) is a first domain comprising an extracellular domain of a Type I transmembrane protein, the transmembrane protein being SIRPα, (b) is a linker comprising at least one cysteine residue capable of forming a disulfide bond, and (c) is a second domain comprising an extracellular domain of Type II transmembrane protein, the transmembrane protein being 4-1BBL, wherein the linker connects the first domain and the second domain and optionally comprises one or more joining linkers, such joining linkers being selected from SEQ ID NOs: 49-95.

In non-limiting embodiments, administering the cancer therapy with an ability to inhibit function and/or activity of PD-1, PD-L1 and/or PD-L2 is not continued if the upregulation of genes associated with a GO pathway selected from positive regulation of cell cycle process, regulation of G1/S transition, regulation of cell division, regulation of cell proliferation, positive regulation of IκB kinase/NFκB signaling, type I IFN signaling pathway, cellular response to IFNγ, positive regulation of IFNα production, positive regulation of defense response, positive regulation of IFNβ production, regulation of inflammatory response, regulation of innate immune response, negative regulation of antigen processing and presentation, and antigen processing, presentation of endogenous peptides via MHC class I is observed and/or if the downregulation of genes associated with a GO pathway selected from phospholipid efflux, negative regulation of fibrinolysis, chylomicron assembly, plasma membrane repair, SRP-dependent co-translational protein targeting to membrane, ribosomal small subunit assembly, phospholipid efflux, regulation of translation, mitochondrial respiratory chain complex I, mitochondrial translational elongation, DNA-dependent DNA replication, and ATP biosynthetic process is observed, wherein the administration of cancer therapy with a chimeric protein of a general structure of: N terminus—(a)—(b)—(c)—C terminus, wherein: (A) (a) is a first domain comprising an extracellular domain of a Type I transmembrane protein, the transmembrane protein being TIGIT, (b) is a linker comprising at least one cysteine residue capable of forming a disulfide bond, and (c) is a second domain comprising an extracellular domain of Type II transmembrane protein, the transmembrane protein being LIGHT, wherein the linker connects the first domain and the second domain and optionally comprises one or more joining linkers, such joining linkers being selected from SEQ ID NOs: 49-95; or (B) (a) is a first domain comprising an extracellular domain of a Type I transmembrane protein, the transmembrane protein being SIRPα, (b) is a linker comprising at least one cysteine residue capable of forming a disulfide bond, and (c) is a second domain comprising an extracellular domain of Type II transmembrane protein, the transmembrane protein being 4-1BBL, wherein the linker connects the first domain and the second domain and optionally comprises one or more joining linkers, such joining linkers being selected from SEQ ID NOs: 49-95.

In embodiments, an upregulation of one or more genes associated with a GO pathway listed in (i) compared to a healthy tissue indicates a lack of response, resistance or recalcitrance to the cancer therapy with an ability to inhibit function and/or activity of PD-1, PD-L1 and/or PD-L2. In embodiments, an upregulation of one or more genes associated with a GO pathway listed in (i) compared to another biological sample from patient that is known to be sensitive to anti-PD-1 therapy indicates a lack of response, resistance or recalcitrance to the cancer therapy with an ability to inhibit function and/or activity of PD-1, PD-L1 and/or PD-L2. In embodiments, an upregulation of one or more genes associated with a GO pathway listed in (i) compared to a prior biological sample obtained from the subject indicates a development of resistance, a lack of response, or recalcitrance to the cancer therapy with an ability to inhibit function and/or activity of PD-1, PD-L1 and/or PD-L2.

In embodiments, a lack of upregulation of one or more genes associated with a GO pathway listed in (i) compared to a healthy tissue indicates a response to the cancer therapy with an ability to inhibit function and/or activity of PD-1, PD-L1 and/or PD-L2. In embodiments, a lack of upregulation of one or more genes associated with a GO pathway listed in (i) compared to another biological sample from patient that is known to be sensitive to anti-PD-1 therapy indicates a response to the cancer therapy with an ability to inhibit function and/or activity of PD-1, PD-L1 and/or PD-L2. In embodiments, a lack of upregulation of one or more genes associated with a GO pathway listed in (i) compared to a prior biological sample obtained from the subject indicates a response to the cancer therapy with an ability to inhibit function and/or activity of PD-1, PD-L1 and/or PD-L2.

In embodiments, a downregulation of one or more genes associated with a GO pathway listed in (ii) compared to a healthy tissue indicates a lack of response, resistance or recalcitrance to the cancer therapy with an ability to inhibit function and/or activity of PD-1, PD-L1 and/or PD-L2. In embodiments, a downregulation of one or more genes associated with a GO pathway listed in (ii) compared to another biological sample from patient that is known to be sensitive to anti-PD-1 therapy indicates a lack of response, resistance or recalcitrance to the cancer therapy with an ability to inhibit function and/or activity of PD-1, PD-L1 and/or PD-L2. In embodiments, a downregulation of one or more genes associated with a GO pathway listed in (ii) compared to a prior biological sample obtained from the subject indicates a development of resistance, a lack of response, or recalcitrance to the cancer therapy with an ability to inhibit function and/or activity of PD-1, PD-L1 and/or PD-L2.

In embodiments, a lack of downregulation of one or more genes associated with a GO pathway listed in (ii) compared to a healthy tissue indicates a response to the cancer therapy with an ability to inhibit function and/or activity of PD-1, PD-L1 and/or PD-L2. In embodiments, a lack of downregulation of one or more genes associated with a GO pathway listed in (ii) compared to another biological sample from patient that is known to be sensitive to anti-PD-1 therapy indicates a response to the cancer therapy with an ability to inhibit function and/or activity of PD-1, PD-L1 and/or PD-L2. In embodiments, a lack of downregulation of one or more genes associated with a GO pathway listed in (ii) compared to a prior biological sample obtained from the subject indicates a response to the cancer therapy with an ability to inhibit function and/or activity of PD-1, PD-L1 and/or PD-L2.

In embodiments, a lack of upregulation of one or more genes associated with a GO pathway compared to a prior biological sample from the subject indicates a response to the cancer therapy with an ability to inhibit function and/or activity of PD-1, PD-L1 and/or PD-L2, wherein the GO pathway is selected from (a) cellular response to IFNγ, (b) negative regulation of antigen processing and presentation, (c) type I IFN signaling pathway, (d) positive regulation of IκB kinase/NFκB signaling, and antigen processing, and (e) presentation of endogenous peptides via MHC class I. In embodiments, a lack of upregulation of one or more genes associated with a GO pathway compared to a patient that is known to be sensitive to anti-PD-1 therapy indicates a response to the cancer therapy with an ability to inhibit function and/or activity of PD-1, PD-L1 and/or PD-L2, wherein the GO pathway is selected from (a) cellular response to IFNγ, (b) negative regulation of antigen processing and presentation, (c) type I IFN signaling pathway, (d) positive regulation of IκB kinase/NFκB signaling, and antigen processing, and (e) presentation of endogenous peptides via MHC class I. In embodiments, a lack of upregulation of one or more genes associated with a GO pathway compared to a standard indicates a response to the cancer therapy with an ability to inhibit function and/or activity of PD-1, PD-L1 and/or PD-L2, wherein the GO pathway is selected from (a) cellular response to IFNγ, (b) negative regulation of antigen processing and presentation, (c) type I IFN signaling pathway, (d) positive regulation of IκB kinase/NFκB signaling, and antigen processing, and (e) presentation of endogenous peptides via MHC class I.

In embodiments, an upregulation of one or more genes associated with a GO pathway compared to a prior biological sample from the subject indicates resistance, a lack of response, or recalcitrance to the cancer therapy with an ability to inhibit function and/or activity of PD-1, PD-L1 and/or PD-L2, wherein the GO pathway is selected from (a) cellular response to IFNγ, (b) negative regulation of antigen processing and presentation, (c) type I IFN signaling pathway, (d) positive regulation of IκB kinase/NFκB signaling, and antigen processing, and (e) presentation of endogenous peptides via MHC class I. In embodiments, an upregulation of one or more genes associated with a GO pathway compared to a patient that is known to be sensitive to anti-PD-1 therapy indicates resistance, a lack of response, or recalcitrance to the cancer therapy with an ability to inhibit function and/or activity of PD-1, PD-L1 and/or PD-L2, wherein the GO pathway is selected from (a) cellular response to IFNγ, (b) negative regulation of antigen processing and presentation, (c) type I IFN signaling pathway, (d) positive regulation of IκB kinase/NFκB signaling, and antigen processing, and (e) presentation of endogenous peptides via MHC class I. In embodiments, an upregulation of one or more genes associated with a GO pathway compared to a standard indicates resistance, a lack of response, or recalcitrance to the cancer therapy with an ability to inhibit function and/or activity of PD-1, PD-L1 and/or PD-L2, wherein the GO pathway is selected from (a) cellular response to IFNγ, (b) negative regulation of antigen processing and presentation, (c) type I IFN signaling pathway, (d) positive regulation of IκB kinase/NFκB signaling, and antigen processing, and (e) presentation of endogenous peptides via MHC class I.

In embodiments, a lack of upregulation of one or more genes associated with cellular response to IFNγ compared to a prior biological sample from the subject indicates a response to the cancer therapy with an ability to inhibit function and/or activity of PD-1, PD-L1 and/or PD-L2. In embodiments, a lack of upregulation of one or more genes associated with cellular response to IFNγ compared to a patient that is known to be sensitive to anti-PD-1 therapy indicates a response to the cancer therapy with an ability to inhibit function and/or activity of PD-1, PD-L1 and/or PD-L2. In embodiments, a lack of upregulation of one or more genes associated with cellular response to IFNγ compared to a standard indicates a response to the cancer therapy with an ability to inhibit function and/or activity of PD-1, PD-L1 and/or PD-L2.

In embodiments, an upregulation of one or more genes associated with cellular response to IFNγ compared to a prior biological sample from the subject indicates resistance, a lack of response, or recalcitrance to the cancer therapy with an ability to inhibit function and/or activity of PD-1, PD-L1 and/or PD-L2. In embodiments, an upregulation of one or more genes associated with cellular response to IFNγ compared to a patient that is known to be sensitive to anti-PD-1 therapy indicates resistance, a lack of response, or recalcitrance to the cancer therapy with an ability to inhibit function and/or activity of PD-1, PD-L1 and/or PD-L2. In embodiments, an upregulation of one or more genes associated with cellular response to IFNγ compared to a standard indicates resistance, a lack of response, or recalcitrance to the cancer therapy with an ability to inhibit function and/or activity of PD-1, PD-L1 and/or PD-L2.

In embodiments, an upregulation of one or more genes associated with type I IFN signaling pathway compared to a prior biological sample from the subject indicates resistance, a lack of response, or recalcitrance to the cancer therapy with an ability to inhibit function and/or activity of PD-1, PD-L1 and/or PD-L2. In embodiments, an upregulation of one or more genes associated with type I IFN signaling pathway compared to a patient that is known to be sensitive to anti-PD-1 therapy indicates resistance, a lack of response, or recalcitrance to the cancer therapy with an ability to inhibit function and/or activity of PD-1, PD-L1 and/or PD-L2. In embodiments, an upregulation of one or more genes associated with type I IFN signaling pathway compared to a standard indicates resistance, a lack of response, or recalcitrance to the cancer therapy with an ability to inhibit function and/or activity of PD-1, PD-L1 and/or PD-L2.

In embodiments, a lack of upregulation of one or more genes associated with type I IFN signaling pathway compared to a prior biological sample from the subject indicates a response to the cancer therapy with an ability to inhibit function and/or activity of PD-1, PD-L1 and/or PD-L2. In embodiments, a lack of upregulation of one or more genes associated with type I IFN signaling pathway compared to a patient that is known to be sensitive to anti-PD-1 therapy indicates a response to the cancer therapy with an ability to inhibit function and/or activity of PD-1, PD-L1 and/or PD-L2. In embodiments, a lack of upregulation of one or more genes associated with type I IFN signaling pathway compared to a standard indicates a response to the cancer therapy with an ability to inhibit function and/or activity of PD-1, PD-L1 and/or PD-L2.

In embodiments, an upregulation of one or more genes associated with positive regulation of cell cycle process compared to a prior biological sample from the subject indicates resistance, a lack of response, or recalcitrance to the cancer therapy with an ability to inhibit function and/or activity of PD-1, PD-L1 and/or PD-L2. In embodiments, an upregulation of one or more genes associated with positive regulation of cell cycle process compared to a patient that is known to be sensitive to anti-PD-1 therapy indicates resistance, a lack of response, or recalcitrance to the cancer therapy with an ability to inhibit function and/or activity of PD-1, PD-L1 and/or PD-L2. In embodiments, an upregulation of one or more genes associated with positive regulation of cell cycle process compared to a standard indicates resistance, a lack of response, or recalcitrance to the cancer therapy with an ability to inhibit function and/or activity of PD-1, PD-L1 and/or PD-L2.

In embodiments, a lack of upregulation of one or more genes associated with positive regulation of cell cycle process compared to a prior biological sample from the subject indicates a response to the cancer therapy with an ability to inhibit function and/or activity of PD-1, PD-L1 and/or PD-L2. In embodiments, a lack of upregulation of one or more genes associated with positive regulation of cell cycle process compared to a patient that is known to be sensitive to anti-PD-1 therapy indicates a response to the cancer therapy with an ability to inhibit function and/or activity of PD-1, PD-L1 and/or PD-L2. In embodiments, a lack of upregulation of one or more genes associated with positive regulation of cell cycle process compared to a standard indicates a response to the cancer therapy with an ability to inhibit function and/or activity of PD-1, PD-L1 and/or PD-L2.

In embodiments, an upregulation of one or more genes associated with regulation of G1/S transition compared to a prior biological sample from the subject indicates resistance, a lack of response, or recalcitrance to the cancer therapy with an ability to inhibit function and/or activity of PD-1, PD-L1 and/or PD-L2. In embodiments, an upregulation of one or more genes associated with regulation of G1/S transition compared to a patient that is known to be sensitive to anti-PD-1 therapy indicates resistance, a lack of response, or recalcitrance to the cancer therapy with an ability to inhibit function and/or activity of PD-1, PD-L1 and/or PD-L2. In embodiments, an upregulation of one or more genes associated with regulation of G1/S transition compared to a standard indicates resistance, a lack of response, or recalcitrance to the cancer therapy with an ability to inhibit function and/or activity of PD-1, PD-L1 and/or PD-L2.

In embodiments, a lack of upregulation of one or more genes associated with regulation of G1/S transition compared to a prior biological sample from the subject indicates a response to the cancer therapy with an ability to inhibit function and/or activity of PD-1, PD-L1 and/or PD-L2. In embodiments, a lack of upregulation of one or more genes associated with regulation of G1/S transition compared to a patient that is known to be sensitive to anti-PD-1 therapy indicates a response to the cancer therapy with an ability to inhibit function and/or activity of PD-1, PD-L1 and/or PD-L2. In embodiments, a lack of upregulation of one or more genes associated with regulation of G1/S transition compared to a standard indicates a response to the cancer therapy with an ability to inhibit function and/or activity of PD-1, PD-L1 and/or PD-L2.

In embodiments, an upregulation of one or more genes associated with regulation of cell division compared to a prior biological sample from the subject indicates resistance, a lack of response, or recalcitrance to the cancer therapy with an ability to inhibit function and/or activity of PD-1, PD-L1 and/or PD-L2. In embodiments, an upregulation of one or more genes associated with regulation of cell division compared to a patient that is known to be sensitive to anti-PD-1 therapy indicates resistance, a lack of response, or recalcitrance to the cancer therapy with an ability to inhibit function and/or activity of PD-1, PD-L1 and/or PD-L2. In embodiments, an upregulation of one or more genes associated with regulation of cell division compared to a standard indicates resistance, a lack of response, or recalcitrance to the cancer therapy with an ability to inhibit function and/or activity of PD-1, PD-L1 and/or PD-L2.

In embodiments, a lack of upregulation of one or more genes associated with regulation of cell division compared to a prior biological sample from the subject indicates a response to the cancer therapy with an ability to inhibit function and/or activity of PD-1, PD-L1 and/or PD-L2. In embodiments, a lack of upregulation of one or more genes associated with regulation of cell division compared to a patient that is known to be sensitive to anti-PD-1 therapy indicates a response to the cancer therapy with an ability to inhibit function and/or activity of PD-1, PD-L1 and/or PD-L2. In embodiments, a lack of upregulation of one or more genes associated with regulation of cell division compared to a standard indicates a response to the cancer therapy with an ability to inhibit function and/or activity of PD-1, PD-L1 and/or PD-L2.

In embodiments, an upregulation of one or more genes associated with regulation of cell proliferation compared to a prior biological sample from the subject indicates resistance, a lack of response, or recalcitrance to the cancer therapy with an ability to inhibit function and/or activity of PD-1, PD-L1 and/or PD-L2. In embodiments, an upregulation of one or more genes associated with regulation of cell proliferation compared to a patient that is known to be sensitive to anti-PD-1 therapy indicates resistance, a lack of response, or recalcitrance to the cancer therapy with an ability to inhibit function and/or activity of PD-1, PD-L1 and/or PD-L2. In embodiments, an upregulation of one or more genes associated with regulation of cell proliferation compared to a standard indicates resistance, a lack of response, or recalcitrance to the cancer therapy with an ability to inhibit function and/or activity of PD-1, PD-L1 and/or PD-L2.

In embodiments, a lack of upregulation of one or more genes associated with regulation of cell proliferation compared to a prior biological sample from the subject indicates a response to the cancer therapy with an ability to inhibit function and/or activity of PD-1, PD-L1 and/or PD-L2. In embodiments, a lack of upregulation of one or more genes associated with regulation of cell proliferation compared to a patient that is known to be sensitive to anti-PD-1 therapy indicates a response to the cancer therapy with an ability to inhibit function and/or activity of PD-1, PD-L1 and/or PD-L2. In embodiments, a lack of upregulation of one or more genes associated with regulation of cell proliferation compared to a standard indicates a response to the cancer therapy with an ability to inhibit function and/or activity of PD-1, PD-L1 and/or PD-L2.

In embodiments, an upregulation of one or more genes associated with positive regulation of IκB kinase/NFκB signaling compared to a prior biological sample from the subject indicates resistance, a lack of response, or recalcitrance to the cancer therapy with an ability to inhibit function and/or activity of PD-1, PD-L1 and/or PD-L2. In embodiments, an upregulation of one or more genes associated with positive regulation of IκB kinase/NFκB signaling compared to a patient that is known to be sensitive to anti-PD-1 therapy indicates resistance, a lack of response, or recalcitrance to the cancer therapy with an ability to inhibit function and/or activity of PD-1, PD-L1 and/or PD-L2. In embodiments, an upregulation of one or more genes associated with positive regulation of IκB kinase/NFκB signaling compared to a standard indicates resistance, a lack of response, or recalcitrance to the cancer therapy with an ability to inhibit function and/or activity of PD-1, PD-L1 and/or PD-L2.

In embodiments, a lack of upregulation of one or more genes associated with positive regulation of IκB kinase/NFκB signaling compared to a prior biological sample from the subject indicates a response to the cancer therapy with an ability to inhibit function and/or activity of PD-1, PD-L1 and/or PD-L2. In embodiments, a lack of upregulation of one or more genes associated with positive regulation of IκB kinase/NFκB signaling compared to a patient that is known to be sensitive to anti-PD-1 therapy indicates a response to the cancer therapy with an ability to inhibit function and/or activity of PD-1, PD-L1 and/or PD-L2. In embodiments, a lack of upregulation of one or more genes associated with positive regulation of IκB kinase/NFκB signaling compared to a standard indicates a response to the cancer therapy with an ability to inhibit function and/or activity of PD-1, PD-L1 and/or PD-L2.

In embodiments, an upregulation of one or more genes associated with regulation of innate immune response compared to a prior biological sample from the subject indicates resistance, a lack of response, or recalcitrance to the cancer therapy with an ability to inhibit function and/or activity of PD-1, PD-L1 and/or PD-L2. In embodiments, an upregulation of one or more genes associated with regulation of innate immune response compared to a patient that is known to be sensitive to anti-PD-1 therapy indicates resistance, a lack of response, or recalcitrance to the cancer therapy with an ability to inhibit function and/or activity of PD-1, PD-L1 and/or PD-L2. In embodiments, an upregulation of one or more genes associated with regulation of innate immune response compared to a standard indicates resistance, a lack of response, or recalcitrance to the cancer therapy with an ability to inhibit function and/or activity of PD-1, PD-L1 and/or PD-L2.

In embodiments, a lack of upregulation of one or more genes associated with regulation of innate immune response compared to a prior biological sample from the subject indicates a response to the cancer therapy with an ability to inhibit function and/or activity of PD-1, PD-L1 and/or PD-L2. In embodiments, a lack of upregulation of one or more genes associated with regulation of innate immune response compared to a patient that is known to be sensitive to anti-PD-1 therapy indicates a response to the cancer therapy with an ability to inhibit function and/or activity of PD-1, PD-L1 and/or PD-L2. In embodiments, a lack of upregulation of one or more genes associated with regulation of innate immune response compared to a standard indicates a response to the cancer therapy with an ability to inhibit function and/or activity of PD-1, PD-L1 and/or PD-L2.

In embodiments, an upregulation of one or more genes associated with negative regulation of antigen processing/presentation compared to a prior biological sample from the subject indicates resistance, a lack of response, or recalcitrance to the cancer therapy with an ability to inhibit function and/or activity of PD-1, PD-L1 and/or PD-L2. In embodiments, an upregulation of one or more genes associated with negative regulation of antigen processing/presentation compared to a patient that is known to be sensitive to anti-PD-1 therapy indicates resistance, a lack of response, or recalcitrance to the cancer therapy with an ability to inhibit function and/or activity of PD-1, PD-L1 and/or PD-L2. In embodiments, an upregulation of one or more genes associated with negative regulation of antigen processing/presentation compared to a standard indicates resistance, a lack of response, or recalcitrance to the cancer therapy with an ability to inhibit function and/or activity of PD-1, PD-L1 and/or PD-L2.

In embodiments, a lack of upregulation of one or more genes associated with negative regulation of antigen processing/presentation compared to a prior biological sample from the subject indicates a response to the cancer therapy with an ability to inhibit function and/or activity of PD-1, PD-L1 and/or PD-L2. In embodiments, a lack of upregulation of one or more genes associated with negative regulation of antigen processing/presentation compared to a patient that is known to be sensitive to anti-PD-1 therapy indicates a response to the cancer therapy with an ability to inhibit function and/or activity of PD-1, PD-L1 and/or PD-L2. In embodiments, a lack of upregulation of one or more genes associated with negative regulation of antigen processing/presentation compared to a standard indicates a response to the cancer therapy with an ability to inhibit function and/or activity of PD-1, PD-L1 and/or PD-L2.

In embodiments, an upregulation of one or more genes associated with antigen processing/presentation of endogenous peptides via MHC class I compared to a prior biological sample from the subject indicates resistance, a lack of response, or recalcitrance to the cancer therapy with an ability to inhibit function and/or activity of PD-1, PD-L1 and/or PD-L2. In embodiments, an upregulation of one or more genes associated with antigen processing/presentation of endogenous peptides via MHC class I compared to a patient that is known to be sensitive to anti-PD-1 therapy indicates resistance, a lack of response, or recalcitrance to the cancer therapy with an ability to inhibit function and/or activity of PD-1, PD-L1 and/or PD-L2. In embodiments, an upregulation of one or more genes associated with antigen processing/presentation of endogenous peptides via MHC class I compared to a standard indicates resistance, a lack of response, or recalcitrance to the cancer therapy with an ability to inhibit function and/or activity of PD-1, PD-L1 and/or PD-L2.

In embodiments, a lack of upregulation of one or more genes associated with antigen processing/presentation of endogenous peptides via MHC class I compared to a prior biological sample from the subject indicates a response to the cancer therapy with an ability to inhibit function and/or activity of PD-1, PD-L1 and/or PD-L2. In embodiments, a lack of upregulation of one or more genes associated with antigen processing/presentation of endogenous peptides via MHC class I compared to a patient that is known to be sensitive to anti-PD-1 therapy indicates a response to the cancer therapy with an ability to inhibit function and/or activity of PD-1, PD-L1 and/or PD-L2. In embodiments, a lack of upregulation of one or more genes associated with antigen processing/presentation of endogenous peptides via MHC class I compared to a standard indicates a response to the cancer therapy with an ability to inhibit function and/or activity of PD-1, PD-L1 and/or PD-L2.

In embodiments, an upregulation of one or more genes associated with positive regulation of IFNα production compared to a prior biological sample from the subject indicates resistance, a lack of response, or recalcitrance to the cancer therapy with an ability to inhibit function and/or activity of PD-1, PD-L1 and/or PD-L2. In embodiments, an upregulation of one or more genes associated with positive regulation of IFNα production compared to a patient that is known to be sensitive to anti-PD-1 therapy indicates resistance, a lack of response, or recalcitrance to the cancer therapy with an ability to inhibit function and/or activity of PD-1, PD-L1 and/or PD-L2. In embodiments, an upregulation of one or more genes associated with positive regulation of IFNα production compared to a standard indicates resistance, a lack of response, or recalcitrance to the cancer therapy with an ability to inhibit function and/or activity of PD-1, PD-L1 and/or PD-L2.

In embodiments, a lack of upregulation of one or more genes associated with positive regulation of IFNα production compared to a prior biological sample from the subject indicates a response to the cancer therapy with an ability to inhibit function and/or activity of PD-1, PD-L1 and/or PD-L2. In embodiments, a lack of upregulation of one or more genes associated with positive regulation of IFNα production compared to a patient that is known to be sensitive to anti-PD-1 therapy indicates a response to the cancer therapy with an ability to inhibit function and/or activity of PD-1, PD-L1 and/or PD-L2. In embodiments, a lack of upregulation of one or more genes associated with positive regulation of IFNα production compared to a standard indicates a response to the cancer therapy with an ability to inhibit function and/or activity of PD-1, PD-L1 and/or PD-L2.

In embodiments, an upregulation of one or more genes associated with positive regulation of defense response compared to a prior biological sample from the subject indicates resistance, a lack of response, or recalcitrance to the cancer therapy with an ability to inhibit function and/or activity of PD-1, PD-L1 and/or PD-L2. In embodiments, an upregulation of one or more genes associated with positive regulation of defense response compared to a patient that is known to be sensitive to anti-PD-1 therapy indicates resistance, a lack of response, or recalcitrance to the cancer therapy with an ability to inhibit function and/or activity of PD-1, PD-L1 and/or PD-L2. In embodiments, an upregulation of one or more genes associated with positive regulation of defense response compared to a standard indicates resistance, a lack of response, or recalcitrance to the cancer therapy with an ability to inhibit function and/or activity of PD-1, PD-L1 and/or PD-L2.

In embodiments, a lack of upregulation of one or more genes associated with positive regulation of defense response compared to a prior biological sample from the subject indicates a response to the cancer therapy with an ability to inhibit function and/or activity of PD-1, PD-L1 and/or PD-L2. In embodiments, a lack of upregulation of one or more genes associated with positive regulation of defense response compared to a patient that is known to be sensitive to anti-PD-1 therapy indicates a response to the cancer therapy with an ability to inhibit function and/or activity of PD-1, PD-L1 and/or PD-L2. In embodiments, a lack of upregulation of one or more genes associated with positive regulation of defense response compared to a standard indicates a response to the cancer therapy with an ability to inhibit function and/or activity of PD-1, PD-L1 and/or PD-L2.

In embodiments, an upregulation of one or more genes associated with positive regulation of IFNβ production compared to a prior biological sample from the subject indicates resistance, a lack of response, or recalcitrance to the cancer therapy with an ability to inhibit function and/or activity of PD-1, PD-L1 and/or PD-L2. In embodiments, an upregulation of one or more genes associated with positive regulation of IFNβ production compared to a patient that is known to be sensitive to anti-PD-1 therapy indicates resistance, a lack of response, or recalcitrance to the cancer therapy with an ability to inhibit function and/or activity of PD-1, PD-L1 and/or PD-L2. In embodiments, an upregulation of one or more genes associated with positive regulation of IFNβ production compared to a standard indicates resistance, a lack of response, or recalcitrance to the cancer therapy with an ability to inhibit function and/or activity of PD-1, PD-L1 and/or PD-L2.

In embodiments, a lack of upregulation of one or more genes associated with positive regulation of IFNβ production compared to a prior biological sample from the subject indicates a response to the cancer therapy with an ability to inhibit function and/or activity of PD-1, PD-L1 and/or PD-L2. In embodiments, a lack of upregulation of one or more genes associated with positive regulation of IFNβ production compared to a patient that is known to be sensitive to anti-PD-1 therapy indicates a response to the cancer therapy with an ability to inhibit function and/or activity of PD-1, PD-L1 and/or PD-L2. In embodiments, a lack of upregulation of one or more genes associated with positive regulation of IFNβ production compared to a standard indicates a response to the cancer therapy with an ability to inhibit function and/or activity of PD-1, PD-L1 and/or PD-L2.

In embodiments, an upregulation of one or more genes associated with regulation of inflammatory response compared to a prior biological sample from the subject indicates resistance, a lack of response, or recalcitrance to the cancer therapy with an ability to inhibit function and/or activity of PD-1, PD-L1 and/or PD-L2. In embodiments, an upregulation of one or more genes associated with regulation of inflammatory response compared to a patient that is known to be sensitive to anti-PD-1 therapy indicates resistance, a lack of response, or recalcitrance to the cancer therapy with an ability to inhibit function and/or activity of PD-1, PD-L1 and/or PD-L2. In embodiments, an upregulation of one or more genes associated with regulation of inflammatory response compared to a standard indicates resistance, a lack of response, or recalcitrance to the cancer therapy with an ability to inhibit function and/or activity of PD-1, PD-L1 and/or PD-L2.

In embodiments, a lack of upregulation of one or more genes associated with regulation of inflammatory response compared to a prior biological sample from the subject indicates a response to the cancer therapy with an ability to inhibit function and/or activity of PD-1, PD-L1 and/or PD-L2. In embodiments, a lack of upregulation of one or more genes associated with regulation of inflammatory response compared to a patient that is known to be sensitive to anti-PD-1 therapy indicates a response to the cancer therapy with an ability to inhibit function and/or activity of PD-1, PD-L1 and/or PD-L2. In embodiments, a lack of upregulation of one or more genes associated with regulation of inflammatory response compared to a standard indicates a response to the cancer therapy with an ability to inhibit function and/or activity of PD-1, PD-L1 and/or PD-L2.

In embodiments, a downregulation of one or more genes associated with phospholipid efflux compared to a prior biological sample from the subject indicates resistance, a lack of response, or recalcitrance to the cancer therapy with an ability to inhibit function and/or activity of PD-1, PD-L1 and/or PD-L2. In embodiments, a downregulation of one or more genes associated with phospholipid efflux compared to a patient that is known to be sensitive to anti-PD-1 therapy indicates resistance, a lack of response, or recalcitrance to the cancer therapy with an ability to inhibit function and/or activity of PD-1, PD-L1 and/or PD-L2. In embodiments, a downregulation of one or more genes associated with phospholipid efflux compared to a standard indicates resistance, a lack of response, or recalcitrance to the cancer therapy with an ability to inhibit function and/or activity of PD-1, PD-L1 and/or PD-L2.

In embodiments, a lack of downregulation of one or more genes associated with phospholipid efflux compared to a prior biological sample from the subject indicates a response to the cancer therapy with an ability to inhibit function and/or activity of PD-1, PD-L1 and/or PD-L2. In embodiments, a lack of downregulation of one or more genes associated with phospholipid efflux compared to a patient that is known to be sensitive to anti-PD-1 therapy indicates a response to the cancer therapy with an ability to inhibit function and/or activity of PD-1, PD-L1 and/or PD-L2. In embodiments, a lack of downregulation of one or more genes associated with phospholipid efflux compared to a standard indicates a response to the cancer therapy with an ability to inhibit function and/or activity of PD-1, PD-L1 and/or PD-L2.

In embodiments, a downregulation of one or more genes associated with negative regulation of fibrinolysis compared to a prior biological sample from the subject indicates resistance, a lack of response, or recalcitrance to the cancer therapy with an ability to inhibit function and/or activity of PD-1, PD-L1 and/or PD-L2. In embodiments, a downregulation of one or more genes associated with negative regulation of fibrinolysis compared to a patient that is known to be sensitive to anti-PD-1 therapy indicates resistance, a lack of response, or recalcitrance to the cancer therapy with an ability to inhibit function and/or activity of PD-1, PD-L1 and/or PD-L2. In embodiments, a downregulation of one or more genes associated with negative regulation of fibrinolysis compared to a standard indicates resistance, a lack of response, or recalcitrance to the cancer therapy with an ability to inhibit function and/or activity of PD-1, PD-L1 and/or PD-L2.

In embodiments, a lack of downregulation of one or more genes associated with negative regulation of fibrinolysis compared to a prior biological sample from the subject indicates a response to the cancer therapy with an ability to inhibit function and/or activity of PD-1, PD-L1 and/or PD-L2. In embodiments, a lack of downregulation of one or more genes associated with negative regulation of fibrinolysis compared to a patient that is known to be sensitive to anti-PD-1 therapy indicates a response to the cancer therapy with an ability to inhibit function and/or activity of PD-1, PD-L1 and/or PD-L2. In embodiments, a lack of downregulation of one or more genes associated with negative regulation of fibrinolysis compared to a standard indicates a response to the cancer therapy with an ability to inhibit function and/or activity of PD-1, PD-L1 and/or PD-L2.

In embodiments, a downregulation of one or more genes associated with chylomicron assembly compared to a prior biological sample from the subject indicates resistance, a lack of response, or recalcitrance to the cancer therapy with an ability to inhibit function and/or activity of PD-1, PD-L1 and/or PD-L2. In embodiments, a downregulation of one or more genes associated with chylomicron assembly compared to a patient that is known to be sensitive to anti-PD-1 therapy indicates resistance, a lack of response, or recalcitrance to the cancer therapy with an ability to inhibit function and/or activity of PD-1, PD-L1 and/or PD-L2. In embodiments, a downregulation of one or more genes associated with chylomicron assembly compared to a standard indicates resistance, a lack of response, or recalcitrance to the cancer therapy with an ability to inhibit function and/or activity of PD-1, PD-L1 and/or PD-L2.

In embodiments, a lack of downregulation of one or more genes associated with chylomicron assembly compared to a prior biological sample from the subject indicates a response to the cancer therapy with an ability to inhibit function and/or activity of PD-1, PD-L1 and/or PD-L2. In embodiments, a lack of downregulation of one or more genes associated with chylomicron assembly compared to a patient that is known to be sensitive to anti-PD-1 therapy indicates a response to the cancer therapy with an ability to inhibit function and/or activity of PD-1, PD-L1 and/or PD-L2. In embodiments, a lack of downregulation of one or more genes associated with chylomicron assembly compared to a standard indicates a response to the cancer therapy with an ability to inhibit function and/or activity of PD-1, PD-L1 and/or PD-L2.

In embodiments, a downregulation of one or more genes associated with plasma membrane repair compared to a prior biological sample from the subject indicates resistance, a lack of response, or recalcitrance to the cancer therapy with an ability to inhibit function and/or activity of PD-1, PD-L1 and/or PD-L2. In embodiments, a downregulation of one or more genes associated with plasma membrane repair compared to a patient that is known to be sensitive to anti-PD-1 therapy indicates resistance, a lack of response, or recalcitrance to the cancer therapy with an ability to inhibit function and/or activity of PD-1, PD-L1 and/or PD-L2. In embodiments, a downregulation of one or more genes associated with plasma membrane repair compared to a standard indicates resistance, a lack of response, or recalcitrance to the cancer therapy with an ability to inhibit function and/or activity of PD-1, PD-L1 and/or PD-L2.

In embodiments, a lack of downregulation of one or more genes associated with plasma membrane repair compared to a prior biological sample from the subject indicates a response to the cancer therapy with an ability to inhibit function and/or activity of PD-1, PD-L1 and/or PD-L2. In embodiments, a lack of downregulation of one or more genes associated with plasma membrane repair compared to a patient that is known to be sensitive to anti-PD-1 therapy indicates a response to the cancer therapy with an ability to inhibit function and/or activity of PD-1, PD-L1 and/or PD-L2. In embodiments, a lack of downregulation of one or more genes associated with plasma membrane repair compared to a standard indicates a response to the cancer therapy with an ability to inhibit function and/or activity of PD-1, PD-L1 and/or PD-L2.

In embodiments, a downregulation of one or more genes associated with SRP-dependent co-translational protein targeting to membrane compared to a prior biological sample from the subject indicates resistance, a lack of response, or recalcitrance to the cancer therapy with an ability to inhibit function and/or activity of PD-1, PD-L1 and/or PD-L2. In embodiments, a downregulation of one or more genes associated with SRP-dependent co-translational protein targeting to membrane compared to a patient that is known to be sensitive to anti-PD-1 therapy indicates resistance, a lack of response, or recalcitrance to the cancer therapy with an ability to inhibit function and/or activity of PD-1, PD-L1 and/or PD-L2. In embodiments, a downregulation of one or more genes associated with SRP-dependent co-translational protein targeting to membrane compared to a standard indicates resistance, a lack of response, or recalcitrance to the cancer therapy with an ability to inhibit function and/or activity of PD-1, PD-L1 and/or PD-L2.

In embodiments, a lack of downregulation of one or more genes associated with SRP-dependent co-translational protein targeting to membrane compared to a prior biological sample from the subject indicates a response to the cancer therapy with an ability to inhibit function and/or activity of PD-1, PD-L1 and/or PD-L2. In embodiments, a lack of downregulation of one or more genes associated with SRP-dependent co-translational protein targeting to membrane compared to a patient that is known to be sensitive to anti-PD-1 therapy indicates a response to the cancer therapy with an ability to inhibit function and/or activity of PD-1, PD-L1 and/or PD-L2. In embodiments, a lack of downregulation of one or more genes associated with SRP-dependent co-translational protein targeting to membrane compared to a standard indicates a response to the cancer therapy with an ability to inhibit function and/or activity of PD-1, PD-L1 and/or PD-L2.

In embodiments, a downregulation of one or more genes associated with ribosomal small subunit assembly compared to a prior biological sample from the subject indicates resistance, a lack of response, or recalcitrance to the cancer therapy with an ability to inhibit function and/or activity of PD-1, PD-L1 and/or PD-L2. In embodiments, a downregulation of one or more genes associated with ribosomal small subunit assembly compared to a patient that is known to be sensitive to anti-PD-1 therapy indicates resistance, a lack of response, or recalcitrance to the cancer therapy with an ability to inhibit function and/or activity of PD-1, PD-L1 and/or PD-L2. In embodiments, a downregulation of one or more genes associated with ribosomal small subunit assembly compared to a standard indicates resistance, a lack of response, or recalcitrance to the cancer therapy with an ability to inhibit function and/or activity of PD-1, PD-L1 and/or PD-L2.

In embodiments, a lack of downregulation of one or more genes associated with ribosomal small subunit assembly compared to a prior biological sample from the subject indicates a response to the cancer therapy with an ability to inhibit function and/or activity of PD-1, PD-L1 and/or PD-L2. In embodiments, a lack of downregulation of one or more genes associated with ribosomal small subunit assembly compared to a patient that is known to be sensitive to anti-PD-1 therapy indicates a response to the cancer therapy with an ability to inhibit function and/or activity of PD-1, PD-L1 and/or PD-L2. In embodiments, a lack of downregulation of one or more genes associated with ribosomal small subunit assembly compared to a standard indicates a response to the cancer therapy with an ability to inhibit function and/or activity of PD-1, PD-L1 and/or PD-L2.

In embodiments, a downregulation of one or more genes associated with phospholipid efflux compared to a prior biological sample from the subject indicates resistance, a lack of response, or recalcitrance to the cancer therapy with an ability to inhibit function and/or activity of PD-1, PD-L1 and/or PD-L2. In embodiments, a downregulation of one or more genes associated with phospholipid efflux compared to a patient that is known to be sensitive to anti-PD-1 therapy indicates resistance, a lack of response, or recalcitrance to the cancer therapy with an ability to inhibit function and/or activity of PD-1, PD-L1 and/or PD-L2. In embodiments, a downregulation of one or more genes associated with phospholipid efflux compared to a standard indicates resistance, a lack of response, or recalcitrance to the cancer therapy with an ability to inhibit function and/or activity of PD-1, PD-L1 and/or PD-L2.

In embodiments, a lack of downregulation of one or more genes associated with phospholipid efflux compared to a prior biological sample from the subject indicates a response to the cancer therapy with an ability to inhibit function and/or activity of PD-1, PD-L1 and/or PD-L2. In embodiments, a lack of downregulation of one or more genes associated with phospholipid efflux compared to a patient that is known to be sensitive to anti-PD-1 therapy indicates a response to the cancer therapy with an ability to inhibit function and/or activity of PD-1, PD-L1 and/or PD-L2. In embodiments, a lack of downregulation of one or more genes associated with phospholipid efflux compared to a standard indicates a response to the cancer therapy with an ability to inhibit function and/or activity of PD-1, PD-L1 and/or PD-L2.

In embodiments, a downregulation of one or more genes associated with regulation of translation compared to a prior biological sample from the subject indicates resistance, a lack of response, or recalcitrance to the cancer therapy with an ability to inhibit function and/or activity of PD-1, PD-L1 and/or PD-L2. In embodiments, a downregulation of one or more genes associated with regulation of translation compared to a patient that is known to be sensitive to anti-PD-1 therapy indicates resistance, a lack of response, or recalcitrance to the cancer therapy with an ability to inhibit function and/or activity of PD-1, PD-L1 and/or PD-L2. In embodiments, a downregulation of one or more genes associated with regulation of translation compared to a standard indicates resistance, a lack of response, or recalcitrance to the cancer therapy with an ability to inhibit function and/or activity of PD-1, PD-L1 and/or PD-L2.

In embodiments, a lack of downregulation of one or more genes associated with regulation of translation compared to a prior biological sample from the subject indicates a response to the cancer therapy with an ability to inhibit function and/or activity of PD-1, PD-L1 and/or PD-L2. In embodiments, a lack of downregulation of one or more genes associated with regulation of translation compared to a patient that is known to be sensitive to anti-PD-1 therapy indicates a response to the cancer therapy with an ability to inhibit function and/or activity of PD-1, PD-L1 and/or PD-L2. In embodiments, a lack of downregulation of one or more genes associated with regulation of translation compared to a standard indicates a response to the cancer therapy with an ability to inhibit function and/or activity of PD-1, PD-L1 and/or PD-L2.

In embodiments, a downregulation of one or more genes associated with mitochondrial respiratory chain complex I compared to a prior biological sample from the subject indicates resistance, a lack of response, or recalcitrance to the cancer therapy with an ability to inhibit function and/or activity of PD-1, PD-L1 and/or PD-L2. In embodiments, a downregulation of one or more genes associated with mitochondrial respiratory chain complex I compared to a patient that is known to be sensitive to anti-PD-1 therapy indicates resistance, a lack of response, or recalcitrance to the cancer therapy with an ability to inhibit function and/or activity of PD-1, PD-L1 and/or PD-L2. In embodiments, a downregulation of one or more genes associated with mitochondrial respiratory chain complex I compared to a standard indicates resistance, a lack of response, or recalcitrance to the cancer therapy with an ability to inhibit function and/or activity of PD-1, PD-L1 and/or PD-L2.

In embodiments, a lack of downregulation of one or more genes associated with mitochondrial respiratory chain complex I compared to a prior biological sample from the subject indicates a response to the cancer therapy with an ability to inhibit function and/or activity of PD-1, PD-L1 and/or PD-L2. In embodiments, a lack of downregulation of one or more genes associated with mitochondrial respiratory chain complex I compared to a patient that is known to be sensitive to anti-PD-1 therapy indicates a response to the cancer therapy with an ability to inhibit function and/or activity of PD-1, PD-L1 and/or PD-L2. In embodiments, a lack of downregulation of one or more genes associated with mitochondrial respiratory chain complex I compared to a standard indicates a response to the cancer therapy with an ability to inhibit function and/or activity of PD-1, PD-L1 and/or PD-L2.

In embodiments, a downregulation of one or more genes associated with mitochondrial translational elongation compared to a prior biological sample from the subject indicates resistance, a lack of response, or recalcitrance to the cancer therapy with an ability to inhibit function and/or activity of PD-1, PD-L1 and/or PD-L2. In embodiments, a downregulation of one or more genes associated with mitochondrial translational elongation compared to a patient that is known to be sensitive to anti-PD-1 therapy indicates resistance, a lack of response, or recalcitrance to the cancer therapy with an ability to inhibit function and/or activity of PD-1, PD-L1 and/or PD-L2. In embodiments, a downregulation of one or more genes associated with mitochondrial translational elongation compared to a standard indicates resistance, a lack of response, or recalcitrance to the cancer therapy with an ability to inhibit function and/or activity of PD-1, PD-L1 and/or PD-L2.

In embodiments, a lack of downregulation of one or more genes associated with mitochondrial translational elongation compared to a prior biological sample from the subject indicates a response to the cancer therapy with an ability to inhibit function and/or activity of PD-1, PD-L1 and/or PD-L2. In embodiments, a lack of downregulation of one or more genes associated with mitochondrial translational elongation compared to a patient that is known to be sensitive to anti-PD-1 therapy indicates a response to the cancer therapy with an ability to inhibit function and/or activity of PD-1, PD-L1 and/or PD-L2. In embodiments, a lack of downregulation of one or more genes associated with mitochondrial translational elongation compared to a standard indicates a response to the cancer therapy with an ability to inhibit function and/or activity of PD-1, PD-L1 and/or PD-L2.

In embodiments, a downregulation of one or more genes associated with DNA-dependent DNA replication compared to a prior biological sample from the subject indicates resistance, a lack of response, or recalcitrance to the cancer therapy with an ability to inhibit function and/or activity of PD-1, PD-L1 and/or PD-L2. In embodiments, a downregulation of one or more genes associated with DNA-dependent DNA replication compared to a patient that is known to be sensitive to anti-PD-1 therapy indicates resistance, a lack of response, or recalcitrance to the cancer therapy with an ability to inhibit function and/or activity of PD-1, PD-L1 and/or PD-L2. In embodiments, a downregulation of one or more genes associated with DNA-dependent DNA replication compared to a standard indicates resistance, a lack of response, or recalcitrance to the cancer therapy with an ability to inhibit function and/or activity of PD-1, PD-L1 and/or PD-L2.

In embodiments, a lack of downregulation of one or more genes associated with DNA-dependent DNA replication compared to a prior biological sample from the subject indicates a response to the cancer therapy with an ability to inhibit function and/or activity of PD-1, PD-L1 and/or PD-L2. In embodiments, a lack of downregulation of one or more genes associated with DNA-dependent DNA replication compared to a patient that is known to be sensitive to anti-PD-1 therapy indicates a response to the cancer therapy with an ability to inhibit function and/or activity of PD-1, PD-L1 and/or PD-L2. In embodiments, a lack of downregulation of one or more genes associated with DNA-dependent DNA replication compared to a standard indicates a response to the cancer therapy with an ability to inhibit function and/or activity of PD-1, PD-L1 and/or PD-L2.

In embodiments, a downregulation of one or more genes associated with ATP biosynthetic process compared to a prior biological sample from the subject indicates resistance, a lack of response, or recalcitrance to the cancer therapy with an ability to inhibit function and/or activity of PD-1, PD-L1 and/or PD-L2. In embodiments, a downregulation of one or more genes associated with ATP biosynthetic process compared to a patient that is known to be sensitive to anti-PD-1 therapy indicates resistance, a lack of response, or recalcitrance to the cancer therapy with an ability to inhibit function and/or activity of PD-1, PD-L1 and/or PD-L2. In embodiments, a downregulation of one or more genes associated with ATP biosynthetic process compared to a standard indicates resistance, a lack of response, or recalcitrance to the cancer therapy with an ability to inhibit function and/or activity of PD-1, PD-L1 and/or PD-L2.

In embodiments, a lack of downregulation of one or more genes associated with ATP biosynthetic process compared to a prior biological sample from the subject indicates a response to the cancer therapy with an ability to inhibit function and/or activity of PD-1, PD-L1 and/or PD-L2. In embodiments, a lack of downregulation of one or more genes associated with ATP biosynthetic process compared to a patient that is known to be sensitive to anti-PD-1 therapy indicates a response to the cancer therapy with an ability to inhibit function and/or activity of PD-1, PD-L1 and/or PD-L2. In embodiments, a lack of downregulation of one or more genes associated with ATP biosynthetic process compared to a standard indicates a response to the cancer therapy with an ability to inhibit function and/or activity of PD-1, PD-L1 and/or PD-L2.

In embodiments, a cancer therapy with an ability to inhibit function and/or activity of PD-1, PD-L1 and/or PD-L2 may be indicated, for example, when expression of one or more of Rpl41, Rps15 and Rps8 is high and/or the expression of one or more of Cd274, B2M, Tap1, Tap2, Casp1, and Gasta3 is low. In embodiments, a cancer therapy with an ability to inhibit function and/or activity of PD-1, PD-L1 and/or PD-L2 may not be indicated, for example, when expression of one or more of Rpl41, Rps15 and Rps8 is low and/or the expression of one or more of Cd274, B2M, Tap1, Tap2, Casp1, and Gasta3 is high. In embodiments, a patient featuring low expression of one or more of Rpl41, Rps15 and Rps8 and/or high expression of one or more of Cd274, B2M, Tap1, Tap2, Casp1, and Gasta3 are likely to benefit from adjuvant or neoadjuvant therapies that eliminate the PD-1-nonresponsive cells.

In embodiments, the biological sample is a fresh tissue sample, frozen tumor tissue specimen, cultured cells, circulating tumor cells, or a formalin-fixed paraffin-embedded tumor tissue specimen. In embodiments, the biological sample is a biopsy sample. In embodiments, the biopsy sample is selected from endoscopic biopsy, bone marrow biopsy, endoscopic biopsy (e.g., cystoscopy, bronchoscopy and colonoscopy), needle biopsy (e.g., fine-needle aspiration, core needle biopsy, vacuum-assisted biopsy, X-ray-assisted biopsy, computerized tomography (CT)-assisted biopsy, magnetic resonance imaging (MRI)-assisted biopsy and ultrasound-assisted biopsy), skin biopsy (e.g., shave biopsy, punch biopsy, and incisional biopsy) and surgical biopsy.

In embodiments, the biological sample comprises a body fluid selected from blood, plasma, serum, lacrimal fluid, tears, bone marrow, blood, blood cells, ascites, tissue or fine needle biopsy sample, cell-containing body fluid, free floating nucleic acids, sputum, saliva, urine, cerebrospinal fluid, peritoneal fluid, pleural fluid, feces, lymph, gynecological fluid, skin swab, vaginal swab, oral swab, nasal swab, washing or lavage such as a ductal lavage or broncheoalveolar lavage, aspirate, scraping, bone marrow specimen, tissue biopsy specimen, surgical specimen, feces, other body fluids, secretions, and/or excretions, and/or cells therefrom. In embodiments, the biological sample is obtained by a technique selected from scrapes, swabs, and biopsy. In embodiments, the biological sample is obtained by use of brushes, (cotton) swabs, spatula, rinse/wash fluids, punch biopsy devices, puncture of cavities with needles or surgical instrumentation.

In embodiments, the biological sample comprises at least one tumor cell. In embodiments, the tumor is selected from Hodgkin's and non-Hodgkin's lymphoma, B-cell lymphoma (including low grade/follicular non-Hodgkin's lymphoma (NHL); small lymphocytic (SL) NHL; intermediate grade/follicular NHL; intermediate grade diffuse NHL; high grade immunoblastic NHL; high grade lymphoblastic NHL; high grade small non-cleaved cell NHL; bulky disease NHL; mantle cell lymphoma; AIDS-related lymphoma; and Waldenstrom's Macroglobulinemia; chronic lymphocytic leukemia (CLL); acute lymphoblastic leukemia (ALL); Hairy cell leukemia; or chronic myeloblastic leukemia. In some embodiments, the cancer is basal cell carcinoma, biliary tract cancer; bladder cancer; bone cancer; brain and central nervous system cancer; breast cancer; cancer of the peritoneum; cervical cancer; choriocarcinoma; colon and rectum cancer; connective tissue cancer; cancer of the digestive system; endometrial cancer; esophageal cancer; eye cancer; cancer of the head and neck; gastric cancer (including gastrointestinal cancer); glioblastoma; hepatic carcinoma; hepatoma; intra-epithelial neoplasm; kidney or renal cancer; larynx cancer; leukemia; liver cancer; lung cancer (e.g., small-cell lung cancer, non-small cell lung cancer, adenocarcinoma of the lung, and squamous carcinoma of the lung); melanoma; myeloma; neuroblastoma; oral cavity cancer (lip, tongue, mouth, and pharynx); ovarian cancer; pancreatic cancer; prostate cancer; retinoblastoma; rhabdomyosarcoma; rectal cancer; cancer of the respiratory system; salivary gland carcinoma; sarcoma; skin cancer; squamous cell cancer; stomach cancer; testicular cancer; thyroid cancer; uterine or endometrial cancer; cancer of the urinary system; vulval cancer; lymphoma including Hodgkin's and non-Hodgkin's lymphoma, as well as B-cell lymphoma (including low grade/follicular non-Hodgkin's lymphoma (NHL); small lymphocytic (SL) NHL; intermediate grade/follicular NHL; intermediate grade diffuse NHL; high grade immunoblastic NHL; high grade lymphoblastic NHL; high grade small non-cleaved cell NHL; bulky disease NHL; mantle cell lymphoma; AIDS-related lymphoma; and Waldenstrom's Macroglobulinemia; chronic lymphocytic leukemia (CLL); acute lymphoblastic leukemia (ALL); Hairy cell leukemia; chronic myeloblastic leukemia; as well as other carcinomas and sarcomas; and post-transplant lymphoproliferative disorder (PTLD), as well as abnormal vascular proliferation associated with phakomatoses, edema (e.g., that associated with brain tumors), Meigs' syndrome cancer; renal carcinoma; colorectal cancer; and adrenal cancer.

In embodiments, the evaluating is performed by DNA sequencing, RNA sequencing, immunohistochemical staining, western blotting, in cell western, immunofluorescent staining, ELISA, and fluorescent activating cell sorting (FACS) or a combination thereof.

In embodiments, the evaluating is performed by contacting the sample with an agent that specifically binds to one or more proteins encoded by one or more genes associated with a gene ontology (GO) pathway selected from: (i) positive regulation of cell cycle process, regulation of G1/S transition, regulation of cell division, regulation of cell proliferation, positive regulation of IκB kinase/NFκB signaling, type I IFN signaling pathway, cellular response to IFNγ, positive regulation of IFNα production, positive regulation of defense response, positive regulation of IFNβ production, regulation of inflammatory response, regulation of innate immune response, negative regulation of antigen processing and presentation, and antigen processing, presentation of endogenous peptides via MHC class I; and/or (ii) phospholipid efflux, negative regulation of fibrinolysis, chylomicron assembly, plasma membrane repair, SRP-dependent co-translational protein targeting to membrane, ribosomal small subunit assembly, phospholipid efflux, regulation of translation, mitochondrial respiratory chain complex I, mitochondrial translational elongation, DNA-dependent DNA replication, and ATP biosynthetic process.

In embodiments, the evaluating is performed by contacting the sample with an agent that specifically binds to one or more proteins encoded by one or more genes associated with a gene ontology (GO) pathway selected from (a) cellular response to IFNγ, (b) negative regulation of antigen processing and presentation, (c) type I IFN signaling pathway, (d) positive regulation of IκB kinase/NFκB signaling, and antigen processing, and (e) presentation of endogenous peptides via MHC class I. In embodiments, the evaluating is performed by contacting the sample with an agent that specifically binds to one or more proteins encoded by one or more genes associated with cellular response to IFNγ. In embodiments, the evaluating is performed by contacting the sample with an agent that specifically binds to one or more proteins encoded by one or more genes associated with type I IFN signaling pathway. In embodiments, the evaluating is performed by contacting the sample with an agent that specifically binds to one or more proteins encoded by one or more genes associated with positive regulation of IFNα production. In embodiments, the evaluating is performed by contacting the sample with an agent that specifically binds to one or more proteins encoded by one or more genes associated with positive regulation of defense response. In embodiments, the evaluating is performed by contacting the sample with an agent that specifically binds to one or more proteins encoded by one or more genes associated with positive regulation of I IFNβ production. In embodiments, the evaluating is performed by contacting the sample with an agent that specifically binds to one or more proteins encoded by one or more genes associated with regulation of inflammatory response.

In embodiments, the evaluating is performed by contacting the sample with an agent that specifically binds to one or more of nucleic acids of one or more genes associated with a gene ontology (GO) pathway selected from: (i) positive regulation of cell cycle process, regulation of G1/S transition, regulation of cell division, regulation of cell proliferation, positive regulation of IκB kinase/NFκB signaling, type I IFN signaling pathway, cellular response to IFNγ, positive regulation of IFNα production, positive regulation of defense response, positive regulation of IFNβ production, regulation of inflammatory response, regulation of innate immune response, negative regulation of antigen processing and presentation, and antigen processing, presentation of endogenous peptides via MHC class I; and/or (ii) phospholipid efflux, negative regulation of fibrinolysis, chylomicron assembly, plasma membrane repair, SRP-dependent co-translational protein targeting to membrane, ribosomal small subunit assembly, phospholipid efflux, regulation of translation, mitochondrial respiratory chain complex I, mitochondrial translational elongation, DNA-dependent DNA replication, and ATP biosynthetic process.

In embodiments, the evaluating is performed by contacting the sample with an agent that specifically binds to one or more nucleic acid of one or more genes associated with a gene ontology (GO) pathway selected from (a) cellular response to IFNγ, (b) negative regulation of antigen processing and presentation, (c) type I IFN signaling pathway, (d) positive regulation of IκB kinase/NFκB signaling, and antigen processing, and (e) presentation of endogenous peptides via MHC class I. In embodiments, the evaluating is performed by contacting the sample with an agent that specifically binds to one or more nucleic acid of one or more genes associated with cellular response to IFNγ. In embodiments, the evaluating is performed by contacting the sample with an agent that specifically binds to one or more nucleic acid of one or more genes associated with type I IFN signaling pathway. In embodiments, the evaluating is performed by contacting the sample with an agent that specifically binds to one or more nucleic acid of one or more genes associated with positive regulation of IFNα production. In embodiments, the evaluating is performed by contacting the sample with an agent that specifically binds to one or more nucleic acid of one or more genes associated with positive regulation of defense response. In embodiments, the evaluating is performed by contacting the sample with an agent that specifically binds to one or more nucleic acid of one or more genes associated with positive regulation of IFNβ production. In embodiments, the evaluating is performed by contacting the sample with an agent that specifically binds to one or more nucleic acid of one or more genes associated with regulation of inflammatory response. In embodiments, the agent that specifically binds to one or more of the nucleic acids is a nucleic acid primer or probe.

In embodiments, the evaluating informs classifying the patient into a high or low risk group. In embodiments, the high risk classification comprises a high level of tumor cells having resistance to the cancer therapy with an ability to inhibit function and/or activity of PD-1, PD-L1 and/or PD-L2. In embodiments, the low risk classification comprises a low level of tumor cells having resistance to the cancer therapy with an ability to inhibit function and/or activity of PD-1, PD-L1 and/or PD-L2.

In embodiments, the low risk or high risk classification is indicative of withholding of the neoadjuvant therapy. In embodiments, the low risk or high risk classification is indicative of withholding of the adjuvant therapy. In embodiments, the evaluating is predictive of a positive response to and/or benefit from the cancer treatment. In embodiments, the evaluating is predictive of a negative or neutral response to and/or benefit from the cancer treatment. In embodiments, the evaluating is predictive of a positive response to and/or benefit from neoadjuvant chemotherapy or a non-responsiveness to and/or lack of benefit from neoadjuvant chemotherapy. In embodiments, the evaluating is predictive of a positive response to and/or benefit from adjuvant chemotherapy or a non-responsiveness to and/or lack of benefit from adjuvant chemotherapy. In embodiments, the evaluating is predictive of a negative or neutral response to and/or benefit from neoadjuvant chemotherapy or a non-responsiveness to and/or lack of benefit from neoadjuvant chemotherapy. In embodiments, the evaluating is predictive of a negative or neutral response to and/or benefit from adjuvant chemotherapy or a non-responsiveness to and/or lack of benefit from adjuvant chemotherapy. In embodiments, the evaluating informs administration or withholding of the cancer treatment. In embodiments, the evaluating informs administration of neoadjuvant therapy. In embodiments, the evaluating informs administration of adjuvant therapy. In embodiments, the evaluating informs withholding of neoadjuvant therapy. In embodiments, the evaluating informs withholding of adjuvant therapy. In embodiments, the neoadjuvant therapy and/or the adjuvant therapy is a chemotherapeutic agent. In embodiments, the neoadjuvant therapy and/or the adjuvant therapy is a cytotoxic agent. In embodiments, the neoadjuvant therapy and/or the adjuvant therapy is a checkpoint inhibitor.

In embodiments, the neoadjuvant therapy and/or the adjuvant therapy is selected from a protein translation inhibitor (e.g., silvestrol and omacetaxine) ribosome biogenesis inhibitors (e.g., diazaborine, lamotrigine and ribozinoindoles), inhibitors of rRNA and/or tRNA synthesis (e.g., quarfloxin (CX-3543) and CX-5461), an inhibitor of synthesis of amino acids (e.g., GLUD1 inhibitor R162, BCAT1 inhibitor gabapentin, glutaminase inhibitor bis-2-(5-phenylacetamido-1,2,4-thiadiazol-2-yl)ethyl sulfide (BPTES), PAGDH inhibitor NCT-503), an inhibitor of uptake of amino acids (e.g., SLC7A11 inhibitors sulfasalazine, erastin or sorafenib), a modulator of post-translational modification (e.g., glycosylation inhibitor tunicamycin, ppGalNAc-T3), a modulator of protein degradation, and a modulator of protein transport (e.g., cyclosporin A, fendiline, parbendazole, paroxetine, parthenolide, quinacrine, sertraline, spiperone, thimerosal, astemizole, perhexiline, HUN-7293, CAM741, CK147, and cotransin).

In one aspect, the present disclosure relates to a method of determining a cancer treatment for a patient, the method comprising: (i) obtaining a biological sample from a subject; (ii) evaluating the sample for the upregulation of one or more genes associated with a gene ontology (GO) pathway selected from positive regulation of cell cycle process, regulation of G1/S transition, regulation of cell division, regulation of cell proliferation, positive regulation of IκB kinase/NFκB signaling, type I IFN signaling pathway, cellular response to IFNγ, positive regulation of IFNα production, positive regulation of defense response, positive regulation of IFNβ production, regulation of inflammatory response, regulation of innate immune response, negative regulation of antigen processing/presentation, and antigen processing/presentation of endogenous peptides via MHC class I; and/or downregulation of one or more genes associated with a gene ontology (GO) pathway selected from phospholipid efflux, negative regulation of fibrinolysis, chylomicron assembly, plasma membrane repair, SRP-dependent co-translational protein targeting to membrane, ribosomal small subunit assembly, phospholipid efflux, regulation of translation, mitochondrial respiratory chain complex I, mitochondrial translational elongation, DNA-dependent DNA replication, and ATP biosynthetic process; and (iii) selecting the cancer therapy based on the evaluation of step (b), wherein the cancer therapy comprises a chimeric protein of a general structure of: N terminus—(a)—(b)—(c)—C terminus, wherein: (A) (a) is a first domain comprising an extracellular domain of a Type I transmembrane protein, the transmembrane protein being TIGIT, (b) is a linker comprising at least one cysteine residue capable of forming a disulfide bond, and (c) is a second domain comprising an extracellular domain of Type II transmembrane protein, the transmembrane protein being LIGHT, wherein the linker connects the first domain and the second domain and optionally comprises one or more joining linkers, such joining linkers being selected from SEQ ID NOs: 49-95; or (B) (a) is a first domain comprising an extracellular domain of a Type I transmembrane protein, the transmembrane protein being SIRPα, (b) is a linker comprising at least one cysteine residue capable of forming a disulfide bond, and (c) is a second domain comprising an extracellular domain of Type II transmembrane protein, the transmembrane protein being 4-1BBL, wherein the linker connects the first domain and the second domain and optionally comprises one or more joining linkers, such joining linkers being selected from SEQ ID NOs: 49-95.

In one aspect, the present disclosure relates to a method for selecting a patient for a cancer treatment, the method comprising: (i) obtaining a biological sample from a subject; (ii) evaluating the sample for the upregulation of one or more genes associated with a gene ontology (GO) pathway selected from positive regulation of cell cycle process, regulation of G1/S transition, regulation of cell division, regulation of cell proliferation, positive regulation of IκB kinase/NFκB signaling, type I IFN signaling pathway, cellular response to IFNγ, positive regulation of IFNα production, positive regulation of defense response, positive regulation of IFNβ production, regulation of inflammatory response, regulation of innate immune response, negative regulation of antigen processing/presentation, and antigen processing/presentation of endogenous peptides via MHC class I; and/or downregulation of one or more genes associated with a gene ontology (GO) pathway selected from phospholipid efflux, negative regulation of fibrinolysis, chylomicron assembly, plasma membrane repair, SRP-dependent co-translational protein targeting to membrane, ribosomal small subunit assembly, phospholipid efflux, regulation of translation, mitochondrial respiratory chain complex I, mitochondrial translational elongation, DNA-dependent DNA replication, and ATP biosynthetic process; and (iii) selecting a cancer therapy wherein the cancer therapy comprises a chimeric protein of a general structure of: N terminus—(a)—(b)—(c)—C terminus, wherein: (A) (a) is a first domain comprising an extracellular domain of a Type I transmembrane protein, the transmembrane protein being TIGIT, (b) is a linker comprising at least one cysteine residue capable of forming a disulfide bond, and (c) is a second domain comprising an extracellular domain of Type II transmembrane protein, the transmembrane protein being LIGHT, wherein the linker connects the first domain and the second domain and optionally comprises one or more joining linkers, such joining linkers being selected from SEQ ID NOs: 49-95; or (B) (a) is a first domain comprising an extracellular domain of a Type I transmembrane protein, the transmembrane protein being SIRPα, (b) is a linker comprising at least one cysteine residue capable of forming a disulfide bond, and (c) is a second domain comprising an extracellular domain of Type II transmembrane protein, the transmembrane protein being 4-1BBL, wherein the linker connects the first domain and the second domain and optionally comprises one or more joining linkers, such joining linkers being selected from SEQ ID NOs: 49-95.

In one aspect, the present disclosure relates to a method of treating cancer, the method comprising: (i) obtaining a biological sample from a subject; (ii) evaluating the sample for the upregulation of one or more genes associated with a gene ontology (GO) pathway selected from positive regulation of cell cycle process, regulation of G1/S transition, regulation of cell division, regulation of cell proliferation, positive regulation of IκB kinase/NFκB signaling, type I IFN signaling pathway, cellular response to IFNγ, positive regulation of IFNα production, positive regulation of defense response, positive regulation of IFNβ production, regulation of inflammatory response, regulation of innate immune response, negative regulation of antigen processing/presentation, and antigen processing/presentation of endogenous peptides via MHC class I; and/or downregulation of one or more genes associated with a gene ontology (GO) pathway selected from phospholipid efflux, negative regulation of fibrinolysis, chylomicron assembly, plasma membrane repair, SRP-dependent co-translational protein targeting to membrane, ribosomal small subunit assembly, phospholipid efflux, regulation of translation, mitochondrial respiratory chain complex I, mitochondrial translational elongation, DNA-dependent DNA replication, and ATP biosynthetic process; and (iii) selecting the cancer therapy wherein the cancer therapy comprises a chimeric protein of a general structure of: N terminus—(a)—(b)—(c)—C terminus, wherein: (a) is a first domain comprising an extracellular domain of a Type I transmembrane protein, the transmembrane protein being TIGIT, (b) is a linker comprising at least one cysteine residue capable of forming a disulfide bond, and (c) is a second domain comprising an extracellular domain of Type II transmembrane protein, the transmembrane protein being LIGHT, wherein the linker connects the first domain and the second domain and optionally comprises one or more joining linkers, such joining linkers being selected from SEQ ID NOs: 49-95.

In embodiments, the upregulation is in comparison to a healthy tissue. In embodiments, the upregulation is in comparison to another biological sample from patient that is known to be sensitive to anti-PD-1 therapy. In embodiments, the upregulation is in comparison to a prior biological sample obtained from the subject. In embodiments, the downregulation is in comparison to a healthy tissue. In embodiments, the downregulation is in comparison to another biological sample from patient that is known to be sensitive to anti-PD-1 therapy. In embodiments, the downregulation is in comparison to a prior biological sample obtained from the subject.

In embodiments, the biological sample is a fresh tissue sample, frozen tumor tissue specimen, cultured cells, circulating tumor cells, or a formalin-fixed paraffin-embedded tumor tissue specimen. In embodiments, the biological sample is a biopsy sample. In embodiments, the biopsy sample is selected from endoscopic biopsy, bone marrow biopsy, endoscopic biopsy (e.g., cystoscopy, bronchoscopy and colonoscopy), needle biopsy (e.g., fine-needle aspiration, core needle biopsy, vacuum-assisted biopsy, X-ray-assisted biopsy, computerized tomography (CT)-assisted biopsy, magnetic resonance imaging (MRI)-assisted biopsy and ultrasound-assisted biopsy), skin biopsy (e.g., shave biopsy, punch biopsy, and incisional biopsy) and surgical biopsy. In embodiments, the biological sample comprises a body fluid selected from blood, plasma, serum, lacrimal fluid, tears, bone marrow, blood, blood cells, ascites, tissue or fine needle biopsy sample, cell-containing body fluid, free floating nucleic acids, sputum, saliva, urine, cerebrospinal fluid, peritoneal fluid, pleural fluid, feces, lymph, gynecological fluid, skin swab, vaginal swab, oral swab, nasal swab, washing or lavage such as a ductal lavage or broncheoalveolar lavage, aspirate, scraping, bone marrow specimen, tissue biopsy specimen, surgical specimen, feces, other body fluids, secretions, and/or excretions, and/or cells therefrom. In embodiments, the biological sample is obtained by a technique selected from scrapes, swabs, and biopsy. In embodiments, the biological sample is obtained by use of brushes, (cotton) swabs, spatula, rinse/wash fluids, punch biopsy devices, puncture of cavities with needles or surgical instrumentation. In embodiments, the biological sample comprises at least one tumor cell.

In embodiments, the tumor is selected from Hodgkin's and non-Hodgkin's lymphoma, B-cell lymphoma (including low grade/follicular non-Hodgkin's lymphoma (NHL); small lymphocytic (SL) NHL; intermediate grade/follicular NHL; intermediate grade diffuse NHL; high grade immunoblastic NHL; high grade lymphoblastic NHL; high grade small non-cleaved cell NHL; bulky disease NHL; mantle cell lymphoma; AIDS-related lymphoma; and Waldenstrom's Macroglobulinemia; chronic lymphocytic leukemia (CLL); acute lymphoblastic leukemia (ALL); Hairy cell leukemia; or chronic myeloblastic leukemia, basal cell carcinoma, biliary tract cancer; bladder cancer; bone cancer; brain and central nervous system cancer; breast cancer; cancer of the peritoneum; cervical cancer; choriocarcinoma; colon and rectum cancer; connective tissue cancer; cancer of the digestive system; endometrial cancer; esophageal cancer; eye cancer; cancer of the head and neck; gastric cancer (including gastrointestinal cancer); glioblastoma; hepatic carcinoma; hepatoma; intra-epithelial neoplasm; kidney or renal cancer; larynx cancer; leukemia; liver cancer; lung cancer (e.g., small-cell lung cancer, non-small cell lung cancer, adenocarcinoma of the lung, and squamous carcinoma of the lung); melanoma; myeloma; neuroblastoma; oral cavity cancer (lip, tongue, mouth, and pharynx); ovarian cancer; pancreatic cancer; prostate cancer; retinoblastoma; rhabdomyosarcoma; rectal cancer; cancer of the respiratory system; salivary gland carcinoma; sarcoma; skin cancer; squamous cell cancer; stomach cancer; testicular cancer; thyroid cancer; uterine or endometrial cancer; cancer of the urinary system; vulval cancer; lymphoma including Hodgkin's and non-Hodgkin's lymphoma, as well as B-cell lymphoma (including low grade/follicular non-Hodgkin's lymphoma (NHL); small lymphocytic (SL) NHL; intermediate grade/follicular NHL; intermediate grade diffuse NHL; high grade immunoblastic NHL; high grade lymphoblastic NHL; high grade small non-cleaved cell NHL; bulky disease NHL; mantle cell lymphoma; AIDS-related lymphoma; and Waldenstrom's Macroglobulinemia; chronic lymphocytic leukemia (CLL); acute lymphoblastic leukemia (ALL); Hairy cell leukemia; chronic myeloblastic leukemia; as well as other carcinomas and sarcomas; and post-transplant lymphoproliferative disorder (PTLD), as well as abnormal vascular proliferation associated with phakomatoses, edema (e.g., that associated with brain tumors), Meigs' syndrome cancer; renal carcinoma; colorectal cancer; and adrenal cancer.

In embodiments, the evaluating is performed by DNA sequencing, RNA sequencing, immunohistochemical staining, western blotting, in cell western, immunofluorescent staining, ELISA, and fluorescent activating cell sorting (FACS) or a combination thereof. In embodiments, the evaluating is performed by contacting the sample with an agent that specifically binds to one or more proteins encoded by one or more genes associated with a gene ontology (GO) pathway selected from: (i) positive regulation of cell cycle process, regulation of G1/S transition, regulation of cell division, regulation of cell proliferation, positive regulation of IκB kinase/NFκB signaling, type I IFN signaling pathway, cellular response to IFNγ, positive regulation of IFNα production, positive regulation of defense response, positive regulation of IFNβ production, regulation of inflammatory response, regulation of innate immune response, negative regulation of antigen processing/presentation, and antigen processing/presentation of endogenous peptides via MHC class I; and/or (ii) phospholipid efflux, negative regulation of fibrinolysis, chylomicron assembly, plasma membrane repair, SRP-dependent co-translational protein targeting to membrane, ribosomal small subunit assembly, phospholipid efflux, regulation of translation, mitochondrial respiratory chain complex I, mitochondrial translational elongation, DNA-dependent DNA replication, and ATP biosynthetic process.

In embodiments, the evaluating is performed by contacting the sample with an agent that specifically binds to one or more proteins encoded by one or more genes associated with a gene ontology (GO) pathway selected from (a) cellular response to IFNγ, (b) negative regulation of antigen processing/presentation, (c) type I IFN signaling pathway, (d) positive regulation of IκB kinase/NFκB signaling, and antigen processing, and (e) presentation of endogenous peptides via MHC class I. In embodiments, the evaluating is performed by contacting the sample with an agent that specifically binds to one or more proteins encoded by one or more genes associated with cellular response to IFNγ. In embodiments, the evaluating is performed by contacting the sample with an agent that specifically binds to one or more proteins encoded by one or more genes associated with type I IFN signaling pathway.

In embodiments, the evaluating is performed by contacting the sample with an agent that specifically binds to one or more of nucleic acids of one or more genes associated with a gene ontology (GO) pathway selected from: (i) positive regulation of cell cycle process, regulation of G1/S transition, regulation of cell division, regulation of cell proliferation, positive regulation of IκB kinase/NFκB signaling, type I IFN signaling pathway, cellular response to IFNγ, positive regulation of IFNα production, positive regulation of defense response, positive regulation of IFNβ production, regulation of inflammatory response, regulation of innate immune response, negative regulation of antigen processing/presentation, and antigen processing/presentation of endogenous peptides via MHC class I; and/or (ii) phospholipid efflux, negative regulation of fibrinolysis, chylomicron assembly, plasma membrane repair, SRP-dependent co-translational protein targeting to membrane, ribosomal small subunit assembly, phospholipid efflux, regulation of translation, mitochondrial respiratory chain complex I, mitochondrial translational elongation, DNA-dependent DNA replication, and ATP biosynthetic process. In embodiments, the evaluating is performed by contacting the sample with an agent that specifically binds to one or more nucleic acid of one or more genes associated with a gene ontology (GO) pathway selected from (a) cellular response to IFNγ, (b) negative regulation of antigen processing/presentation, (c) type I IFN signaling pathway, (d) positive regulation of IκB kinase/NFκB signaling, and antigen processing, and (e) presentation of endogenous peptides via MHC class I. In embodiments, the evaluating is performed by contacting the sample with an agent that specifically binds to one or more nucleic acid of one or more genes associated with cellular response to IFNγ. In embodiments, the evaluating is performed by contacting the sample with an agent that specifically binds to one or more nucleic acid of one or more genes associated with type I IFN signaling pathway. In embodiments, the agent that specifically binds to one or more of the nucleic acids is a nucleic acid primer or probe.

In embodiments, the evaluating informs classifying the patient into a high or low risk group. In embodiments, the high risk classification comprises a high level of tumor cells having resistance to the cancer therapy with an ability to inhibit function and/or activity of PD-1, PD-L1 and/or PD-L2. In embodiments, the low risk classification comprises a low level of tumor cells having resistance to the cancer therapy with an ability to inhibit function and/or activity of PD-1, PD-L1 and/or PD-L2. In embodiments, the low risk classification is indicative of withholding of the cancer therapy. In embodiments, the high risk classification is indicative of administering the cancer therapy.

In one aspect, the present disclosure relates to a method of determining a cancer treatment for a patient, the method comprising: (a) obtaining a biological sample from a subject; (b) evaluating the biological sample for the expression of: (i) a gene selected from CD274, B2M, STAT1, STAT2, TRIM7, IRF1, TAP1, TAP2, CASP1, IRF, LTBR, PVR, GASTA3, LRG1, SPRY2, ARG1, TRIM8, TRIM2, MAPK8IP1, TRIM6, and KRT1; and/or (ii) a gene selected from RPL41, RPS15, RPS8, TRIM7 and LRG1; and (c) selecting the cancer therapy based on the evaluation of step (b).

In one aspect, the present disclosure relates to a method of determining a cancer treatment for a patient, the method comprising: (I) obtaining a biological sample from a subject; (II) evaluating the biological sample for the expression of: (i) a gene selected from CD274, B2M, STAT1, STAT2, TRIM7, IRF1, TAP1, TAP2, CASP1, IRF, LTBR, PVR, GASTA3, LRG1, SPRY2, ARG1, TRIM8, TRIM2, MAPK8IP1, TRIM6, and KRT1; and/or (ii) a gene selected from RPL41, RPS15, RPS8, TRIM7 and LRG1; and (III) selecting the cancer therapy based on the evaluation of step (II), wherein the cancer therapy comprises a chimeric protein of a general structure of: N terminus—(a)—(b)—(c)—C terminus, wherein: (A) (a) is a first domain comprising an extracellular domain of a Type I transmembrane protein, the transmembrane protein being TIGIT, (b) is a linker comprising at least one cysteine residue capable of forming a disulfide bond, and (c) is a second domain comprising an extracellular domain of Type II transmembrane protein, the transmembrane protein being LIGHT, wherein the linker connects the first domain and the second domain and optionally comprises one or more joining linkers, such joining linkers being selected from SEQ ID NOs: 49-95; or (B) (a) is a first domain comprising an extracellular domain of a Type I transmembrane protein, the transmembrane protein being SIRPα, (b) is a linker comprising at least one cysteine residue capable of forming a disulfide bond, and (c) is a second domain comprising an extracellular domain of Type II transmembrane protein, the transmembrane protein being 4-1BBL, wherein the linker connects the first domain and the second domain and optionally comprises one or more joining linkers, such joining linkers being selected from SEQ ID NOs: 49-95.

In one aspect, the present disclosure relates to a method for selecting a patient for a cancer treatment, the method comprising: (a) obtaining a biological sample from a subject; (b) evaluating the biological sample for the expression of: (i) a gene selected from CD274, B2M, STAT1, STAT2, TRIM7, IRF1, TAP1, TAP2, CASP1, IRF, LTBR, PVR, GASTA3, LRG1, SPRY2, ARG1, TRIM8, TRIM2, MAPK81P1, TRIM6, and KRT1; and/or (ii) a gene selected from RPL41, RPS15, RPS8, TRIM7 and LRG1; and (c) selecting the cancer therapy based on the evaluation of step (b).

In one aspect, the present disclosure relates to a method for selecting a patient for a cancer treatment, the method comprising: (I) obtaining a biological sample from a subject; (II) evaluating the biological sample for the expression of: (i) a gene selected from CD274, B2M, STAT1, STAT2, TRIM7, IRF1, TAP1, TAP2, CASP1, IRF, LTBR, PVR, GASTA3, LRG1, SPRY2, ARG1, TRIM8, TRIM2, MAPK81P1, TRIM6, and KRT1; and/or (ii) a gene selected from RPL41, RPS15, RPS8, TRIM7 and LRG1; and (III) selecting the cancer therapy based on the evaluation of step (II), wherein the cancer therapy comprises a chimeric protein of a general structure of: N terminus—(a)—(b)—(c)—C terminus, wherein: (A) (a) is a first domain comprising an extracellular domain of a Type I transmembrane protein, the transmembrane protein being TIGIT, (b) is a linker comprising at least one cysteine residue capable of forming a disulfide bond, and (c) is a second domain comprising an extracellular domain of Type II transmembrane protein, the transmembrane protein being LIGHT, wherein the linker connects the first domain and the second domain and optionally comprises one or more joining linkers, such joining linkers being selected from SEQ ID NOs: 49-95; or (B) (a) is a first domain comprising an extracellular domain of a Type I transmembrane protein, the transmembrane protein being SIRPα, (b) is a linker comprising at least one cysteine residue capable of forming a disulfide bond, and (c) is a second domain comprising an extracellular domain of Type II transmembrane protein, the transmembrane protein being 4-1BBL, wherein the linker connects the first domain and the second domain and optionally comprises one or more joining linkers, such joining linkers being selected from SEQ ID NOs: 49-95.

In one aspect, the present disclosure relates to a method of treating cancer, the method comprising: (I) obtaining a biological sample from a subject; (II) evaluating the biological sample for the expression of: (i) a gene selected from CD274, B2M, STAT1, STAT2, TRIM7, IRF1, TAP1, TAP2, CASP1, IRF, LTBR, PVR, GASTA3, LRG1, SPRY2, ARG1, TRIM8, TRIM2, MAPK8IP1, TRIM6, and KRT1; and/or (ii) a gene selected from RPL41, RPS15, RPS8, TRIM7 and LRG1; and (III) wherein the cancer therapy comprises a chimeric protein of a general structure of: N terminus—(a)—(b)—(c)—C terminus, wherein: (A) (a) is a first domain comprising an extracellular domain of a Type I transmembrane protein, the transmembrane protein being TIGIT, (b) is a linker comprising at least one cysteine residue capable of forming a disulfide bond, and (c) is a second domain comprising an extracellular domain of Type II transmembrane protein, the transmembrane protein being LIGHT, wherein the linker connects the first domain and the second domain and optionally comprises one or more joining linkers, such joining linkers being selected from SEQ ID NOs: 49-95; or (B) (a) is a first domain comprising an extracellular domain of a Type I transmembrane protein, the transmembrane protein being SIRPα, (b) is a linker comprising at least one cysteine residue capable of forming a disulfide bond, and (c) is a second domain comprising an extracellular domain of Type II transmembrane protein, the transmembrane protein being 4-1BBL, wherein the linker connects the first domain and the second domain and optionally comprises one or more joining linkers, such joining linkers being selected from SEQ ID NOs: 49-95; (IV) optionally selecting and/or a neoadjuvant therapy and/or an adjuvant therapy; N) optionally administering the neoadjuvant therapy and/or the adjuvant therapy; and (VI) administering the cancer therapy with an ability to inhibit function and/or activity of PD-1, PD-L1 and/or PD-L2.

In non-limiting embodiments, administering the cancer therapy with an ability to inhibit function and/or activity of PD-1, PD-L1 and/or PD-L2 is continued if the upregulation of the overexpression of a gene selected from CD274, B2M, STAT1, STAT2, TRIM7, IRF1, TAP1, TAP2, CASP1, IRF, LTBR, PVR, GASTA3, LRG1, SPRY2, ARG1, TRIM8, TRIM2, MAPK8IP1, TRIM6, and KRT1 is not observed; and/or downregulation a gene selected from RPL41, RPS15, RPS8, TRIM7 and LRG1 is not observed.

In non-limiting embodiments, administering the cancer therapy with an ability to inhibit function and/or activity of PD-1, PD-L1 and/or PD-L2 is continued if the upregulation of the overexpression of a gene selected from CD274, B2M, STAT1, STAT2, TRIM7, IRF1, TAP1, TAP2, CASP1, IRF, LTBR, PVR, GASTA3, LRG1, SPRY2, ARG1, TRIM8, TRIM2, MAPK81P1, TRIM6, and KRT1 is observed; and/or downregulation a gene selected from RPL41, RPS15, RPS8, TRIM7 and LRG1 is observed, wherein the supplementation of administration of the cancer therapy comprises a chimeric protein of a general structure of: N terminus—(a)—(b)—(c)—C terminus, wherein: (A) (a) is a first domain comprising an extracellular domain of a Type I transmembrane protein, the transmembrane protein being TIGIT, (b) is a linker comprising at least one cysteine residue capable of forming a disulfide bond, and (c) is a second domain comprising an extracellular domain of Type II transmembrane protein, the transmembrane protein being LIGHT, wherein the linker connects the first domain and the second domain and optionally comprises one or more joining linkers, such joining linkers being selected from SEQ ID NOs: 49-95; or (B) (a) is a first domain comprising an extracellular domain of a Type I transmembrane protein, the transmembrane protein being SIRPα, (b) is a linker comprising at least one cysteine residue capable of forming a disulfide bond, and (c) is a second domain comprising an extracellular domain of Type II transmembrane protein, the transmembrane protein being 4-1BBL, wherein the linker connects the first domain and the second domain and optionally comprises one or more joining linkers, such joining linkers being selected from SEQ ID NOs: 49-95.

In non-limiting embodiments, administering the cancer therapy with an ability to inhibit function and/or activity of PD-1, PD-L1 and/or PD-L2 is not continued the upregulation of the overexpression of a gene selected from CD274, B2M, STAT1, STAT2, TRIM7, IRF1, TAP1, TAP2, CASP1, IRF, LTBR, PVR, GASTA3, LRG1, SPRY2, ARG1, TRIM8, TRIM2, MAPK81P1, TRIM6, and KRT1 is observed; and/or downregulation a gene selected from RPL41, RPS15, RPS8, TRIM7 and LRG1 is observed, wherein the administration of the cancer therapy comprises a chimeric protein of a general structure of: N terminus—(a)—(b)—(c)—C terminus, wherein: (A) (a) is a first domain comprising an extracellular domain of a Type I transmembrane protein, the transmembrane protein being TIGIT, (b) is a linker comprising at least one cysteine residue capable of forming a disulfide bond, and (c) is a second domain comprising an extracellular domain of Type II transmembrane protein, the transmembrane protein being LIGHT, wherein the linker connects the first domain and the second domain and optionally comprises one or more joining linkers, such joining linkers being selected from SEQ ID NOs: 49-95; or (B) (a) is a first domain comprising an extracellular domain of a Type I transmembrane protein, the transmembrane protein being SIRPα, (b) is a linker comprising at least one cysteine residue capable of forming a disulfide bond, and (c) is a second domain comprising an extracellular domain of Type II transmembrane protein, the transmembrane protein being 4-1BBL, wherein the linker connects the first domain and the second domain and optionally comprises one or more joining linkers, such joining linkers being selected from SEQ ID NOs: 49-95.

In embodiments, the cancer therapy with an ability to inhibit function and/or activity of PD-1, PD-L1 and/or PD-L2 is selected when the biological sample comprises at least one tumor cell, and a gene selected from CD274, B2M, STAT1, STAT2, TRIM7, IRF1, TAP1, TAP2, CASP1, IRF, LTBR, PVR, GASTA3, LRG1, SPRY2, ARG1, TRIM8, TRIM2, MAPK8IP1, TRIM6, and KRT1 is not upregulated in the at least one tumor cell compared to a healthy tissue, a prior biological sample obtained from the subject, or another biological sample from patient that is known to be sensitive to anti-PD-1 therapy; and/or a gene selected from RPL41, RPS15, RPS8, TRIM7 and LRG1 is not downregulated in the at least one tumor cell compared to a healthy tissue, a prior biological sample obtained from the subject, or another biological sample from patient that is known to be sensitive to anti-PD-1 therapy.

In embodiments, when the biological sample comprises at least one tumor cell, and a gene selected from CD274, B2M, STAT1, STAT2, TRIM7, IRF1, TAP1, TAP2, CASP1, IRF, LTBR, PVR, GASTA3, LRG1, SPRY2, ARG1, TRIM8, TRIM2, MAPK8IP1, TRIM6, and KRT1 is upregulated in the at least one tumor cell compared to a healthy tissue, a prior biological sample obtained from the subject, or another biological sample from patient that is known to be sensitive to anti-PD-1 therapy; and/or a gene selected from RPL41, RPS15, RPS8, TRIM7 and LRG1 is downregulated in the at least one tumor cell compared to a healthy tissue, a prior biological sample obtained from the subject, or another biological sample from patient that is known to be sensitive to anti-PD-1 therapy, the cancer therapy comprises a chimeric protein of a general structure of: N terminus—(a)—(b)—(c)—C terminus, wherein: (A) (a) is a first domain comprising an extracellular domain of a Type I transmembrane protein, the transmembrane protein being TIGIT, (b) is a linker comprising at least one cysteine residue capable of forming a disulfide bond, and (c) is a second domain comprising an extracellular domain of Type II transmembrane protein, the transmembrane protein being LIGHT, wherein the linker connects the first domain and the second domain and optionally comprises one or more joining linkers, such joining linkers being selected from SEQ ID NOs: 49-95; or (B) (a) is a first domain comprising an extracellular domain of a Type I transmembrane protein, the transmembrane protein being SIRPα, (b) is a linker comprising at least one cysteine residue capable of forming a disulfide bond, and (c) is a second domain comprising an extracellular domain of Type II transmembrane protein, the transmembrane protein being 4-1BBL, wherein the linker connects the first domain and the second domain and optionally comprises one or more joining linkers, such joining linkers being selected from SEQ ID NOs: 49-95.

In embodiments, an upregulation of one or more genes listed in (b)(i) compared to a healthy tissue indicates a lack of response, resistance or recalcitrance to the cancer therapy with an ability to inhibit function and/or activity of PD-1, PD-L1 and/or PD-L2. In embodiments, an downregulation of one or more genes listed in (b)(ii) compared to a healthy tissue indicates a lack of response, resistance or recalcitrance to the cancer therapy with an ability to inhibit function and/or activity of PD-1, PD-L1 and/or PD-L2.

In embodiments, an upregulation of one or more genes listed in (b)(i) compared to another biological sample from patient that is known to be sensitive to anti-PD-1 therapy indicates a lack of response, resistance or recalcitrance to the cancer therapy with an ability to inhibit function and/or activity of PD-1, PD-L1 and/or PD-L2. In embodiments, an downregulation of one or more genes listed in (b)(ii) compared to another biological sample from patient that is known to be sensitive to anti-PD-1 therapy indicates a lack of response, resistance or recalcitrance to the cancer therapy with an ability to inhibit function and/or activity of PD-1, PD-L1 and/or PD-L2.

In embodiments, an upregulation of one or more genes listed in (b)(i) compared to a prior biological sample obtained from the subject indicates a development of a lack of response, resistance or recalcitrance to the cancer therapy with an ability to inhibit function and/or activity of PD-1, PD-L1 and/or PD-L2. In embodiments, an downregulation of one or more genes listed in (b)(ii) compared to a prior biological sample obtained from the subject indicates a development of a lack of response, resistance or recalcitrance to the cancer therapy with an ability to inhibit function and/or activity of PD-1, PD-L1 and/or PD-L2.

In embodiments, a lack of upregulation of one or more genes listed in (b)(i) compared to a healthy tissue indicates a response to the cancer therapy with an ability to inhibit function and/or activity of PD-1, PD-L1 and/or PD-L2. In embodiments, a lack of downregulation of one or more genes listed in (b)(ii) compared to a healthy tissue indicates a response to the cancer therapy with an ability to inhibit function and/or activity of PD-1, PD-L1 and/or PD-L2.

In embodiments, a lack of upregulation of one or more genes listed in (b)(i) compared to another biological sample from patient that is known to be sensitive to anti-PD-1 therapy indicates a response to the cancer therapy with an ability to inhibit function and/or activity of PD-1, PD-L1 and/or PD-L2. In embodiments, a lack of downregulation of one or more genes listed in (b)(ii) compared to another biological sample from patient that is known to be sensitive to anti-PD-1 therapy indicates a response to the cancer therapy with an ability to inhibit function and/or activity of PD-1, PD-L1 and/or PD-L2.

In embodiments, a lack of upregulation of one or more genes listed in (b)(i) compared to a prior biological sample obtained from the subject indicates a development of lack of a response to the cancer therapy with an ability to inhibit function and/or activity of PD-1, PD-L1 and/or PD-L2. In embodiments, a lack of downregulation of one or more genes listed in (b)(ii) compared to a prior biological sample obtained from the subject indicates a development of lack of a response to the cancer therapy with an ability to inhibit function and/or activity of PD-1, PD-L1 and/or PD-L2.

In embodiments, the biological sample is a fresh tissue sample, frozen tumor tissue specimen, cultured cells, circulating tumor cells, or a formalin-fixed paraffin-embedded tumor tissue specimen. In embodiments, the biological sample is a biopsy sample. In embodiments, the biopsy sample is selected from endoscopic biopsy, bone marrow biopsy, endoscopic biopsy (e.g., cystoscopy, bronchoscopy and colonoscopy), needle biopsy (e.g., fine-needle aspiration, core needle biopsy, vacuum-assisted biopsy, X-ray-assisted biopsy, computerized tomography (CT)-assisted biopsy, magnetic resonance imaging (MRI)-assisted biopsy and ultrasound-assisted biopsy), skin biopsy (e.g., shave biopsy, punch biopsy, and incisional biopsy) and surgical biopsy. In embodiments, the biological sample comprises a body fluid selected from blood, plasma, serum, lacrimal fluid, tears, bone marrow, blood, blood cells, ascites, tissue or fine needle biopsy sample, cell-containing body fluid, free floating nucleic acids, sputum, saliva, urine, cerebrospinal fluid, peritoneal fluid, pleural fluid, feces, lymph, gynecological fluid, skin swab, vaginal swab, oral swab, nasal swab, washing or lavage such as a ductal lavage or broncheoalveolar lavage, aspirate, scraping, bone marrow specimen, tissue biopsy specimen, surgical specimen, feces, other body fluids, secretions, and/or excretions, and/or cells therefrom. In embodiments, the biological sample is obtained by a technique selected from scrapes, swabs, and biopsy. In embodiments, the biological sample is obtained by use of brushes, (cotton) swabs, spatula, rinse/wash fluids, punch biopsy devices, puncture of cavities with needles or surgical instrumentation.

In embodiments, the biological sample comprises at least one tumor cell. In embodiments, the tumor is selected from Hodgkin's and non-Hodgkin's lymphoma, B-cell lymphoma (including low grade/follicular non-Hodgkin's lymphoma (NHL); small lymphocytic (SL) NHL; intermediate grade/follicular NHL; intermediate grade diffuse NHL; high grade immunoblastic NHL; high grade lymphoblastic NHL; high grade small non-cleaved cell NHL; bulky disease NHL; mantle cell lymphoma; AIDS-related lymphoma; and Waldenstrom's Macroglobulinemia; chronic lymphocytic leukemia (CLL); acute lymphoblastic leukemia (ALL); Hairy cell leukemia; or chronic myeloblastic leukemia, basal cell carcinoma, biliary tract cancer; bladder cancer; bone cancer; brain and central nervous system cancer; breast cancer; cancer of the peritoneum; cervical cancer; choriocarcinoma; colon and rectum cancer; connective tissue cancer; cancer of the digestive system; endometrial cancer; esophageal cancer; eye cancer; cancer of the head and neck; gastric cancer (including gastrointestinal cancer); glioblastoma; hepatic carcinoma; hepatoma; intra-epithelial neoplasm; kidney or renal cancer; larynx cancer; leukemia; liver cancer; lung cancer (e.g., small-cell lung cancer, non-small cell lung cancer, adenocarcinoma of the lung, and squamous carcinoma of the lung); melanoma; myeloma; neuroblastoma; oral cavity cancer (lip, tongue, mouth, and pharynx); ovarian cancer; pancreatic cancer; prostate cancer; retinoblastoma; rhabdomyosarcoma; rectal cancer; cancer of the respiratory system; salivary gland carcinoma; sarcoma; skin cancer; squamous cell cancer; stomach cancer; testicular cancer; thyroid cancer; uterine or endometrial cancer; cancer of the urinary system; vulval cancer; lymphoma including Hodgkin's and non-Hodgkin's lymphoma, as well as B-cell lymphoma (including low grade/follicular non-Hodgkin's lymphoma (NHL); small lymphocytic (SL) NHL; intermediate grade/follicular NHL; intermediate grade diffuse NHL; high grade immunoblastic NHL; high grade lymphoblastic NHL; high grade small non-cleaved cell NHL; bulky disease NHL; mantle cell lymphoma; AIDS-related lymphoma; and Waldenstrom's Macroglobulinemia; chronic lymphocytic leukemia (CLL); acute lymphoblastic leukemia (ALL); Hairy cell leukemia; chronic myeloblastic leukemia; as well as other carcinomas and sarcomas; and post-transplant lymphoproliferative disorder (PTLD), as well as abnormal vascular proliferation associated with phakomatoses, edema (e.g., that associated with brain tumors), Meigs' syndrome cancer; renal carcinoma; colorectal cancer; and adrenal cancer.

In embodiments, the evaluating is performed by DNA sequencing, RNA sequencing, immunohistochemical staining, western blotting, in cell western, immunofluorescent staining, ELISA, and fluorescent activating cell sorting (FACS) or a combination thereof. In embodiments, the evaluating is performed by contacting the sample with an agent that specifically binds to one or more proteins encoded by one or more genes listed in (b)(i) and/or (b)(ii). In embodiments, the agent that specifically binds to one or proteins comprises an antibody, antibody-like molecule or binding a fragment thereof. In embodiments, the evaluating is performed by contacting the sample with an agent that specifically binds to one or more of nucleic acids of one or more genes associated with a gene listed in (b)(i) and/or (b)(ii). In embodiments, the agent that specifically binds to one or more of the nucleic acids is a nucleic acid primer or probe.

In one aspect, the present disclosure relates to a method of determining a cancer treatment for a patient, the method comprising: (I) obtaining a biological sample from a subject; (II) evaluating the biological sample for the activation of a pathway selected from Mapk8ip1, Trim7, Elk1, Lrg1, Arg1, Rap1, and Ras; and (III) selecting the cancer therapy based on the evaluation of step (II), wherein the cancer therapy comprises a chimeric protein of a general structure of: N terminus—(a)—(b)—(c)—C terminus, wherein: (A) (a) is a first domain comprising an extracellular domain of a Type I transmembrane protein, the transmembrane protein being TIGIT, (b) is a linker comprising at least one cysteine residue capable of forming a disulfide bond, and (c) is a second domain comprising an extracellular domain of Type II transmembrane protein, the transmembrane protein being LIGHT, wherein the linker connects the first domain and the second domain and optionally comprises one or more joining linkers, such joining linkers being selected from SEQ ID NOs: 49-95; or (B) (a) is a first domain comprising an extracellular domain of a Type I transmembrane protein, the transmembrane protein being SIRPα, (b) is a linker comprising at least one cysteine residue capable of forming a disulfide bond, and (c) is a second domain comprising an extracellular domain of Type II transmembrane protein, the transmembrane protein being 4-1BBL, wherein the linker connects the first domain and the second domain and optionally comprises one or more joining linkers, such joining linkers being selected from SEQ ID NOs: 49-95.

In one aspect, the present disclosure relates to a method for selecting a patient for a cancer treatment, the method comprising: (I) obtaining a biological sample from a subject; (II) evaluating the biological sample for the activation of a pathway selected from Mapk8ip1, Trim7, Elk1, Lrg1, Arg1, Rap1, and Ras; and (III) selecting the cancer therapy based on the evaluation of step (II), wherein the cancer therapy comprises a chimeric protein of a general structure of: N terminus—(a)—(b)—(c)—C terminus, wherein: (A) (a) is a first domain comprising an extracellular domain of a Type I transmembrane protein, the transmembrane protein being TIGIT, (b) is a linker comprising at least one cysteine residue capable of forming a disulfide bond, and (c) is a second domain comprising an extracellular domain of Type II transmembrane protein, the transmembrane protein being LIGHT, wherein the linker connects the first domain and the second domain and optionally comprises one or more joining linkers, such joining linkers being selected from SEQ ID NOs: 49-95; or (B) (a) is a first domain comprising an extracellular domain of a Type I transmembrane protein, the transmembrane protein being SIRPα, (b) is a linker comprising at least one cysteine residue capable of forming a disulfide bond, and (c) is a second domain comprising an extracellular domain of Type II transmembrane protein, the transmembrane protein being 4-1BBL, wherein the linker connects the first domain and the second domain and optionally comprises one or more joining linkers, such joining linkers being selected from SEQ ID NOs: 49-95.

In one aspect, the present disclosure relates to a method of treating cancer, the method comprising: (I) obtaining a biological sample from a subject; (II) evaluating the biological sample for the activation of a pathway selected from Mapk8ip1, Trim7, Elk1, Lrg1, Arg1, Rap1, and Ras; and (II) wherein the cancer therapy comprises a chimeric protein of a general structure of: N terminus—(a)—(b)—(c)—C terminus, wherein: (A) (a) is a first domain comprising an extracellular domain of a Type I transmembrane protein, the transmembrane protein being TIGIT, (b) is a linker comprising at least one cysteine residue capable of forming a disulfide bond, and (c) is a second domain comprising an extracellular domain of Type II transmembrane protein, the transmembrane protein being LIGHT, wherein the linker connects the first domain and the second domain and optionally comprises one or more joining linkers, such joining linkers being selected from SEQ ID NOs: 49-95; or (B) (a) is a first domain comprising an extracellular domain of a Type I transmembrane protein, the transmembrane protein being SIRPα, (b) is a linker comprising at least one cysteine residue capable of forming a disulfide bond, and (c) is a second domain comprising an extracellular domain of Type II transmembrane protein, the transmembrane protein being 4-1BBL, wherein the linker connects the first domain and the second domain and optionally comprises one or more joining linkers, such joining linkers being selected from SEQ ID NOs: 49-95; and (IV) optionally administering the cancer therapy with an ability to inhibit function and/or activity of PD-1, PD-L1 and/or PD-L2.

In embodiments, the cancer therapy with an ability to inhibit function and/or activity of PD-1, PD-L1 and/or PD-L2 is selected when the biological sample comprises at least one tumor cell, and pathway selected from Mapk8ip1, Trim7, Elk1, Lrg1, Arg1, Rap1, and Ras is not upregulated in the at least one tumor cell compared to a healthy tissue, a prior biological sample obtained from the subject, or another biological sample from patient that is known to be sensitive to anti-PD-1 therapy.

In embodiments, when the biological sample comprises at least one tumor cell, and pathway selected from Mapk8ip1, Trim7, Elk1, Lrg1, Arg1, Rap1, and Ras is upregulated in the at least one tumor cell compared to a healthy tissue, a prior biological sample obtained from the subject, or another biological sample from patient that is known to be sensitive to anti-PD-1 therapy, and the cancer therapy comprises a chimeric protein of a general structure of: N terminus—(a)—(b)—(c)—C terminus, wherein: (A) (a) is a first domain comprising an extracellular domain of a Type I transmembrane protein, the transmembrane protein being TIGIT, (b) is a linker comprising at least one cysteine residue capable of forming a disulfide bond, and (c) is a second domain comprising an extracellular domain of Type II transmembrane protein, the transmembrane protein being LIGHT, wherein the linker connects the first domain and the second domain and optionally comprises one or more joining linkers, such joining linkers being selected from SEQ ID NOs: 49-95; or (B) (a) is a first domain comprising an extracellular domain of a Type I transmembrane protein, the transmembrane protein being SIRPα, (b) is a linker comprising at least one cysteine residue capable of forming a disulfide bond, and (c) is a second domain comprising an extracellular domain of Type II transmembrane protein, the transmembrane protein being 4-1BBL, wherein the linker connects the first domain and the second domain and optionally comprises one or more joining linkers, such joining linkers being selected from SEQ ID NOs: 49-95.

In embodiments, an upregulation of pathway selected from Mapk8ip1, Trim7, Elk1, Lrg1, Arg1, Rap1, and Ras compared to a healthy tissue indicates a lack of response, resistance or recalcitrance to the cancer therapy with an ability to inhibit function and/or activity of PD-1, PD-L1 and/or PD-L2. In embodiments, an upregulation of pathway selected from Mapk8ip1, Trim7, Elk1, Lrg1, Arg1, Rap1, and Ras compared to another biological sample from patient that is known to be sensitive to anti-PD-1 therapy indicates a lack of response, resistance or recalcitrance to the cancer therapy with an ability to inhibit function and/or activity of PD-1, PD-L1 and/or PD-L2. In embodiments, an upregulation of pathway selected from Mapk8ip1, Trim7, Elk1, Lrg1, Arg1, Rap1, and Ras compared to a prior biological sample obtained from the subject indicates a development of a lack of response, resistance or recalcitrance to the cancer therapy with an ability to inhibit function and/or activity of PD-1, PD-L1 and/or PD-L2.

In embodiments, a lack of upregulation of pathway selected from Mapk8ip1, Trim7, Elk1, Lrg1, Arg1, Rap1, and Ras compared to a healthy tissue indicates a response to the cancer therapy with an ability to inhibit function and/or activity of PD-1, PD-L1 and/or PD-L2. In embodiments, a lack of upregulation of pathway selected from Mapk8ip1, Trim7, Elk1, Lrg1, Arg1, Rap1, and Ras compared to another biological sample from patient that is known to be sensitive to anti-PD-1 therapy indicates a response to the cancer therapy with an ability to inhibit function and/or activity of PD-1, PD-L1 and/or PD-L2. In embodiments, in a lack of upregulation of pathway selected from Mapk8ip1, Trim7, Elk1, Lrg1, Arg1, Rap1, and Ras compared to a prior biological sample obtained from the subject indicates a development of lack of a response to the cancer therapy with an ability to inhibit function and/or activity of PD-1, PD-L1 and/or PD-L2.

In embodiments, the biological sample is a fresh tissue sample, frozen tumor tissue specimen, cultured cells, circulating tumor cells, or a formalin-fixed paraffin-embedded tumor tissue specimen. In embodiments, the biological sample is a biopsy sample. In embodiments, the biopsy sample is selected from endoscopic biopsy, bone marrow biopsy, endoscopic biopsy (e.g., cystoscopy, bronchoscopy and colonoscopy), needle biopsy (e.g., fine-needle aspiration, core needle biopsy, vacuum-assisted biopsy, X-ray-assisted biopsy, computerized tomography (CT)-assisted biopsy, magnetic resonance imaging (MRI)-assisted biopsy and ultrasound-assisted biopsy), skin biopsy (e.g., shave biopsy, punch biopsy, and incisional biopsy) and surgical biopsy. In embodiments, the biological sample comprises a body fluid selected from blood, plasma, serum, lacrimal fluid, tears, bone marrow, blood, blood cells, ascites, tissue or fine needle biopsy sample, cell-containing body fluid, free floating nucleic acids, sputum, saliva, urine, cerebrospinal fluid, peritoneal fluid, pleural fluid, feces, lymph, gynecological fluid, skin swab, vaginal swab, oral swab, nasal swab, washing or lavage such as a ductal lavage or broncheoalveolar lavage, aspirate, scraping, bone marrow specimen, tissue biopsy specimen, surgical specimen, feces, other body fluids, secretions, and/or excretions, and/or cells therefrom. In embodiments, the biological sample is obtained by a technique selected from scrapes, swabs, and biopsy. In embodiments, the biological sample is obtained by use of brushes, (cotton) swabs, spatula, rinse/wash fluids, punch biopsy devices, puncture of cavities with needles or surgical instrumentation.

In embodiments, the biological sample comprises at least one tumor cell. In embodiments, the tumor is selected from Hodgkin's and non-Hodgkin's lymphoma, B-cell lymphoma (including low grade/follicular non-Hodgkin's lymphoma (NHL); small lymphocytic (SL) NHL; intermediate grade/follicular NHL; intermediate grade diffuse NHL; high grade immunoblastic NHL; high grade lymphoblastic NHL; high grade small non-cleaved cell NHL; bulky disease NHL; mantle cell lymphoma; AIDS-related lymphoma; and Waldenstrom's Macroglobulinemia; chronic lymphocytic leukemia (CLL); acute lymphoblastic leukemia (ALL); Hairy cell leukemia; or chronic myeloblastic leukemia, basal cell carcinoma, biliary tract cancer; bladder cancer; bone cancer; brain and central nervous system cancer; breast cancer; cancer of the peritoneum; cervical cancer; choriocarcinoma; colon and rectum cancer; connective tissue cancer; cancer of the digestive system; endometrial cancer; esophageal cancer; eye cancer; cancer of the head and neck; gastric cancer (including gastrointestinal cancer); glioblastoma; hepatic carcinoma; hepatoma; intra-epithelial neoplasm; kidney or renal cancer; larynx cancer; leukemia; liver cancer; lung cancer (e.g., small-cell lung cancer, non-small cell lung cancer, adenocarcinoma of the lung, and squamous carcinoma of the lung); melanoma; myeloma; neuroblastoma; oral cavity cancer (lip, tongue, mouth, and pharynx); ovarian cancer; pancreatic cancer; prostate cancer; retinoblastoma; rhabdomyosarcoma; rectal cancer; cancer of the respiratory system; salivary gland carcinoma; sarcoma; skin cancer; squamous cell cancer; stomach cancer; testicular cancer; thyroid cancer; uterine or endometrial cancer; cancer of the urinary system; vulval cancer; lymphoma including Hodgkin's and non-Hodgkin's lymphoma, as well as B-cell lymphoma (including low grade/follicular non-Hodgkin's lymphoma (NHL); small lymphocytic (SL) NHL; intermediate grade/follicular NHL; intermediate grade diffuse NHL; high grade immunoblastic NHL; high grade lymphoblastic NHL; high grade small non-cleaved cell NHL; bulky disease NHL; mantle cell lymphoma; AIDS-related lymphoma; and Waldenstrom's Macroglobulinemia; chronic lymphocytic leukemia (CLL); acute lymphoblastic leukemia (ALL); Hairy cell leukemia; chronic myeloblastic leukemia; as well as other carcinomas and sarcomas; and post-transplant lymphoproliferative disorder (PTLD), as well as abnormal vascular proliferation associated with phakomatoses, edema (e.g., that associated with brain tumors), Meigs' syndrome cancer; renal carcinoma; colorectal cancer; and adrenal cancer.

In embodiments, the evaluating is performed by DNA sequencing, RNA sequencing, immunohistochemical staining, western blotting, in cell western, immunofluorescent staining, ELISA, and fluorescent activating cell sorting (FACS) or a combination thereof. In embodiments, the evaluating is performed by contacting the sample with an agent that specifically binds to one or more proteins encoded by a pathway selected from Mapk8ip1, Trim7, Elk1, Lrg1, Arg1, Rap1, and Ras. In embodiments, the agent that specifically binds to one or proteins comprises an antibody, antibody-like molecule or binding a fragment thereof. In embodiments, the evaluating is performed by contacting the sample with an agent that specifically binds to one or more of nucleic acids of one or more genes associated with a pathway selected from Mapk8ip1, Trim7, Elk1, Lrg1, Arg1, Rap1, and Ras. In embodiments, the agent that specifically binds to one or more of the nucleic acids is a nucleic acid primer or probe.

Trim7 is a member of a large family comprising approximately 80 distinct tripartite motif proteins (Tripartite motif-containing (TRIM)). The protein family comprises 80 TRIM protein members in humans. In embodiments, the Trim-family activation may be assayed by modulation of innate immune responses through the control of IFN responsive genes (i.e. IRF1/IRF3/IRF7, JAK/STAT, and NFκB. In embodiments, the Trim-family members have a C-terminal SPRY domain (e.g., in Trim6 and Trim7), In embodiments, Trim7 is assayed based on induction of proliferation, EMT, and acquisition of a chemo-resistant phenotype.

In embodiments, Trim7 pathway activation may be assayed using E3 ubiquitin ligase activity. In embodiments, Trim7 pathway activation may be assayed c-Jun/AP1 activation by Ras-Raf-MEK-ERK signaling. In embodiments, Trim7 pathway activation may be assayed by assaying protein ubiquitylation.

In embodiments, Trim7 pathway activation may be assayed using the ubiquitination and stabilization of AP1 co-activator RACO-1 and/or an increase in AP1 mediated gene expression. AP1-mediated gene expression signature is well known in the art.

In embodiments, Trim7 pathway activation may be assayed based on K63-linked ubiquitylation of target proteins, including proteins involved with cell proliferation and innate immune responses. In embodiments, Trim7 pathway activation may be assayed based on Trim7 phosphorylation, K63-linked ubiquitylation and/or protein level of the AP-1 co-activator known as RACO-1. In embodiments, Trim7 pathway activation may be assayed based on the level or activity of STING (stimulator of interferon genes, MITA, ERIS, MPYS, TMEM173). In embodiments, Trim7 pathway activation may be assayed via K48-linked ubiquitylation of STING. In embodiments, Trim7 pathway activation may be assayed upregulation of IFNb, IP-10 and Rantes.

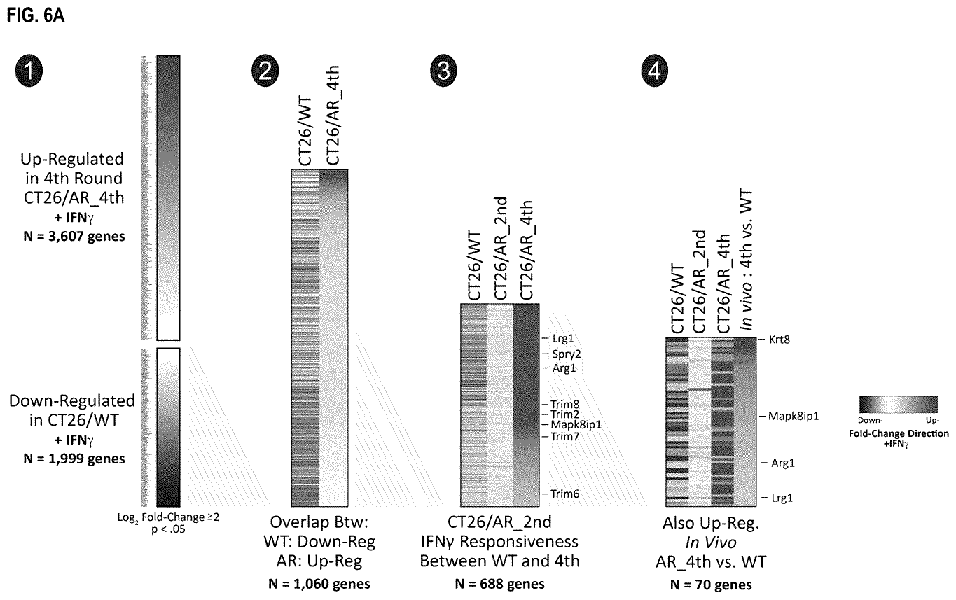

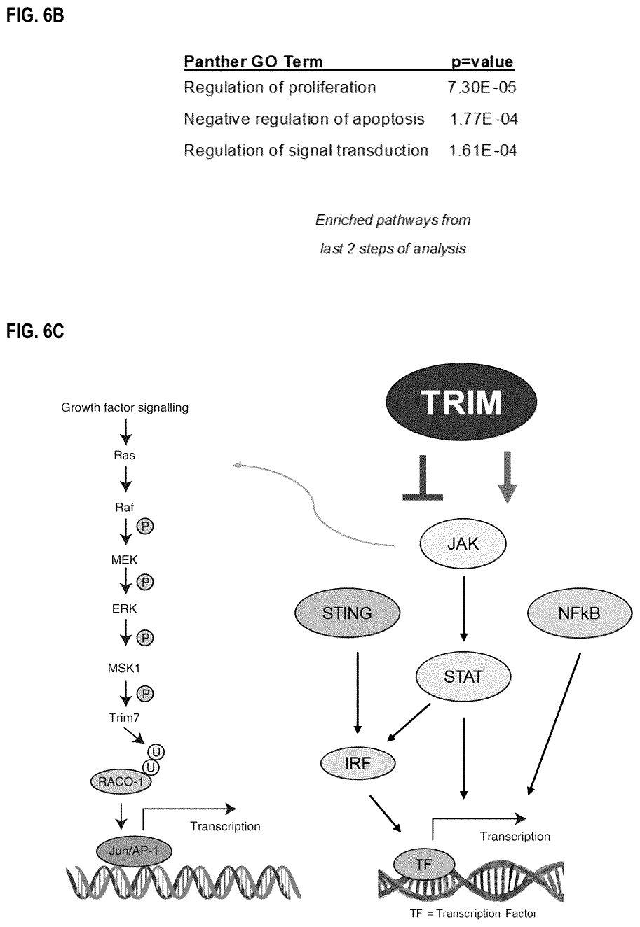

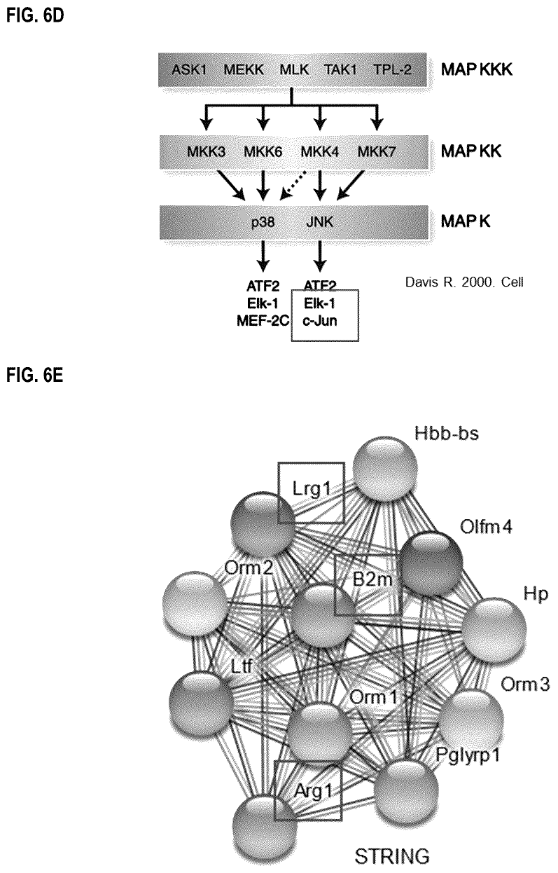

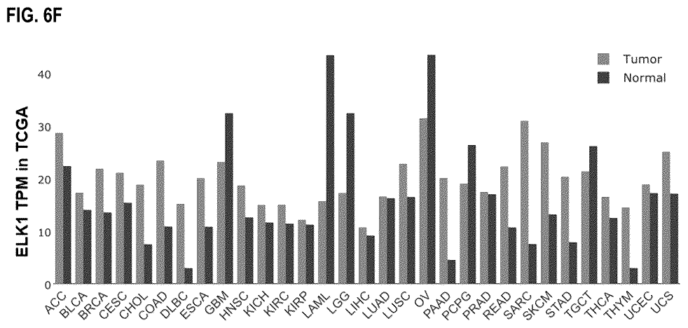

In embodiments, Trim7 pathway activation may be assayed based on the activation of a protein shown in FIG. 6C.

Mitogen-activated protein kinase 8 interacting protein 1 (Mapk8ip1) is also known as c-Jun-amino-terminal kinase-interacting protein 1. In embodiments, Mapk8ip1 pathway activation may be assayed based on assay of functional multiprotein complex in different components of the JNK pathway, including RAC1 or RAC2, MAP3K11/MLK3 or MAP3K7/TAK1, MAP2K7/MKK7, MAPK8/JNK1 and/or MAPK9/JNK2. In embodiments, Mapk8ip1 pathway activation may be assayed based on levels of and/or sensitivity to programmed cell death by apoptosis.

In embodiments, ETS Like-1 protein (Elk1) pathway activation may be assayed based on the activation of Ras-Raf-MEK-ERK signaling, which is well known in the art. In embodiments, Elk1 pathway activation may be assayed based on Elk1 phosphorylation. In embodiments, Elk1 pathway activation may be assayed based on activation of Elk1 target genes, which are well-known in the art (see, e.g., Odrowaz and Sharrocks, ELK1 Uses Different DNA Binding Modes to Regulate Functionally Distinct Classes of Target Genes, PLOS Genetics 8(5):e1002694 (2012).

In embodiments, leucine-rich alpha-2-glycoprotein 1 (Lrg1) pathway activation may be assayed based on the induction of TGFβ and/or SMAD1/5/8 signaling.

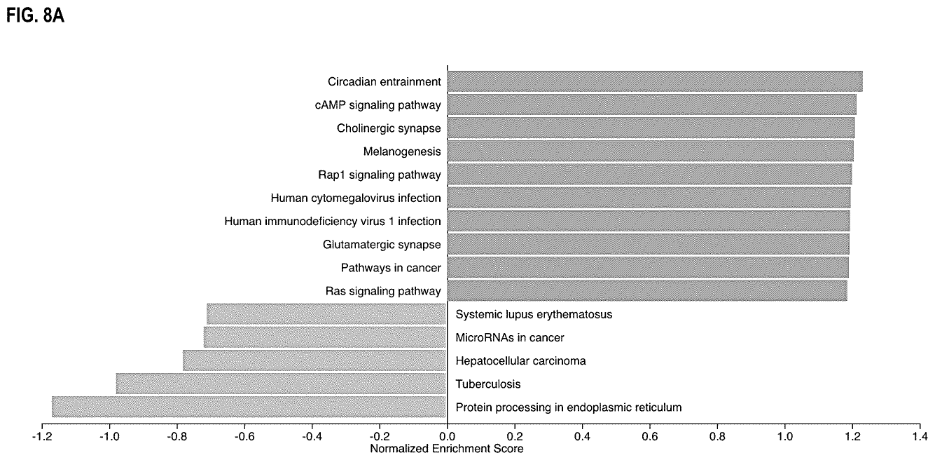

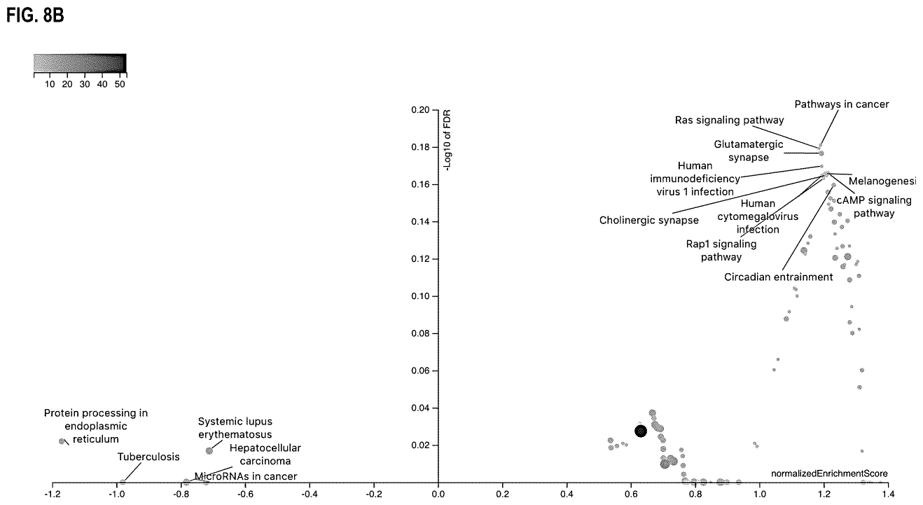

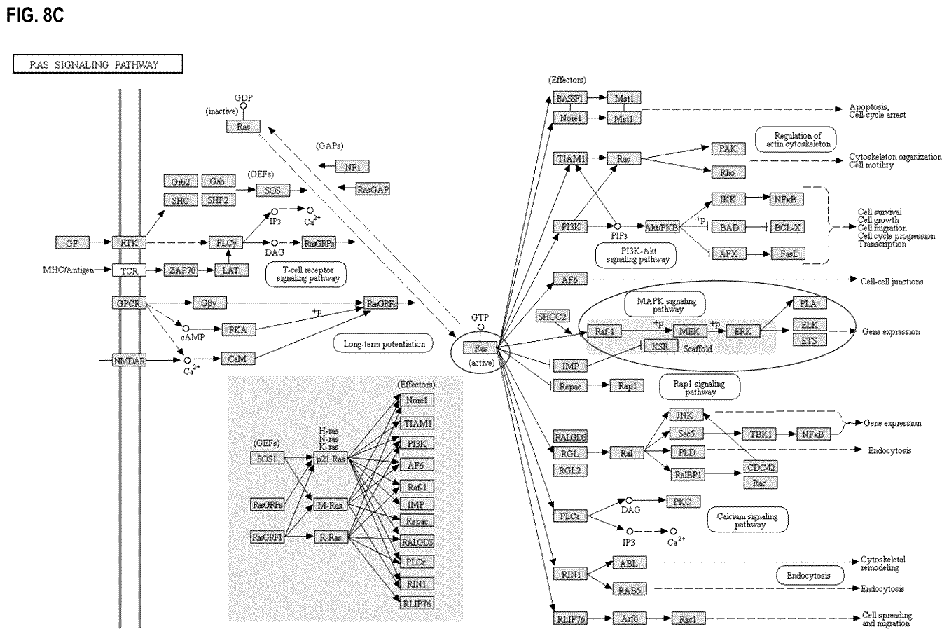

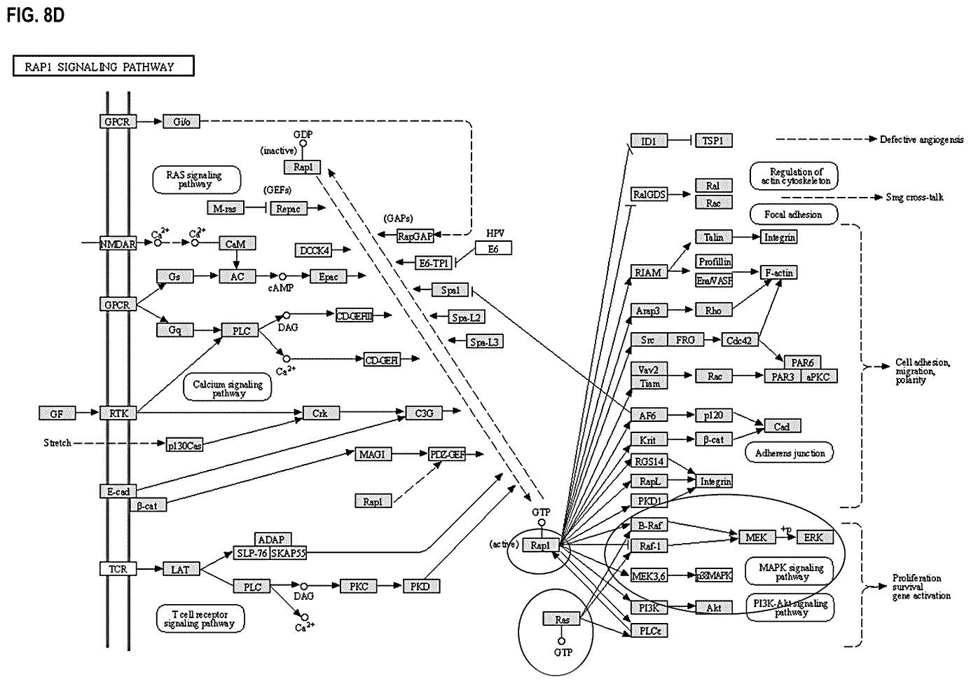

In embodiments, Ras pathway activation may be assayed based on the activation of a protein shown in FIG. 8C.

In embodiments, Rap1 pathway activation may be assayed based on the activation of a protein shown in FIG. 8D.

In embodiments, Arginase 1 (Arg1) pathway activation may be assayed based on the assay hydrolysis of arginine to ornithine and urea. In embodiments, Arginase 1 (Arg1) pathway activation may be assayed based on the suppression of tumor infiltrating lymphocytes (TILs). In embodiments, Arginase 1 (Arg1) pathway activation may be assayed based on the levels or synthesis of polyamines (via L-ornithine).

In one aspect, the present disclosure relates to a method of treating a cancer in a subject in need thereof, the method comprising administering a cancer therapy comprises a chimeric protein of a general structure of: N terminus—(a)—(b)—(c)—C terminus, wherein: (A) (a) is a first domain comprising an extracellular domain of a Type I transmembrane protein, the transmembrane protein being TIGIT, (b) is a linker comprising at least one cysteine residue capable of forming a disulfide bond, and (c) is a second domain comprising an extracellular domain of Type II transmembrane protein, the transmembrane protein being LIGHT, wherein the linker connects the first domain and the second domain and optionally comprises one or more joining linkers, such joining linkers being selected from SEQ ID NOs: 49-95; or (B) (a) is a first domain comprising an extracellular domain of a Type I transmembrane protein, the transmembrane protein being SIRPα, (b) is a linker comprising at least one cysteine residue capable of forming a disulfide bond, and (c) is a second domain comprising an extracellular domain of Type II transmembrane protein, the transmembrane protein being 4-1BBL, wherein the linker connects the first domain and the second domain and optionally comprises one or more joining linkers, such joining linkers being selected from SEQ ID NOs: 49-95, wherein the subject has received or is receiving an anticancer treatment with an ability to inhibit function and/or activity of PD-1, PD-L1 and/or PD-L2, and wherein the subject has developed a lack of response, resistance or recalcitrance to the cancer therapy with an ability to inhibit function and/or activity of PD-1, PD-L1 and/or PD-L2.

In one aspect, the present disclosure relates to a method of treating a cancer in a subject in need thereof, the method comprising: (a) administering an anticancer treatment with an ability to inhibit function and/or activity of PD-1, PD-L1 and/or PD-L2; (b) evaluating anti-tumor response with the anticancer treatment with an ability to inhibit function and/or activity of PD-1, PD-L1 and/or PD-L2 by monitoring a tumor size reduction in the subject; (c) if a lack of tumor size reduction is observed, administering a chimeric protein of a general structure of: N terminus—(a)—(b)—(c)—C terminus, wherein: (A) (a) is a first domain comprising an extracellular domain of a Type I transmembrane protein, the transmembrane protein being TIGIT, (b) is a linker comprising at least one cysteine residue capable of forming a disulfide bond, and (c) is a second domain comprising an extracellular domain of Type II transmembrane protein, the transmembrane protein being LIGHT, wherein the linker connects the first domain and the second domain and optionally comprises one or more joining linkers, such joining linkers being selected from SEQ ID NOs: 49-95; or (B) (a) is a first domain comprising an extracellular domain of a Type I transmembrane protein, the transmembrane protein being SIRPα, (b) is a linker comprising at least one cysteine residue capable of forming a disulfide bond, and (c) is a second domain comprising an extracellular domain of Type II transmembrane protein, the transmembrane protein being 4-1BBL, wherein the linker connects the first domain and the second domain and optionally comprises one or more joining linkers, such joining linkers being selected from SEQ ID NOs: 49-95; (d) re-evaluating anti-tumor response with the anticancer treatment with an ability to inhibit function and/or activity of PD-1, PD-L1 and/or PD-L2 by monitoring the tumor reduction in the subject; and (e) if a tumor size reduction is not observed, withdrawing the administration of a chimeric protein of a general structure of: N terminus—(a)—(b)—(c)—C terminus, wherein: (A) (a) is a first domain comprising an extracellular domain of a Type I transmembrane protein, the transmembrane protein being TIGIT, (b) is a linker comprising at least one cysteine residue capable of forming a disulfide bond, and (c) is a second domain comprising an extracellular domain of Type II transmembrane protein, the transmembrane protein being LIGHT, wherein the linker connects the first domain and the second domain and optionally comprises one or more joining linkers, such joining linkers being selected from SEQ ID NOs: 49-95; or (B) (a) is a first domain comprising an extracellular domain of a Type I transmembrane protein, the transmembrane protein being SIRPα, (b) is a linker comprising at least one cysteine residue capable of forming a disulfide bond, and (c) is a second domain comprising an extracellular domain of Type II transmembrane protein, the transmembrane protein being 4-1BBL, wherein the linker connects the first domain and the second domain and optionally comprises one or more joining linkers, such joining linkers being selected from SEQ ID NOs: 49-95.

In one aspect, the present disclosure relates to a method of treating a cancer in a subject in need thereof, the method comprising: (a) administering an anticancer treatment with an ability to inhibit function and/or activity of PD-1, PD-L1 and/or PD-L2; (b) evaluating anti-tumor response with the anticancer treatment with an ability to inhibit function and/or activity of PD-1, PD-L1 and/or PD-L2 using the steps of: (i) obtaining a biological sample from the subject; (ii) evaluating the biological sample for the overexpression and/or activation of TRIM7; (c) administering a chimeric protein of a general structure of: N terminus—(a)—(b)—(c)—C terminus, wherein: (A) (a) is a first domain comprising an extracellular domain of a Type I transmembrane protein, the transmembrane protein being TIGIT, (b) is a linker comprising at least one cysteine residue capable of forming a disulfide bond, and (c) is a second domain comprising an extracellular domain of Type II transmembrane protein, the transmembrane protein being LIGHT, wherein the linker connects the first domain and the second domain and optionally comprises one or more joining linkers, such joining linkers being selected from SEQ ID NOs: 49-95; or (B) (a) is a first domain comprising an extracellular domain of a Type I transmembrane protein, the transmembrane protein being SIRPα, (b) is a linker comprising at least one cysteine residue capable of forming a disulfide bond, and (c) is a second domain comprising an extracellular domain of Type II transmembrane protein, the transmembrane protein being 4-1BBL, wherein the linker connects the first domain and the second domain and optionally comprises one or more joining linkers, such joining linkers being selected from SEQ ID NOs: 49-95; (d) re-evaluating anti-tumor response with the anticancer treatment with an ability to inhibit function and/or activity of PD-1, PD-L1 and/or PD-L2 using the steps of: (i) obtaining a biological sample from the subject; (ii) evaluating the biological sample for the overexpression and/or activation of TRIM7; and (e) if an overexpression and/or activation of TRIM7 is not observed, withdrawing the administration of a chimeric protein of a general structure of: N terminus—(a)—(b)—(c)—C terminus, wherein: (A) (a) is a first domain comprising an extracellular domain of a Type I transmembrane protein, the transmembrane protein being TIGIT, (b) is a linker comprising at least one cysteine residue capable of forming a disulfide bond, and (c) is a second domain comprising an extracellular domain of Type II transmembrane protein, the transmembrane protein being LIGHT, wherein the linker connects the first domain and the second domain and optionally comprises one or more joining linkers, such joining linkers being selected from SEQ ID NOs: 49-95; or (B) (a) is a first domain comprising an extracellular domain of a Type I transmembrane protein, the transmembrane protein being SIRPα, (b) is a linker comprising at least one cysteine residue capable of forming a disulfide bond, and (c) is a second domain comprising an extracellular domain of Type II transmembrane protein, the transmembrane protein being 4-1BBL, wherein the linker connects the first domain and the second domain and optionally comprises one or more joining linkers, such joining linkers being selected from SEQ ID NOs: 49-95.

In one aspect, the present disclosure relates to a method of treating a cancer in a subject in need thereof, the method comprising: (a) administering an anticancer treatment with an ability to inhibit function and/or activity of PD-1, PD-L1 and/or PD-L2; (b) evaluating overexpression and/or activation of TRIM7 using the steps of: (i) obtaining a biological sample from the subject; and (ii) evaluating the biological sample for the overexpression and/or activation of TRIM7; (c) if an overexpression and/or activation of TRIM7 is observed, administering a chimeric protein of a general structure of: N terminus—(a)—(b)—(c)—C terminus, wherein: (A) (a) is a first domain comprising an extracellular domain of a Type I transmembrane protein, the transmembrane protein being TIGIT, (b) is a linker comprising at least one cysteine residue capable of forming a disulfide bond, and (c) is a second domain comprising an extracellular domain of Type II transmembrane protein, the transmembrane protein being LIGHT, wherein the linker connects the first domain and the second domain and optionally comprises one or more joining linkers, such joining linkers being selected from SEQ ID NOs: 49-95; or (B) (a) is a first domain comprising an extracellular domain of a Type I transmembrane protein, the transmembrane protein being SIRPα, (b) is a linker comprising at least one cysteine residue capable of forming a disulfide bond, and (c) is a second domain comprising an extracellular domain of Type II transmembrane protein, the transmembrane protein being 4-1BBL, wherein the linker connects the first domain and the second domain and optionally comprises one or more joining linkers, such joining linkers being selected from SEQ ID NOs: 49-95; (d) re-evaluating overexpression and/or activation of TRIM7 using the steps of: (i) obtaining a biological sample from the subject; (ii) evaluating the biological sample for the overexpression and/or activation of TRIM7; and (e) if an overexpression and/or activation of TRIM7 is not observed, withdrawing the administration of a chimeric protein of a general structure of: N terminus—(a)—(b)—(c)—C terminus, wherein: (A) (a) is a first domain comprising an extracellular domain of a Type I transmembrane protein, the transmembrane protein being TIGIT, (b) is a linker comprising at least one cysteine residue capable of forming a disulfide bond, and (c) is a second domain comprising an extracellular domain of Type II transmembrane protein, the transmembrane protein being LIGHT, wherein the linker connects the first domain and the second domain and optionally comprises one or more joining linkers, such joining linkers being selected from SEQ ID NOs: 49-95; or (B) (a) is a first domain comprising an extracellular domain of a Type I transmembrane protein, the transmembrane protein being SIRPα, (b) is a linker comprising at least one cysteine residue capable of forming a disulfide bond, and (c) is a second domain comprising an extracellular domain of Type II transmembrane protein, the transmembrane protein being 4-1BBL, wherein the linker connects the first domain and the second domain and optionally comprises one or more joining linkers, such joining linkers being selected from SEQ ID NOs: 49-95.

In embodiments, the overexpression and/or activation of TRIM7 is observed by treating the biological sample with a Trim7 modulator. In embodiments, the Trim7 modulator is a Trim 7 inhibitor. In embodiments, the Trim7 modulator is selected a small interference RNA (siRNA), a short hairpin RNA (shRNA), a microRNA (miRNA), an antisense RNA, a guide RNA (gRNA), a small molecule, an antibody, a peptide, and a peptidomimetic. In embodiments, the small interference RNA (siRNA), the short hairpin RNA (shRNA), the microRNA (miRNA), the antisense RNA, or the guide RNA (gRNA) inhibit the production of Trim7 protein. In embodiments, the peptidomimetic mimics a target of Trim7 and thereby inhibits the activity of Trim7. In embodiments, the Trim 7 inhibitor is a small molecule or peptide inhibitor that targets the areas bracketed by one or more Trim7 protein segments selected from MAAVGPRTGPGTGAEALALAAEL (SEQ ID NO: 104), AATRAPPFPLPCP (SEQ ID NO: 105), HGSQAAAARAAAARCG (SEQ ID NO: 106) and NVSLKTFVLKGMLKKFKEDLRGELEKEEKV (SEQ ID NO: 107). In embodiments, the Trim7 modulator is a mitogen- and stress-activated kinase 1 (MSK1) inhibitor, wherein the MSK1 inhibitor modulates Trim7 via downstream effect of an inhibition of MSK1. In embodiments, the MSK1 inhibitor is selected from Ro 31-8220, SB-747651A, and H89. In embodiments, the MSK1 inhibitor is SB-747651A.

In embodiments, the evaluating is performed by DNA sequencing, RNA sequencing, immunohistochemical staining, western blotting, in cell western, immunofluorescent staining, ELISA, and fluorescent activating cell sorting (FACS) or a combination thereof. In embodiments, the evaluating is performed by contacting the sample with an agent that specifically binds to one or more proteins encoded by the Trim7 pathway. In embodiments, the agent that specifically binds to one or proteins comprises an antibody, antibody-like molecule or binding a fragment thereof. In embodiments, the evaluating is performed by contacting the sample with an agent that specifically binds to one or more of nucleic acids of one or more genes associated with the Trim7 pathway. In embodiments, the agent that specifically binds to one or more of the nucleic acids is a nucleic acid primer or probe.

In embodiments, the evaluating is performed by assaying a E3 ubiquitin ligase activity. In embodiments, the evaluating is performed by assaying protein ubiquitylation and/or K48-linked ubiquitylation of stimulator of interferon genes (STING) and/or AP-1 co-activator RACO-1. In embodiments, the evaluating is performed by assaying c-Jun/AP1 activation via Ras-Raf-MEK-ERK signaling and/or an increase in AP1 mediated gene expression. In embodiments, the evaluating is performed by assaying ubiquitination and stabilization of AP1 co-activator RACO-1. In embodiments, the evaluating is performed by assaying K63-linked ubiquitylation of target proteins, including proteins involved with cell proliferation and innate immune responses. In embodiments, the evaluating is performed by assaying Trim7 phosphorylation, K63-linked ubiquitylation and/or protein level of the AP-1 co-activator RACO-1. In embodiments, the evaluating is performed by assaying the upregulation of IFNβ, IP-10 and/or Rantes.

In one aspect, the present disclosure relates to a method for treating cancer in a subject in need thereof comprising administering an effective amount of a pharmaceutical composition to a subject in need thereof, the pharmaceutical composition comprising a chimeric protein comprising: N terminus—(a)—(b)—(c)—C terminus, wherein: (A) (a) is a first domain comprising an extracellular domain of a Type I transmembrane protein, the transmembrane protein being TIGIT, (b) is a linker comprising at least one cysteine residue capable of forming a disulfide bond, and (c) is a second domain comprising an extracellular domain of Type II transmembrane protein, the transmembrane protein being LIGHT, wherein the linker connects the first domain and the second domain and optionally comprises one or more joining linkers, such joining linkers being selected from SEQ ID NOs: 49-95; or (B) (a) is a first domain comprising an extracellular domain of a Type I transmembrane protein, the transmembrane protein being SIRPα, (b) is a linker comprising at least one cysteine residue capable of forming a disulfide bond, and (c) is a second domain comprising an extracellular domain of Type II transmembrane protein, the transmembrane protein being 4-1BBL, wherein the linker connects the first domain and the second domain and optionally comprises one or more joining linkers, such joining linkers being selected from SEQ ID NOs: 49-95, wherein the cancer is or is believed to be resistant to an anti-checkpoint agent having an ability to inhibit function and/or activity of PD-1, PD-L1 and/or PD-L2.

In embodiments, the anti-checkpoint agent an antibody selected from nivolumab (OPDIVO), pembrolizumab (KEYTRUDA), pidilizumab (CT-011, CURE TECH), MK-3475 (MERCK), BMS 936559, MPDL328OA (ROCHE), Cemiplimab (LIBTAYO), Atezolizumab (TECENTRIQ), Avelumab (BAVENCIO), and Durvalumab (imfinzi).

In embodiments, the method further comprises administration of an anti-checkpoint agent. In embodiments, the anti-checkpoint agent an antibody selected from nivolumab (OPDIVO), pembrolizumab (KEYTRUDA), pidilizumab (CT-011, CURE TECH), MK-3475 (MERCK), BMS 936559, MPDL328OA (ROCHE), Cemiplimab (LIBTAYO), Atezolizumab (TECENTRIQ), Avelumab (BAVENCIO), and Durvalumab (imfinzi). In embodiments, the pharmaceutical composition comprising the chimeric protein and the anti-checkpoint agent are administered simultaneously or contemporaneously. In embodiments, the pharmaceutical composition comprising the chimeric protein is administered after the anti-checkpoint agent is administered. In embodiments, the pharmaceutical composition comprising the chimeric protein is administered before the anti-checkpoint agent is administered.

In embodiments, the dose of the pharmaceutical composition comprising the chimeric protein is less than the dose of the pharmaceutical composition comprising the chimeric protein administered to a subject who has not undergone or is not undergoing treatment with the anti-checkpoint agent. In embodiments, the dose of the anti-checkpoint agent administered is less than the dose of the anti-checkpoint agent administered to a subject who has not undergone or is not undergoing treatment with the pharmaceutical composition comprising the chimeric protein. In embodiments, the subject has an increased chance of survival, without gastrointestinal inflammation and weight loss, and/or a reduction in tumor size or cancer prevalence when compared to a subject who has only undergone or is only undergoing treatment with the pharmaceutical composition comprising the chimeric protein. In embodiments, the subject has an increased chance of survival, without gastrointestinal inflammation and weight loss, and/or a reduction in tumor size or cancer prevalence when compared to a subject who has only undergone or is only undergoing treatment with the anti-checkpoint agent.

Formulations

The chimeric proteins (and/or additional agents) described herein can possess a sufficiently basic functional group, which can react with an inorganic or organic acid, or a carboxyl group, which can react with an inorganic or organic base, to form a pharmaceutically acceptable salt. A pharmaceutically acceptable acid addition salt is formed from a pharmaceutically acceptable acid, as is well known in the art. Such salts include the pharmaceutically acceptable salts listed in, for example, Journal of Pharmaceutical Science, 66, 2-19 (1977) and The Handbook of Pharmaceutical Salts; Properties, Selection, and Use. P. H. Stahl and C. G. Wermuth (eds.), Verlag, Zurich (Switzerland) 2002, which are hereby incorporated by reference in their entirety.

In embodiments, the compositions described herein are in the form of a pharmaceutically acceptable salt.

Further, any chimeric protein (and/or additional agents) described herein can be administered to a subject as a component of a composition that comprises a pharmaceutically acceptable carrier or vehicle. Such compositions can optionally comprise a suitable amount of a pharmaceutically acceptable excipient so as to provide the form for proper administration. Pharmaceutical excipients can be liquids, such as water and oils, including those of petroleum, animal, vegetable, or synthetic origin, such as peanut oil, soybean oil, mineral oil, sesame oil and the like. The pharmaceutical excipients can be, for example, saline, gum acacia, gelatin, starch paste, talc, keratin, colloidal silica, urea and the like. In addition, auxiliary, stabilizing, thickening, lubricating, and coloring agents can be used. In one embodiment, the pharmaceutically acceptable excipients are sterile when administered to a subject. Water is a useful excipient when any agent described herein is administered intravenously. Saline solutions and aqueous dextrose and glycerol solutions can also be employed as liquid excipients, specifically for injectable solutions. Suitable pharmaceutical excipients also include starch, glucose, lactose, sucrose, gelatin, malt, rice, flour, chalk, silica gel, sodium stearate, glycerol monostearate, talc, sodium chloride, dried skim milk, glycerol, propylene, glycol, water, ethanol and the like. Any agent described herein, if desired, can also comprise minor amounts of wetting or emulsifying agents, or pH buffering agents.

In embodiments, the compositions described herein are resuspended in a saline buffer (including, without limitation TBS, PBS, and the like).

In embodiments, the chimeric proteins may by conjugated and/or fused with another agent to extend half-life or otherwise improve pharmacodynamic and pharmacokinetic properties. In embodiments, the chimeric proteins may be fused or conjugated with one or more of PEG, XTEN (e.g., as rPEG), polysialic acid (POLYXEN), albumin (e.g., human serum albumin or HAS), elastin-like protein (ELP), PAS, HAP, GLK, CTP, transferrin, and the like. In embodiments, each of the individual chimeric proteins is fused to one or more of the agents described in BioDrugs (2015) 29:215-239, the entire contents of which are hereby incorporated by reference.

Administration, Dosing, and Treatment Regimens

The present disclosure includes the described chimeric protein (and/or additional agents) in various formulations. Any chimeric protein (and/or additional agents) described herein can take the form of solutions, suspensions, emulsion, drops, tablets, pills, pellets, capsules, capsules containing liquids, powders, sustained-release formulations, suppositories, emulsions, aerosols, sprays, suspensions, or any other form suitable for use. DNA or RNA constructs encoding the protein sequences may also be used. In one embodiment, the composition is in the form of a capsule (see, e.g., U.S. Pat. No. 5,698,155). Other examples of suitable pharmaceutical excipients are described in Remington's Pharmaceutical Sciences 1447-1676 (Alfonso R. Gennaro eds., 19th ed. 1995), incorporated herein by reference.

Where necessary, the formulations comprising the chimeric protein (and/or additional agents) can also include a solubilizing agent. Also, the agents can be delivered with a suitable vehicle or delivery device as known in the art. Combination therapies outlined herein can be co-delivered in a single delivery vehicle or delivery device. Compositions for administration can optionally include a local anesthetic such as, for example, lignocaine to lessen pain at the site of the injection.

The formulations comprising the chimeric protein (and/or additional agents) of the present disclosure may conveniently be presented in unit dosage forms and may be prepared by any of the methods well known in the art of pharmacy. Such methods generally include the step of bringing therapeutic agents into association with a carrier, which constitutes one or more accessory ingredients. Typically, the formulations are prepared by uniformly and intimately bringing therapeutic agent into association with a liquid carrier, a finely divided solid carrier, or both, and then, if necessary, shaping the product into dosage forms of the desired formulation (e.g., wet or dry granulation, powder blends, etc., followed by tableting using conventional methods known in the art)

In one embodiment, any chimeric protein (and/or additional agents) described herein is formulated in accordance with routine procedures as a composition adapted for a mode of administration described herein.

Routes of administration include, for example: intradermal, intramuscular, intraperitoneal, intravenous, subcutaneous, intranasal, epidural, oral, sublingual, intranasal, intracerebral, intravaginal, transdermal, rectally, by inhalation, or topically, particularly to the ears, nose, eyes, or skin. In embodiments, the administering is effected orally or by parenteral injection. In most instances, administration results in the release of any agent described herein into the bloodstream.

Any chimeric protein (and/or additional agents) described herein can be administered orally. Such chimeric proteins (and/or additional agents) can also be administered by any other convenient route, for example, by intravenous infusion or bolus injection, by absorption through epithelial or mucocutaneous linings (e.g., oral mucosa, rectal and intestinal mucosa, etc.) and can be administered together with another biologically active agent. Administration can be systemic or local. Various delivery systems are known, e.g., encapsulation in liposomes, microparticles, microcapsules, capsules, etc., and can be used to administer.

In specific embodiments, it may be desirable to administer locally to the area in need of treatment. In one embodiment, for instance in the treatment of cancer, the chimeric protein (and/or additional agents) are administered in the tumor microenvironment (e.g., cells, molecules, extracellular matrix and/or blood vessels that surround and/or feed a tumor cell, inclusive of, for example, tumor vasculature; tumor-infiltrating lymphocytes; fibroblast reticular cells; endothelial progenitor cells (EPC); cancer-associated fibroblasts; pericytes; other stromal cells; components of the extracellular matrix (ECM); dendritic cells; antigen presenting cells; T-cells; regulatory T cells; macrophages; neutrophils; and other immune cells located proximal to a tumor) or lymph node and/or targeted to the tumor microenvironment or lymph node. In embodiments, for instance in the treatment of cancer, the chimeric protein (and/or additional agents) are administered intratumorally.

In the various embodiments, the present chimeric protein allows for a dual effect that provides less side effects than are seen in conventional immunotherapy (e.g., treatments with one or more of OPDIVO, KEYTRUDA, YERVOY, and TECENTRIQ). For example, the present chimeric proteins reduce or prevent commonly observed immune-related adverse events that affect various tissues and organs including the skin, the gastrointestinal tract, the kidneys, peripheral and central nervous system, liver, lymph nodes, eyes, pancreas, and the endocrine system; such as hypophysitis, colitis, hepatitis, pneumonitis, rash, and rheumatic disease. Further, the present local administration, e.g., intratumorally, obviate adverse event seen with standard systemic administration, e.g., IV infusions, as are used with conventional immunotherapy (e.g., treatments with one or more of OPDIVO, KEYTRUDA, YERVOY, and TECENTRIQ).

Dosage forms suitable for parenteral administration (e.g., intravenous, intramuscular, intraperitoneal, subcutaneous and intra-articular injection and infusion) include, for example, solutions, suspensions, dispersions, emulsions, and the like. They may also be manufactured in the form of sterile solid compositions (e.g., lyophilized composition), which can be dissolved or suspended in sterile injectable medium immediately before use. They may contain, for example, suspending or dispersing agents known in the art.

The dosage of any chimeric protein (and/or additional agents) described herein as well as the dosing schedule can depend on various parameters, including, but not limited to, the disease being treated, the subject's general health, and the administering physician's discretion. Any chimeric protein described herein, can be administered prior to (e.g., 5 minutes, 15 minutes, 30 minutes, 45 minutes, 1 hour, 2 hours, 4 hours, 6 hours, 12 hours, 24 hours, 48 hours, 72 hours, 96 hours, 1 week, 2 weeks, 3 weeks, 4 weeks, 5 weeks, 6 weeks, 8 weeks, or 12 weeks before), concurrently with, or subsequent to (e.g., 5 minutes, 15 minutes, 30 minutes, 45 minutes, 1 hour, 2 hours, 4 hours, 6 hours, 12 hours, 24 hours, 48 hours, 72 hours, 96 hours, 1 week, 2 weeks, 3 weeks, 4 weeks, 5 weeks, 6 weeks, 8 weeks, or 12 weeks after) the administration of an additional agent, to a subject in need thereof. In embodiments any chimeric protein and additional agent described herein are administered 1 minute apart, 10 minutes apart, 30 minutes apart, less than 1 hour apart, 1 hour apart, 1 hour to 2 hours apart, 2 hours to 3 hours apart, 3 hours to 4 hours apart, 4 hours to 5 hours apart, 5 hours to 6 hours apart, 6 hours to 7 hours apart, 7 hours to 8 hours apart, 8 hours to 9 hours apart, 9 hours to 10 hours apart, 10 hours to 11 hours apart, 11 hours to 12 hours apart, 1 day apart, 2 days apart, 3 days apart, 4 days apart, 5 days apart, 6 days apart, 1 week apart, 2 weeks apart, 3 weeks apart, or 4 weeks apart.

In embodiments, the present disclosure relates to the co-administration of a chimeric protein which induces an innate immune response and another chimeric protein which induces an adaptive immune response. In such embodiments, the chimeric protein which induces an innate immune response may be administered before, concurrently with, or subsequent to administration of the chimeric protein which induces an adaptive immune response. For example, the chimeric proteins may be administered 1 minute apart, 10 minutes apart, minutes apart, less than 1 hour apart, 1 hour apart, 1 hour to 2 hours apart, 2 hours to 3 hours apart, 3 hours to 4 hours apart, 4 hours to 5 hours apart, 5 hours to 6 hours apart, 6 hours to 7 hours apart, 7 hours to 8 hours apart, 8 hours to 9 hours apart, 9 hours to 10 hours apart, 10 hours to 11 hours apart, 11 hours to 12 hours apart, 1 day apart, 2 days apart, 3 days apart, 4 days apart, 5 days apart, 6 days apart, 1 week apart, 2 weeks apart, 3 weeks apart, or 4 weeks apart. In an illustrative embodiment, the chimeric protein which induces an innate immune response and the chimeric protein which induces an adaptive response are administered 1 week apart, or administered on alternate weeks (i.e., administration of the chimeric protein inducing an innate immune response is followed 1 week later with administration of the chimeric protein which induces an adaptive immune response and so forth).

The dosage of any chimeric protein (and/or additional agents) described herein can depend on several factors including the severity of the condition, whether the condition is to be treated or prevented, and the age, weight, and health of the subject to be treated. Additionally, pharmacogenomic (the effect of genotype on the pharmacokinetic, pharmacodynamic or efficacy profile of a therapeutic) information about a particular subject may affect dosage used. Furthermore, the exact individual dosages can be adjusted somewhat depending on a variety of factors, including the specific combination of the agents being administered, the time of administration, the route of administration, the nature of the formulation, the rate of excretion, the particular disease being treated, the severity of the disorder, and the anatomical location of the disorder. Some variations in the dosage can be expected.

For administration of any chimeric protein (and/or additional agents) described herein by parenteral injection, the dosage may be about 0.1 mg to about 250 mg per day, about 1 mg to about 20 mg per day, or about 3 mg to about 5 mg per day. Generally, when orally or parenterally administered, the dosage of any agent described herein may be about 0.1 mg to about 1500 mg per day, or about 0.5 mg to about 10 mg per day, or about 0.5 mg to about 5 mg per day, or about 200 to about 1,200 mg per day (e.g., about 200 mg, about 300 mg, about 400 mg, about 500 mg, about 600 mg, about 700 mg, about 800 mg, about 900 mg, about 1,000 mg, about 1,100 mg, about 1,200 mg, about 1300 mg, about 1400 mg, about 1500 mg, about 1600 mg, about 1700 mg, about 1800 mg, about 1900 mg, about 2,000 mg, about 2,100 mg, about 2,200 mg, about 2300 mg, about 2400 mg, about 2500 mg, about 2600 mg, about 2700 mg, about 2800 mg, about 2900 mg, about 3,000 mg, about 3,100 mg, about 3,200 mg, about 3300 mg, about 3400 mg, about 3500 mg, about 3600 mg, about 3700 mg, about 3800 mg, about 3900 mg, about 4,000 mg, about 4,100 mg, about 4,200 mg, about 4300 mg, about 4400 mg, about 4500 mg, about 4600 mg, about 4700 mg, about 4800 mg, about 4900 mg, about 5000 mg per day or per week).

In embodiments, administration of the chimeric protein (and/or additional agents) described herein is by parenteral injection or infusion at a dosage of about 0.1 mg to about 1500 mg per treatment, or about 0.5 mg to about 10 mg per treatment, or about 0.5 mg to about 5 mg per treatment, or about 200 to about 1,200 mg per treatment (e.g., about 200 mg, about 300 mg, about 400 mg, about 500 mg, about 600 mg, about 700 mg, about 800 mg, about 900 mg, about 1,000 mg, about 1,100 mg, about 1,200 mg, about 1300 mg, about 1400 mg, about 1500 mg, about 1600 mg, about 1700 mg, about 1800 mg, about 1900 mg, about 2,000 mg, about 2,100 mg, about 2,200 mg, about 2300 mg, about 2400 mg, about 2500 mg, about 2600 mg, about 2700 mg, about 2800 mg, about 2900 mg, about 3,000 mg, about 3,100 mg, about 3,200 mg, about 3300 mg, about 3400 mg, about 3500 mg, about 3600 mg, about 3700 mg, about 3800 mg, about 3900 mg, about 4,000 mg, about 4,100 mg, about 4,200 mg, about 4300 mg, about 4400 mg, about 4500 mg, about 4600 mg, about 4700 mg, about 4800 mg, about 4900 mg, about 5000 mg, per treatment).

In embodiments, a suitable dosage of the chimeric protein (and/or additional agents) is in a range of about mg/kg to about 100 mg/kg of body weight, or about 0.01 mg/kg to about 10 mg/kg of body weight of the subject, for example, or about 0.01 mg/kg, or about 0.02 mg/kg, or about 0.03 mg/kg, or about 0.04 mg/kg, or about 0.05 mg/kg, or about 0.06 mg/kg, or about 0.07 mg/kg, or about 0.08 mg/kg, or about 0.09 mg/kg, or about 0.1 mg/kg, or about 0.2 mg/kg, or about 0.3 mg/kg, or about 0.4 mg/kg, or about 0.5 mg/kg, or about mg/kg, or about 0.7 mg/kg, or about 0.8 mg/kg, or about 0.9 mg/kg, or about 1 mg/kg, or about 1.1 mg/kg, or about 1.2 mg/kg, or about 1.3 mg/kg, or about 1.4 mg/kg, or about 1.5 mg/kg, or about 1.6 mg/kg, or about 1.7 mg/kg, or about 1.8 mg/kg, 1.9 mg/kg, or about 2 mg/kg, or about 3 mg/kg, or about 4 mg/kg, or about 5 mg/kg, or about 6 mg/kg, or about 7 mg/kg, or about 8 mg/kg, or about 9 mg/kg, or about 10 mg/kg, or about 11 mg/kg, or about 12 mg/kg, or about 13 mg/kg, or about 14 mg/kg, or about 15 mg/kg, or about 16 mg/kg, or about 17 mg/kg, or about 18 mg/kg, or about 19 mg/kg, or about 20 mg/kg, or about 21 mg/kg, or about 22 mg/kg, or about 23 mg/kg, or about 24 mg/kg, or about 25 mg/kg, or about 26 mg/kg, or about 27 mg/kg, or about 28 mg/kg, or about 29 mg/kg, or about 30 mg/kg, or about 31 mg/kg, or about 32 mg/kg, or about 33 mg/kg, or about 34 mg/kg, or about 35 mg/kg, or about 36 mg/kg, or about 37 mg/kg, or about 38 mg/kg, or about 39 mg/kg, or about 40 mg/kg, or about 41 mg/kg, or about 42 mg/kg, or about 43 mg/kg, or about 44 mg/kg, or about 45 mg/kg, or about 46 mg/kg, or about 47 mg/kg, or about 48 mg/kg, or about 49 mg/kg, or about 50 mg/kg, or about 75 mg/kg, body weight, inclusive of all values and ranges therebetween.

In another embodiment, delivery can be in a vesicle, in particular a liposome (see Langer, 1990, Science 249:1527-1533; Treat et al., in Liposomes in therapy of Infectious Disease and Cancer, Lopez-Berestein and Fidler (eds.), Liss, New York, pp. 353-365 (1989).

Any chimeric protein (and/or additional agents) described herein can be administered by controlled-release or sustained-release means or by delivery devices that are well known to those of ordinary skill in the art. Examples include, but are not limited to, those described in U.S. Pat. Nos. 3,845,770; 3,916,899; 3,536,809; 3,598,123; 4,008,719; 5,674,533; 5,059,595; 5,591,767; 5,120,548; 5,073,543; 5,639,476; 5,354,556; and 5,733,556, each of which is incorporated herein by reference in its entirety. Such dosage forms can be useful for providing controlled- or sustained-release of one or more active ingredients using, for example, hydropropylmethyl cellulose, other polymer matrices, gels, permeable membranes, osmotic systems, multilayer coatings, microparticles, liposomes, microspheres, or a combination thereof to provide the desired release profile in varying proportions. Controlled- or sustained-release of an active ingredient can be stimulated by various conditions, including but not limited to, changes in pH, changes in temperature, stimulation by an appropriate wavelength of light, concentration or availability of enzymes, concentration or availability of water, or other physiological conditions or compounds.

In another embodiment, polymeric materials can be used (see Medical Applications of Controlled Release, Langer and Wise (eds.), CRC Pres., Boca Raton, Florida (1974); Controlled Drug Bioavailability, Drug Product Design and Performance, Smolen and Ball (eds.), Wiley, New York (1984); Ranger and Peppas, 1983, J. Macromol. Sci. Rev. Macromol. Chem. 23:61; see also Levy et al., 1985, Science 228:190; During et al., 1989, Ann. Neurol. 25:351; Howard et al., 1989, J. Neurosurg. 71:105).

In another embodiment, a controlled-release system can be placed in proximity of the target area to be treated, thus requiring only a fraction of the systemic dose (see, e.g., Goodson, in Medical Applications of Controlled Release, supra, vol. 2, pp. 115-138 (1984)). Other controlled-release systems discussed in the review by Langer, 1990, Science 249:1527-1533) may be used.

Administration of any chimeric protein (and/or additional agents) described herein can, independently, be one to four times daily or one to four times per month or one to six times per year or once every two, three, four or five years. Administration can be for the duration of one day or one month, two months, three months, six months, one year, two years, three years, and may even be for the life of the subject.

The dosage regimen utilizing any chimeric protein (and/or additional agents) described herein can be selected in accordance with a variety of factors including type, species, age, weight, sex and medical condition of the subject; the severity of the condition to be treated; the route of administration; the renal or hepatic function of the subject; the pharmacogenomic makeup of the individual; and the specific compound of the invention employed. Any chimeric protein (and/or additional agents) described herein can be administered in a single daily dose, or the total daily dosage can be administered in divided doses of two, three or four times daily. Furthermore, any chimeric protein (and/or additional agents) described herein can be administered continuously rather than intermittently throughout the dosage regimen.

Methods of Treatment, Inducing Lymphocyte Margination, Evaluating Efficacy, and Selecting Patients

In one aspect, the present disclosure relates to a method for treating a cancer in a subject in need thereof the method comprising a step of administering to the subject an effective amount of a chimeric protein having a general structure of: N terminus—(a)—(b)—(c)—C terminus, wherein: (a) is a first domain comprising an extracellular domain of human T cell immunoreceptor with Ig and ITIM domains (TIGIT), (b) is a linker adjoining the first and second domains, wherein the linker comprises a hinge-CH2-CH3 Fc domain, and (c) is a second domain comprising an extracellular domain of human LIGHT (lymphotoxin-like, exhibits inducible expression and competes with HSV glycoprotein D for herpesvirus entry mediator (HVEM), a receptor expressed by T lymphocytes, also known as CD258 or TNFSF14) with a dosing regimen selected from about every 3 days to about every 10 days, about every week to about every 2 weeks, about every 10 days to about every 3 weeks, about every 2 weeks to about every 4 weeks, about every 3 weeks to about every 5 weeks, about every 4 weeks to about every 6 weeks, about every 5 weeks to about every 7 weeks, about every 6 weeks to about every 8 weeks, and about every 6 weeks to about every 2 months. In some embodiments, the dosing regimen is about every week to about every 2 weeks, about every 10 days to about every 3 weeks, or about every 2 weeks to about every 4 weeks.

In some embodiments, the present chimeric proteins are capable of, or find use in methods involving, enhancing, restoring, promoting and/or stimulating immune modulation. In some embodiments, the present chimeric proteins described herein, restore, promote and/or stimulate the activity or activation of one or more immune cells against tumor cells including, but not limited to: T cells, cytotoxic T lymphocytes, T helper cells, natural killer (NK) cells, natural killer T (NKT) cells, anti-tumor macrophages (e.g., M1 macrophages), B cells, and dendritic cells. In some embodiments, the present chimeric proteins enhance, restore, promote and/or stimulate the activity and/or activation of T cells, including, by way of a non-limiting example, activating and/or stimulating one or more T-cell intrinsic signals, including a pro-survival signal; an autocrine or paracrine growth signal; a p38 MAPK-, ERK-, STAT-, JAK-, AKT- or PI3K-mediated signal; an anti-apoptotic signal; and/or a signal promoting and/or necessary for one or more of: pro-inflammatory cytokine production or T cell migration or T cell tumor infiltration.

In some embodiments, the present chimeric proteins are capable of, or find use in methods involving, causing an increase of one or more of T cells (including without limitation cytotoxic T lymphocytes, T helper cells, natural killer T (NKT) cells), B cells, natural killer (NK) cells, natural killer T (NKT) cells, dendritic cells, monocytes, and macrophages (e.g., one or more of M1 and M2) into a tumor or the tumor microenvironment. In some embodiments, the chimeric protein enhances recognition of tumor antigens by CD8+ T cells, particularly those T cells that have infiltrated into the tumor microenvironment. In some embodiments, the present chimeric protein induces CD19 expression and/or increases the number of CD19 positive cells (e.g., CD19 positive B cells). In some embodiments, the present chimeric protein induces IL-15Rα expression and/or increases the number of IL-15Rα positive cells (e.g., IL-15Rα positive dendritic cells).

In some embodiments, the present chimeric proteins are capable of, or find use in methods involving, inhibiting and/or causing a decrease in immunosuppressive cells (e.g., myeloid-derived suppressor cells (MDSCs), regulatory T cells (Tregs), tumor associated neutrophils (TANs), M2 macrophages, and tumor associated macrophages (TAMs)), and particularly within the tumor and/or tumor microenvironment (TME). In some embodiments, the present therapies may alter the ratio of M1 versus M2 macrophages in the tumor site and/or TME to favor M1 macrophages.

In some embodiments, the present chimeric proteins are able to increase the serum levels of various cytokines including, but not limited to, one or more of IL-2, IL-10, IP-10 (CXCL10), MCP-1, MIP-1β (CCL4) TARC (CCL17), IFNγ, IL-8, IL-12, and SDF1a (CXCL12). In some embodiments, the present chimeric proteins are capable of enhancing IL-2, IL-10, IP-10 (CXCL10), MCP-1, MIP-1β (CCL4) TARC (CCL17), IFNγ, IL-8, IL-12, and SDF1a (CXCL12) in the serum of a treated subject.

In one aspect, the present disclosure relates to a method for treating a cancer in a subject in need thereof the method comprising a step of administering to the subject an effective amount of a chimeric protein having a general structure of: N terminus—(a)—(b)—(c)—C terminus, wherein: (a) is a first domain comprising an extracellular domain of human T cell immunoreceptor with Ig and ITIM domains (TIGIT), (b) is a linker adjoining the first and second domains, wherein the linker comprises a hinge-CH2-CH3 Fc domain, and (c) is a second domain comprising an extracellular domain of human LIGHT (lymphotoxin-like, exhibits inducible expression and competes with HSV glycoprotein D for herpesvirus entry mediator (HVEM), a receptor expressed by T lymphocytes, also known as CD258 or TNFSF14). In some embodiments, the subject is dosed with a dosing regimen selected from about every 3 days to about every 10 days, about every week to about every 2 weeks, about every 10 days to about every 3 weeks, about every 2 weeks to about every 4 weeks, about every 3 weeks to about every 5 weeks, about every 4 weeks to about every 6 weeks, about every 5 weeks to about every 7 weeks, about every 6 weeks to about every 8 weeks, and about every 6 weeks to about every 2 months. In some embodiments, the dosing regimen is about every week to about every 2 weeks, about every 10 days to about every 3 weeks, or about every 2 weeks to about every 4 weeks. In some embodiments, the initial dose is selected from about 0.03 mg/kg, about 0.1 mg/kg, about 0.3 mg/kg, about 1 mg/kg, about 2 mg/kg, about 3 mg/kg, about 4 mg/kg, about 5 mg/kg, about 6 mg/kg, about 7 mg/kg, about 8 mg/kg, about 9 mg/kg, about 10 mg/kg, about 12 mg/kg, about 15 mg/kg, about 18 mg/kg, about 21 mg/kg, about 24 mg/kg, about 25 mg/kg, about 27 mg/kg, about 30 mg/kg, about 33 mg/kg, about 35 mg/kg, about 36 mg/kg, about 40 mg/kg, about 42 mg/kg, about 45 mg/kg, about 48 mg/kg, and about 50 mg/kg. In some embodiments, the dose is selected from about 0.03 mg/kg, about 0.1 mg/kg, about 0.3 mg/kg, about 1 mg/kg, about 2 mg/kg, about 3 mg/kg, about 4 mg/kg, about 5 mg/kg, about 6 mg/kg, about 7 mg/kg, about 8 mg/kg, about 9 mg/kg, about 10 mg/kg, about 12 mg/kg, about 15 mg/kg, about 18 mg/kg, about 21 mg/kg, about 24 mg/kg, about 25 mg/kg, about 27 mg/kg, about 30 mg/kg, about 33 mg/kg, about 35 mg/kg, about 36 mg/kg, about 40 mg/kg, about 42 mg/kg, about 45 mg/kg, about 48 mg/kg, and about 50 mg/kg. In embodiments, the dose is administered with a once a week or a once every two weeks schedule. In embodiments, the method further comprising administration of a priming dose to the subject.

In one aspect, the present disclosure relates to a method for inducing lymphocyte expansion in a subject in need thereof the method comprising a step of administering to the subject an effective amount of a chimeric protein having a general structure of: N terminus—(a)—(b)—(c)—C terminus, wherein: (a) is a first domain comprising an extracellular domain of human T cell immunoreceptor with Ig and ITIM domains (TIGIT), (b) is a linker adjoining the first and second domains, wherein the linker comprises a hinge-CH2-CH3 Fc domain, and (c) is a second domain comprising an extracellular domain of human LIGHT (lymphotoxin-like, exhibits inducible expression and competes with HSV glycoprotein D for herpesvirus entry mediator (HVEM), a receptor expressed by T lymphocytes, also known as CD258 or TNFSF14). In some embodiments, the subject is dosed with a dosing regimen selected from about every 3 days to about every 10 days, about every week to about every 2 weeks, about every 10 days to about every 3 weeks, about every 2 weeks to about every 4 weeks, about every 3 weeks to about every 5 weeks, about every 4 weeks to about every 6 weeks, about every 5 weeks to about every 7 weeks, about every 6 weeks to about every 8 weeks, and about every 6 weeks to about every 2 months. In some embodiments, the dosing regimen is about every week to about every 2 weeks, about every 10 days to about every 3 weeks, or about every 2 weeks to about every 4 weeks. In some embodiments, the initial dose is selected from about 0.03 mg/kg, about 0.1 mg/kg, about 0.3 mg/kg, about 1 mg/kg, about 2 mg/kg, about 3 mg/kg, about 4 mg/kg, about 5 mg/kg, about 6 mg/kg, about 7 mg/kg, about 8 mg/kg, about 9 mg/kg, about 10 mg/kg, about 12 mg/kg, about 15 mg/kg, about 18 mg/kg, about 21 mg/kg, about 24 mg/kg, about 25 mg/kg, about 27 mg/kg, about 30 mg/kg, about 33 mg/kg, about 35 mg/kg, about 36 mg/kg, about 40 mg/kg, about 42 mg/kg, about 45 mg/kg, about 48 mg/kg, and about 50 mg/kg. In some embodiments, the dose is selected from about 0.03 mg/kg, about 0.1 mg/kg, about 0.3 mg/kg, about 1 mg/kg, about 2 mg/kg, about 3 mg/kg, about 4 mg/kg, about 5 mg/kg, about 6 mg/kg, about 7 mg/kg, about 8 mg/kg, about 9 mg/kg, about 10 mg/kg, about 12 mg/kg, about 15 mg/kg, about 18 mg/kg, about 21 mg/kg, about 24 mg/kg, about 25 mg/kg, about 27 mg/kg, about 30 mg/kg, about 33 mg/kg, about 35 mg/kg, about 36 mg/kg, about 40 mg/kg, about 42 mg/kg, about 45 mg/kg, about 48 mg/kg, and about 50 mg/kg. In embodiments, the dose is administered with a once a week or a once every two weeks schedule. In embodiments, the method further comprising administration of a priming dose to the subject.

In one aspect, the present disclosure relates to a method for inducing lymphocyte margination in a subject in need thereof the method comprising a step of administering to the subject an effective amount of a chimeric protein having a general structure of: N terminus—(a)—(b)—(c)—C terminus, wherein: (a) is a first domain comprising an extracellular domain of human T cell immunoreceptor with Ig and ITIM domains (TIGIT), (b) is a linker adjoining the first and second domains, wherein the linker comprises a hinge-CH2-CH3 Fc domain, and (c) is a second domain comprising an extracellular domain of human LIGHT (lymphotoxin-like, exhibits inducible expression and competes with HSV glycoprotein D for herpesvirus entry mediator (HVEM), a receptor expressed by T lymphocytes, also known as CD258 or TNFSF14). In some embodiments, the subject is dosed with a dosing regimen selected from about every 3 days to about every 10 days, about every week to about every 2 weeks, about every 10 days to about every 3 weeks, about every 2 weeks to about every 4 weeks, about every 3 weeks to about every 5 weeks, about every 4 weeks to about every 6 weeks, about every 5 weeks to about every 7 weeks, about every 6 weeks to about every 8 weeks, and about every 6 weeks to about every 2 months. In some embodiments, the dosing regimen is about every week to about every 2 weeks, about every 10 days to about every 3 weeks, or about every 2 weeks to about every 4 weeks. In some embodiments, the initial dose is selected from about 0.03 mg/kg, about 0.1 mg/kg, about 0.3 mg/kg, about 1 mg/kg, about 2 mg/kg, about 3 mg/kg, about 4 mg/kg, about 5 mg/kg, about 6 mg/kg, about 7 mg/kg, about 8 mg/kg, about 9 mg/kg, about 10 mg/kg, about 12 mg/kg, about 15 mg/kg, about 18 mg/kg, about 21 mg/kg, about 24 mg/kg, about 25 mg/kg, about 27 mg/kg, about 30 mg/kg, about 33 mg/kg, about 35 mg/kg, about 36 mg/kg, about 40 mg/kg, about 42 mg/kg, about 45 mg/kg, about 48 mg/kg, and about 50 mg/kg. In some embodiments, the dose is selected from about 0.03 mg/kg, about 0.1 mg/kg, about 0.3 mg/kg, about 1 mg/kg, about 2 mg/kg, about 3 mg/kg, about 4 mg/kg, about 5 mg/kg, about 6 mg/kg, about 7 mg/kg, about 8 mg/kg, about 9 mg/kg, about 10 mg/kg, about 12 mg/kg, about 15 mg/kg, about 18 mg/kg, about 21 mg/kg, about 24 mg/kg, about 25 mg/kg, about 27 mg/kg, about 30 mg/kg, about 33 mg/kg, about 35 mg/kg, about 36 mg/kg, about 40 mg/kg, about 42 mg/kg, about 45 mg/kg, about 48 mg/kg, and about 50 mg/kg. In embodiments, the dose is administered with a once a week or a once every two weeks schedule. In embodiments, the method further comprising administration of a priming dose to the subject.

In one aspect, the present disclosure relates to a method of evaluating the efficacy of a cancer treatment in a subject in need thereof, the method comprising the steps of: administering a dose of a chimeric protein having a general structure of: N terminus—(a)—(b)—(c)—C terminus, wherein: (a) is a first domain comprising an extracellular domain of human T cell immunoreceptor with Ig and ITIM domains (TIGIT), (b) is a linker adjoining the first and second domains, wherein the linker comprises a hinge-CH2-CH3 Fc domain, and (c) is a second domain comprising an extracellular domain of human LIGHT (lymphotoxin-like, exhibits inducible expression and competes with HSV glycoprotein D for herpesvirus entry mediator (HVEM), a receptor expressed by T lymphocytes, also known as CD258 or TNFSF14), wherein the dose is from about mg/kg to about 50 mg/kg; obtaining a biological sample from the subject; performing an assay on the biological sample to determine level and/or activity of a cytokine selected from IL-2, IL-10, IP-10 (CXCL10), MCP-1, MIP-1β (CCL4) TARC (CCL17), IFNγ, IL-8, IL-12, and SDF1a (CXCL12); and continuing dosing if the subject has an increase in the level and/or activity of at least one cytokine selected from IL-2, IL-10, IP-10 (CXCL10), MCP-1, MIP-1β (CCL4) TARC (CCL17), IFNγ, IL-8, IL-12, and SDF1a (CXCL12).

In one aspect, the present disclosure relates to a method of selecting a subject for treatment with a therapy for cancer, the method comprising the steps of: administering a dose of a chimeric protein having a general structure of: N terminus—(a)—(b)—(c)—C terminus, wherein: (a) is a first domain comprising an extracellular domain of human T cell immunoreceptor with Ig and ITIM domains (TIGIT), (b) is a linker adjoining the first and second domains, wherein the linker comprises a hinge-CH2-CH3 Fc domain, and (c) is a second domain comprising an extracellular domain of human LIGHT (lymphotoxin-like, exhibits inducible expression and competes with HSV glycoprotein D for herpesvirus entry mediator (HVEM), a receptor expressed by T lymphocytes, also known as CD258 or TNFSF14), wherein the dose is from about 0.03 mg/kg to about 50 mg/kg; obtaining a biological sample from the subject; performing an assay on the biological sample to determine level and/or activity of a cytokine selected from IL-2, IL-10, IP-10 (CXCL10), MCP-1, MIP-1β (CCL4) TARC (CCL17), IFNγ, IL-8, IL-12, and SDF1a (CXCL12); and selecting the subject for treatment with the therapy for cancer if the subject has an increase in the level and/or activity of at least one cytokine selected from IL-2, IL-10, IP-10 (CXCL10), MCP-1, MIP-1β (CCL4) TARC (CCL17), IFNγ, IL-8, IL-12, and SDF1a (CXCL12).

### EXAMPLES

The examples herein are provided to illustrate advantages and benefits of the present disclosure and to further assist a person of ordinary skill in the art with preparing or using cells that are resistant to anti-PD-1, anti-PD-L1 and/or anti-PD-L2 therapy. The examples herein are also presented in order to more fully illustrate the preferred aspects of the present disclosure. The examples should in no way be construed as limiting the scope of the present disclosure, as defined by the appended claims. The examples can include or incorporate any of the variations, aspects or embodiments of the present disclosure described above. The variations, aspects or embodiments described above may also further each include or incorporate the variations of any or all other variations, aspects or embodiments of the present disclosure.

### Example 1: Generation of Anti-PD-1-Resistant CT26 Tumors

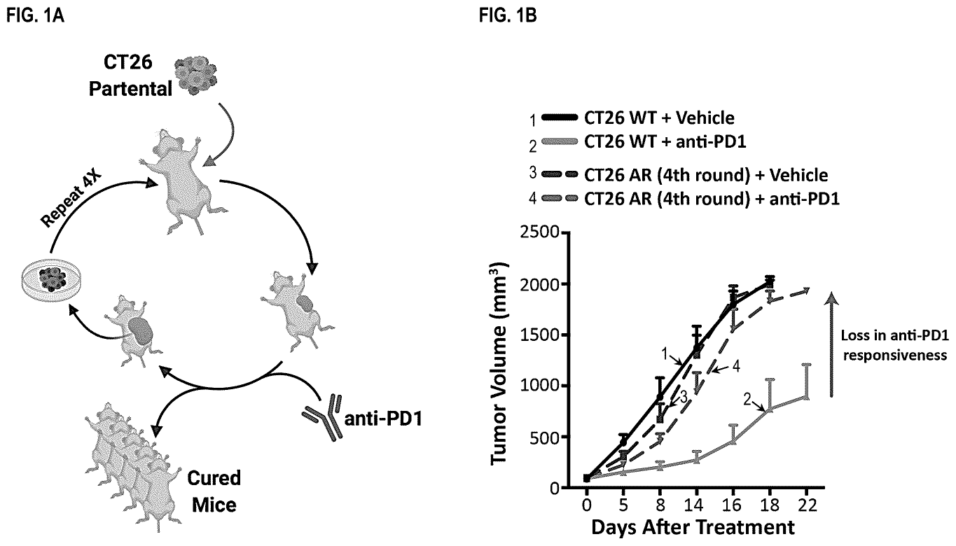

Murine colon carcinoma CT26 cell were subjected to selection for surviving the anti-PD-1 treatment to generate anti-PD-1-antibody resistant CT26 cells. The method used to generation of the anti-PD-1-resistant CT26 tumor cells is illustrated in FIG. 1A. Briefly, BALB/C mice were acquired from the Jackson Laboratory, and after several days of acclimation, mice were inoculated with 500,000 murine colon carcinoma CT26 cells on the rear flank. When average tumor volume reached 80-100 mm³ (indicating day 0), mice were given a series of intraperitoneal injections of 100 μg anti-PD-1 (clone RMP1-14; BioXcell) on days 0, 3, and 6. Tumors were excised from mice that did not respond to anti-PD-1 therapy, approximately 10-14 days following the initial treatment. Tumors were dissociated using collagenase (StemCell Technologies), washed in 1×PBS, and plated in IMDM culture media supplemented with 10% fetal bovine serum, 1% GLUTiMAX, and 1% Antibiotic-Antimycotic (all GIBCO). Cells were passaged at least 5 times and were then inoculated into new recipient mice according as described above (round 2). Again, when tumors reached 80-100 mm³, another treatment course of anti-PD-1 was initiated: intraperitoneal injections of 100 μg anti-PD-1 (clone RMP1-14; BioXcell) were administered on days 0, 3, and 6. This process was repeated for a total of 4 rounds, at which point none of the treated mice responded to anti-PD-1 therapy. The cell lines generated after 2 rounds of anti-PD-1 selection are referred to throughout this disclosure as “2nd round,” “2nd generation,” or “F2 generation.” The cell lines generated after 4 rounds of anti-PD-1 selection are referred to as “4th round,” “4th generation,” or “F4 generation.”

The efficacy of anti-PD-1 antibody in CT26 cell- or anti-PD-1-antibody resistant CT26 cell-allografts was compared. Briefly, BALB/C mice were inoculated with CT26 parental cells and PD-1 resistant 4th generation cells in rear flanks. When the starting tumor volume (STV) reached 80-100 mm³, mice were randomly divided in the following two treatment groups: (1) vehicle (PBS), and (2) anti-PD-1 antibody. Mice were given a series of intraperitoneal injections of vehicle or 100 μg anti-PD-1 (clone RMP1-14; BioXcell) on days 0, 3, and 6. Tumor volumes were measured on indicated days and plotted as a function of time. As shown in FIG. 1B, the growth of CT26 parental cell tumors was significantly retarded when treated with the anti-PD-1 antibody, compared to vehicle-only control (compare the grey solid line with the black solid line in FIG. 1B). In contrast, the PD-1 resistant cells showed very little retardation of tumor, if any. These results demonstrate, inter alia, that the anti-PD-1 resistance of anti-PD-1 resistant cells was developed after therapy (acquired resistance) in immune competent mice.

Therefore, anti-PD-1 antibody-resistant cells were developed. Also, a mouse model of cells harboring anti-PD-1 resistant cells was developed. Accordingly, the mouse model of disclosed herein is useful for testing an anti-cancer drug candidate by administering the anti-cancer drug candidate to mice bearing anti-PD-1-antibody resistant CT26 cell-allografts, and evaluating whether the anti-cancer drug candidate is effective in slowing or inhibiting cancer growth. An anti-cancer drug or candidate that is effective in slowing or inhibiting cancer growth may be formulated for administration to a human patient.

### Example 2: Transcriptomic Profiling of Anti-PD-1 Resistant Cell Lines Using RNA-Seq

Three distinct vials of parental CT26 cells (ATCC; “experimental replicates”), two independently isolated tumors from “2nd round” mice, four independently isolated tumors from “4th round” mice (both “biological replicates”) were cultured with or without 20 ng/mL of mouse IFNγ (Biolegend) for 24 hours at 37° 015%002. The following day, RNA was isolated from cells using Qiagen RNeasy reagents according to manufacturer's instructions, including QIASHREDDER homogenization and on-column DNaseI digestion. Isolated RNA was subjected to RNA-seq and data analysis. Briefly, sequencing libraries were generated and sequenced on an ILLUMINA HISEQ (2×150 paired end reads), targeting >20×10⁶ reads per sample. Sequences were trimmed using TRIMMOMATIC v.0.36 and mapped to the Mus musculus GRCm38 reference genome using the STAR ALIGNER v.2.5.2b. Unique gene hit counts were calculated by using FEATURECOUNTS from the SUBREAD package v.1.5.2. Only unique reads that fell within exon regions were counted. Differential gene expression was determined using DESeq2, and the Wald test was used to generate p-values and log 2 fold changes; with log 2 fold change >1 and adjusted p-values <0.05 as cutoffs for significance.

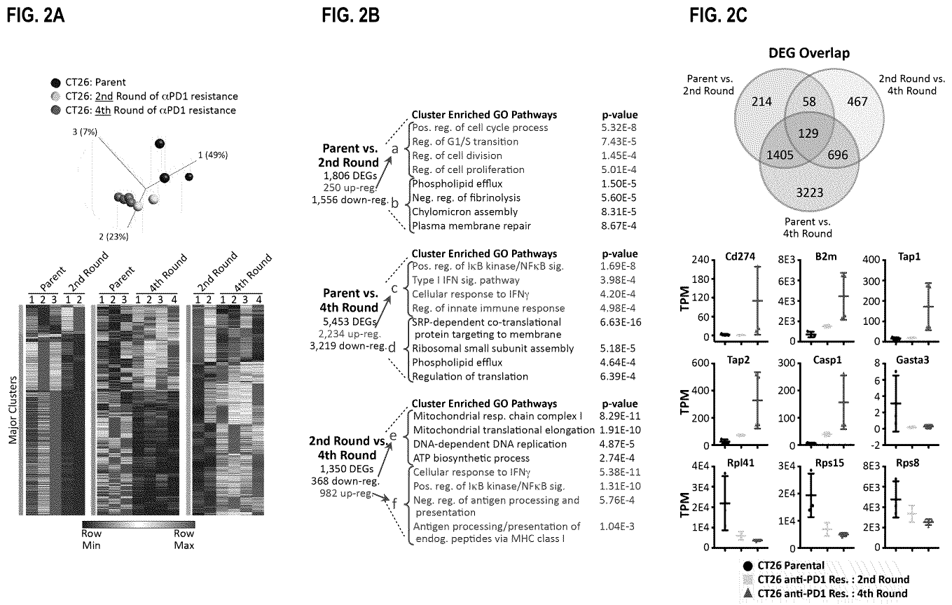

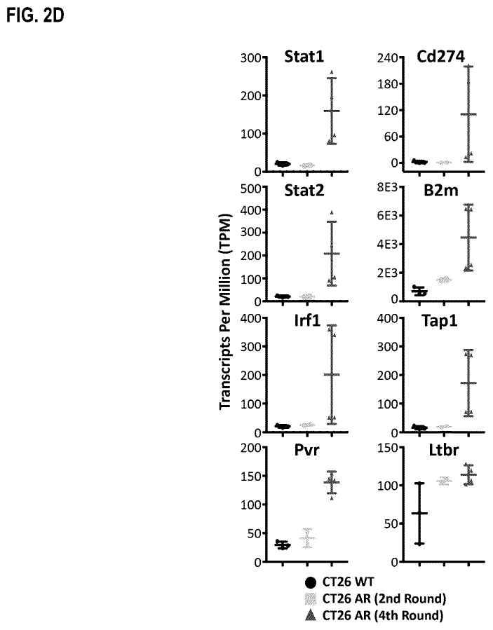

As shown in FIG. 2A (Top Panel), Principal Component Analysis (PCA) illustrated that that the samples could be spatially separated based on transcriptome expression. Differentially Expressed Genes (DEGs) were determined between the groups (parent vs. 2nd generation, parent vs. 4th generation, 2nd generation vs. 4th generation), and plotted in the heatmap. As shown in FIG. 2A (Bottom Panel), Hierarchical clustering performed to rank order genes on each row, separated genes into 2 major clusters in each comparison, where a subset of gene expression was lower (blue) in one dataset and higher (red) in the other. The genes were then subjected to analysis using the PANTHER application to identify Gene Ontologies (GO) associated with each gene set. Gene sets are shown with associated p-values. Up- or down-regulated genes were identified in each dataset. As shown in FIG. 2B, genes associated with the following GO were upregulated in 2nd generation cells compared to parental CT26 cells: positive regulation of cell cycle process, regulation of G1/S transition, regulation of cell division, and regulation of cell proliferation. Genes associated with the following GO were downregulated in 2nd generation cells compared to parental CT26 cells: phospholipid efflux, negative regulation of fibrinolysis, chylomicron assembly, and plasma membrane repair (FIG. 2B). Further, as shown in FIG. 2B, genes associated with the following GO were upregulated in 4th generation cells compared to parental CT26 cells: positive regulation of IκB kinase/NFκB signaling, type I IFN signaling pathway, cellular response to IFNγ, regulation of innate immune response. This was surprising, inter alia, because it has been widely known that cellular response to IFNγ is involved in sensitivity to anti-PD-1 and other immunotherapeutics. Genes associated with the following GO were downregulated in 4th generation cells compared to parental CT26 cells: SRP-dependent co-translational protein targeting to membrane, ribosomal small subunit assembly, phospholipid efflux, regulation of translation (FIG. 2B). Interestingly, the genes associated with the following GO were upregulated in 4th generation cells compared to 2nd generation cells: cellular response to IFNγ, positive regulation of IκB kinase/NFκB signaling, negative regulation of antigen processing/presentation, and antigen processing/presentation of endogenous peptides via MHC class I (FIG. 2B). Genes associated with the following GO were downregulated in 4th generation cells compared to 2nd generation cells: mitochondrial respiratory chain complex I, mitochondrial translational elongation, DNA-dependent DNA replication, and ATP biosynthetic process (FIG. 2B). A Venn diagram of overlap in gene expression between all datasets was created. As shown in FIG. 2C (Top Panel), the DEGs showed a significant overlap between 4th generation cells and 2nd generation cells. FIG. 2A (Bottom Panel) shows the transcripts per million (TPM; normalized expression data) at select genes, demonstrating higher baseline expression of genes associated with PD-L1, antigen processing/presentation, protein translation, ER trafficking; in some datasets over others. As shown in FIG. 2C (Bottom Panel), the genes Cd274, B2M, Tap1, Tap2, Casp1, and Gasta3 showed progressively increasing expression from CT26 parental cells to 2nd generation cells and to 4th generation cells. Further, genes Rpl41, Rps15 and Rps8 showed progressively decreasing expression from CT26 parental cells to 2nd generation cells and to 4th generation cells (FIG. 2C (Bottom Panel)). As shown in FIG. 2D, the genes Stat1, Stat2, Irf, Ltbr, and Pvr showed progressively increasing expression from CT26 parental cells to 2nd generation cells and to 4th generation cells.

These results, inter alia, establish biomarkers associated with acquired resistance to anti-PD-1 therapy. Such biomarkers may be used to identify patients that that may benefit from anti-PD-1 therapy or those that will not benefit from anti-PD-1 therapy. Therefore, based on these biomarkers, a patient may be selected for treatment with an anti-PD-1 therapy based on evaluating the sample for the presence, absence, or level of genes associated with one or more gene ontology (GO) pathways disclosed herein from a biological sample from the patient. A cancer therapy with an ability to inhibit function and/or activity of PD-1, PD-L1 and/or PD-L2 may be indicated, for example, when expression of one or more of Rpl41, Rps15 and Rps8 is high and/or the expression of one or more of Cd274, B2m, Tap1, Tap2, Casp1, and Gasta3 is low. In contrast, a cancer therapy with an ability to inhibit function and/or activity of PD-1, PD-L1 and/or PD-L2 may not be indicated, for example, when expression of one or more of Rpl41, Rps15 and Rps8 is low and/or the expression of one or more of Cd274, B2m, Tap1, Tap2, Casp1, and Gasta3 is high. The patients featuring low expression of one or more of Rpl41, Rps15 and Rps8 and/or high expression of one or more of Cd274, B2m, Tap1, Tap2, Casp1, and Gasta3 are likely to benefit from adjuvant or neoadjuvant therapies that eliminate the PD-1-nonresponsive cells.

### Example 3: Cell Surface Expression of PD-L1, PD-L2, MHC Class I and/32 Microglobulin in Anti-PD-1 Resistant CT26

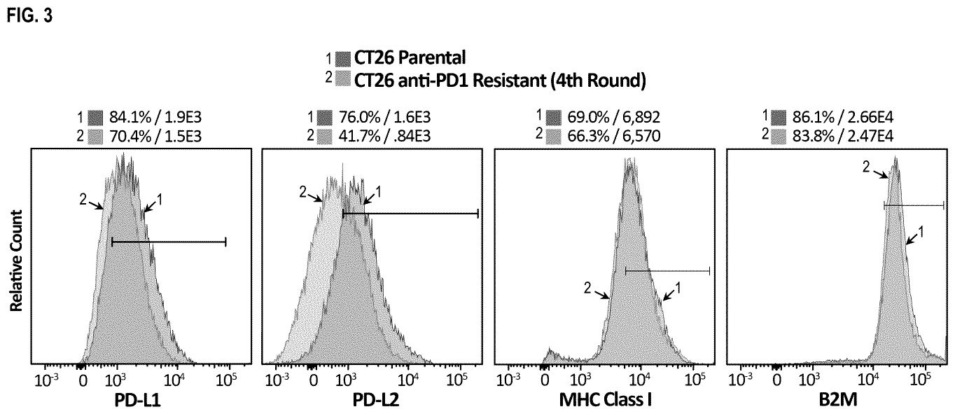

Since Cd274 (PD-L1), β2 microglobulin (B2m), and Tap1 and Tap2 genes (potentially involved in the processing and transport of major histocompatibility complex class I-associated antigen to the endoplasmic reticulum) were progressively downregulated from CT26 parental cells to 2nd generation cells and to 4th generation cells, surface expression of PD-L1, PD-L2, MHC Class I and β2 microglobulin (B2m) was studied. Briefly, parental and were harvested from culture and analyzed by flow cytometry for surface expression of PD-L1, PD-L2, MHC Class I, and β2 microglobulin (B2M). Gates were drawn as shown, and shown above each plot is the percentage of cells in each gate, and to the right of each percentage, the MFI (mean fluorescent intensity) of each marker. As shown in FIG. 3, the surface expression of PD-L1, MHC Class I and β2 microglobulin (B2M) was not downregulated in 4th generation anti-PD-1 resistant cells compared to parental cells. on the other hand, the surface expression of PD-L2 was downregulated in 4th generation anti-PD-1 resistant cells compared to parental cells. These data show that a discordance in gene expression and cell surface protein expression was observed surface expression in PD-L1/2 MHC Class I and B2M compared to RNA expression.

### Example 4: Transcriptomic Profiling of the Anti-PD-1 Resistant Cell Lines and a Primarily Resistant Anti-PD-1 Resistant Cell Line

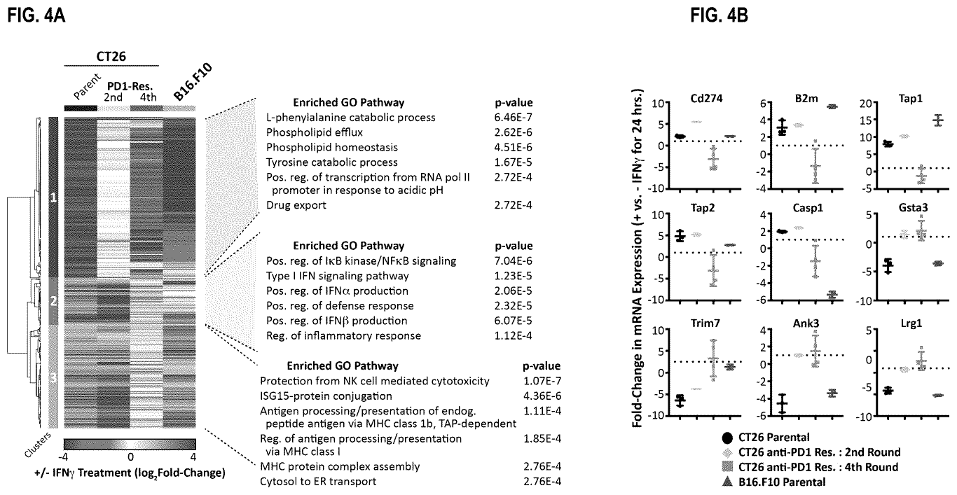

Next the transcriptomic profile of the 2nd and 4th generation anti-PD-1 resistant cell lines was studied by comparison with each other and with an anti-PD-1 primarily resistant cell line. The B16.F10 murine melanoma tumor cell line was used as a model of anti-PD-1 “primary resistance,” as these tumors are not responsive to anti-PD-1 therapy. Three distinct vials of parental CT26 cells (ATCC; “experimental replicates”), two independently isolated tumors from “2nd round” mice, four independently isolated tumors from “4th round” mice (both “biological replicates”), and two distinct vials of parental B16.F10 cells (ATCC; “experimental replicates were cultured with or without 20 ng/mL of mouse IFNγ (Biolegend) for 24 hours at 37° 015%002 to assess in vitro responsiveness. This mimics how tumor cells respond in vivo, as immune cell infiltrate and secrete effector cytokines like IFNγ. The following day, RNA was isolated from cells using Qiagen RNeasy reagents according to manufacturers instructions, including QIASHREDDER homogenization and on-column DNaseI digestion. Isolated RNA was subjected to RNA-seq and data analysis. Briefly, sequencing libraries were generated and sequenced on an ILLUMINA HISEQ (2×150 paired end reads), targeting >20×10⁶ reads per sample. Sequences were trimmed using TRIMMOMATIC v.0.36 and mapped to the Mus musculus GRCm38 reference genome using the STAR ALIGNER v.2.5.2b. Unique gene hit counts were calculated by using FEATURECOUNTS from the SUBREAD package v.1.5.2. Only unique reads that fell within exon regions were counted. Differential gene expression was determined using DESeq2, and the Wald test was used to generate p-values and log 2 fold changes; with log 2 fold change >1 and adjusted p-values <0.05 as cutoffs for significance. The DEGs were identified between untreated and IFNγ treated parental CT26. As shown in FIG. 4A (Left Panel), Log 2 fold-change was plotted in the heatmap and genes are hierarchical clustered based on parental CT26. Of these, 338 genes had usable data from the other datasets; and those values are shown in the other columns (FIG. 4A (Left Panel)). Genes separated into 3 major clusters (FIG. 4A (Left Panel)). Associated genes were input into PANTHER to identify pathways associated with the dysregulated genes and the GO pathways associated with the DEGs were identified. As shown in FIG. 4A (Right Panel), the upregulation of expression of the genes associated with following GO pathways was enriched in 2nd generation cells and 4th generation cells compared to both parental CT26 cells and B16.F10 cells: L-phenyl alanine catabolic process, phospholipid efflux, tyrosine catabolic process, positive regulation of transcription from RNA polymerase II promoter in response to acidic pH, and drug export. Therefore, it is likely that upregulation of genes associated with one or more of those GO pathways is associated with acquired resistance to anti-PD-1 therapy. Further, the downregulation of expression of the genes associated with following GO pathways was observed in 4th generation cells compared to both parental CT26 cells and B16.F10 cells: protection of NK cell mediated cytotoxicity, IGS15-protein conjugation, antigen processing/presentation via MHC class I, MHC protein complex assembly, and cytosol to ER transport (FIG. 4A (Right Panel)). Therefore, it is likely that downregulation of one or more of those GO pathways is associated with acquired resistance to anti-PD-1 therapy. As shown in FIG. 4A (Right Panel), the downregulation of expression of the genes associated with following GO pathways was enriched in 4th generation cells and B16.F10 cells compared to both parental CT26 cells: positive regulation of IκB kinase/NFκB signaling, type I IFN signaling pathways, positive regulation of IFNα production, positive regulation of defense response, positive regulation of IFNβ production, and regulation of inflammatory response. Therefore, it is likely that downregulation of one or more of those GO pathways is associated with resistance to anti-PD-1 therapy. FIG. 4B shows the transcripts per million (TPM; normalized expression data) at select genes. As shown in FIG. 4B, although CT26 anti-PD-1 resistant cells have baseline hyperactivation of type I and type II interferons, when those cells are challenged with IFNγ, those cells downregulate these genes. This was surprising, inter alia, because it has been widely known that positive regulation of IκB kinase/NFκB signaling, type I IFN signaling pathways, positive regulation of IFNα production, positive regulation of defense response, positive regulation of IFNβ production, and regulation of inflammatory response are all involved in sensitivity to anti-PD-1 and other immunotherapeutics.

These results, inter alia, establish biomarkers associated with acquired resistance to anti-PD-1 therapy, and biomarkers associated with resistance to anti-PD-1 therapy (acquired or primary resistance). Such biomarkers may be used to identify patients that that may benefit from anti-PD-1 therapy or those that will not benefit from anti-PD-1 therapy. Therefore, based on these biomarkers, a patient may be selected for treatment with an anti-PD-1 therapy based on evaluating the sample for the presence, absence, or level of genes associated with one or more gene ontology (GO) pathways disclosed herein from a biological sample from the patient. A cancer therapy with an ability to inhibit function and/or activity of PD-1, PD-L1 and/or PD-L2 may be indicated, for example, when expression of Trim7 and/or Ank3 is high and/or the expression of Tap2, and/or Casp1 is low. In contrast, a cancer therapy with an ability to inhibit function and/or activity of PD-1, PD-L1 and/or PD-L2 may not be indicated, for example, when expression of Trim7 and/or Ank3 is high and/or the expression of Tap2, and/or Casp1 is low. The patients featuring when high expression of Trim7 and/or Ank3, and/or the low expression of Tap2, and/or Casp1 is are likely to benefit from adjuvant or neoadjuvant therapies that eliminate the PD-1-nonresponsive cells.

### Example 5: Paradoxical Dysregulation of Genes that are Differentially Regulated in Anti-PD-1 Resistant Cells Compared to Parental Cells

Next, the effect of IFNγ on the expression of certain genes that are differentially expressed in the 4th generation anti-PD-1 resistant cells compared to the wild type CT26 cells was studied. Briefly, the parental CT26 cells, second generation anti-PD-1 resistant cells and 4th generation anti-PD-1 resistant cells were cultured with or without 20 ng/mL of mouse IFNγ (Biolegend) for 24 hours at 37° 015%002. The following day, RNA was isolated from cells using Qiagen RNeasy reagents according to manufacturer's instructions, including QIASHREDDER homogenization and on-column DNaseI digestion. Isolated RNA was subjected to RNA-seq and data analysis. Briefly, sequencing libraries were generated and sequenced on an ILLUMINA HISEQ (2×150 paired end reads), targeting >20×10⁶ reads per sample. Sequences were trimmed using TRIMMOMATIC v.0.36 and mapped to the Mus musculus GRCm38 reference genome using the STAR ALIGNER v.2.5.2b. Unique gene hit counts were calculated by using FEATURECOUNTS from the SUBREAD package v.1.5.2. Only unique reads that fell within exon regions were counted. Differential gene expression was determined using DESeq2, and the Wald test was used to generate p-values and log 2 fold changes; with log 2 fold change >1 and adjusted p-values <0.05 as cutoffs for significance.

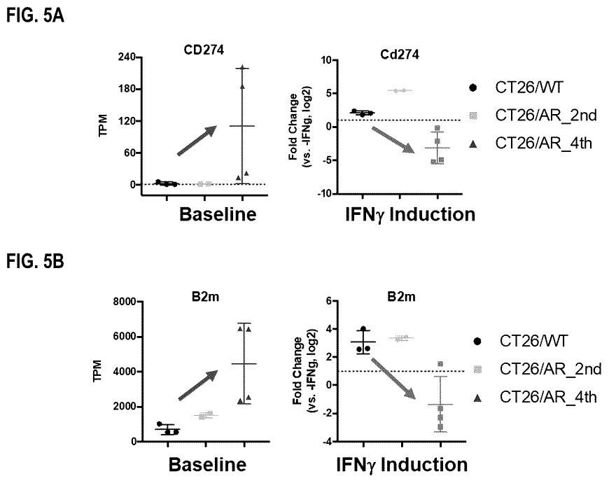

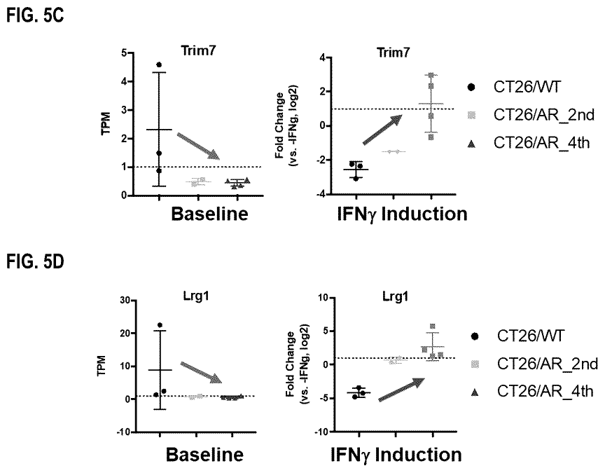

To understand the effect of IFNγ, the transcripts per million (TPM; normalized expression data) of representative genes that are either overexpressed (Cd274 and B2m) or repressed (Trim7 and Lrg1) were analyzed. As expected, the expression of Cd274 (PD-L1) increased from wild type CT26 cells to second generation anti-PD-1 resistant cells and to 4th generation anti-PD-1 resistant cells (FIG. 5A (left panel)). The expression of Cd274 increased in wild type CT26 cells increased when the wild type CT26 cells were induced with IFNγ (compare closed circles data in FIG. 5A (left and right panels)). Paradoxically, the expression of Cd274 (PD-L1) decreased from wild type CT26 cells to second generation anti-PD-1 resistant cells and to 4th generation anti-PD-1 resistant cells in response to IFNγ (FIG. 5A (right panel)).

The expression of B2M also increased from wild type CT26 cells to second generation anti-PD-1 resistant cells and to 4th generation anti-PD-1 resistant cells, as expected (FIG. 5B (left panel)). The expression of B2M increased in wild type CT26 cells increased when the wild type CT26 cells were induced with IFNγ (compare closed circles data in FIG. 5B (left and right panels)). Paradoxically, the expression of B2M decreased from wild type CT26 cells to second generation anti-PD-1 resistant cells and to 4th generation anti-PD-1 resistant cells in response to IFNγ (FIG. 5B (right panel)).

As expected, the expression of Trim7 decreased from wild type CT26 cells to second generation anti-PD-1 resistant cells and to 4th generation anti-PD-1 resistant cells (FIG. 5C (left panel)). The expression of Trim7 increased in wild type CT26 cells increased when the wild type CT26 cells were induced with IFNγ (compare closed circles data in FIG. 5C (left and right panels)). Paradoxically, the expression of Trim7 decreased from wild type CT26 cells to second generation anti-PD-1 resistant cells and to 4th generation anti-PD-1 resistant cells in response to IFNγ (FIG. 5C (right panel)).

Similarly, the expression of Lrg1 decreased from wild type CT26 cells to second generation anti-PD-1 resistant cells and to 4th generation anti-PD-1 resistant cells (FIG. 5D (left panel)). The expression of Lrg1 increased in wild type CT26 cells increased when the wild type CT26 cells were induced with IFNγ (compare closed circles data in FIG. 5D (left and right panels)). Paradoxically, the expression of Lrg1 decreased from wild type CT26 cells to second generation anti-PD-1 resistant cells and to 4th generation anti-PD-1 resistant cells in response to IFNγ (FIG. 5D (right panel)).

These results suggest, inter alia, a paradoxical dysregulation of genes like Cd274, B2m, Trim7 and Lrg1 compared to parental cancer cells when grown in the presence or absence of IFNγ.

### Example 6: Identifying Driver Genes of Resistance

Since the dysregulation was observed with multiple genes, without being bound by theory, it was hypothesized that the paradoxical dysregulation is the functional consequence of the acquired resistance to anti-PD-1 agents. To identify the driver genes involved in the observed paradoxical dysregulation, the analysis disclosed in FIG. 6A was conducted. Briefly, the Differentially Expressed Genes (DEGs) in 4th generation anti-PD-1 resistant cells compared to parental CT 26 cells when grown in the presence of IFNγ were identified. As shown in FIG. 6A (Panel 1), 1,999 genes are downregulated and 3607 genes are upregulated the 4th generation anti-PD-1 resistant cells in the presence of IFNγ compared to the CT26 cells. From these genes, those that were downregulated the 4th generation anti-PD-1 resistant cells but were upregulated the CT26 cells were identified. As shown in FIG. 6A (Panel 2), 1,060 genes were narrowed using this criterion (downregulated the 4th generation anti-PD-1 resistant cells but are upregulated the CT26 cells. these genes were further narrowed based on responsiveness to IFNγ. As shown in FIG. 6A (Panel 3), 688 genes that are downregulated the 4th generation anti-PD-1 resistant cells compared to the CT26 cells as revealed by sorting according to responsiveness to IFNγ. These genes include Lrg1, Spry2, Arg1, Trim8, Trim2, Mapk8ip1, Trim7, Trim6, etc. (FIG. 6A (Panel 3)).

Then to further narrow these genes, the parental CT26 cells, second generation anti-PD-1 resistant cells and 4th generation anti-PD-1 resistant cells were grown in vivo in mice. RNA was isolated from tumors and subjected to RNA-seq and data analysis. From the 688 genes from (FIG. 6A (Panel 3). Those genes that were also upregulated in vivo were identified. As shown in FIG. 6A (Panel 4), 70 genes were upregulated in vivo in the 4th generation anti-PD-1 resistant cells compared to the CT26 cells. these genes included Krt8, Mapk8ip1, Arg1 and Lrg1. FIG. 6B shows the gene ontology (GO) pathways that were enriched in at least two steps in FIG. 6A. This analysis identified TRIM family of proteins (especially, Trim7), Mapk8ip1, Elk1, Lrg1, Arg1, to be some of the drivers. FIG. 6C shows the functional pathways affected by the TRIM family of proteins. FIG. 6D shows the functional pathways in which Elk1 and c-Jun play a role. FIG. 6E shows the functional connections between Lrg1, B2m and Arg1 with other genes. FIG. 6F shows the levels of expression of Elk1 in tumors and surrounding normal tissue in the Cancer Genome Atlas (TOGA) cancer genomics program.

### Example 7: Paradoxical Dysregulation of Additional Differentially Regulated Genes

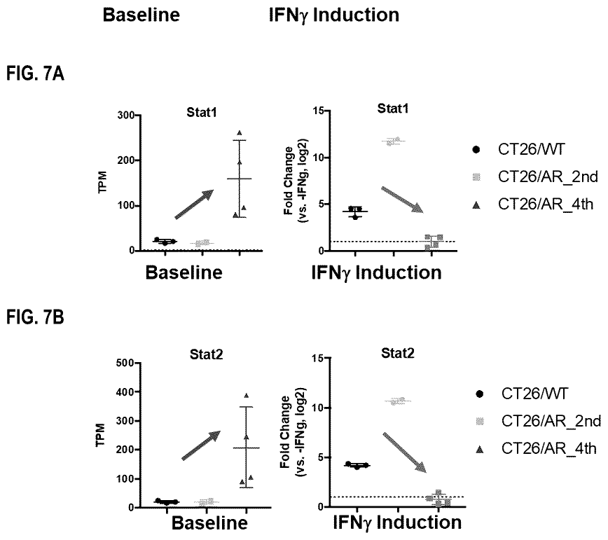

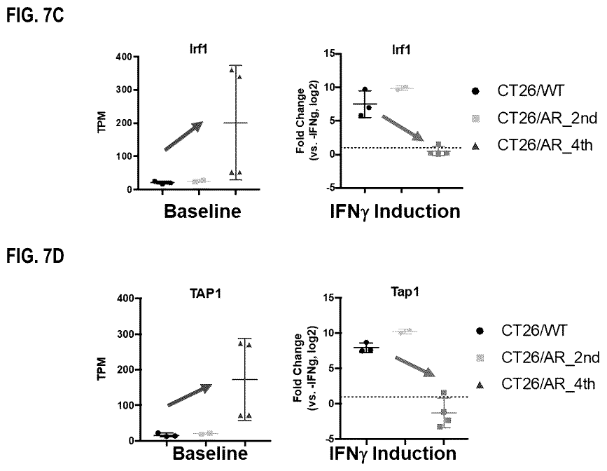

Transcripts per million (TPM; normalized expression data) for additional representative genes that are overexpressed (Stat1, Stat2, Irf1 and Tap1) were analyzed. The expression of Stat1 increased from wild type CT26 cells to second generation anti-PD-1 resistant cells and to 4th generation anti-PD-1 resistant cells (FIG. 7A (left panel)). The expression of Stat1 increased in wild type CT26 cells increased when the wild type CT26 cells were induced with IFNγ (compare closed circles data in FIG. 7A (left and right panels)). Paradoxically, the expression of Stat1 decreased from wild type CT26 cells to second generation anti-PD-1 resistant cells and to 4th generation anti-PD-1 resistant cells in response to IFNγ (FIG. 7A (right panel)).

The expression of Stat2 also increased from wild type CT26 cells to second generation anti-PD-1 resistant cells and to 4th generation anti-PD-1 resistant cells (FIG. 7B (left panel)). The expression of Stat2 increased in wild type CT26 cells increased when the wild type CT26 cells were induced with IFNγ (compare closed circles data in FIG. 7B (left and right panels)). Paradoxically, the expression of Stat2 decreased from wild type CT26 cells to second generation anti-PD-1 resistant cells and to 4th generation anti-PD-1 resistant cells in response to IFNγ (FIG. 7B (right panel)).

Similarly, the expression of Irf1 increased from wild type CT26 cells to second generation anti-PD-1 resistant cells and to 4th generation anti-PD-1 resistant cells (FIG. 7C (left panel)). The expression of Irf1 increased in wild type CT26 cells increased when the wild type CT26 cells were induced with IFNγ (compare closed circles data in FIG. 7C (left and right panels)). Paradoxically, the expression of Irf1 decreased from wild type CT26 cells to second generation anti-PD-1 resistant cells and to 4th generation anti-PD-1 resistant cells in response to IFNγ (FIG. 7C (right panel)).

As expected, the expression of Tap1 increased from wild type CT26 cells to second generation anti-PD-1 resistant cells and to 4th generation anti-PD-1 resistant cells (FIG. 7D (left panel)). The expression of Tap1 increased in wild type CT26 cells increased when the wild type CT26 cells were induced with IFNγ (compare closed circles data in FIG. 7D (left and right panels)). Paradoxically, the expression of Tap1 decreased from wild type CT26 cells to second generation anti-PD-1 resistant cells and to 4th generation anti-PD-1 resistant cells in response to IFNγ (FIG. 7D (right panel)).

### Example 8: Pathway Analysis of the Differentially regulated Genes

To perform pathway analysis was performed using the WEB-based GEne SeT AnaLysis Toolkit (WebGestalt). Briefly, top 1,000 genes that were downregulated the 4th generation anti-PD-1 resistant cells but were upregulated the CT26 cells were (see FIG. 6A (Panel 2)), were rank ordered based on the fold-difference in expression between the 4th generation anti-PD-1 resistant cells and the parental CT-26 samples. These genes and rankings were input into the WebGestalt to identify enriched/de-enriched pathways associated with these genes, using gene set enrichment analysis (GSEA) and the Kyoto Encyclopedia of Genes and Genomes (KEGG) pathway functional database. Benjamini-Hochberg false discovery rate (FDR) was used for statistical significance. This analysis showed that the following pathways were overactive in the 4th generation anti-PD-1 resistant cells compared to the parental CT-26 cells: circardian entrainment, cAMP signaling pathway, cholinergic synapse, melanogenesis, Rap1 signaling pathway, human cytomegalovirus infection, human immunodeficiency 1 virus infection, glutamatergic synapse, pathways in cancer and Ras signaling pathway (FIG. 8A). The following pathways were repressed in in the 4th generation anti-PD-1 resistant cells compared to the parental CT-26 cells: systematic lupus erythematosus, microRNAs in cancer, hepatocellular carcinoma, tuberculosis and protein processing in endoplasmic reticulum. To visualize of the enrichment score and significance, a volcano plat was prepared based on the data presented in FIG. 8A. FIG. 8B shows the volcano plot. As shown in FIG. 8B, although circadian entrainment was the farthest ‘dot’ to the right, pathways in cancer and Ras signaling were the highest up (i.e. having the most significant FDR values). Although there wasn't strong significance (All FDR >0.05, which often happens in the noise of genomics), Ras/Rap1 signaling pathways were identified in this unbiased analysis. FIG. 8C shows the RAS signaling pathway, illustrating the convergence with Raf/Mek/Erk signaling. FIG. 8D shows the RAP1 signaling pathway, illustrating the convergence with Raf/Mek/Erk signaling.

### Example 9: Paradoxical Dysregulation of Ccl5 (RANTES), Cxcl10 (IP-10) and Ifnb1

Nest transcripts per million (TPM; normalized expression data) for Ccl5 (RANTES), Cxcl10 (IP-10) and Ifnb1 were analyzed. These genes are induced by the activation of Trim7.

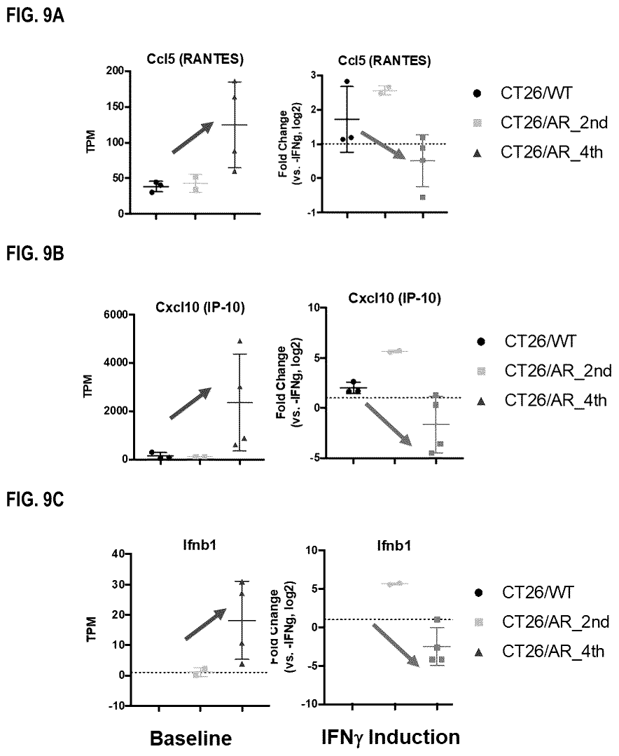

Ccl5 (RANTES) and Cxcl10 (IP-10) are among genes that are regulated by IFNγ. The promoters regulating Ccl5 (RANTES) and Cxcl10 (IP-10) contain binding sites for STAT1 dimers and/or ISGF3. As expected, the expression of Ccl5 (RANTES) increased from wild type CT26 cells to second generation anti-PD-1 resistant cells and to 4th generation anti-PD-1 resistant cells (FIG. 9A (left panel)). The expression of Ccl5 (RANTES) increased in wild type CT26 cells increased when the wild type CT26 cells were induced with IFNγ (compare closed circles data in FIG. 9A (left and right panels)). Paradoxically, the expression of Ccl5 (RANTES) decreased from wild type CT26 cells to second generation anti-PD-1 resistant cells and to 4th generation anti-PD-1 resistant cells in response to IFNγ (FIG. 9A (right panel)).

The expression of Cxcl10 (IP-10) increased from wild type CT26 cells to second generation anti-PD-1 resistant cells and to 4th generation anti-PD-1 resistant cells, as expected (FIG. 9B (left panel)). The expression of Cxcl10 (IP-10) increased in wild type CT26 cells increased when the wild type CT26 cells were induced with IFNγ (compare closed circles data in FIG. 9B (left and right panels)). Paradoxically, the expression of Cxcl10 (IP-10) decreased from wild type CT26 cells to second generation anti-PD-1 resistant cells and to 4th generation anti-PD-1 resistant cells in response to IFNγ (FIG. 9B (right panel)).

The expression of Ifnb1 also increased from wild type CT26 cells to second generation anti-PD-1 resistant cells and to 4th generation anti-PD-1 resistant cells (FIG. 9C (left panel)). The expression of Ifnb1 increased in wild type CT26 cells increased when the wild type CT26 cells were induced with IFNγ (compare closed circles data in FIG. 9C (left and right panels)). Paradoxically, the expression of Ifnb1 decreased from wild type CT26 cells to second generation anti-PD-1 resistant cells and to 4th generation anti-PD-1 resistant cells in response to IFNγ (FIG. 9C (right panel)).

These results illustrate, inter alia, that acquired resistance to anti-PD-1 is associated with a paradoxical regulation of Trim7-regulated genes such as Ccl5 (RANTES), Cxcl10 (IP-10) and Ifnb1.

### Example 10: Materials and Methods

Construct Generation and Protein Purification

The sequences of human and mouse TIGIT-Fc-LIGHT were codon-optimized and directionally cloned into mammalian expression vectors. Vectors were then either transiently transfected into Expi293 cells or stably transfected into CHO cells, and the resulting fusion protein was purified using affinity chromatography.

Western Blot

Human (h) and mouse (m) TIGIT-Fc-LIGHT proteins were treated +/− the deglycosylase PNGase F (NEB) for 1 hour at 37° C. according to manufacturers recommendations, and then +/− the reducing agent beta-mercaptoethanol (BME), and diluted in SDS loading buffer prior to separation by SDS-PAGE. Primary antibodies used for probing hTIGIT-Fc-LIGHT and mTIGIT-Fc-LIGHT were obtained from Cell Signaling Technologies, Jackson ImmunoResearch Laboratories, Inc. and RandD Systems.

MSD and ELISA Detection

Dual Binding Potency Assay

For the TIGIT-Fc-LIGHT chimeric protein; MSD Multi-Array plates (MSD) were pre-coated overnight with recombinant human LTβR-Fc or HVEM-Fc (Acro Biosystems) at 4° C. The plates were blocked with Diluent 100 (MSD) for at least one hour at room temperature on a shaker. After washing, analysis samples were added and then serially diluted 3-fold and loaded in duplicate wells to generate 8-10 test concentrations. The bound the TIGIT-Fc-LIGHT chimeric protein was detected with recombinant biotinylated human PVR (Acro Biosystems). Streptavidin conjugated to ruthenium (MSD), was added to bind the biotin labeled detector. MSD Gold Reading buffer (MSD) was added to the plate, and upon application of an electrical charge, an electrochemiluminescent signal was produced and detected with the MSD MESO QuickPlex SQ 120MM, Model 1300, to generate relative light units (RLU). Analogous assays were performed using mTIGIT-Fc-LIGHT and murine recombinant PVR, HVEM, and LOUR (Acro Biosystems).

Antibody-Based Binding Assay

The same methodology was performed as described for the dual potency assay above, however the TIGIT-Fc-LIGHT chimeric protein was captured with anti-human TIGIT and detected with anti-human LIGHT-biotin (all antibodies from RandD Systems) followed by Streptavidin sulfo-tag.

DcR3 Blocking Assay

Human healthy donor (2 distinct donors) and cancer patient (kidney, non-small cell lung cancer, and prostate) serum samples were obtained from Innovative Research. MSD DcR3 detection reagents were used to profile the levels of soluble DcR3 in each sample, according to manufacturer instructions. To assess whether serum levels of DcR3 were sufficient to inhibit the TIGIT-Fc-LIGHT chimeric protein binding to PVR or HVEM, the TIGIT-Fc-LIGHT chimeric protein was individually incubated in each serum sample for 20 minutes on ice. Following this incubation, samples were loaded onto MSD plates pre-coated with human recombinant HVEM. Plates were then processed according to the dual potency assay described above, where human recombinant PVR was used as the detection reagent.

Bio-Layer Interferometry-Based Affinity Testing of Receptor/Ligand Interactions

Bio-Layer interferometry-based affinity testing was used to determine the on-rates (Ka), off-rates (Kd) and binding affinities (KD) of the TIGIT-Fc-LIGHT chimeric protein to intended binding targets, using histidine- or biotin-tagged versions of the human recombinant proteins (PVR, HVEM, LTβR, PVRL2, PVRL3, and Nectin-4; purchased from Acro Biosystems or Sino Biological). Commercially available or internally produced single-sided fusion protein controls (TIGIT-Fc and Fc-LIGHT) were also tested in parallel. Targets were immobilized at a concentration of 3 μg/mL in Kinetics buffer (PBS/0.1% Tween-20/1% BSA; pH7.0) to anti-pentaHis or streptavidin coated biosensors. Direct binding of fusion proteins to recombinant target proteins was performed on an Octet-Red96 BLI instrument, using association and dissociation times of 90 and 120 seconds, respectively.

Cell Culture

CHO-K1, CT26/WT, B16.F10, CT26/AR, Jurkat, and A375 cells were obtained from ATCC and cultured according to their guidelines; maintained at 37° C. in 5% CO₂. All parental cell lines in active culture are tested monthly using the Venor GeM Mycoplasma Detection Kit (Sigma). All transfected cell lines are tested an additional two times, separated by at least 2 weeks, post transfection and confirmed to remain negative for mycoplasma.

In Vitro Cell Line Generation

Stable cell lines were generated to assess in vitro binding of the human or mouse TIGIT-Fc-LIGHT chimeric protein; including CHO-K1/hPVR, CHO-K1/hHVEM, and CHO-K1/mLTβR. Briefly, cDNA vectors were obtained from RandD systems or Origene and cloned into pcDNA3.1(−) (Thermo Fisher), and then parental CHO-K1 or Jurkat cells were nucleofected with the 4D-Nucleofector and Cell Line Nucleofector Kit SE (Lonza) according to manufacturer's directions. Following antibiotic selection and single-cell cloning using limiting dilution, receptor expression was verified using flow cytometry, and the resulting cell lines were used for in vitro binding assays.

In Vitro Functional Assays

NFkB-Signaling: Non-canonical

U2OS/NIK/NFκB reporter cells expressing LTβR were purchased from Eurofins/DiscoverX and cultured according to their recommendations. On the day of the assay, 1×10⁴ U2OS/NIK/NFκB reporter cells were plated into each well of a 96-well plate with either Fc-LIGHT or the TIGIT-Fc-LIGHT chimeric protein. After 6 hours in culture, luminescence activity was assessed on a luminometer (Promega).

TIGIT/PVR/DNAM1 Reporter Assay

The CD155(PVR)/TIGIT blocking assay (Promega) was used according to manufacturer's instructions. The reagents consist of CHO-K1 target cells that express human CD155(PVR) and effector Jurkat cells that express human TIGIT, CD226(DNAM1), and a co-stimulator-responsive luciferase expression vector. Jurkat effectors were also confirmed to express human HVEM using flow cytometry. For the assay, Jurkat effector cells were plated into white 96 well plates (Costar) and incubated overnight at 37° C./5% CO₂. The next day, cells were co-cultured with CHO-K1 target cells and the following test articles—recombinant human IgG4 (negative control), anti-DNAM1 blocking antibody, Fc-LIGHT, or the TIGIT-Fc-LIGHT chimeric protein. An anti-LIGHT blocking antibody was also used to block the function of the costimulatory domain of the TIGIT-Fc-LIGHT chimeric protein. All antibodies and reagents were purchased from Acro Biosystems, Sino Biologics, or RandD Systems. After 6 hours of additional culture, Bio-Glo reagent (Promega) was added to the wells using automatic injectors on a Promega Navigator luminometer, and relative luminescence (RLU) was determined.

NK/T Cell Killing Assay

CT26 tumor cells were plated into clear 96 well plates and cultured at 37° C./5% CO₂ overnight. NK cells were isolated from the spleens of BALB/c mice using an EasySep Mouse NK Cell Isolation Kit (StemCell Technologies). Total T cells were also isolated from the spleens of BALB/c mice using an EasySep Mouse T Cell Isolation Kit (StemCell Technologies). T cells were sub-optimally stimulated with anti-mouse CD3/CD28 magnetic beads (StemCell Technologies; at 1/10th the recommended concentration) for 48 hours. On the day of co-culture, NK (2.5 effector:1 target cell ratio) or T (5 effector:1 target cell ratio) cells were added to the plates containing CT26 tumors cells +/− mTIGIT-Fc-LIGHT, and a caspase 3/7-green reagent (Essen Bioscience) for assessing tumor cell killing. Images were taken on the Incucyte S3 platform, and fluorescent signal (increase in cell death) was quantitated over time, using Incucyte software.

T Naïve (Tn) and T Stem Cell Memory (Tscm) Differentiation and Analysis

Healthy human donor PBMCs were obtained and CD8+ T cells were isolated using a naïve CD8 magnetic isolation kit (cells and isolation kit from StemCell Technology). Isolated cells were cultured in AIMV media (GIBCO) in the presence of anti-human CD3/CD28 magnetic beads (Invitrogen) at a 1:3 cell to bead ratio, 20 IU ml⁻¹ human recombinant IL-2 (RandD Systems), and 5 □M TWS119 (SelleckChem), for 9 days. Gattinoni et al., A human memory T cell subset with stem cell-like properties. Nat Med 17, 1290-1297 (2011). Following incubation, cells were isolated and Fc receptors were blocked using Human TruStain FcX Fc Receptor Blocking Solution (BioLegend). Cells were then analyzed by flow cytometry using fluorescently conjugated antibodies to CD3, CD8, CCR7, CD45RO, CD62L, CD45RA, CD27, IL7Rα, IL2Rβ, and CD95 (all antibodies from BioLegend).

AIMV Proliferation Assay and Cytokine Analysis

Healthy human donor PBMCs were plated at a density of 2 million cells per mL in AIM-V medium (Gibco) in 24 well plates with vehicle (PBS), TIGIT-Fc(IgG4)-LIGHT (150 nM), TIGIT-Fc(IgG1)-LIGHT (150 nM), anti-TIGIT (150 nM, IgG1 clone #HuTIG1-IgG1.AA, Creative Biolabs), anti-PD-1 (150 nM, pembrolizumab), or the combination of anti-TIGIT and anti-PD-1. For TIGIT-Fc(IgG4)-LIGHT, confluency of human donor treated PBMCs was assessed over a 6-day time-course. For both TIGIT-Fc(IgG1)-LIGHT and TIGIT-Fc(IgG4)-LIGHT, the Promega MTS proliferation assay was performed following 7 days of culture, according to manufacturer recommendations. Plates were imaged on an Incucyte S3 imager over a −7-day period. On days 2 and 7, static images were taken to demonstrate differences in morphology between treatment groups. At the end of the experiment, percent confluency was determined using Incucyte software. Replicate plates with the same treatment groups were seeded at the same density of cells, and were placed in a standard cell culture incubator at 37° C./5% CO₂ for 2 days. At the end of the time-course, media was removed and assessed for levels of IFN□, IL-8, IL-10, IL-12/p70, and SDF-1a(CXCL12) using an MSD multiplex array, according to manufacturers recommendations.

AIMV Single Cell RNA-Seq (scRNA-Seq)

To assess the immune transcriptomic profile of PBMCs cultured for 2 days with 150 nM of either TIGIT-Fc(IgG4)-LIGHT or TIGIT-Fc(IgG1)-LIGHT, single cells from treated healthy donor human PBMCs were isolated using the 10× Genomics Chromium handler into individual oil droplets. Single cell libraries were generated using 10× Genomics Chromium Next GEM Single Cell 3′ v3.1: Dual Index reagents according to manufacturer instructions. Libraries were sequenced on an Illumina NovaSeq 6000 and processed with the 10× cellranger pipeline (v 6.0.2) for demultiplexing, barcode and UMI counting and read alignments. Reads (>350×10⁶) from >10,000 individual cells were mapped to the human hg38 reference genome (average 12K cells/sample). The filtered gene count matrices were then analyzed by Seurat (v 4.0.2) for quality control, normalization, integration, dimension reduction, visualization, cell clustering and DEG analysis. Cells with less than 200, more than 2500 genes or have >15% mitochondrial counts were filtered. Gene counts were normalized to total read counts and scaled to 10,000, then log transformed. All samples were integrated using 2000 anchor features to remove batch effect. Integrated data was then scaled and the top 30 principal components (PCs) were used for the uniform manifold approximation and projection (UMAP) visualization. Shared nearest neighbor (SNN) was identified using the top 30 PCs and resolution was set to 0.4 for cell clustering. We then used SingleR (v 1.6.1) for cell type annotations. Differential expression analysis for treated cells vs. control within each cluster was performed using the Wilcox method. We limited tests to genes with log fold change threshold of 0.25 between groups.

SEB Super Antigen Assay

Primary PBMCs (or mouse splenocytes) were obtained from healthy donors (Stemcell technologies) or BALB/c mice, and incubated with 200 ng/mL of the super-antigen SEB (List Biological Laboratories), in the presence of a human or mouse IgG control (10 μg/mL; Jackson Immunoresearch, Inc.) or the TIGIT-Fc-LIGHT chimeric protein. After 3 days, culture supernatants were collected and assessed by ELISA for levels of human or mouse IL-2 (BioLegend).

A375 Cell Stimulation

The human A375 cell line that expresses LTβR has been reported to respond to costimulation via LIGHT (Hehlgans and Mannel, 2001). Briefly, A375 cells were plated at 2×10⁵ cells per well in 24 well plates overnight. The following morning, cells were either untreated, or cultured with 100 nM of either human anti-LTβR (clone 71315, RandD Systems) or the TIGIT-Fc-LIGHT chimeric protein. After 3 hours, media was removed from the cells and Qiagen RLT buffer containing 5% beta mercaptoethanol was added directly to the wells to lyse cells for RNA isolation. Cellular material was passed through QiaShredder columns (Qiagen) and then RNA was purified using a Qiagen RNeasy kit, including on-column DNaseI digestion. 0.5-1 μg of RNA was reverse transcribed using the Origene First-Strand cDNA Synthesis reagents. Validated qPCR primers to human GAPDH, ACTB, CXCL8, and CCL2 were obtained from Origene. cDNA was amplified using Sybr Green reagents and the BioRad CFX96 Touch Real-Time PCR Detection System. Fold-change in gene expression was determined using the delta-delta Ct method, where target gene expression of the no treatment control versus the housekeeping gene ACTB, was set at a value of 1. A second housekeeping gene (GAPDH) was also used, as an example of a gene that did not change expression in response to treatment.

Flow Cytometry

Briefly, cells were incubated with an Fc receptor blocking reagent (BioLegend) when appropriate and were subsequently stained with fluorescent antibodies for 30 minutes on ice in the dark. Indicated antibodies were purchased from BioLegend or Abcam. The AH1-tetramer reagent was purchased from MBL International and was incubated with cells for 1 hour on ice in the dark before adding the rest of the antibody cocktail. Following this incubation period, stained cells were washed and resuspended in FACS buffer (1×PBS buffer containing 1% bovine serum albumin (BSA), 0.02% sodium azide and 2 mM EDTA). Flow cytometry was performed on a BD LSRII Fortessa.

Tumor Model Systems

For CT26/WT, anti-PD-1 resistant CT26 tumors (CT26/AR), and B16.F10 studies, BALB/c or C57BL/6 mice, respectively, were subcutaneously implanted with 5×10⁵ tumor cells into the rear flank. When tumor volume reached ˜80-115 mm³, indicating day 0, the mice were randomized by tumor volume and treatment was initiated. The mean starting tumor volume (STV) from each individual experiment is listed in the corresponding figure. On treatment days, mice were treated by intraperitoneal (IP) injection with vehicle (sterile PBS), anti-PD-1 (clone RMP1-14), anti-PD-L1 (clone 10F.9G2), anti-TIGIT (clone 1G9), anti-LTβR (clone 4H8 WH2), Fc-LIGHT, or mTIGIT-Fc-LIGHT. The LTβR antibody was obtained from Adipogen and LSBio, and all other therapeutic antibodies were obtained from BioXCell. The dose and schedule of each agent are listed in the corresponding figures and figure legends. Tumor volume (mm³) and overall survival was assessed throughout the time-course. Survival criteria included total tumor volume less than 1800 mm³ with no sign of tumor ulceration. Complete responders, in which tumors established and were subsequently rejected are listed in the appropriate figures. Cohorts of CT26 experimental mice were euthanized during various experiments for immune profiling in tumor tissue using flow cytometry and for cytokine analysis in the serum and isolated tumors using Luminex multiplex arrays according to manufacturer instructions. Tumors were excised from these mice and dissociated using a tumor dissociation kit (Miltenyi) and homogenized through a 100 μM strainer to isolate tumor cells and infiltrating immune cells. Experimental group sizes are described in each figure and are generated from a minimum of two independent experiments.

CD4, CD8, and NK Depletion Experiments

Mice were treated via IP injection of 100 μg of anti-CD4 (clone GK1.5), 100 μg of anti-CD8 (clone 2.43), or 500 μg of anti-NK (clone NK1.1) on days −1, 1, and 7. CD4, CD8, and NK cell populations in the peripheral blood were assessed by flow cytometry to verify depletion.

Generation of Anti-PD-1-Resistant CT26 Tumors

As above, mice were inoculated with 5×10⁵ CT26 on the rear flank. When average tumor volume reached mm³ (indicating day 0), mice were given intraperitoneal injections of anti-PD-1 (clone RMP1-14; BioXcell), consisting of 100 ug each on days 0, 3, and 6. Tumors were excised from mice that did not respond to anti-PD-1 therapy, dissociated using collagenase (StemCell Technologies), washed in 1×PBS, and plated in culture media. Cells were passaged 2-4 times and were then inoculated into new recipient mice according to the same protocol as above. Again, when tumors reached 80-100 mm³, another treatment course of anti-PD-1 began. This process was repeated for a total of 5 rounds, at which point none of the treated mice responded to anti-PD-1 therapy. The cell lines generated after this in vivo pressure to develop anti-PD-1 acquired resistance are referred to as CT26/AR (previously characterized herein.

Administration of the Human TIGIT-Fc-LIGHT Chimeric Protein in Cynomolgus Macaques

Purpose-bred, Asian-origin cynomolgus macaques (Macaca fascicularis) were housed at Charles River Laboratories (Mattawan, MI) and experiments with these animals were approved by the Institutional Animal Care and Use Committee for Charles River Laboratories, adhering to the principles outlined in the Guide for the Care and Use of Laboratory Animals. Vehicle control or the TIGIT-Fc-LIGHT chimeric protein were administered via intravenous infusion over 30 minutes. Vehicle (5 males, 5 females), 0.1 mg/kg (3 males, 3 females), 1 mg/kg (3 males, 3 females), 10 mg/kg (3 males, 3 females), or 40 mg/kg of the TIGIT-Fc-LIGHT chimeric protein (5 males, 5 females) were administered every 7 days (Day 1, 8, 15, 22) for a total of 4 doses. Before and following test article administration, all animals were observed for potential clinical observations, body weight, food consumption, veterinary physical examinations, ophthalmic examinations, electrocardiology exams, blood pressure assessment and neurologic assessment. Pre- and post-dose clinical pathology assessments included hematology, coagulation, clinical chemistry, and urinalysis were also performed. Pharmacodynamic assessments including pre- and post-dose collection of peripheral blood using potassium EDTA anticoagulation for serum cytokines, and sodium heparin anticoagulation for peripheral blood flow cytometry studies. Antibody panels used to stain peripheral immune cells for flow cytometry analysis included reagents targeting CD45 and CD3 from BD Biosciences, and CD8 and HVEM from Biolegend.

Experimental Animal Guidelines

All murine animal studies have been conducted in accordance with, and with the approval of an Institutional Animal Care and Use Committee (IACUC); and reviewed and approved by a licensed veterinarian. Experimental mice were monitored daily and euthanized by CO₂ asphyxiation followed by cervical dislocation prior to any signs of distress.

Bioinformatics and Statistical Analysis

TCGA data was accessed through NIH GDC (EBPlusPlusAdjustPANCAN_IlluminaHiSeq_RNASeqV2.geneExp.tsv). Read counts were normalized to set the upper quartile count at 1000 for gene expression.

Experimental replicates (N) are shown in figures and figure legends. Unless noted otherwise, values plotted represent the mean from a minimum of 2 distinct experiments and error is SEM. Statistical significance (p-value) was determined using unpaired t-tests or One-Way ANOVA with multiple comparisons. Significant p-values are labeled with one or more ‘*’, denoting *p<0.05, **p<0.01, ***p<0.001 and ****p<0.0001. Mantel-Cox statistical tests were used to determine the significance between the survival curves. P-values are noted in the legends to these figures and the supplement.

### Example 11: Efficacy of the TIGIT-Fc-LIGHT Chimeric Protein Against the Anti-PD-1 Resistant Cell Lines and a Primarily Resistant Anti-PD-1 Resistant Cell Line

A construct encoding a TIGIT- and LIGHT-based chimeric protein was generated. The “TIGIT-Fc-LIGHT” construct included an extracellular domain (ECD) of human TIGIT fused to an ECD of human LIGHT via a hinge-CH2-CH3 Fc domain derived from IgG1. See, FIG. 10A.

The construct was codon optimized for expression in Chinese Hamster Ovary (CHO) cells, transfected into CHO cells and individual clones were selected for high expression. High expressing clones were then used for small-scale manufacturing in stirred bioreactors in serum-free media and the relevant chimeric fusion proteins were purified with Protein A binding resin columns.

The TIGIT-Fc-LIGHT construct was codon optimized and transiently expressed in 293 cells and purified using protein-A affinity chromatography. To understand the native structure of the TIGIT-Fc-LIGHT chimeric protein, untreated denatured samples (i.e., boiled in the presence of SDS, without a treatment with a reducing agent or a deglycosylation agent) were compared with (i) reduced samples, which were not deglycosylated (i.e. treated only with β-mercaptoethanol, and boiled in the presence of SDS); and (ii) reduced and deglycosylated samples (i.e. treated both with β-mercaptoethanol and a deglycosylation agent, and boiled in the presence of SDS). In addition, to confirm the presence of each domain of the TIGIT-Fc-LIGHT chimeric protein, the gels were run in triplicates and probed using an anti-TIGIT antibody (FIG. 10B, left blot), an anti-Fc antibody (FIG. 10B, center blot), or an anti-LIGHT antibody (FIG. 10B, right blot). The Western blots indicated the presence of a dominant dimer band in the non-reduced lanes (FIG. 10B, lane NR in each blot), which was reduced to a glycosylated monomeric band in the presence of the reducing agent, β-mercaptoethanol (FIG. 10B, lane R in each blot). As shown in FIG. 10B, lane DG in each blot, the chimeric protein ran as a monomer at the predicted molecular weight of about 73 kDa in the presence of both a reducing agent (β-mercaptoethanol) and a deglycosylation agent.

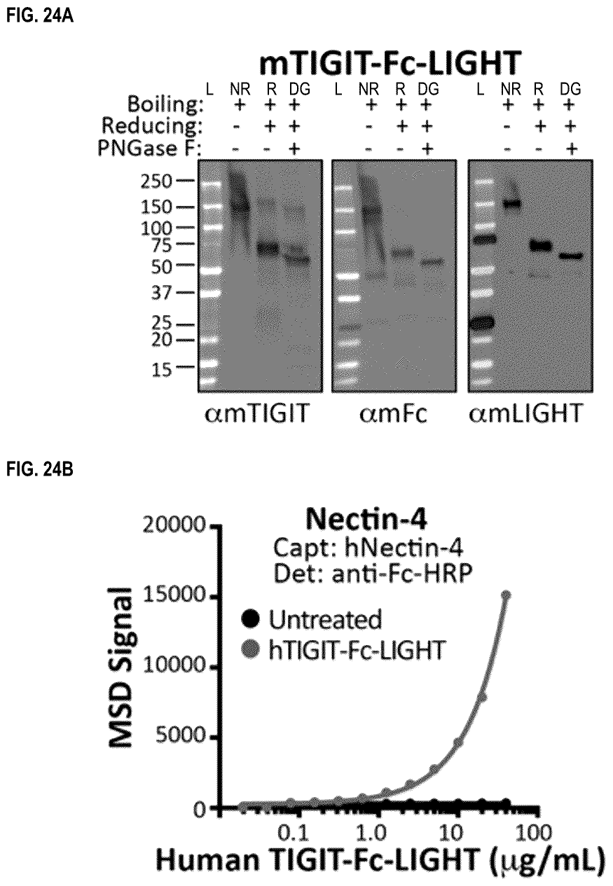

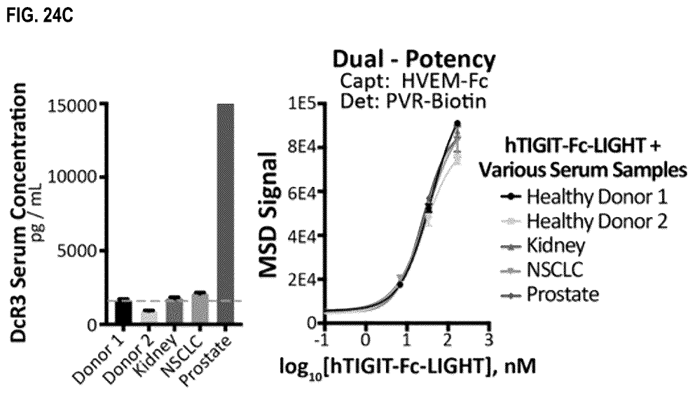

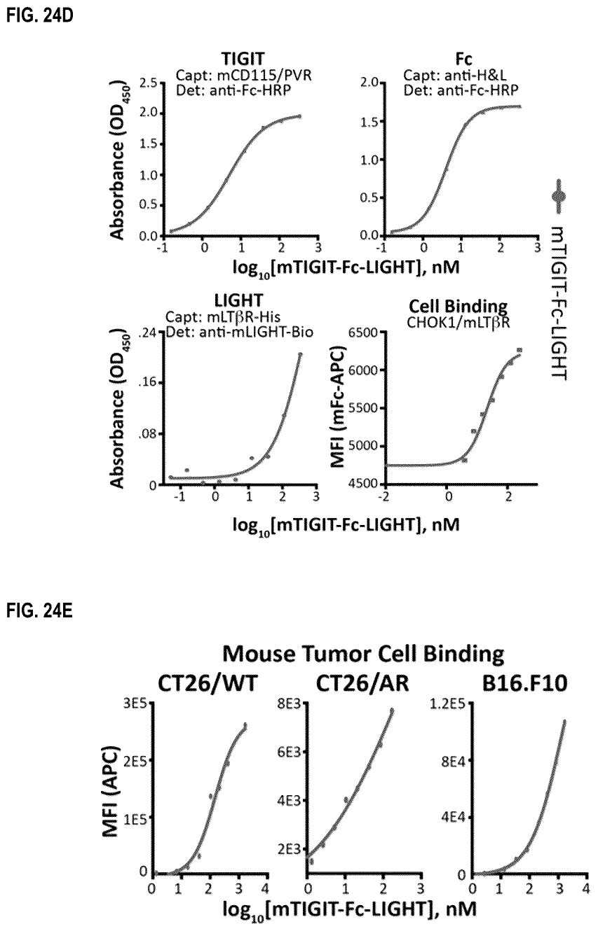

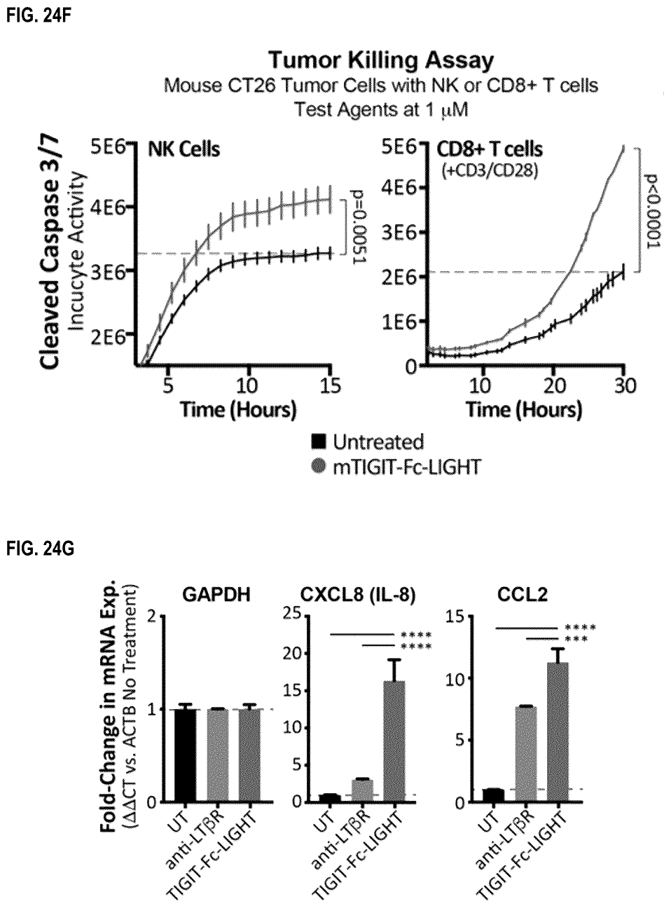

The TIGIT-Fc-LIGHT chimeric protein is a bi-functional Fc-linked fusion protein synthesized from a single expression vector in mammalian production cell lines and purified using affinity chromatography. Previously, size-exclusion chromatography/multi-angle light scattering (SEC-MALS) and electron microscopy (EM) were used to study the structure of this class of TNF-ligand containing fusion protein therapeutics. In some cases, disulfide induced dimerization of the central Fc domain, and charge-based trimerization of the TNF-ligand domain; is believed, without wishing to be bound by theory, to yield a dimer of trimers in solution. de Silva et al., CD40 Enhances Type I Interferon Responses Downstream of CD47 Blockade, Bridging Innate and Adaptive Immunity. Cancer Immunol Res 8: 230-245(2020); Fromm et al., Agonist redirected checkpoint, PD-1-Fc-OX40L, for cancer immunotherapy. J Immunother Cancer 6: 149 (2018). The TIGIT-Fc-LIGHT chimeric protein was predicted to have an hexameric structure (FIG. 10A). Analysis of the TIGIT-Fc-LIGHT chimeric protein under non-reduced SDS-PAGE confirmed the presence of a glycosylated disulfide-linked dimer of −140 kDa (SDS neutralizes the charge-based trimerization of the TIGIT-Fc-LIGHT chimeric protein), which could be reduced to a deglycosylated monomer with an expected molecular weight of 59.3 kDa following incubation with β-mecaptoethanol and PNGase-F (FIG. 10B). All three protein domains of both the human and the mouse TIGIT-Fc-LIGHT chimeric protein constructs were recognized by commercial antibodies via Western blot (FIG. 10B and FIG. 24A).

Protein-protein interactions (PPI) between the fusion proteins and their cognate binding partners were characterized using both recombinant protein and cell-based methods. First, bio-layer interferometry-based affinity testing was used to assess the kinetics of receptor/ligand binding. The TIGIT-Fc-LIGHT chimeric protein bound to recombinant human LTβR at 3.52 nM affinity (approximately 8-fold greater than a commercially available recombinant Fc-LIGHT control) and human PVR at 4.07 nM (consistent with human TIGIT-Fc). The TIGIT-Fc-LIGHT chimeric protein bound to human HVEM at 6.49 nM, similar to Fc-LIGHT binding (2.12 nM) (See the Table below).

| Target | Molecule | KD (nM) | Kₒₙ (1/Ms) | K_{off} (1/s) | R² |
|---|---|---|---|---|---|
| rhLTβR | Fc-LIGHT | 2.84E−08 | 2.66E+05 | 7.54E−03 | .7412 |
| rhLTβR | TIGIT-Fc-LIGHT | 3.52E−09 | 2.12E+04 | 7.44E−05 | .9992 |
| rhHVEM | Fc-LIGHT | 2.12E−09 | 4.10E+05 | 8.76E−04 | .9679 |
| rhHVEM | TIGIT-Fc-LIGHT | 6.49E−09 | 2.61E+04 | 1.73E−04 | .9942 |
| rhPVR | TIGIT-Fc | 5.24E−09 | 2.96E+05 | 1.54E−03 | .9633 |
| rhPVR | TIGIT-Fc-LIGHT | 4.07E−09 | 2.53E+04 | 1.03E−04 | .9984 |
| rhCD112 (PVRL2) | TIGIT-Fc-LIGHT | 3.00E−09 | 1.72E+05 | 4.71E−04 | .7148 |
| rhCD113 (PVRL3) | TIGIT-Fc-LIGHT | 5.14E−09 | 1.30E+05 | 1.26E−03 | .3389 |

The Table above shows bio-Layer Interferometry (Octet) binding kinetics of the human TIGIT-Fc-LIGHT chimeric protein or Fc-LIGHT and TIGIT-Fc single-sided fusion protein controls, to recombinant human (rh) LTβR, HVEM, PVR, PVRL2, and PVRL3.

A combination of bio-layer interferometry and Meso Scale Discovery (MSD) assays were developed to demonstrate binding to other PVR ligand family members, including PVRL2 (CD112), PVRL3 (CD113), and Nectin-4 (See the Table above and FIG. 24B). The binding of the TIGIT-Fc-LIGHT chimeric protein to its receptors was characterized by Meso Scale Discovery (MSD) ELISA assays. Briefly, LTβR-Fc was coated on plates and increasing amounts of the TIGIT-Fc-LIGHT chimeric protein were added to the plates for capture by the plate-bound LTβR-Fc. Binding of the chimeric protein to LTβR-Fc was detected using an PVR-Biotin and using an electrochemiluminescence (ECL) readout. As shown in FIG. 10C, the TIGIT-Fc-LIGHT chimeric protein bound to both LTβR-Fc and PVR. HVEM-Fc was coated on plates and increasing amounts of the TIGIT-Fc-LIGHT chimeric protein were added to the plates for capture by the plate-bound HVEM-Fc. Binding of the chimeric protein to HVEM-Fc was detected using an PVR-Biotin and using an electrochemiluminescence (ECL) readout. As shown in FIG. 10C, the TIGIT-Fc-LIGHT chimeric protein bound to both HVEM-Fc and PVR. An anti-TIGIT antibody was coated on plates and increasing amounts of the TIGIT-Fc-LIGHT chimeric protein were added to the plates for capture by the plate-bound anti-TIGIT antibody. Binding of the chimeric protein to anti-TIGIT antibody was detected using an anti-LIGHT antibody and using an electrochemiluminescence (ECL) readout. As shown in FIG. 10C, the TIGIT-Fc-LIGHT chimeric protein bound to both the anti-TIGIT and anti-LIGHT antibodies. These results showed a dose-dependent and saturable binding. Therefore, FIG. 10C demonstrates simultaneous binding of the TIGIT-Fc-LIGHT chimeric protein to checkpoint targets (PVR) and immune co-stimulatory receptors found on myeloid, CD8+T, and NK cells (LTβR and HVEM).

MSD was also used to evaluate binding to individual targets, as well as the simultaneous binding of the human TIGIT-Fc-LIGHT chimeric protein to both PVR and HVEM (EC₅₀ of 28.31 nM) or PVR and LTβR (EC₅₀ of 85.99 nM); indicating the entire fusion protein was intact and able to engage its targets (FIG. 10C). LIGHT is also able to bind soluble decoy receptor 3 (DcR3), which can be elevated is certain autoinflammatory diseases. To assess whether soluble serum DcR3 in cancer patients would reach levels sufficient to interfere with the LIGHT domain of the TIGIT-Fc-LIGHT chimeric protein, the concentration of DcR3 was quantitated in a range of healthy and cancerous human donor serum samples. In most serum samples, the DcR3 levels were 2,000 pg/mL or less, with the exception of a prostate cancer sample that surpassed the upper limit of quantitation of the assay (>15,000 pg/mL)). In all cases when the TIGIT-Fc-LIGHT chimeric protein was preincubated in each serum sample, these levels of DcR3 were not sufficient to interfere with the TIGIT-Fc-LIGHT chimeric protein binding in the dual HVEM/PVR potency assay (FIG. 24C).

Cell surface binding assays were developed to characterize the binding of both human and mouse fusion proteins to their targets expressed in cellular membranes using CHO-K1 cells engineered to stably express human PVR or HVEM. Flow cytometry analysis demonstrated that the human TIGIT-Fc-LIGHT chimeric protein bound cell surface PVR (EC₅₀=35.42 nM), cell surface LTβR (EC₅₀=87.12 nM) and cell surface HVEM (EC₅₀=65.77 nM) (FIG. 10D). Furthermore, the mouse TIGIT-Fc-LIGHT chimeric protein was shown to bind in a dose dependent manner to its targets using ELISA and to wild type CT26 (CT26/WT), a CPI-acquired resistant CT26 (CT26/AR), and B16.F10 tumor cells (FIG. 24D-FIG. 24E). Thus, these 3 preclinical syngeneic mouse tumor models, when treated with the mouse TIGIT-Fc-LIGHT chimeric protein, are relevant for the evaluation of anti-tumor efficacy.

Additional cell-based functional assays were utilized to inform on the TIGIT-Fc-LIGHT chimeric protein activity. First, a NI K dependent non-canonical NFkB signaling assay, using the human bone osteosarcoma cell line, U2OS, that expresses high levels of LTβR (FIG. 10E), was employed to test the activity of the LIGHT domain of the TIGIT-Fc-LIGHT chimeric protein. Upon treatment with the TIGIT-Fc-LIGHT chimeric protein, bioluminescence was detected at levels significantly greater than what was generated with commercially available recombinant Fc-LIGHT (FIG. 10E). Second, the TIGIT-Fc-LIGHT chimeric protein is expected to provide costimulation via HVEM to CD8+ T cells in an antigen-dependent manner. To evaluate this, a Staphylococcal enterotoxin B (SEB) superantigen assay was used wherein human PBMCs or mouse splenocytes were cultured with SEB and either the human or mouse TIGIT-Fc-LIGHT chimeric protein for 3 days, respectively. IL-2 secretion was then assessed in the culture media and indicated that human and the mouse TIGIT-Fc-LIGHT chimeric protein were able to induce the production of adaptive cytokines (FIG. 10F). Third, ligation of LIGHT to HVEM on the surface of CD8+ T cells and NK cells is predicted to enhance killing of target tumor cells. Isolated murine T or NK effector cells were co-cultured with CT26 tumor cells in the presence of a fluorescently activated cleaved caspase 3/7 reporter. The addition of the mouse TIGIT-Fc-LIGHT chimeric protein stimulated an increase in caspase activity in target tumor cells, indicating, inter alia, that the TIGIT-Fc-LIGHT chimeric protein actively increased the cytotoxic potential of the effector cells in culture (FIG. 24F). Fourth, the human melanoma cell line A375 has been used as a direct functional readout for LIGHT/LTβR signaling, via LTβR-dependent production of IL-8 associated genes following stimulation with LIGHT. Hehlgans and Mannel Recombinant, soluble LIGHT (HVEM ligand) induces increased IL-8 secretion and growth arrest in A375 melanoma cells. J Interferon Cytokine Res 21: 333-338 (2001). When the TIGIT-Fc-LIGHT chimeric protein or a commercially available LTβR agonist antibody was cultured with A375 cells, CXCL8 (gene encoding IL-8) and CCL2 were significantly up-regulated within 3 hours, at a magnitude that was significantly greater with the TIGIT-Fc-LIGHT chimeric protein as compared to the anti-LTβR control antibody (FIG. 24G). Lastly, a commercially available TIGIT/DNAM-1 reporter system was utilized to determine whether HVEM costimulation of effector lymphocytes utilized redundant signaling pathways to DNAM-1. Because the TIGIT domain of the TIGIT-Fc-LIGHT chimeric protein binds to PVR, this interaction will competitively inhibit the ability of PVR to interact with DNAM-1. Thus, DNAM-1 mediated costimulation is expected to be inhibited with recombinant constructs containing the extracellular domain of TIGIT. In this assay, TIGIT and DNAM-1 expressing Jurkat T cells (effector) were co-cultured with PVR expressing CHO-K1 cells. The effector cells also contain a luciferase reporter that is responsive to both T-cell receptor (TCR) and DNAM-1 co-stimulation. Since the effector cells used in this assay were Jurkat T cells, it was confirmed that these cells endogenously expressed human HVEM (FIG. 10G). In the CHO-K1 reporter cells, the TIGIT-Fc-LIGHT chimeric protein was able to bypass DNAM-1 co-stimulation through direct HVEM costimulation by LIGHT (FIG. 10H). A DNAM-1 blocking antibody control was shown to decrease the fluorescence of the signal reporter in the assay, as expected. In contrast, incubation of these cells with the TIGIT-Fc-LIGHT chimeric protein led to a significant increase in signal fluorescence, which was confirmed to be specific to the LIGHT domain of the TIGIT-Fc-LIGHT chimeric protein as it could be completely inhibited with a LIGHT blocking antibody (FIG. 10H).

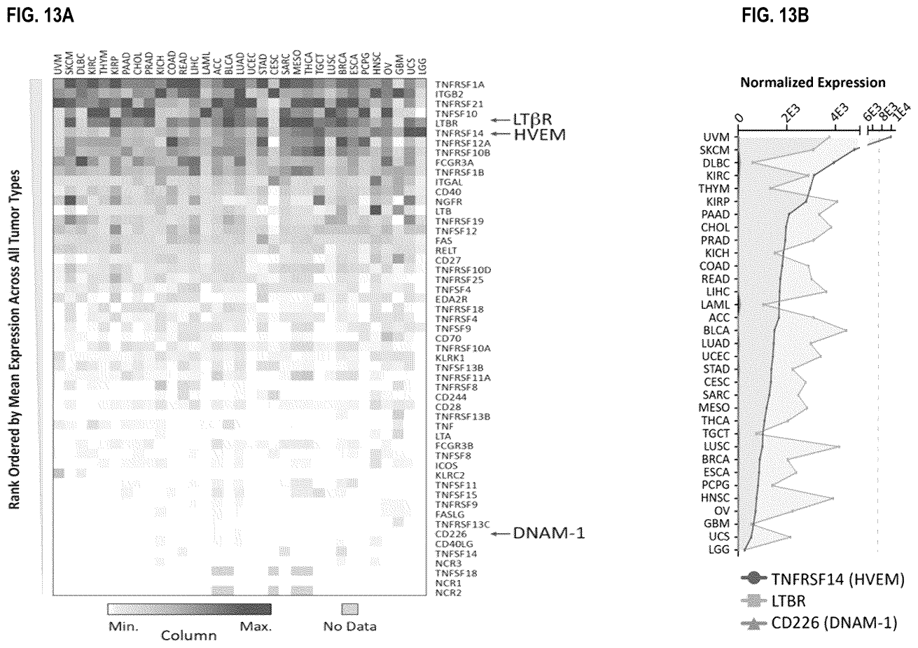

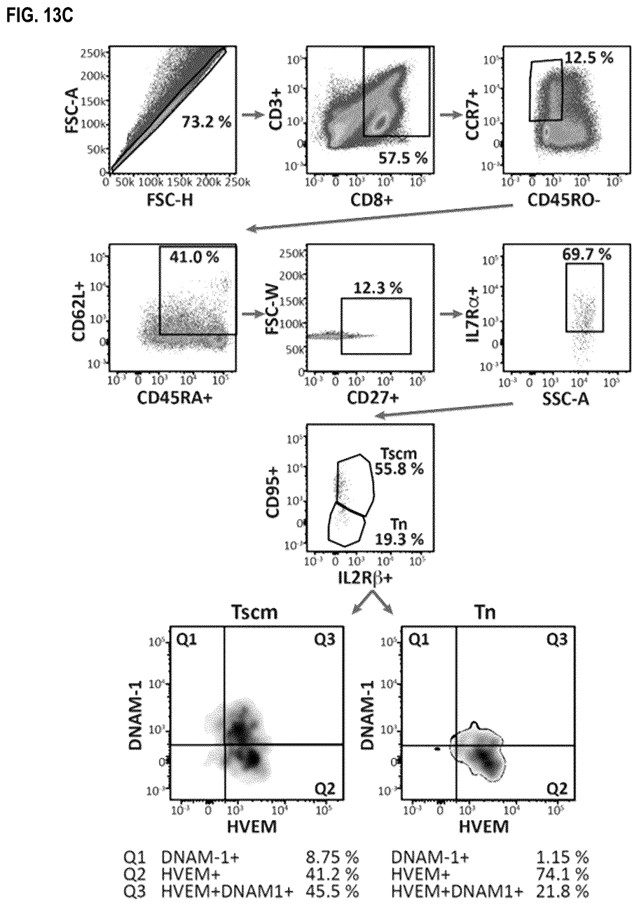

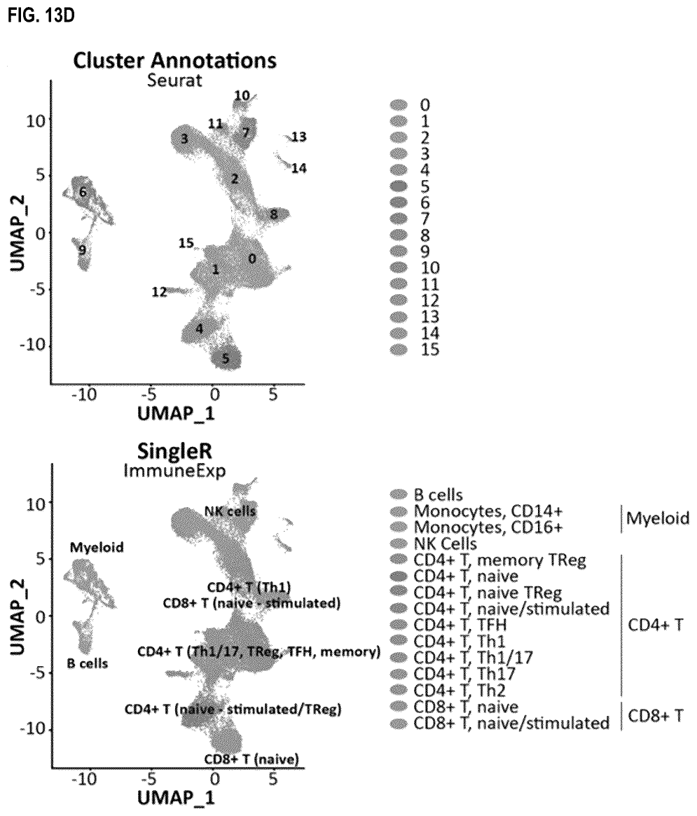

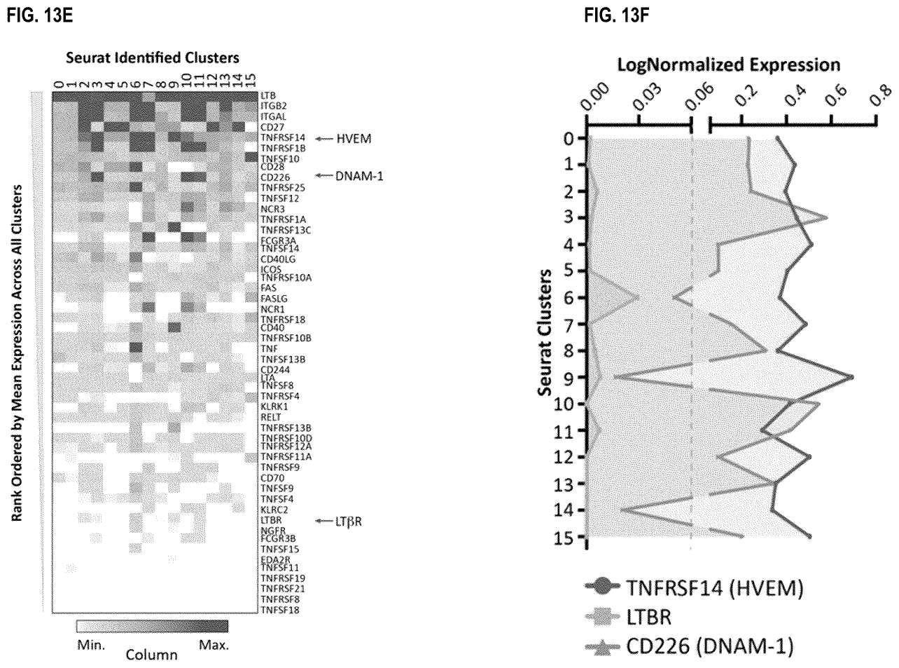

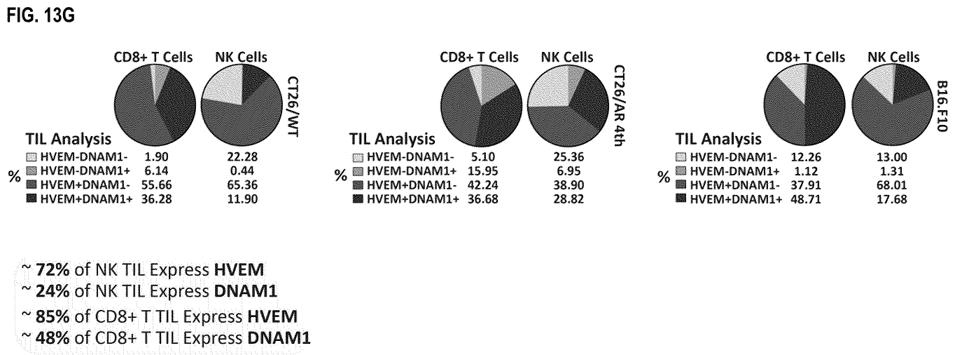

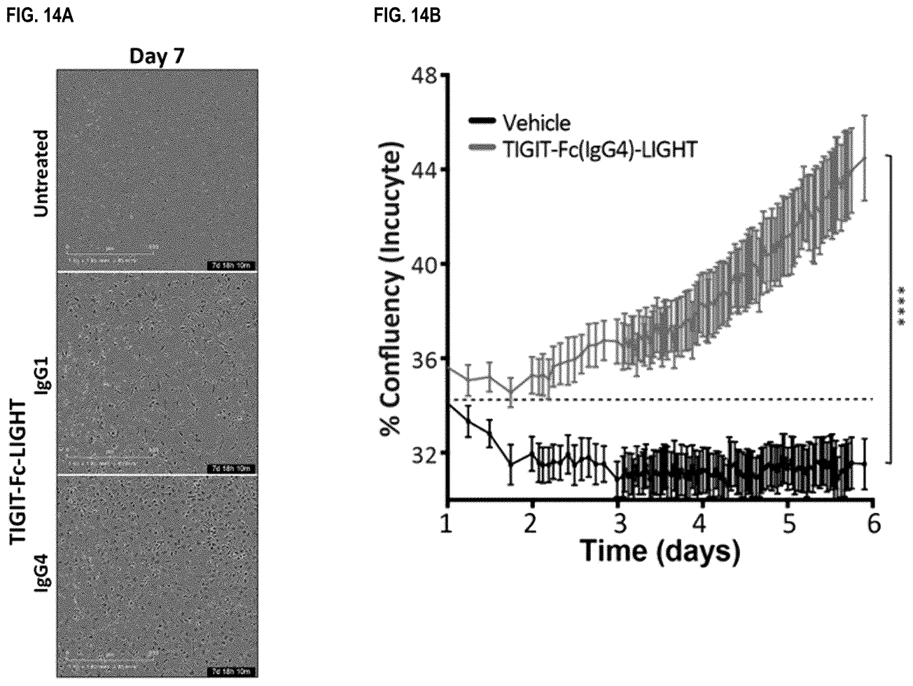

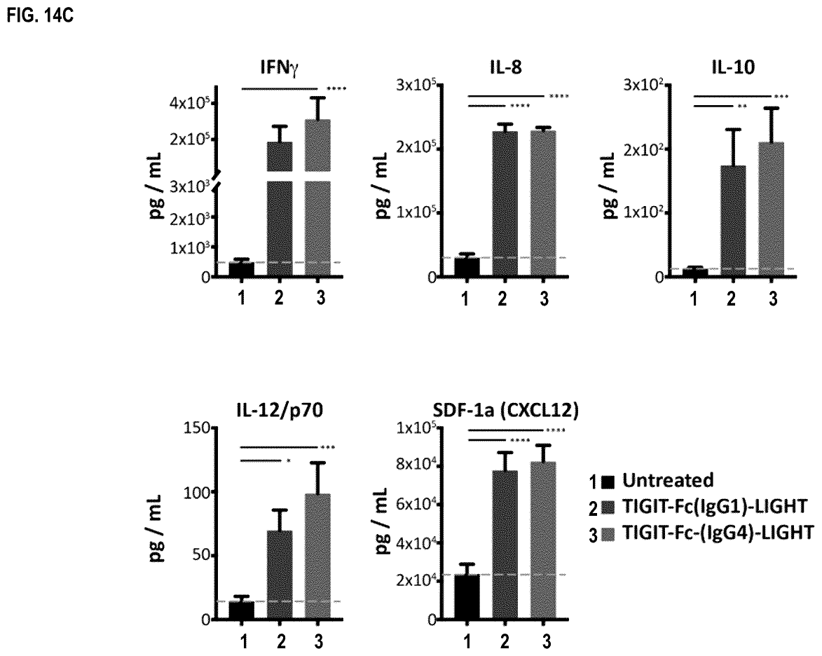

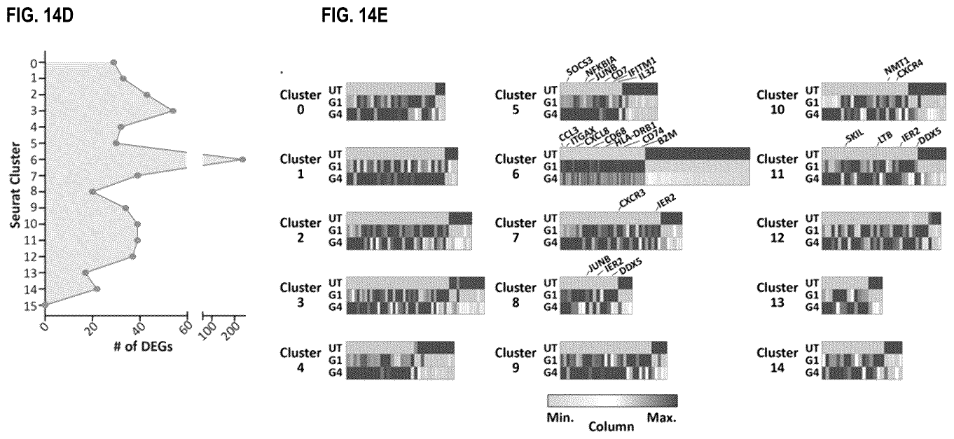

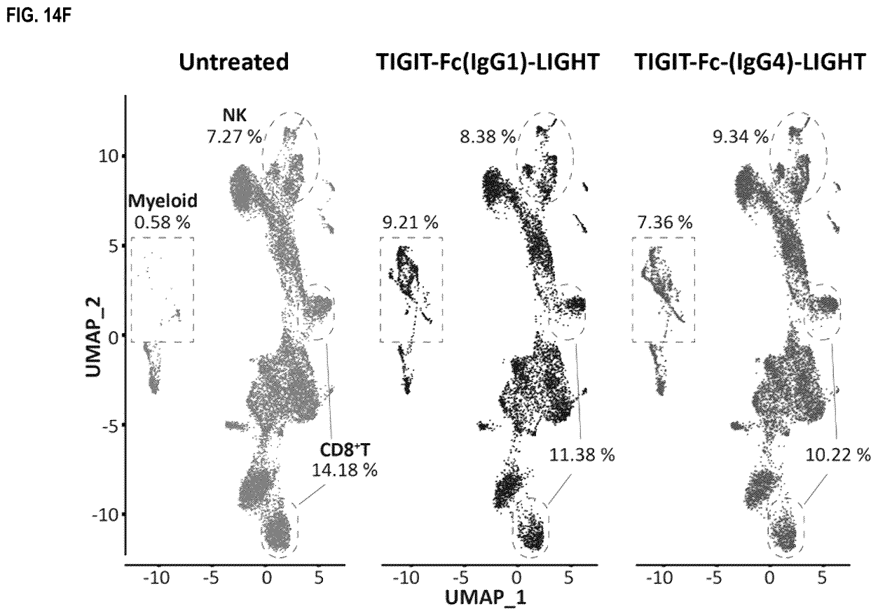

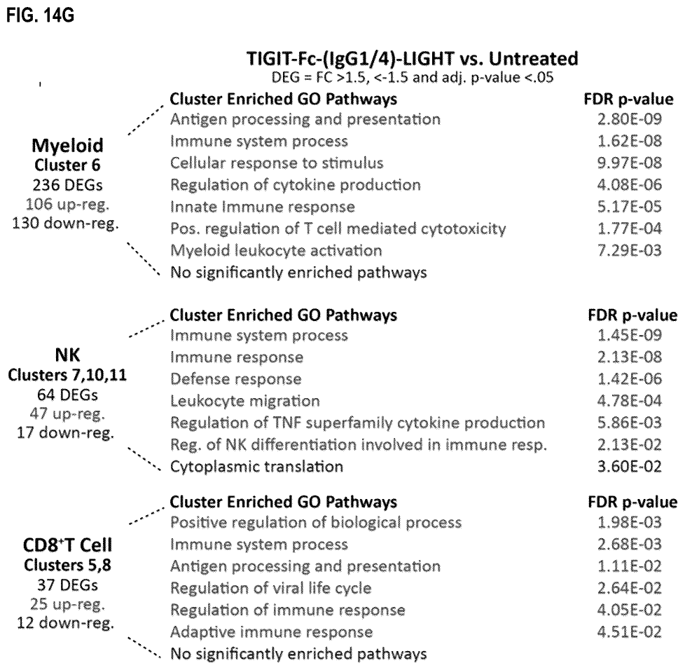

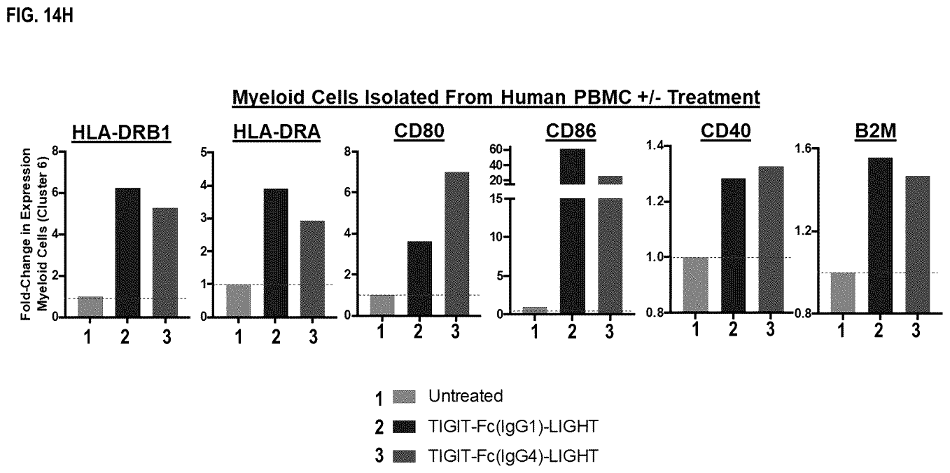

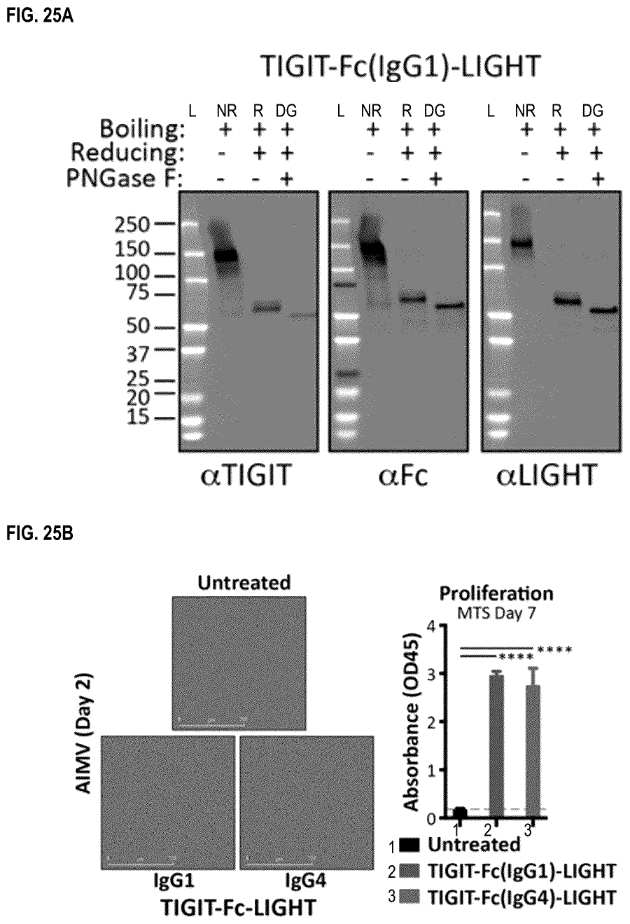

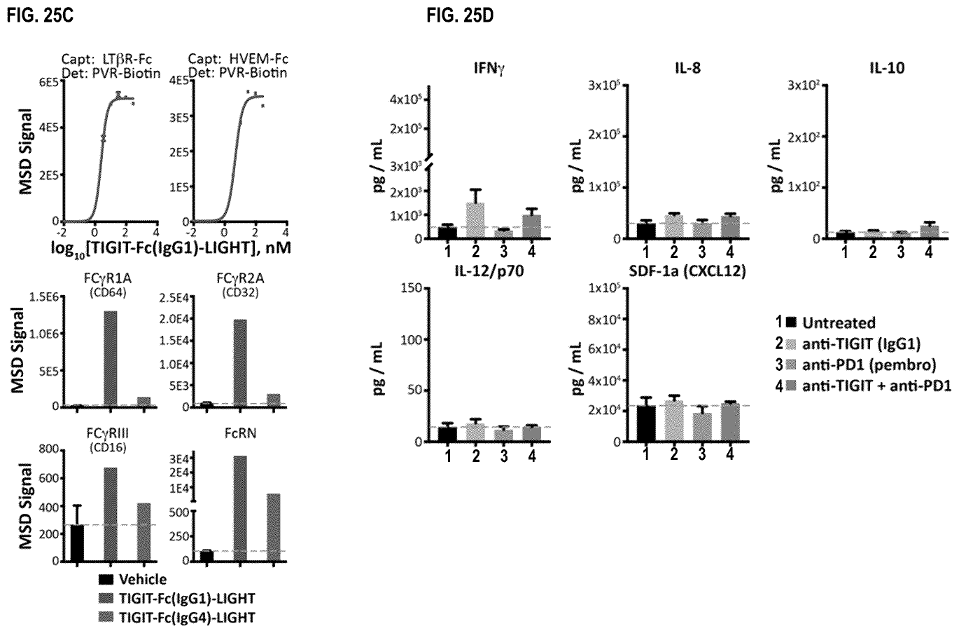

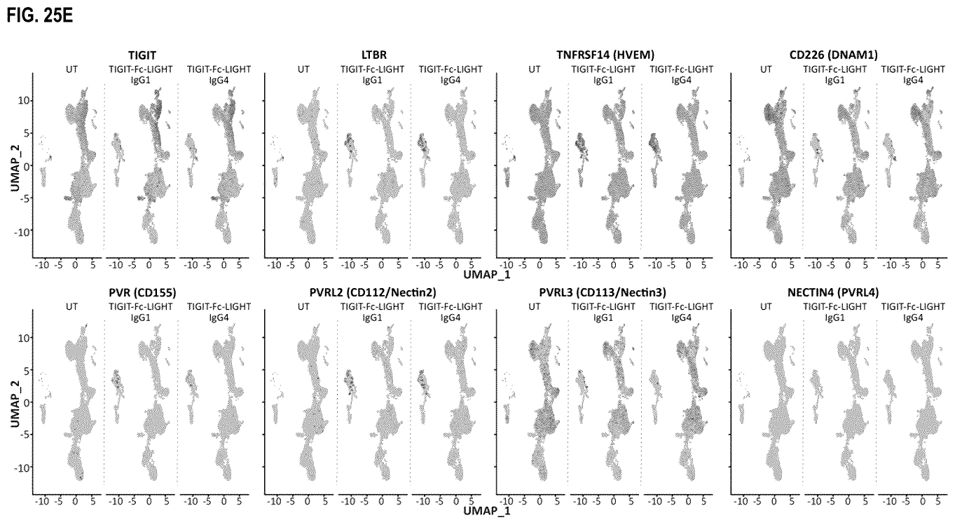

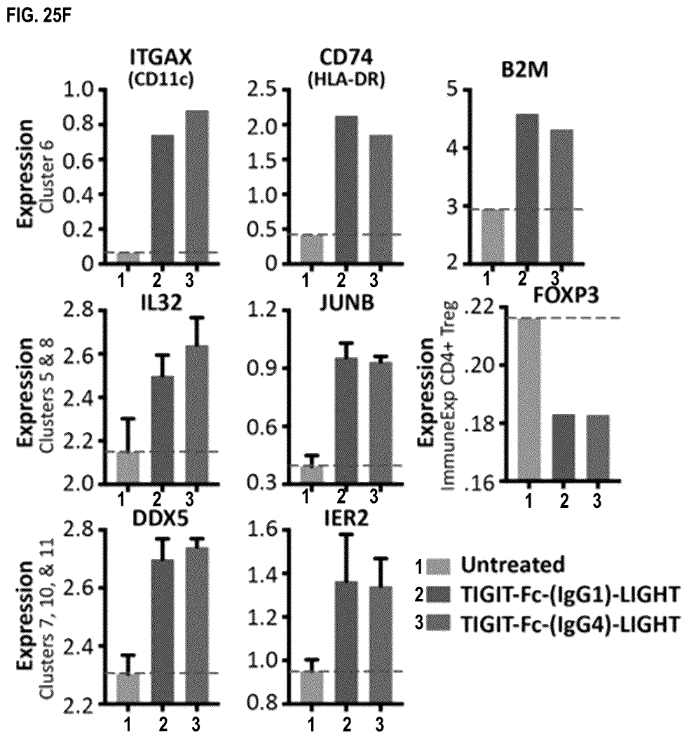

The expression of LTβR was observed both within solid tumor tissues and specifically by myeloid cells (FIG. 13A-FIG. 13G) indicating, inter alia, that LIGHT could provide myeloid cell activation in a similar manner to what has been reported via engagement of Fcγ receptors. To test this further, native IgG1 and effector FcγR silent IgG4 variants of human the TIGIT-Fc-LIGHT chimeric protein, the TIGIT-Fc(IgG1)-LIGHT chimeric protein were generated. The TIGIT-Fc(IgG1)-LIGHT chimeric protein was shown to have similar structural characteristics to the TIGIT-Fc(IgG4)-LIGHT chimeric protein variant, including the ability to engage effector Fcγ receptors (FIG. 25A-FIG. 25C). The IgG1 and IgG4 variants were cultured with human PBMC, and after one week, both fusion proteins induced the adherence of cells to the cell culture plate, differentiation into a morphology consistent with myeloid cell activation, and stimulated significant proliferation (FIG. 14A-FIG. 14B). In fact, the clear morphological impact of both the TIGIT-Fc(IgG1)-LIGHT chimeric protein and the TIGIT-Fc(IgG4)-LIGHT chimeric protein on myeloid differentiation of human PBMC was evident after only 2 days in culture (FIG. 25B). Both the TIGIT-Fc(IgG1)-LIGHT chimeric protein and the TIGIT-Fc(IgG4)-LIGHT chimeric protein also induced both effector (IFNγ) and myeloid-specific (IL-8, IL12/p70, and CXCL12) cytokines (FIG. 14C). No significant impact on cellular morphology, magnitude of proliferation, nor cytokine induction was found between the IgG1 and IgG4 fusion proteins (FIG. 14A-FIG. 14C). Cytokine response was assessed in PBMC treated with a commercially available Fc-competent IgG1 TIGIT antibody both alone and in combination with the anti-PD-1 antibody, pembrolizumab. Interestingly, in these studies only small inductions of IFNγ and IL-8 were observed, however they were not significantly different from control treated cultures (FIG. 25D).

To further assess the immune stimulatory activity of the TIGIT-Fc-LIGHT chimeric protein and potential functional differences between the IgG1 and IgG4 variants, single-cell RNA sequencing was performed two days after stimulation of human PBMC cultures. The distribution of cells expressing TIGIT, HVEM, LTβR, DNAM1, and all PVR ligands was visualized using UMAP (FIG. 25E). Differentially expressed genes (DEGs) were identified between both the TIGIT-Fc(IgG1)-LIGHT and TIGIT-Fc(IgG4)-LIGHT chimeric protein variants and the untreated controls, across all 16 Seurat clusters identified (FIG. 14D). DEGs across clusters associated with myeloid, NK, and CD8+ T cells included CCL3, ITGAX (gene encoding CD11c), CXCL8, CD68, CD74 (gene encoding HLA-DR), B2M, IL32, CD7, JUNB, IER2, and CXCR4 (FIG. 14E and FIG. 25F). The up-regulation of myeloid activating genes was accompanied by an expansion in the cluster 6 population (the cluster with high expression of LTβR, see FIG. 13F) following treatment with either the TIGIT-Fc(IgG1)-LIGHT or TIGIT-Fc(IgG4)-LIGHT versions (FIG. 14F). The NK population (found in clusters 7, 10, and 11 in FIG. 14D-FIG. 14E) modestly increased, and despite a slight decrease in CD8+ T cells (clusters 5 and 8, note that T cell supportive cytokines were not added to the culture), genes associated with T cell and adaptive immune activation were up-regulated (FIG. 14F). Gene ontology pathway analysis on DEGs associated with the myeloid, NK, and CD8+ T cell clusters confirmed that both the TIGIT-Fc(IgG1)-LIGHT or TIGIT-Fc(IgG4)-LIGHT chimeric protein stimulated broad immune cell activation across a range of immune cell fractions (FIG. 14G). Interestingly, out of all DEGs assessed across all Seurat clusters, only 2 genes in total were found to be differentially expressed between the TIGIT-Fc(IgG1)-LIGHT or TIGIT-Fc(IgG4)-LIGHT chimeric proteins (FIG. 25G). Collectively, these results, inter alia, provided evidence for direct myeloid cell activation by LIGHT, which was not further impacted by the selection of an FcγR binding or non-binding Fc domain.

The change in myeloid cells isolated from human PBMC from mice treated with the TIGIT-Fc(IgG1)-LIGHT and TIGIT-Fc(IgG4)-LIGHT chimeric proteins was evaluated in comparison with untreated mice. specifically, HLADRB1m HLA-DRB, CD80, CD86, CD40 or B2M expressing cells were evaluated. As shown in FIG. 14H, the induction was greater than what has been observed with an Fc-competent murine antibody (see e.g., Han, et al., Effective Anti-tumor Response by TIGIT Blockade Associated With FcγR Engagement and Myeloid Cell Activation Frontiers in Immunology 11:573405 (2020). These results suggest, without being bound by theory that, the TIGIT-Fc-LIGHT chimeric protein activates myeloid cells through LIGHT:LTbR interactions and does not depend upon the composition of the Fc domain

The in vivo anti-tumor activity of the murine TIGIT-Fc-LIGHT chimeric protein (mTIGIT-Fc-LIGHT) was evaluated in models of colorectal carcinoma (CT26/WT) and melanoma (B16.F10). Both are widely used as models that are sensitive (CT26/WT) or insensitive (B16.F10) to CPI. The B16.F10 model can be used to mimic primary resistance to anti-PD-1/L1 blockade.

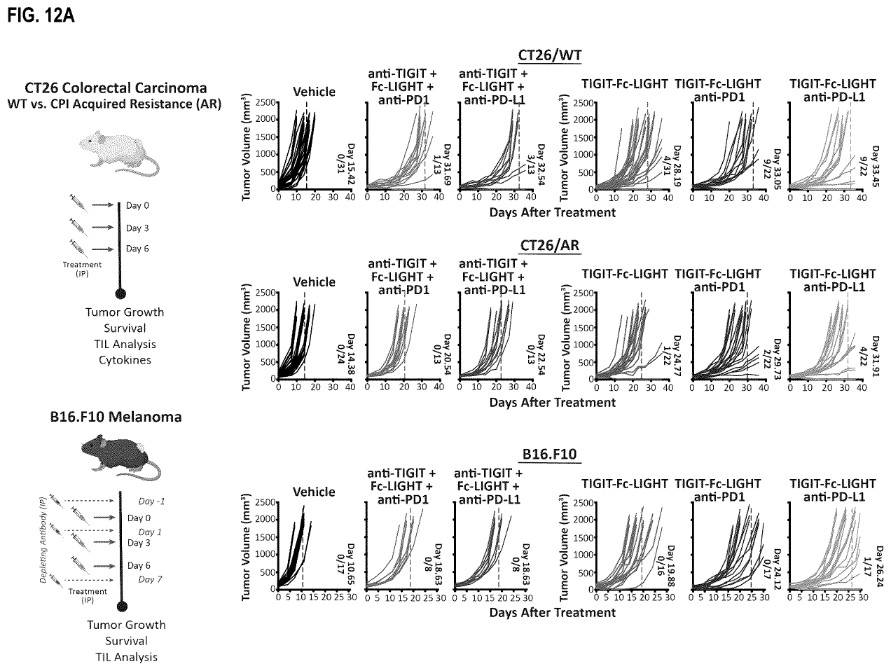

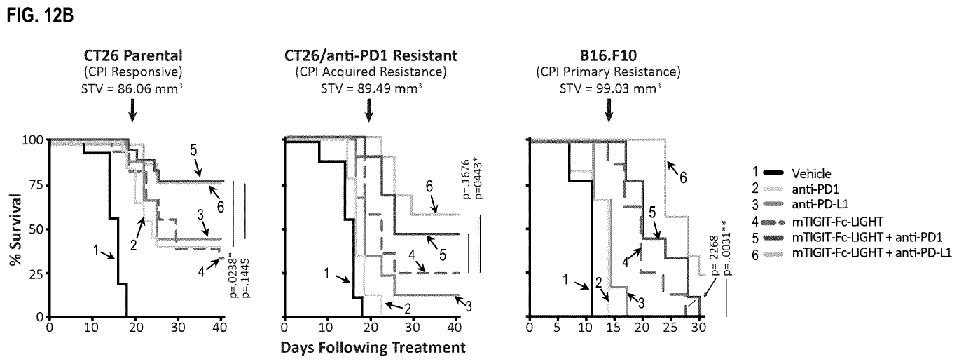

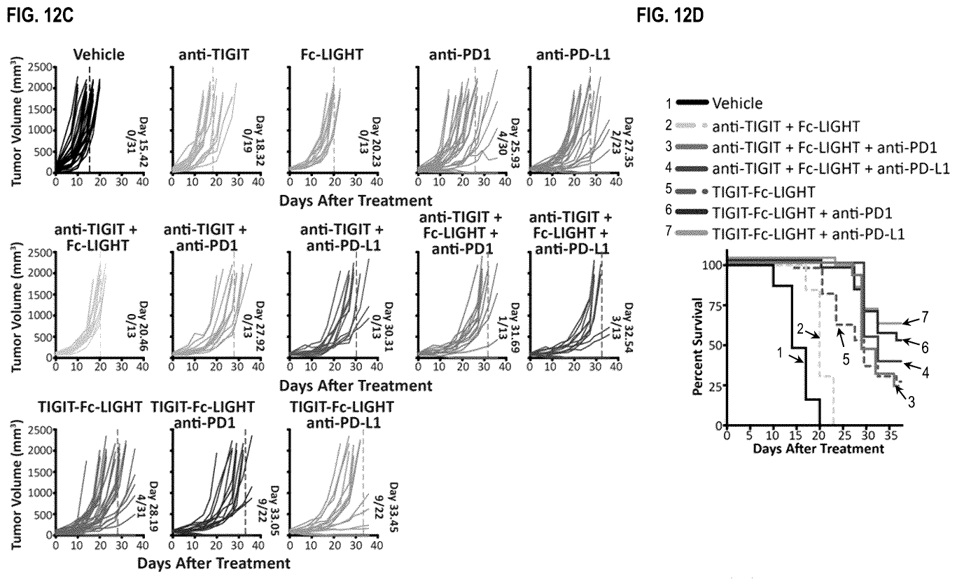

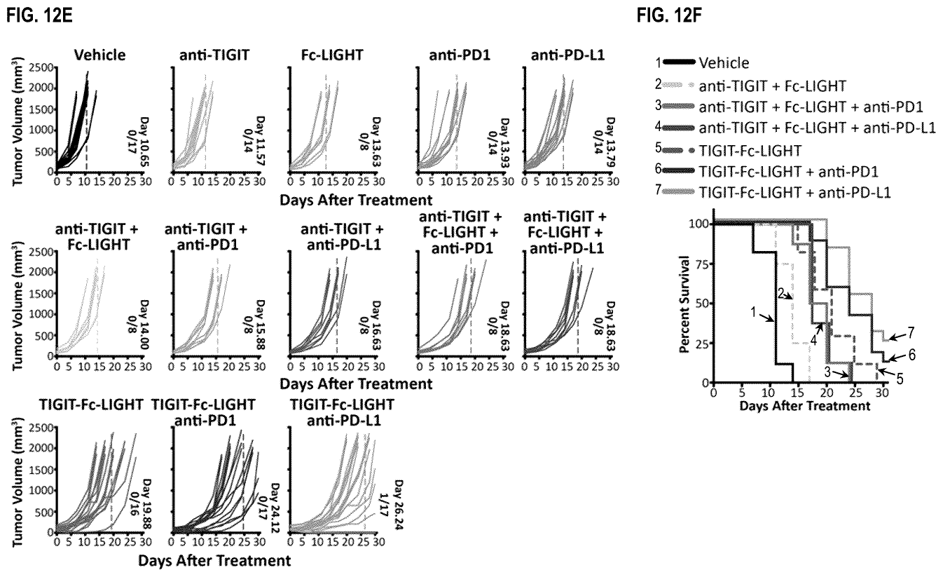

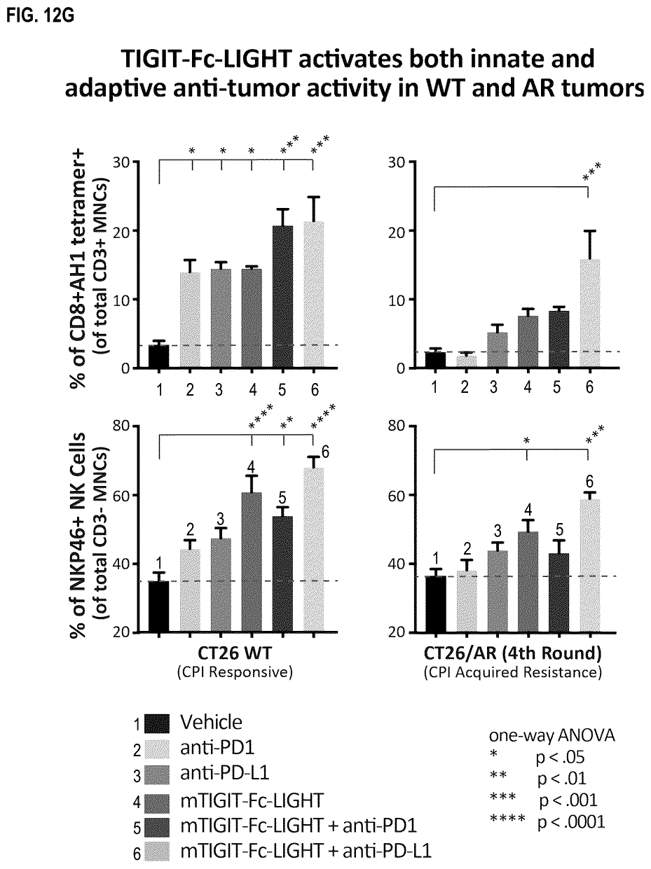

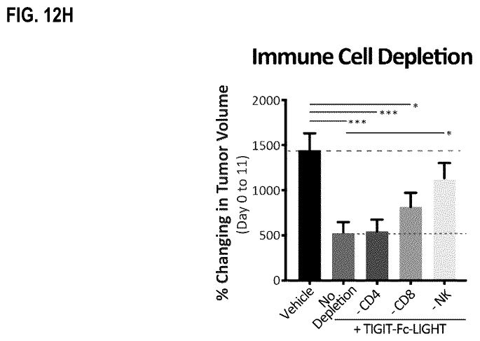

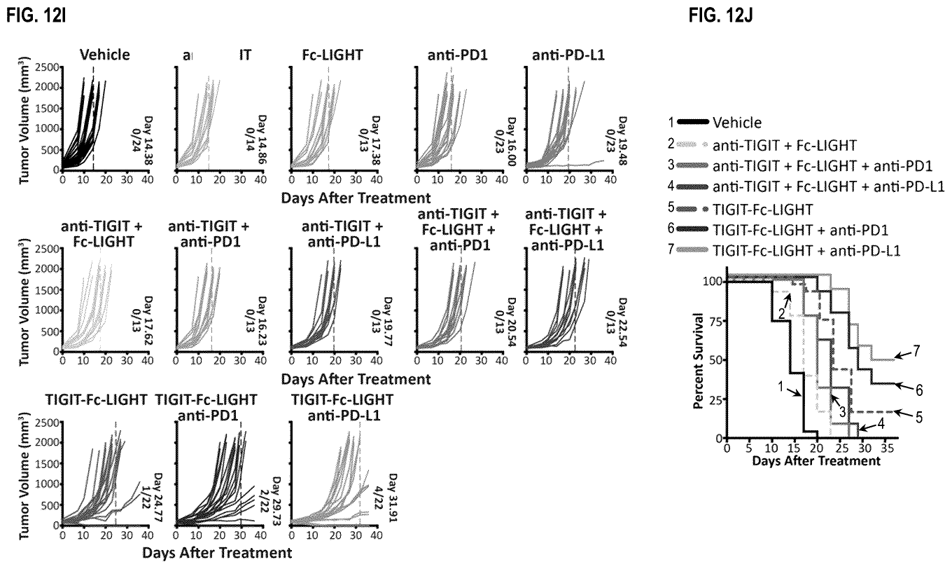

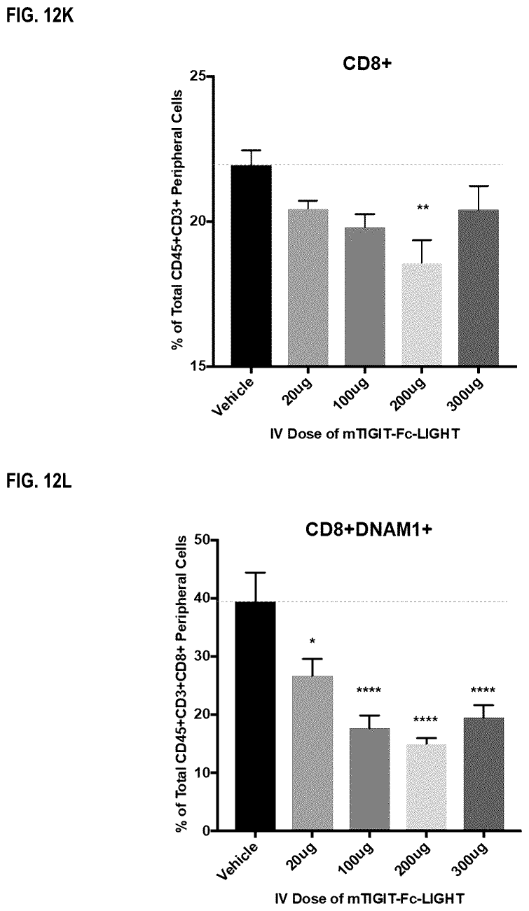

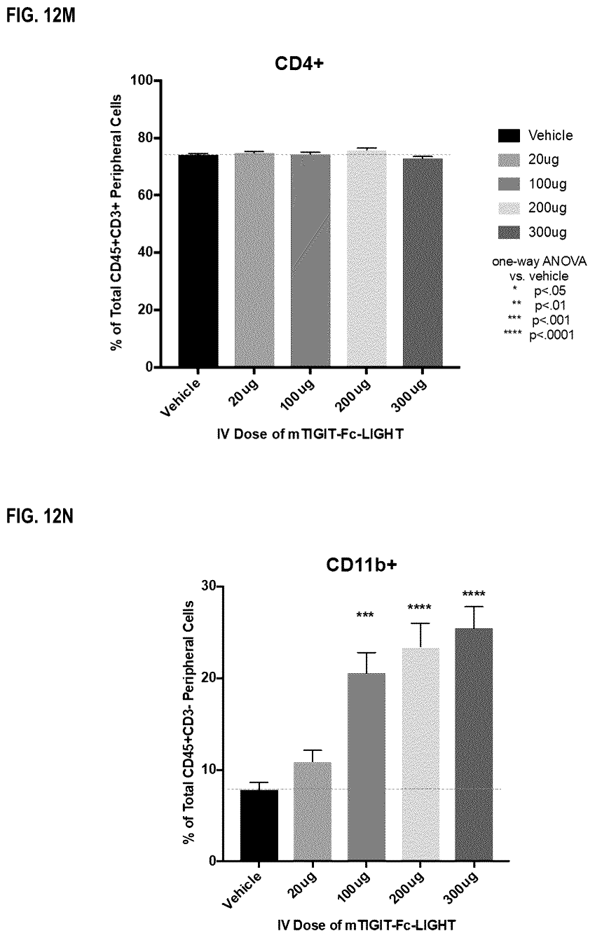

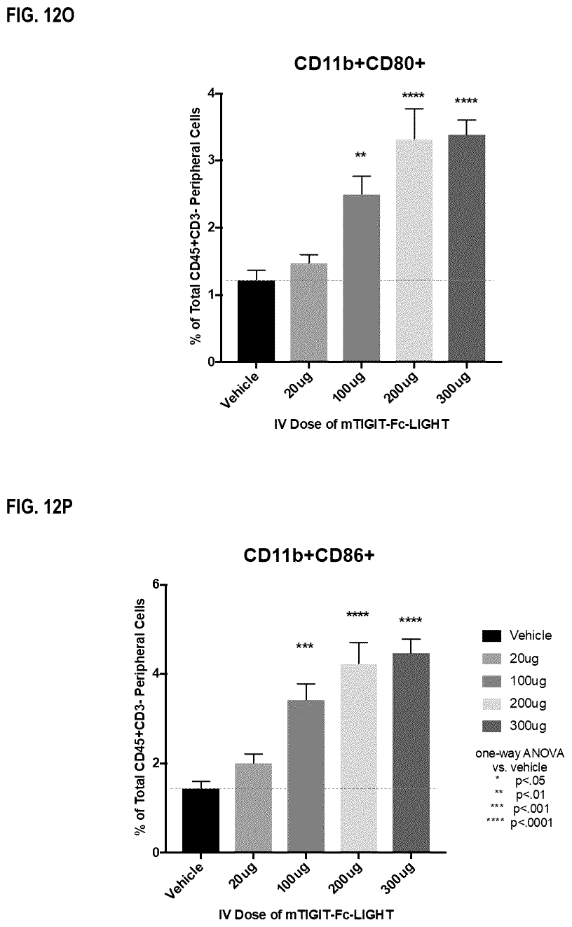

BALB/c mice were inoculated on the hind flank with CT26/WT tumors and when the mean starting tumor volume (STV) reached ˜84 mm³, the mice were randomized and treated with mTIGIT-Fc-LIGHT or benchmark antibodies (FIG. 12A). mTIGIT-Fc-LIGHT significantly delayed tumor growth; following initial treatment, the average day in which the fusion protein treated group reached tumor burden was day 28 (+/−6.87 days) following the initial treatment, compared to day 15 (+/−3.01 days) for the vehicle control group (FIG. 12C). the TIGIT-Fc-LIGHT chimeric protein activity was compared to that of antibodies targeting murine TIGIT, PD-1, PD-L1, LTβR, an Fc-LIGHT fusion protein, and the combination of these agents shown in the Tables below:

| Target | Clone | Dose (ug) | Schedule |
|---|---|---|---|
| anti-TIGIT | 1G9 | 100 | days 0, 3, 6 |
| anti-PD-1 | RMP1-14 | 100 | days 0, 3, 6 |
| anti-PD-L1 | 10F.9G2 | 100 | days 0, 3, 6 |
| Anti-LTβR | 4H8 WH2 | 100 | days 0, 3, 6 |
| Fc-LIGHT | — | 100 | days 0, 3, 6 |
| TIGIT-Fc-LIGHT | — | 200 | days 0, 3, 6 |

The Table above shows test agents, dose, and schedule for CT26 WT tumor efficacy experiment.

| Group | Sample Size (n) | Rejection |
|---|---|---|
| Vehicle | 31 | 0 |
| anti-PD-1 | 30 | 4 |
| anti-PD-L1 | 23 | 2 |
| anti-TIGIT | 19 | 0 |
| Fc-LIGHT | 13 | 0 |
| anti-TIGIT + Fc-LIGHT | 13 | 0 |
| anti-TIGIT + anti-PD-1 | 13 | 0 |
| anti-TIGIT + anti-PD-L1 | 13 | 0 |
| anti-TIGIT + Fc-LIGHT + anti-PD-1 | 13 | 1 |
| anti-TIGIT + Fc-LIGHT + anti-PD-L1 | 13 | 3 |
| TIGIT-Fc-LIGHT | 31 | 3 |
| TIGIT-Fc-LIGHT + anti-PD-1 | 22 | 9 |
| TIGIT-Fc-LIGHT + anti-PDL1 | 22 | 9 |
| anti-LTβR | 5 | 0 |

The Table above shows test agents, dose, and schedule for CT26 WT tumor efficacy experiment.

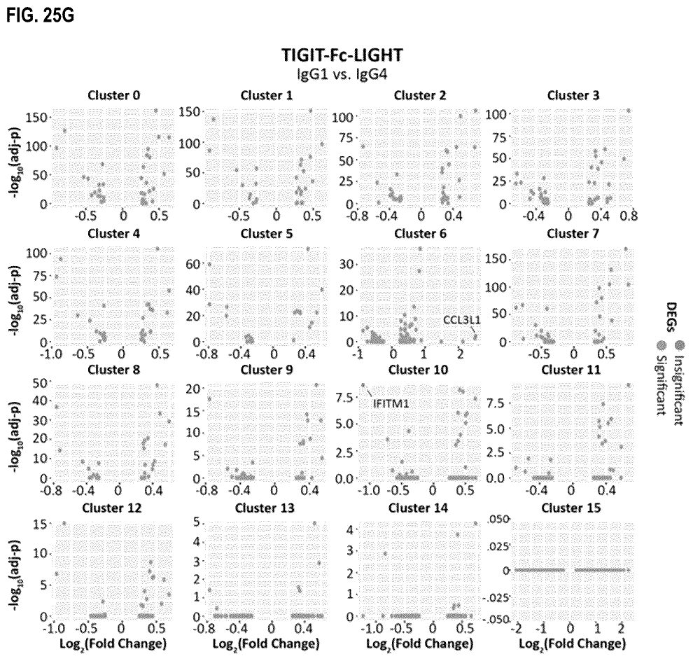

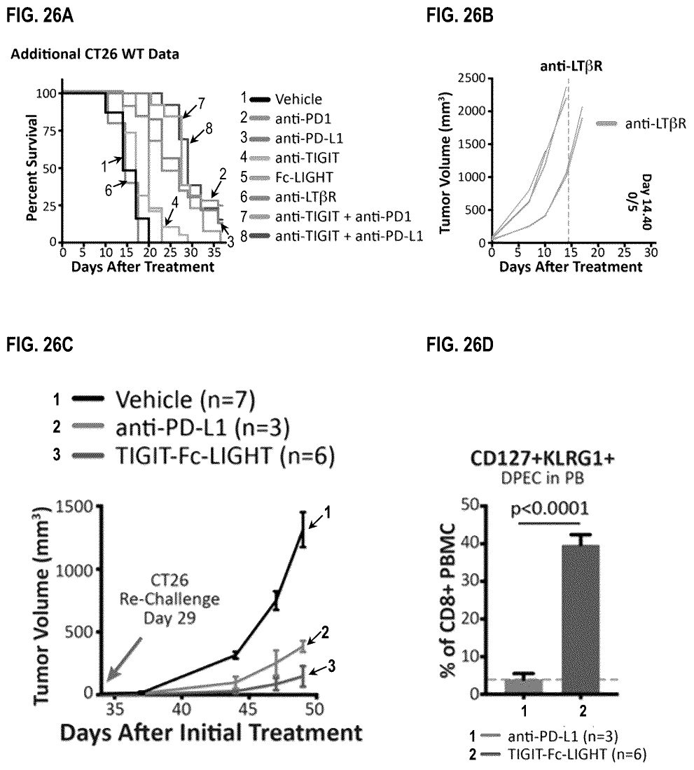

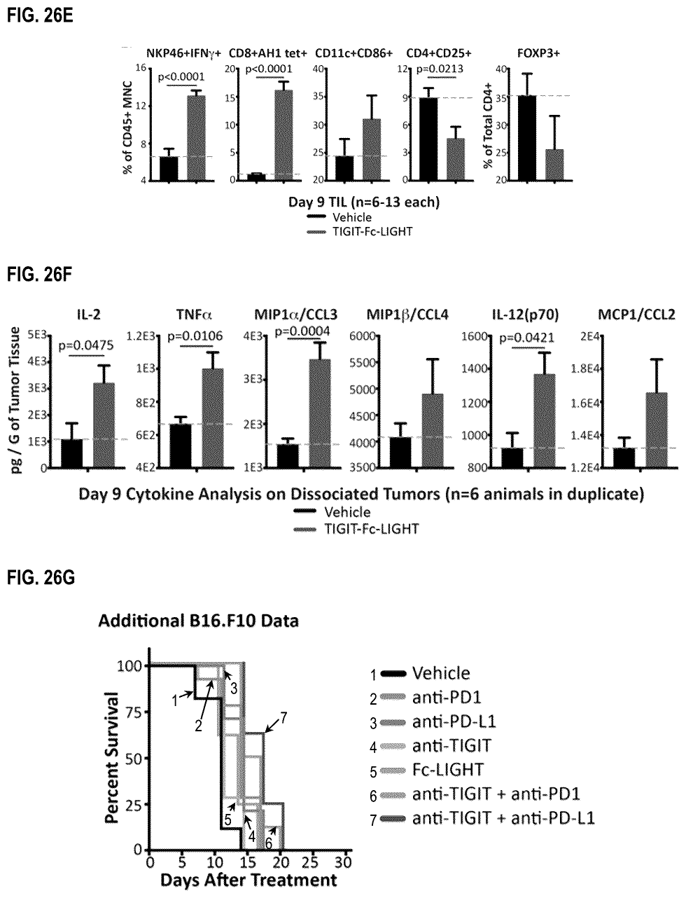

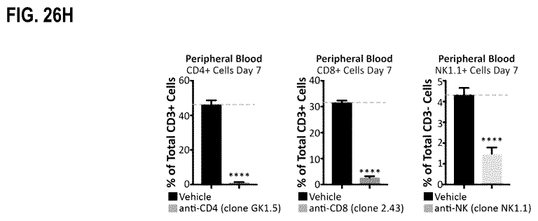

The only commercially available anti-TIGIT (clone 1G9) is an effector silent mouse IgG1 and as a monotherapy still demonstrates modest activity, increasing the time to tumor burden to day 18 (+/−4.38 days). Fc-LIGHT on its own delayed the time to tumor burden to day 20 (+/−1.92 days), but the combination with anti-TIGIT was unable to extend this further, suggesting, without wishing to be bound by theory, that the majority of anti-tumor activity was derived from LIGHT. Mouse TIGIT-Fc-LIGHT significantly improved survival over the separate administration of anti-TIGIT and Fc-LIGHT, indicating that the co-localization of these inhibitory and co-stimulatory signals is critical for maximizing anti-tumor efficacy (FIG. 12D, FIG. 26A, and the table below).

| | | Mantel |
|---|---|---|
| | | Cox |
| Group | Comparator | p-value |
| Vehicle vs. | anti-PD-1 | <0.0001 |
| | anti-PD-L1 | <0.0001 |
| | anti-TIGIT | 0.0135 |
| | Fc-LIGHT | <0.0001 |
| | anti-LTβR | 0.419 |
| | anti-TIGIT + Fc-LIGHT | <0.0001 |
| | anti-TIGIT + anti-PD-1 | <0.0001 |
| | anti-TIGIT + anti-PD-L1 | <0.0001 |
| | anti-TIGIT + Fc-LIGHT + anti-PD-1 | <0.0001 |
| | anti-TIGIT + Fc-LIGHT + anti-PD-L1 | <0.0001 |
| | TIGIT-Fc-LIGHT | <0.0001 |
| | TIGIT-Fc-LIGHT + anti-PD-1 | <0.0001 |
| | TIGIT-Fc-LIGHT + anti-PD-L1 | <0.0001 |
| anti-TIGIT + Fc- | anti-TIGIT + Fc-LIGHT + anti-PD-1 | <0.0001 |
| LIGHT | anti-TIGIT + Fc-LIGHT + anti-PD-L1 | <0.0001 |
| | TIGIT-Fc-LIGHT | <0.0001 |
| | TIGIT-Fc-LIGHT + anti-PD-1 | <0.0001 |
| | TIGIT-Fc-LIGHT + anti-PD-L1 | <0.0001 |
| anti-TIGIT + Fc- | TIGIT-Fc-LIGHT | 0.3647 |
| LIGHT + anti-PD-1 | TIGIT-Fc-LIGHT + anti-PD-1 | 0.1886 |
| anti-TIGIT + Fc- | TIGIT-Fc-LIGHT | 0.1025 |
| LIGHT + anti-PD-L1 | TIGIT-Fc-LIGHT + anti-PD-L1 | 0.4262 |
| Fc-LIGHT | anti-LTβR | <0.0001 |

The Table above shows survival statistics for the CT26 WT tumor efficacy experiment.

A commercially available LT8R antibody (clone 4H8 WH2) was evaluated but did not show any activity in the CT26/WT model (FIG. 26B). Encouragingly, mTIGIT-Fc-LIGHT monotherapy induced complete tumor regression in 12.9% of treated animals.

Individual mice that rejected the primary tumor were rechallenged on day 29 with a second inoculation of CT26/WT tumor cells on the opposing hind flank without subsequent retreatment. Mice observed to have complete tumor rejection following an initial course of treatment with the TIGIT-Fc-LIGHT chimeric protein were found to have protective immunity that prevented the progression of secondary tumors (FIG. 26C). From these animals, which included 6 mice initially treated with the TIGIT-Fc-LIGHT chimeric protein and 3 mice treated with anti-PD-L1 monotherapy, peripheral blood was isolated on day 39 (10 days following the day 29 rechallenge). The comparative levels of double-positive effector memory cells (CD127⁺FKLRG1⁺ out of total CD8⁺ PBMC) were evaluated in these mice, and this population of cells was found to be significantly expanded in animals initially treated with the TIGIT-Fc-LIGHT chimeric protein (FIG. 26D). The initial anti-tumor activity of the TIGIT-Fc-LIGHT chimeric protein was associated with increased tumor infiltration of IFNg+ NK cells, antigen-specific CD8+ T cells, and CD86+ dendritic cells, along with depletion of CD25+ and FOXP3+ CD4 regulatory T cells (FIG. 26E). Interestingly, FOXP3 levels were also observed to be down-regulated in human PBMC following treatment with both IgG1 and IgG4 variants of the human TIGIT-Fc-LIGHT chimeric protein, specifically in the ImmuneExp defined annotation for regulatory T cells (FIG. 25F). In addition, in these murine studies, a series of effector and myeloid-specific cytokines were assessed in the tumor milieu, using supernatant generated from dissociated tumor tissue. Increased tumor levels of IL-2, TNFα, CCL3, CCL4, IL-12(p70), and CCL2 were found within the tumor microenvironment of mTIGIT-Fc-LIGHT treated animals (FIG. 26F). The combination of anti-TIGIT with Fc-LIGHT and either anti-PD-1 or anti-PD-L1 yielded the greatest activity of the benchmark control groups, with tumor burden pushed to 31 (+/−3.75 days) and 32 (+/−3.82 days) days, and complete tumor rejection in 7.7% and 23.1% of treated animals, respectively (FIG. 12C). Combinations of mTIGIT-Fc-LIGHT with either anti-PD-1 or anti-PD-L1 also significantly improved efficacy, with tumor burden reached on day 33 (+/−4.94 or 4.64 days, respectively). Complete tumor rejection was observed in 40.9% of animals for both anti-PD-1 and anti-PD-L1 combinations.

Mouse TIGIT-Fc-LIGHT also demonstrated monotherapy activity in B16.F10 melanoma, where the average time to reach tumor burden was seen at 19 (+/−4.47) days, compared to 10 (+/−2.00) days in the vehicle control group, and 14 (+/−2.27) days in the anti-TIGIT+Fc-LIGHT combination group (FIG. 12A, FIG. 12E, and the Tables below).

| Target | Clone | Dose (ug) | Schedule |
|---|---|---|---|
| anti-TIGIT | 1G9 | 100 | days 0, 3, 6 |
| anti-PD-1 | RMP1-14 | 100 | days 0, 3, 6 |
| anti-PD-L1 | 10F.9G2 | 100 | days 0, 3, 6 |
| Fc-LIGHT | — | 100 | days 0, 3, 6 |
| TIGIT-Fc-LIGHT | — | 200 | days 0, 3, 6 |

The Table above shows test agents, dose, and schedule for B16.F10 tumor efficacy experiment.

| Group | Sample Size (n) | Rejection |
|---|---|---|
| Vehicle | 17 | 0 |
| anti-TIGIT | 14 | 0 |
| anti-PD-1 | 14 | 0 |
| anti-PD-L1 | 14 | 0 |
| Fc-LIGHT | 8 | 0 |
| anti-TIGIT + Fc-LIGHT | 8 | 0 |
| anti-TIGIT + Fc-LIGHT + anti-PD-1 | 8 | 0 |
| anti-TIGIT + Fc-LIGHT + anti-PD-L1 | 8 | 0 |
| anti-TIGIT + anti-PD-1 | 8 | 0 |
| anti-TIGIT + anti-PD-L1 | 8 | 0 |
| TIGIT-Fc-LIGHT | 16 | 0 |
| TIGIT-Fc-LIGHT + anti-PD-1 | 17 | 0 |
| TIGIT-Fc-LIGHT + anti-PD-L1 | 17 | 1 |

The Table above shows group sample size and number of animals that completely rejected the primary tumor in the B16.F10 tumor efficacy experiment.

The triplet combination of anti-TIGIT, Fc-LIGHT, and either anti-PD-1 or anti-PD-L1 provided intermediate benefit, delaying tumor growth to day 18 (+/−3.02 or 2.56 days, respectively). The combination of mTIGIT-Fc-LIGHT with anti-PD-1 or anti-PD-L1 improved efficacy and survival further (days 24 (+/−4.85) and 26 (+/−4.02), respectively), and the combination with anti-PD-L1 induced complete tumor rejection in one animal. These findings are significant given the aggressive tumor type and starting tumor volumes averaging >110 mm³ (FIG. 12E, FIG. 12F, FIG. 26G, and the Table below).

| | | Mantel |
|---|---|---|
| | | Cox |
| Group | Comparator | p-value |
| Vehicle vs. | anti-PD-1 | 0.0004 |
| | anti-PD-L1 | 0.0005 |
| | anti-TIGIT | 0.1859 |
| | Fc-LIGHT | 0.0059 |
| | anti-TIGIT + Fc-LIGHT | 0.0019 |
| | anti-TIGIT + anti-PD-1 | <0.0001 |
| | anti-TIGIT + anti-PD-L1 | <0.0001 |
| | anti-TIGIT + Fc-LIGHT + anti-PD-1 | <0.0001 |
| | anti-TIGIT + Fc-LIGHT + anti-PD-L1 | <0.0001 |
| | TIGIT-Fc-LIGHT | <0.0001 |
| | TIGIT-Fc-LIGHT + anti-PD-1 | <0.0001 |
| | TIGIT-Fc-LIGHT + anti-PD-L1 | <0.0001 |
| anti-TIGIT + Fc- | anti-TIGIT + Fc-LIGHT + anti-PD-1 | 0.0046 |
| LIGHT | anti-TIGIT + Fc-LIGHT + anti-PD-L1 | 0.0021 |
| | TIGIT-Fc-LIGHT | 0.0004 |
| | TIGIT-Fc-LIGHT + anti-PD-1 | <0.0001 |
| | TIGIT-Fc-LIGHT + anti-PD-L1 | <0.0001 |
| anti-TIGIT + Fc- | TIGIT-Fc-LIGHT | 0.3936 |
| LIGHT + anti-PD-1 | TIGIT-Fc-LIGHT + anti-PD-1 | 0.0041 |
| anti-TIGIT + Fc- | TIGIT-Fc-LIGHT | 0.3688 |
| LIGHT + anti-PD-L1 | TIGIT-Fc-LIGHT + anti-PD-L1 | <0.0001 |

The Table above shows survival statistics for the B16.F10 tumor efficacy experiment.

In the B16.F10 model, the contribution of immune cell subsets to anti-tumor activity was evaluated. A cohort of mice were treated with CD4, CD8, or NK depleting antibodies on days −1, 1, and 7 of the time-course, and cell depletion was confirmed by flow cytometry (FIG. 12A, FIG. 12H, and FIG. 26H). The depletion of CD4 cells had no impact on mTIGIT-Fc-LIGHT activity, however the depletion of CD8 cells or NK cells partially reduced tumor growth control, consistent with the expression pattern of TIGIT and HVEM (FIG. 12H). The fact that neither CD8 nor NK depletion completely abrogated anti-tumor responses suggested, without wishing to be bound by theory, that both CD8+ T cells and NK cells contribute independently to anti-tumor activity of the TIGIT-Fc-LIGHT chimeric protein, which was nonetheless enhanced when both cell populations were present.

To evaluate the effect of the antitumor activity of the TIGIT-Fc-LIGHT chimeric protein, mice were inoculated in rear flank with colorectal tumor CT26 cells, CT26 anti-PD-1 resistant cells (acquired resistance), or B16.F10 melanoma tumors (primarily resistant). The mice were randomly divided in the following treatment groups: (1) vehicle-alone, (2) 100 μg per dose of an anti-PD-1 antibody (clone RMP1-14), (3) 100 μg per dose of an anti-PD-L1 antibody (clone 10F.9G2), (4) 200 μg per dose of the TIGIT-Fc-LIGHT chimeric protein, an anti-PD-L1 antibody, (5) 100 μg per dose of the anti-PD-1 antibody+200 μg per dose of the TIGIT-Fc-LIGHT chimeric protein, and (6) 100 μg per dose of the anti-PD-L1 antibody+200 μg per dose of the TIGIT-Fc-LIGHT chimeric protein. Mice were dosed on days 0, 3, and 6 following tumor inoculation via intraperitoneal injections. Tumor sizes were measured on day 11 post-tumor inoculation in the B16.F10 model, and on day 14 post-tumor inoculation in the CT26 and CT26 anti-PD-1 resistant models. Tumor growth inhibition compared to vehicle alone-treated mice was calculated and plotted.

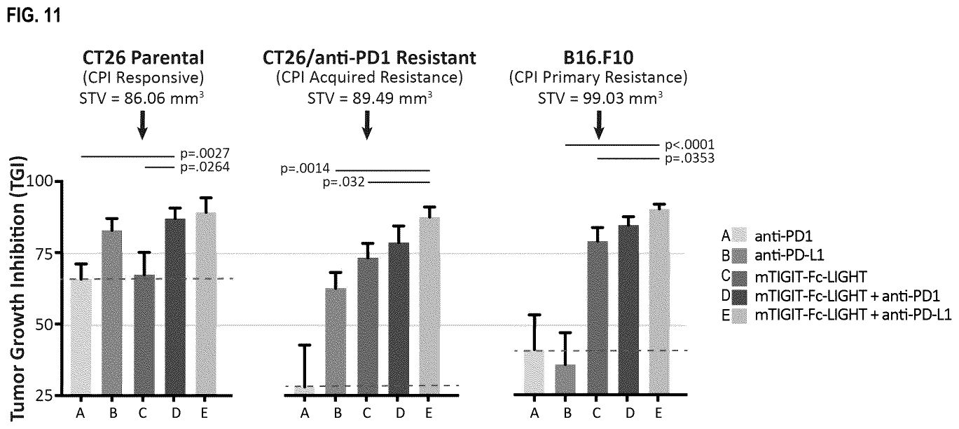

As shown in FIG. 11 (left panel), each of the treatments led to a tumor growth inhibition of CT26 tumors. The combination treatment with the anti-PD-1 antibody and the TIGIT-Fc-LIGHT chimeric protein showed significantly higher tumor growth inhibition compared to the treatment with the anti-PD-1 antibody alone (p=0.0027) as well as the treatment with the TIGIT-Fc-LIGHT chimeric protein alone (p=0.0264) (FIG. 11, left panel). In a similar experiment, identical treatments were initiated when starting average tumor size reached 86.6 mm³ overall survival was measured and plotted. FIG. 12A shows the individual animal tumor growth curves, the average day in which each group reached tumor burden, and the number of mice that completely rejected the tumor in response to treatment. As shown in FIG. 12B (left panel), consistent with the observed tumor growth inhibition, each of the treatments led to increased survival of mice as shown in the Kaplan-Meier curves compared to vehicle treated mice. The combination treatment with the anti-PD-1 antibody and the TIGIT-Fc-LIGHT chimeric protein showed significantly higher survival compared to the treatment with the anti-PD-1 antibody alone (p=0.0238) as well as a higher survival compared to the treatment with the TIGIT-Fc-LIGHT chimeric protein alone as shown in the Kaplan-Meier curves (p=0.1445) (FIG. 12B, left panel).

Similar analysis was performed with the CT26 anti-PD-1 resistant cell allograft model. As shown in FIG. 11 (middle panel), each of the treatments, except the treatment with the anti-PD-1 antibody, led to a tumor growth inhibition of CT26 anti-PD-1 resistant tumor. Interestingly, the combination treatment with the anti-PD-1 antibody and the TIGIT-Fc-LIGHT chimeric protein showed significantly higher tumor growth inhibition compared to the treatment with the TIGIT-Fc-LIGHT chimeric protein alone (p=0.032) (FIG. 11, middle panel). These data are surprising, inter alia, because the treatment with the anti-PD-1 antibody alone was not effective in the CT26 anti-PD-1 resistant tumor model (FIG. 11, middle panel). The combination treatment with the anti-PD-1 antibody and the TIGIT-Fc-LIGHT chimeric protein also showed significantly higher tumor growth inhibition compared to the treatment with the anti-PD-L1 antibody alone (p=0.014) (FIG. 11, middle panel). In a similar experiment, treatments were initiated when starting average tumor size reached 89.49 mm³. Overall survival was measured and plotted. As shown in FIG. 12B (middle panel), the treatment with the anti-PD-1 antibody alone caused very little improvement in survival, if any, in the CT26 anti-PD-1 resistant tumor model, consistent with the observed lack of tumor growth inhibition. The treatment with the anti-PD-L1 antibody alone caused some improvement in survival in the CT26 anti-PD-1 resistant tumor model as shown in the Kaplan-Meier curves compared to vehicle treated mice (FIG. 12B, middle panel). Interestingly, the combination treatment with the TIGIT-Fc-LIGHT chimeric protein and the anti-PD-1 antibody showed an improved survival compared to the treatment with the TIGIT-Fc-LIGHT chimeric protein alone as shown in the Kaplan-Meier curves (p=0.1672) (FIG. 12B, middle panel). Similarly, the combination treatment with the TIGIT-Fc-LIGHT chimeric protein and the anti-PD-L1 antibody showed a significantly improved survival compared to the treatment with the TIGIT-Fc-LIGHT chimeric protein alone (p=0.0443) (FIG. 12B, middle panel). These data are surprising, inter alia, because the treatment with the anti-PD-1 antibody alone was not effective in the CT26 anti-PD-1 resistant tumor model (FIG. 11, middle panel; FIG. 12B, middle panel), and one would not have expected the significantly improved efficacy of the TIGIT-Fc-LIGHT chimeric protein when combined with the anti-PD-1 antibody.

Similar analysis was performed with the B16.F10 cell allograft model. As shown in FIG. 11 (right panel), the treatments with the anti-PD-L1 antibody alone or the anti-PD-L1 antibody alone showed very small improvement in tumor growth inhibition. In contrast, the treatment with the TIGIT-Fc-LIGHT chimeric protein alone caused a greater improvement in tumor growth inhibition compared to the anti-PD-L1 antibody alone or the anti-PD-L1 antibody alone in the B16.F10 cell allograft model. Interestingly, the combination treatment with the anti-PD-L1 antibody and the TIGIT-Fc-LIGHT chimeric protein showed significantly higher tumor growth inhibition in the B16.F10 cell allograft model compared to the treatment with the TIGIT-Fc-LIGHT chimeric protein alone (p=0.0353) or the anti-PD-L1 antibody alone (p<0.0001) (FIG. 11, right panel). The combination treatment with the anti-PD-1 antibody and the TIGIT-Fc-LIGHT chimeric protein also showed a higher tumor growth inhibition in the B16.F10 cell allograft model compared to the treatment with the TIGIT-Fc-LIGHT chimeric protein alone (FIG. 11, right panel). In a similar experiment, treatments were initiated when starting average tumor size reached 99.03 mm³. Overall survival was measured and plotted. As shown in FIG. 12B (right panel), the treatment with the anti-PD-1 antibody alone or the anti-PD-L1 antibody alone caused a slight improvement in survival in the B16.F10 tumor model, consistent with the observed small tumor growth inhibition. Interestingly, the combination treatment with the TIGIT-Fc-LIGHT chimeric protein and the anti-PD-1 antibody showed a significantly improved survival compared to the treatment with the TIGIT-Fc-LIGHT chimeric protein alone (p=0.0031) (FIG. 12B, right panel). Similarly, the combination treatment with the TIGIT-Fc-LIGHT chimeric protein and the anti-PD-L1 antibody showed an improved survival compared to the treatment with the TIGIT-Fc-LIGHT chimeric protein alone (p=0.2268) (FIG. 12B, right panel). These data are surprising, inter alia, because the treatment with the anti-PD-1 antibody or PD-L1 antibody alone was not effective in the B16.F10 tumor model (FIG. 11, right panel; FIG. 12B, right panel), and one would not have expected the significantly improved efficacy when combined with the TIGIT-Fc-LIGHT chimeric protein.

A preclinical model of checkpoint inhibitor acquired resistance was generated and characterized. The model reflects many of the features observed in PD-1/L1 acquired resistant NSCLC patients. This model was developed by treating mice with established CT26/WT tumors with anti-PD-1 antibodies, and excising tumors which were partially controlled but not rejected following treatment. The excised tumors were dissociated, expanded ex vivo, and re-inoculated into new recipient mice for repeat treatment with anti-PD-1 antibodies. This procedure was serially repeated five times in vivo to provide continuous, anti-PD-1 selective pressure, until none of the mice demonstrated benefit from PD-1 blockade. The resulting tumors were named CT26/AR (acquired resistance). A comparison of the transcriptomes of CT26/AR tumors and tumors isolated from PD-1/L1 antibody acquired resistance NSCLC patients were compared and found to share similar perturbations in JAK/STAT and IFN signaling pathways. Therefore, the CT26/AR model, may be a useful tool for assessing the potential anti-tumor activity of novel therapeutics in the setting of PD-1/L1 resistance.

Mouse TIGIT-Fc-LIGHT along with previously presented benchmark control treatment groups presented previously, were evaluated in CT26/AR tumors after reaching a starting tumor volume of >100 mm³ in recipient 5 mice (FIG. 12A and the Tables below).

| Target | Clone | Dose (ug) | Schedule |
|---|---|---|---|
| anti-TIGIT | 1G9 | 100 | days 0, 3, 6 |
| anti-PD-1 | RMP1-14 | 100 | days 0, 3, 6 |
| anti-PD-L1 | 10F.9G2 | 100 | days 0, 3, 6 |
| Fc-LIGHT | — | 100 | days 0, 3, 6 |
| TIGIT-Fc-LIGHT | — | 200 | days 0, 3, 6 |

The Table above shows test agents, dose, and schedule for CT26 AR tumor efficacy experiment.

| Group | Sample Size (n) | Rejection |
|---|---|---|
| Vehicle | 24 | 0 |
| anti-PD-1 | 23 | 0 |
| anti-PD-L1 | 23 | 0 |
| anti-TIGIT | 14 | 0 |
| Fc-LIGHT | 13 | 0 |
| anti-TIGIT + Fc-LIGHT | 13 | 0 |
| anti-TIGIT + anti-PD-1 | 13 | 0 |
| anti-TIGIT + anti-PD-L1 | 13 | 0 |
| anti-TIGIT + Fc-LIGHT + anti-PD-1 | 13 | 0 |
| anti-TIGIT + Fc-LIGHT + anti-PD-L1 | 13 | 0 |
| TIGIT-Fc-LIGHT | 22 | 1 |
| TIGIT-Fc-LIGHT + anti-PD-1 | 22 | 2 |
| TIGIT-Fc-LIGHT + anti-PD-L1 | 22 | 4 |

The Table above shows group sample size and number of animals that completely rejected the primary tumor in the CT26 AR tumor efficacy experiment.

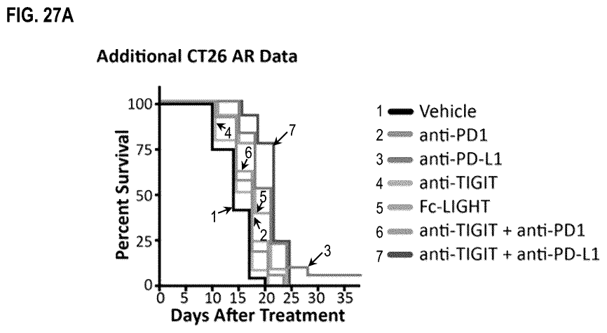

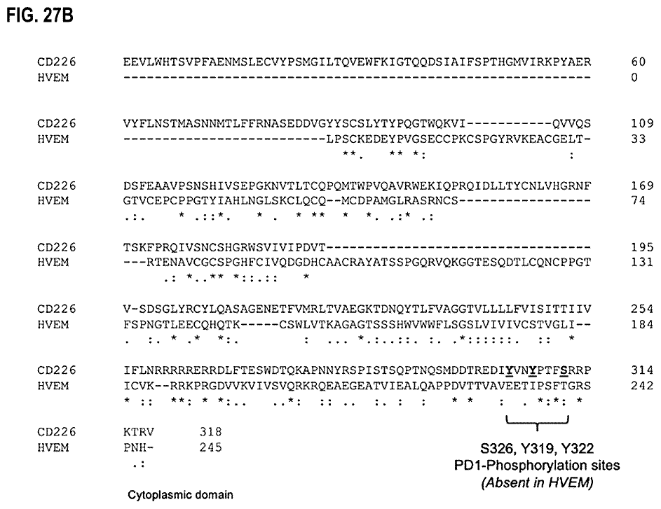

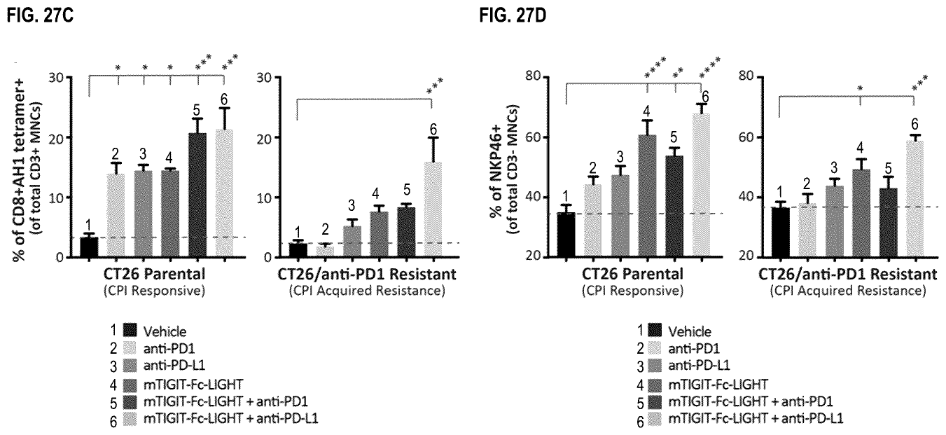

On average, CT26/AR tumors grew more quickly in vivo than their CT26/WT counterparts with vehicle treated animals reaching tumor burden at an average of 14 (+/−3.02) days following the initiation of treatment (FIG. 12I). Treatment with anti-TIGIT or anti-PD-1 did not appreciably delay tumor growth, while Fc-LIGHT and anti-PD-L1 delayed tumor burden to 17 (+/−3.38) and 19 (+/−5.07) days, respectively. The modest monotherapy activity of Fc-LIGHT also suggested, without wishing to be bound by theory, that LIGHT signaling through HVEM and/or LTβR, functions independently from PD-1, and was distinct from monotherapy anti-TIGIT treatment (FIG. 12I). Along these lines, the alignment of the cytoplasmic amino acid sequences between HVEM and DNAM-1 identified minimal homology, whereby HVEM lacked the tyrosine residues found in DNAM-1, which have been reported to become dephosphorylated by the SHP-2 domain of PD-1; involved in PD-1 inhibition of DNAM-1 costimulation (FIG. 27B).

Mouse TIGIT-Fc-LIGHT monotherapy delayed tumor burden until day 24 (+/−6.05 days) and significantly improved survival as compared to the separate administration of anti-TIGIT and Fc-LIGHT (Mantel-Cox p-value <0.0001; FIG. 12I-FIG. 12J, FIG. 27A, and the Table below).

| | | Mantel |
|---|---|---|
| | | Cox |
| Group | Comparator | p-value |
| Vehicle vs. | anti-PD-1 | 0.0889 |
| | anti-PD-L1 | <0.0001 |
| | anti-TIGIT | 0.5928 |
| | Fc-LIGHT | 0.0062 |
| | anti-TIGIT + Fc-LIGHT | 0.0053 |
| | anti-TIGIT + anti-PD-1 | 0.0791 |
| | anti-TIGIT + anti-PD-L1 | <0.0001 |
| | anti-TIGIT + Fc-LIGHT + anti-PD-1 | <0.0001 |
| | anti-TIGIT + Fc-LIGHT + anti-PD-L1 | <0.0001 |
| | TIGIT-Fc-LIGHT | <0.0001 |
| | TIGIT-Fc-LIGHT + anti-PD-1 | <0.0001 |
| | TIGIT-Fc-LIGHT + anti-PD-L1 | <0.0001 |
| anti-TIGIT + Fc- | anti-TIGIT + Fc-LIGHT + anti-PD-1 | 0.0557 |
| LIGHT | anti-TIGIT + Fc-LIGHT + anti-PD-L1 | 0.0042 |
| | TIGIT-Fc-LIGHT | <0.0001 |
| | TIGIT-Fc-LIGHT + anti-PD-1 | <0.0001 |
| | TIGIT-Fc-LIGHT + anti-PD-L1 | <0.0001 |
| anti-TIGIT + Fc- | TIGIT-Fc-LIGHT | 0.0082 |
| LIGHT + anti-PD-1 | TIGIT-Fc-LIGHT + anti-PD-1 | <0.0001 |
| anti-TIGIT + Fc- | TIGIT-Fc-LIGHT | 0.2502 |
| LIGHT + anti-PD-L1 | TIGIT-Fc-LIGHT + anti-PD-L1 | <0.0001 |

The Table above shows survival statistics for the CT26 AR tumor efficacy experiment.

The combination of anti-PD-1 or anti-PD-L1 and the TIGIT-Fc-LIGHT chimeric protein delayed tumor burden to 29 days (+/−5.95 days) and 31 (+/−5.18 days), and induced complete tumor rejection in 9.1% and 18.2% 5 of mice, respectively. Both the TIGIT-Fc-LIGHT chimeric protein combinations significantly improved survival compared to triplet combinations of anti-TIGIT, Fc-LIGHT, and either anti-PD-1 or anti-PD-L1 (FIG. 12J and FIG. 27A). Interestingly, CT26/AR tumors had slightly less CD8+ T cell infiltration than CT26/WT tumors, but similar levels of NK cells (FIG. 27C-FIG. 27D). While the ability to therapeutically enhance T cell infiltrate was constrained in CT26/AR tumors, the TIGIT-Fc-LIGHT chimeric protein monotherapy still modestly enhanced antigen-specific CD8+ T cells into the tumor. Furthermore, in this tumor model, the combination of the TIGIT-Fc-LIGHT chimeric protein with anti-PD-L1, led to a significant increase in effector CD8+ T cell infiltration that was comparable to the level observed in CT26/WT tumors (FIG. 27C). NK cells appear to be less impacted in the CT26/AR model, and both the TIGIT-Fc-LIGHT chimeric protein monotherapy and combinations with anti-PD-L1, led to a significant increase in NK cells (FIG. 27D). These results are consistent with the immune cell contributions identified in the previously described cell depletion studies (FIG. 12H) and suggest, inter alia, that the broad immune stimulating activity of the TIGIT-Fc-LIGHT chimeric protein may potentiate anti-tumor immunity in the setting of checkpoint acquired resistance.

These results, inter alia, demonstrate that the TIGIT-Fc-LIGHT chimeric protein is effective against cancer irrespective of resistance to checkpoint blockade. Moreover, the cancers that are susceptible to PD-1/PD-L1 axis blockade may be treated with a combination of a PD-1/PD-L1 axis blocker and the TIGIT-Fc-LIGHT chimeric protein.

To evaluate the activation of myeloid cells, the murine TIGIT-Fc-LIGHT chimeric protein (mTIGIT-Fc-LIGHT) was injected IV in mice harboring tumors and increasing soses of the the murine TIGIT-Fc-LIGHT chimeric protein (mTIGIT-Fc-LIGHT) were injected in mice. peripheral blood was extracted after one day and immune phenotyping of cells was carried out using flow cytometry.

As shown in FIG. 12K, 200 μg dose of the TIGIT-Fc-LIGHT chimeric protein decreased the counts of CD8+ cells. As shown in FIG. 12L, 20, 100, 200 and 300 μg doses of the TIGIT-Fc-LIGHT chimeric protein significantly decreased the counts of the activated CD8+ DNAM1A-cells. on the other hand, as shown in FIG. 12M, the TIGIT-Fc-LIGHT chimeric protein significantly change the counts of the CD4+ cells. These results suggest the “margination” of CD8+ T cells, specifically the CD8+ DNAM1+ cells, out of the periphery; peaking at doses of 200 ug, which is the human equivalent dose of about ˜10 mg/kg.

As shown in FIG. 12N, 100, 200 and 300 μg doses of the TIGIT-Fc-LIGHT chimeric protein significantly increased the counts of CD11+ cells. As shown in FIG. 12O, 100, 200 and 300 μg doses of the TIGIT-Fc-LIGHT chimeric protein significantly increased the counts of the activated CD11+CD80A-cells. Similarly, as shown in FIG. 12P, 100, 200 and 300 μg doses of the TIGIT-Fc-LIGHT chimeric protein significantly increased the counts of the activated CD11+CD86+ cells. These results suggest an increase in total and activated CD11b+ cells, peaking at doses of 200 μg, which is the human equivalent dose of about −10 mg/kg.

### Example 12: The TIGIT-Fc-LIGHT Chimeric Protein Activates Both Innate and Adaptive Immunity

The expression of 53 immune co-stimulatory receptors was assessed across TOGA tumors (FIG. 13A). Genes were rank ordered from high- to low-based on the mean expression of each gene across all TOGA tumors. Consistent with previously described down-regulation of DNAM-1 expression in advanced tumors, we found that DNAM-1 ranked as one of the least abundant immune co-stimulators (FIG. 13A). Amongst the costimulatory receptors with more consistent expression patterns across tumor types, both herpes virus entry mediator A (HVEM, TNFRSF14) and lymphotoxin beta receptor (LTβR, TNFRSF3) were ranked among the top ten, with significantly higher transcript counts as compared to DNAM-1 (FIG. 13A-FIG. 13B). The expression of HVEM and LTβR as TNF-receptors highly expressed in tumors across all TOGA cancers, identifying HVEM and LTBR in the top 10 (FIG. 13A). HVEM and LTβR are both receptors for a TNF ligand known as LIGHT (TNFSF14, ‘homologous to lymphotoxin, exhibits inducible expression and competes with HSV glycoprotein D for binding to HVEM, a receptor expressed on T lymphocytes).

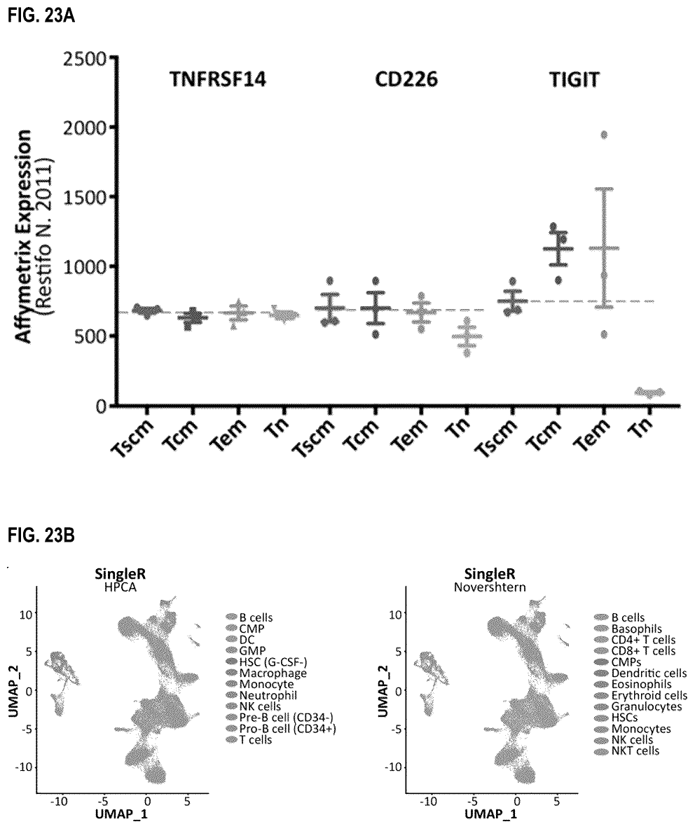

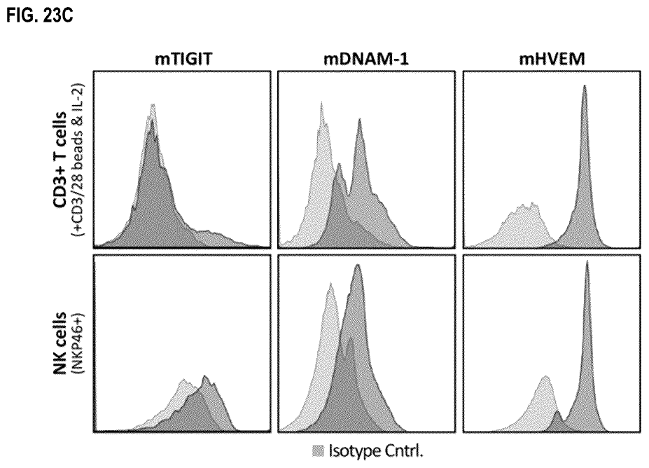

To evaluate the relative expression of HVEM and DNAM-1 on Tscm cells, a previously described in vitro differentiation protocol was used. Gattinoni et al., A human memory T cell subset with stem cell-like properties. Nat Med 17, 1290-1297 (2011). Whereas HVEM and LTβR are receptors for LIGHT, TIGIT antagonizes the activating effects of DNAM-1. Therefore, the expression HVEM and DNAM-1 on T stem cell memory (Tscm) cells was characterized. Naïve CD8+ cells were isolated from healthy donor human PBMC, and were cultured with anti-human CD3/CD28 beads, IL-2, and a GSK36 inhibitor. After 9 days in culture, cells were isolated and phenotyped using a panel of antibodies that separate naïve CD8+ T cells (Tn) from Tscm cells (FIG. 13C). Gattinoni et al., A human memory T cell subset with stem cell-like properties. Nat Med 17, 1290-1297 (2011. HVEM expression was characterized in relation to DNAM-1 expression on T stem cell memory (Tscm) cells induced for 9 days with CD3/CD28, IL-2, and a GSK36 inhibitor. The expression of HVEM and DNAM-1 was assayed. Accordingly, it was found that the majority of Tn cells express HVEM (74.1% express HVEM alone and 21.8% co-express HVEM and DNAM-1), whereas only 1.15% of Tn express DNAM-1 alone. Consistent with recent unpublished findings, Tscm cells expressed more DNAM-1 than their Tn counterparts, but the majority of DNAM-1+ cells also expressed HVEM (45.5% out of 54.25%). Further, 86.7% of total Tscm expressed high levels of HVEM. In this dataset, HVEM was highly expressed on all T cell populations including Tscm, whereas DNAM-1 expression varied between the different memory T cell subsets (FIG. 13C and FIG. 23A). Therefore, HVEM and DNAM-1 as TNF-receptors were highly expressed on naïve CD8+ T cells and CD8+ T stem cell memory cells.

To extend this analysis and systematically evaluate the abundance of co-stimulatory signals on immune cells, single cell RNA sequencing (scRNA-seq) was performed on human PBMC, and bioinformatically defined cell populations through transcriptome clustering (Seurat) and immune cell type predictions (FIG. 13D). The expression of the 53 immune co-stimulatory genes assessed above, were also interrogated across the 16 Seurat defined clusters (FIG. 13D). Genes were rank ordered from high- to low-based on average expression of each gene across all clusters. TNFRSF14 (HVEM) and CD226 (DNAM-1) ranked high in this heatmap, indicating that they were highly expressed in a number of immune cells, notably, in clusters 5 and 8 which corresponded to CD8+ T cells, and clusters 7, 10, and 11 which represented natural killer (NK) cells (FIG. 13E and FIG. 13F). Novershtern and HPCA annotations were also applied to the data and generated similar cell type predictions (FIG. 23B). As expected, LTBR expression was low compared to other TNF-receptors in most PBMC populations, with the exception of cluster 6, which represents myeloid cells (FIG. 13F).

The effect of the TIGIT-Fc-LIGHT chimeric protein on innate and adaptive immunity was studied. Briefly, mice were inoculated in rear flank with colorectal tumor CT26 cells or CT26 anti-PD-1 resistant cell tumors. A cohort of experimental mice were humanely euthanized 14 days after tumor inoculation, tumors were excised and dissociated, and immune infiltrate was assessed by flow cytometry. The HVEM/DNAM1 expressing T and NK cells were assessed by flow cytometry from excised CT26 or CT26 anti-PD-1 resistant tumors. As shown in FIG. 13G, about 93% of the NK tumor infiltrating lymphocytes (TILs) were HVEM+, compared to DNAM1⁺ NK TILs, which were about 61%. Moreover, about 95% of the CD8 T TILs were HVEM+, compared to DNAM1⁺ CD8 T TILs, which were about 58% (FIG. 13G). These results suggest, inter alia, that although the expression of HVEM is more abundant in TILs compared to DNAM, both innate and adaptive immune cells are present among TILs.

To assess whether the preferential expression of HVEM over DNAM-1 on TIL translated preclinically to a range of murine tumor types, Mice were inoculated with CT26 wild-type (CT26/WT), CT26 CPI-acquired resistance (CT26/AR), or B16.F10 tumors (FIG. 13G). Tumors were allowed to establish, and were then isolated from mice, dissociated, and CD8+ T and NK cell infiltrates were assessed by flow cytometry. In all three tumor types, HVEM was found to be much more widely expressed on both CD8+ T and NK cells than DNAM-1, consistent with the human PBMC findings (FIG. 13G). Additionally, HVEM was found to be expressed at high levels on T and NK cells isolated from mouse splenocytes (FIG. 23C). Together these results provided rationale for the exploration of a therapeutic candidate able to provide co-stimulatory signaling through HVEM and LTβR.

To evaluate the effects of the TIGIT-Fc-LIGHT chimeric protein on immune cells, human PBMC were cultured with IgG1 or IgG4 variants of the TIGIT-Fc-LIGHT chimeric protein in AIMV media. Single cell RNA-seq was performed after 2 days of the culture and analyzed with the hi-dimensionality reduction algorithm Uniform Manifold Approximation and Projection (UMAP). As shown in FIG. 14F, both IgG1 or IgG4 variants of the TIGIT-Fc-LIGHT chimeric protein produced similar changes compared to untreated cells. The pathways associated with the TIGIT-Fc-LIGHT chimeric protein induced differentially expressed genes (DEGs) were identified by protein analysis through evolutionary relationships (PANTHER). As shown in FIG. 14G, 106 DEGs associated with myeloid cell functions were up-regulated in PBMCs treated with the TIGIT-Fc-LIGHT chimeric protein compared to untreated cells. 130 DEGs associated with myeloid cell functions were down-regulated in PBMCs treated with the TIGIT-Fc-LIGHT chimeric protein compared to untreated cells (FIG. 14G). 47 DEGs associated with NK cell functions were up-regulated in PBMCs treated with the TIGIT-Fc-LIGHT chimeric protein compared to untreated cells (FIG. 14G). 17 DEGs associated with NK cell functions were down-regulated in PBMCs treated with the TIGIT-Fc-LIGHT chimeric protein compared to untreated cells (FIG. 14G). 37 DEGs associated with CD8+ T cell functions were up-regulated in PBMCs treated with the TIGIT-Fc-LIGHT chimeric protein compared to untreated cells (FIG. 14G). 12 DEGs associated with CD8+ T cell functions were down-regulated in PBMCs treated with the TIGIT-Fc-LIGHT chimeric protein compared to untreated cells (FIG. 14G). These results indicated, inter alia, that the TIGIT-Fc-LIGHT chimeric protein affected the function of cell populations that corresponded to myeloid, CD8+ T, or NK cells. similar results were obtained using SingleR and ImmuneExp (FIG. 13D).

After 2 days of the culture of PBMCs with the TIGIT-Fc-LIGHT chimeric protein, a number of cytokines were assayed using the Meso Scale Discovery (MSD) ELISA assays. As shown in FIG. 14C, the TIGIT-Fc-LIGHT chimeric protein (both versions comprising Fc domain from IgG1 or IgG4) significantly induced IFNγ, IL-8, IL-IL-12/p70, and SDF1a (CXCL12). Collectively, these results suggest, inter alia, that the TIGIT-Fc-LIGHT chimeric protein stimulated the myeloid, CD8+ T, or NK cell populations and induced those cells to produce cytokines.

Jurkat effector cells from a commercially available PVR:DNAM1 reporter assay were assayed for the expression of HVEM. As shown in FIG. 14C (upper panel), these Jurkat effector cells also expressed human HVEM. These cells were incubated with an anti-IgG4 control, an anti-DNAM-1 antibody, Fc-LIGHT single sided protein, or the TIGIT-Fc-LIGHT chimeric protein (with or without LIGHT blockade). Interestingly, the background bioluminiscence of these cells was inhibited by the anti-DNAM-1 antibody (FIG. 14C), indicating a role for DNAM-1 costimulation. As shown in FIG. 14C (lower panel), the Jurkat effector cells showed activation as assayed by bioluminescence when incubated with LIGHT-Fc protein or the TIGIT-Fc-LIGHT chimeric protein. The TIGIT-Fc-LIGHT chimeric protein produced more bioluminescence compared to Fc-LIGHT protein (FIG. 14C). However, the bioluminescence produced by the TIGIT-Fc-LIGHT chimeric protein was blocked by LIGHT antibody blockade (FIG. 14C). These data suggested, without wishing to be bound by theory, that the TIGIT-Fc-LIGHT chimeric protein, inter alia, bypassed the need for DNAM-1 co-stimulation and directly activated downstream signaling via HVEM.

To evaluate the effect of the anti-PD-1, the anti-PD-L1 and the TIGIT-Fc-LIGHT chimeric protein on innate and adaptive immunity was studied next. Briefly, mice were inoculated in rear flank with colorectal tumor CT26 cells or CT26 anti-PD-1 resistant cells. The mice were randomly divided in the following treatment groups: (1) vehicle-alone, (2) 100 μg per dose of an anti-PD-1 antibody (clone RMP1-14), (3) 100 μg per dose of an anti-PD-L1 antibody (clone 10F.9G2), (4) 200 μg per dose of the TIGIT-Fc-LIGHT chimeric protein, an anti-PD-L1 antibody, (5) 100 μg per dose of the anti-PD-1 antibody+200 μg per dose of the TIGIT-Fc-LIGHT chimeric protein, and (6) 100 μg per dose of the anti-PD-L1 antibody+200 μg per dose of the TIGIT-Fc-LIGHT chimeric protein. Mice were dosed on days 0, 3, and 6 following tumor inoculation via intraperitoneal injections. proportion of antigen-specific CD8+ T cells (AH1+) and NK cells were determined across treatment groups.

As shown in FIG. 12G (top left panel), the treatment of wild type CT26 tumors with each of the treatments significantly increased AH1+CD8+ T cells (p<0.05) compared to vehicle treated mice. The combination treatment of wild type CT26 tumors with a combination of the TIGIT-Fc-LIGHT chimeric protein and the anti-PD-1 antibody or the anti-PD-L1 antibody further increased the amount of AH1+ CD8+ T cells compared to single treatments (FIG. 12G, top left panel). Further, as shown in FIG. 12G (bottom left panel), the treatment of wild type CT26 tumors with the TIGIT-Fc-LIGHT chimeric protein, but not the anti-PD-1 antibody or the anti-PD-L1 antibody, significantly increased NKP46+ NK cells compared to vehicle treated mice (p<0.0001). The treatment of wild type CT26 tumors with a combination of the TIGIT-Fc-LIGHT chimeric protein and the anti-PD-1 antibody or the anti-PD-L1 antibody also significantly increased the amount of NKP46+ NK cells compared to vehicle treated mice (FIG. 12G, bottom left panel).

The treatment of CT26 anti-PD-1 resistant cell tumors with the anti-PD-1 antibody did not significantly increase the proportion of either AH1+CD8+ T cells (FIG. 12G, top right panel) or NKP46+ NK cells (FIG. 12G, bottom right panel). The treatment of CT26 anti-PD-1 resistant cell tumors with a combination of the TIGIT-Fc-LIGHT chimeric protein and the anti-PD-L1 antibody significantly increased the proportion of AH1+ CD8+ T cells (FIG. 12G, top right panel). Moreover, the treatment of CT26 anti-PD-1 resistant cell tumors with the TIGIT-Fc-LIGHT chimeric protein, either alone, or in combination with the anti-PD-L1 antibody significantly increased the proportion of NKP46+ NK cells (FIG. 12G, bottom right panel).

These results indicate, inter alia, that the TIGIT-Fc-LIGHT chimeric protein activates both innate and adaptive immunity. Without being bound by theory, these results suggest, inter alia, that the activity of the TIGIT-Fc-LIGHT chimeric protein in the anti-PD-1 acquired resistance CT26 model may be uniquely driven by its ability to activate both an innate and adaptive anti-tumor immune response and promote the infiltration of both NK cells and antigen-specific CD8+ T cells.

To evaluate a role of various types of immune cells on the anti-tumor activity of the TIGIT-Fc-LIGHT chimeric protein, tumor bearing mice were depleted of CD4+ T cells, CD8+ cells, or NK cells, prior to treatment with TIGIT-Fc-LIGHT. Briefly, mice were inoculated in the rear flank with B16.F10 tumors. The mice were randomly divided in the following treatment groups: (1) vehicle-alone, (2) 200 μg per dose of the TIGIT-Fc-LIGHT chimeric protein (no depletion), (3) 200 μg per dose of the TIGIT-Fc-LIGHT chimeric protein+CD4+ T cell depletion (−CD4), (4) 200 μg per dose of the TIGIT-Fc-LIGHT chimeric protein+CD8+ T cell depletion (−CD8), and (5) 200 μg per dose of the TIGIT-Fc-LIGHT chimeric protein +NK cell depletion (−NK). Mice were treated with antibodies that deplete the indicated cells. dosed on days 0, 3, and 6 following tumor inoculation via intraperitoneal injections. Tumor sizes were measured on day 11 post-tumor inoculation. Tumor growth inhibition compared to vehicle alone-treated mice was calculated and plotted. As shown in FIG. 12H, compared to the vehicle-treated controls, the no depletion control, −CD4 and −CD8 groups showed significant tumor reduction. Compared to the no depletion mice, the −CD8 mice and −NK mice (statistically significant) showed a reduced anti-cancer response. CD4− mice had a smaller impact, if any. These data indicate, inter alia, that CD8+ and NK cells play a role in anti-tumor response produced by the TIGIT-Fc-LIGHT chimeric protein.

The tumor infiltrating lymphocytes were then analyzed. Briefly, mice were inoculated in rear flank with colorectal tumor CT26 cells or CT26 anti-PD-1 resistant cells. The mice were randomly divided in the following treatment groups: (1) vehicle-alone, (2) 100 μg per dose of an anti-PD-1 antibody (clone RMP1-14), (3) 100 μg per dose of an anti-PD-L1 antibody (clone 10F.9G2), (4) 200 μg per dose of the TIGIT-Fc-LIGHT chimeric protein, (5) 100 μg per dose of the anti-PD-1 antibody+200 μg per dose of the TIGIT-Fc-LIGHT chimeric protein, and (6) 100 μg per dose of the anti-PD-L1 antibody+200 μg per dose of the TIGIT-Fc-LIGHT chimeric protein. Mice were dosed on days 0, 3, and 6 following tumor inoculation via intraperitoneal injections. The proportion of antigen-specific CD8+ T cells (AH1+) and NK cells were determined across treatment groups. As shown in FIG. 12G (top left panel), the CT26 tumors accumulated increased numbers of CD8+ T cells (AH1 tetramer-F) when treated with each of the agents. The CT26 tumors accumulated increased numbers of CD8+ T cells (AH1 tetramer+) when treated with the TIGIT-Fc-LIGHT chimeric protein, which were further enhanced by the co-treatment with the anti-PD-L1 antibody, which by itself showed a modest increase in CD8+ T cells (AH1 tetramer-F) (FIG. 12G (top right panel)). As shown in FIG. 12G (bottom left panel), the CT26 tumors accumulated increased numbers of NK cells when treated with each of the agents. The CT26 tumors accumulated increased numbers of NK cells when treated with the TIGIT-Fc-LIGHT chimeric protein, which were further enhanced by the co-treatment with the anti-PD-L1 antibody, which by itself showed a modest increase in NK cells (FIG. 12G (top right panel)).

### Example 13: Efficacy of the TIGIT-Fc-LIGHT Chimeric Protein Against the Anti-PD-1 Resistant Cell Lines and a Primarily Resistant Anti-PD-1 Resistant Cell Line in Comparison with anti-TIGIT and/or anti-PD-1 Antibodies

Briefly, mice were inoculated in rear flank with colorectal tumor CT26 cells, CT26 anti-PD-1 resistant cells (acquired resistance), or B16.F10 melanoma tumors (primarily resistant). The mice were randomly divided in the following treatment groups: (1) vehicle-alone, (2) 100 μg per dose of an anti-TIGIT antibody (clone 1G9), (3) 100 μg per dose of an anti-PD-1 antibody (clone RMP1-14), (4) 100 μg per dose of each of the anti-PD-1 antibody+the anti-TIGIT antibody, (5) 200 μg per dose of the TIGIT-Fc-LIGHT chimeric protein, an anti-PD-1 antibody, and (6) 100 μg per dose of the anti-PD-1 antibody+200 μg per dose of the TIGIT-Fc-LIGHT chimeric protein. Mice were dosed on days 0, 3, and 6 following tumor inoculation via intraperitoneal injections. Tumor sizes were measured on day 14 post-tumor inoculation. Tumor volumes were measured and plotted.

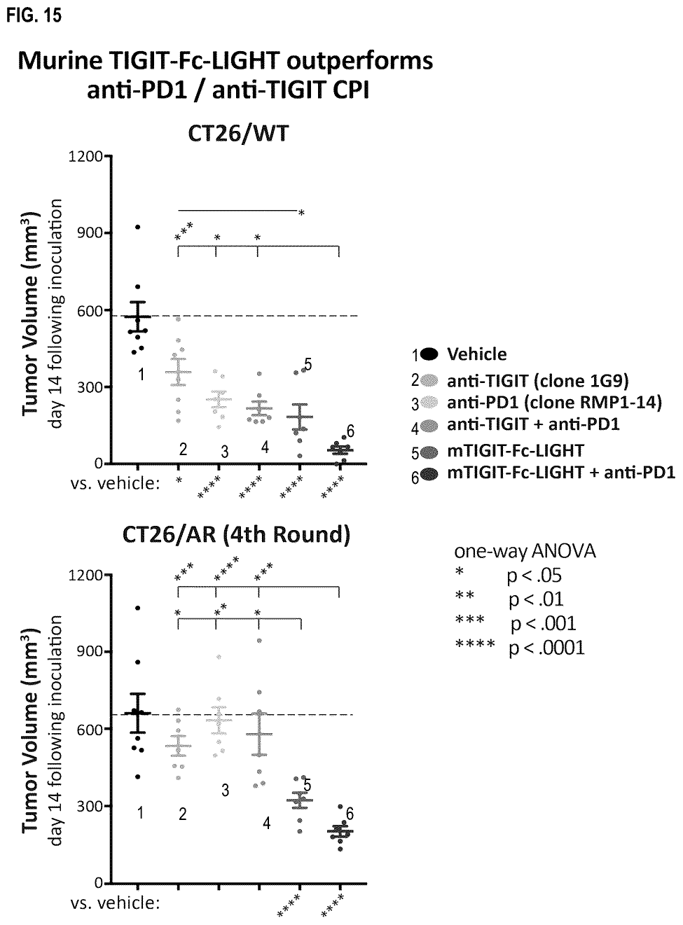

As shown in FIG. 15 (top panel), the treatment with the anti-TIGIT antibody alone significantly decreased tumor size in mice harboring CT26 tumors compared to vehicle alone treated mice (p<0.05). The treatment with the anti-PD-1 antibody, a combination of the anti-TIGIT and the anti-PD-1 antibodies, the TIGIT-Fc-LIGHT chimeric protein, and the combination of the TIGIT-Fc-LIGHT chimeric protein and the anti-PD-1 antibody significantly decreased tumor size in mice harboring CT26 tumors compared to vehicle alone treated mice (p<0.0001) (FIG. 15, top panel). Interestingly, the treatment with the TIGIT-Fc-LIGHT chimeric protein significantly decreased tumor size in mice harboring CT26 tumors compared to the mice treated with the anti-TIGIT antibody alone (p<0.05) (FIG. 15, top panel). Further, the treatment with the combination of the anti-PD-1 antibody and the TIGIT-Fc-LIGHT chimeric protein significantly decreased tumor size in mice harboring CT26 tumors compared to the mice treated with the anti-TIGIT antibody alone (p<0.001), the anti-PD-1 antibody alone (p<0.05), or the combination of the anti-TIGIT antibody and the anti-PD-1 antibody alone (p<0.05) (FIG. 15, top panel).

As shown in FIG. 15 (bottom panel), the treatment with the anti-TIGIT antibody alone, the anti-PD-1 antibody alone, or a combination of the anti-TIGIT and the anti-PD-1 antibodies did not significantly decrease tumor size in mice harboring CT26 anti-PD-1 resistant cell tumors compared to vehicle alone treated mice. on the other hand, the treatment with the TIGIT-Fc-LIGHT chimeric protein, and the combination of the TIGIT-Fc-LIGHT chimeric protein and the anti-PD-1 antibody significantly decreased tumor size in mice harboring CT26 anti-PD-1 resistant cell tumors compared to vehicle alone treated mice (p<0.0001) (FIG. 15, bottom panel). Interestingly, the treatment with the TIGIT-Fc-LIGHT chimeric protein significantly decreased tumor size in mice harboring CT26 anti-PD-1 resistant cell tumors compared to the mice treated with the anti-TIGIT antibody alone (p<0.05), the anti-PD-1 antibody alone (p<0.01), or a combination of the anti-TIGIT and the anti-PD-1 antibodies (p<0.05) (FIG. 15, bottom panel). Further, the treatment with the combination of the anti-PD-1 antibody and the TIGIT-Fc-LIGHT chimeric protein significantly decreased tumor size in mice harboring CT26 anti-PD-1 resistant cell tumors compared to the mice treated with the anti-TIGIT antibody alone (p<0.001), the anti-PD-1 antibody alone (p<0.001), or a combination of the anti-TIGIT and the anti-PD-1 antibodies (p<0.001) (FIG. 15, bottom panel).

These results suggest, inter alia, that the TIGIT-Fc-LIGHT is highly active in both WT CT26 and acquired resistance CT26 tumor models, and outperforms checkpoint blockade of anti-TIGIT, anti-PD-1, anti-PD-L1, or the combination of anti-TIGIT/anti-PD-1.

### Example 14: Construction and Characterization of an Illustrative Human SIRPα-Fc-4-1BBL Chimeric Protein

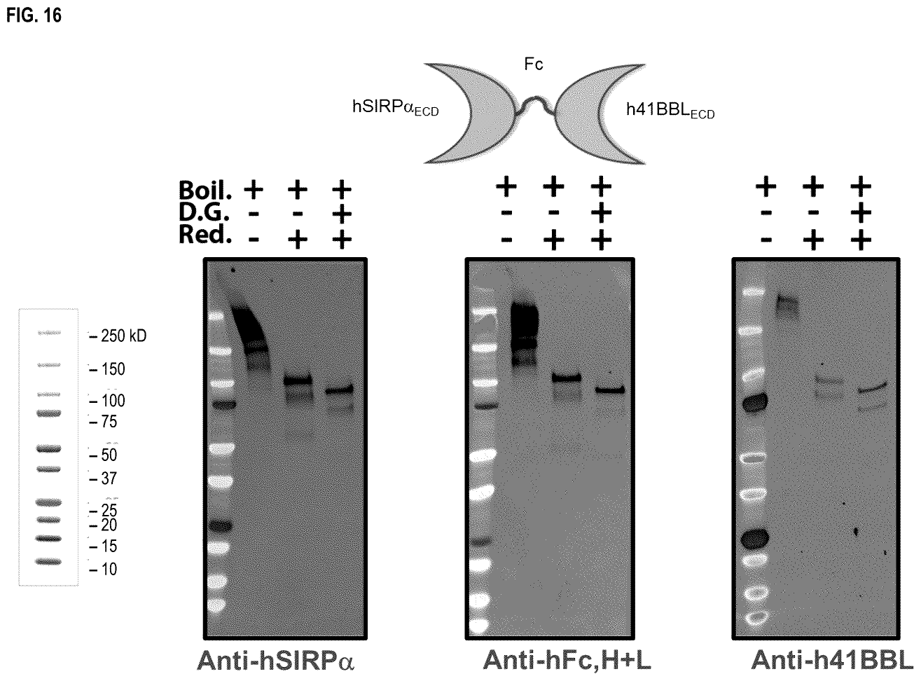

A construct encoding a SIRPα- and 4-1BBL-based chimeric protein was generated. The “hSIRPα-Fc-4-1BBL” construct included an extracellular domain (ECD) of human SIRPα fused to an ECD of human 4-1BBL via a hinge-CH2-CH3 Fc domain. See, FIG. 16 (top panel).

The construct was codon optimized for expression in Chinese Hamster Ovary (CHO) cells, transfected into CHO cells and individual clones were selected for high expression. High expressing clones were then used for small-scale manufacturing in stirred bioreactors in serum-free media and the relevant chimeric fusion proteins were purified with Protein A binding resin columns.

The hSIRPα-Fc-4-1BBL construct was transiently expressed in 293 cells and purified using protein-A affinity chromatography. To understand the native structure of the SIRPα-Fc-4-1BBL chimeric protein, untreated denatured samples (i.e., boiled in the presence of SDS, without a treatment with a reducing agent or a deglycosylation agent) were compared with (i) reduced samples, which were not deglycosylated (i.e. treated only with β-mercaptoethanol, and boiled in the presence of SDS); and (ii) reduced and deglycosylated samples (i.e. treated both with β-mercaptoethanol and a deglycosylation agent, and boiled in the presence of SDS). In addition, to confirm the presence of each domain of the SIRPα-Fc-4-1BBL chimeric protein, the gels were run in triplicates and probed using an anti-SIRPα antibody (FIG. 16, bottom panel, left blot), an anti-human Fc (H+L) antibody (FIG. 16, center blot), or an anti-4-1BBL antibody (FIG. 16, bottom panel, right blot). The western blots indicated the presence of a dominant dimer band in the non-reduced lanes (FIG. 16, bottom panel, second lane in each blot), which was reduced to a glycosylated monomeric band in the presence of the reducing agent, β-mercaptoethanol (FIG. 16, bottom panel, third lane in each blot). As shown in FIG. 16, bottom panel, fourth lane in each blot, the chimeric protein ran as a monomer in the presence of both a reducing agent (β-mercaptoethanol) and a deglycosylation agent. These results demonstrate that the human SIRPα-Fc-4-1BBL chimeric protein is glycosylated and has a tendency to form a multimeric the native state.

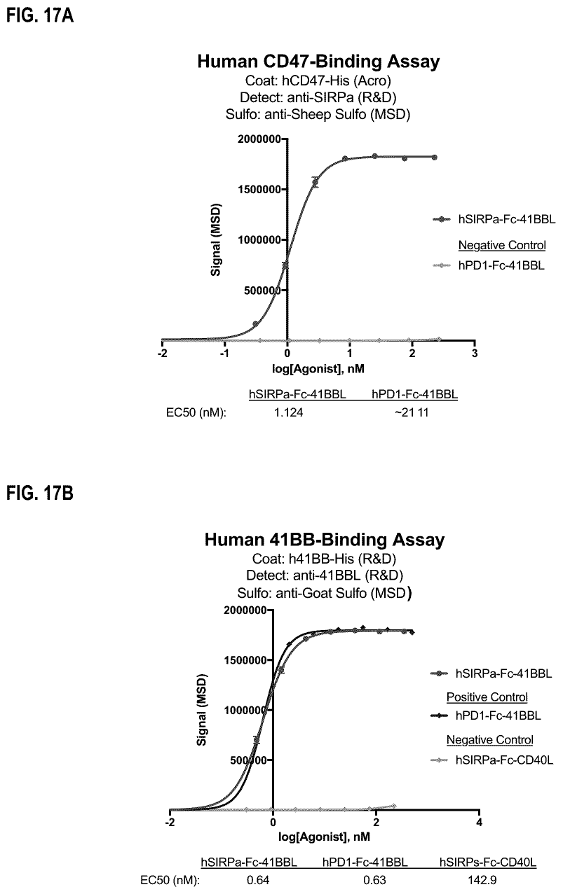

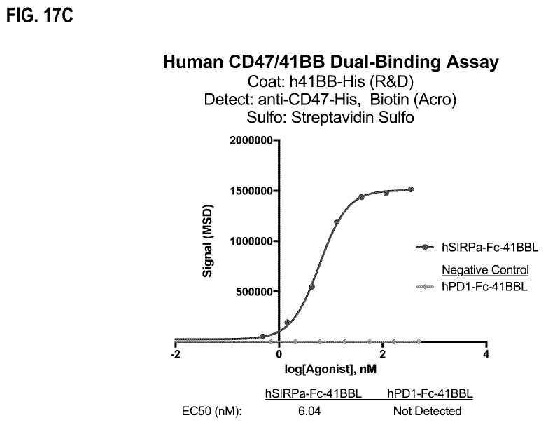

The binding of hSIRPα-Fc-4-1BBL to ligands of the two ends, human CD47, human 4-1BB, was evaluated using the Meso Scale Discovery (MSD) ELISA assays. Toward that, human CD47-His was coated on plates and increasing amounts of the hSIRPα-Fc-4-1BBL or PD-1-Fc-4-1BBL chimeric proteins were added to the plates for capture by the plate-bound human CD47-His. The PD-1-Fc-4-1BBL chimeric protein was used as a negative control for binding to human CD47. Binding of the chimeric proteins to CD47-His was detected using an anti-SIRPα antibody and an anti-sheep SULFO-TAG secondary antibody. Binding was assayed using an electrochemiluminescence (ECL) readout. As shown in FIG. 17A, each of the hSIRPα-Fc-4-1BBL chimeric protein bound to human CD47-His in a dose-dependent and saturable manner. In contrast, the PD-1-Fc-4-1BBL chimeric protein did not bind to human CD47-His. These data indicate that the hSIRPα-Fc-4-1BBL chimeric protein specifically binds to human CD47, the ligand of SIRPα.

In a similar experiment, human 4-1BB-His was coated on plates and increasing amounts of the hSIRPα-Fc-4-1BBL, PD-1-Fc-4-1BBL or hSIRPα-Fc-CD40L chimeric proteins were added to the plates for capture by the plate-bound human 4-1BB-His. The hSIRPα-Fc-CD40L chimeric protein was used as a negative control for binding to human 4-1BB. Binding of the chimeric proteins to 4-1BB-His was detected using an anti-4-1BBL antibody and an anti-sheep SULFO-TAG secondary antibody. Binding was assayed using an electrochemiluminescence (ECL) readout. As shown in FIG. 17B, each of the hSIRPα-Fc-4-1BBL and the PD-1-Fc-4-1BBL chimeric proteins bound to human 4-1BB-His in a dose-dependent and saturable manner. In contrast, the hSIRPα-Fc-CD40L chimeric protein did not bind to human 4-1BB-His. These data indicate that the hSIRPα-Fc-CD40L chimeric protein specifically binds to human 4-1BB, the ligand of 4-1BBL.

To understand whether the human SIRPα-Fc-4-1BBL chimeric protein can contemporaneously bind to human 4-1BB and CD47, human 4-1BB-His was coated on plates and increasing amounts of the hSIRPα-Fc-4-1BBL, or PD-1-Fc-4-1BBL chimeric proteins were added to the plates for capture by the plate-bound human 4-1BB-His. The PD-1-Fc-4-1BBL chimeric protein was used as a negative control. Then, human CD47-His-Biotin was added to the plate for capture by any chimeric protein, which was in turn captured by the human 4-1BB. Streptavidin SULFO-TAG secondary was used for detection. Binding was assayed using an electrochemiluminescence (ECL) readout. As shown in FIG. 17C, the hSIRPα-Fc-4-1BBL chimeric protein produced a signal consistent with contemporaneous, dose-dependent binding to human 4-1BB-His and CD47-His. In contrast, the PD-1-Fc-4-1BBL chimeric protein did not produce a signal. These data indicate that the hSIRPα-Fc-4-1BBL chimeric protein can contemporaneously bind to human 4-1BB and CD47.

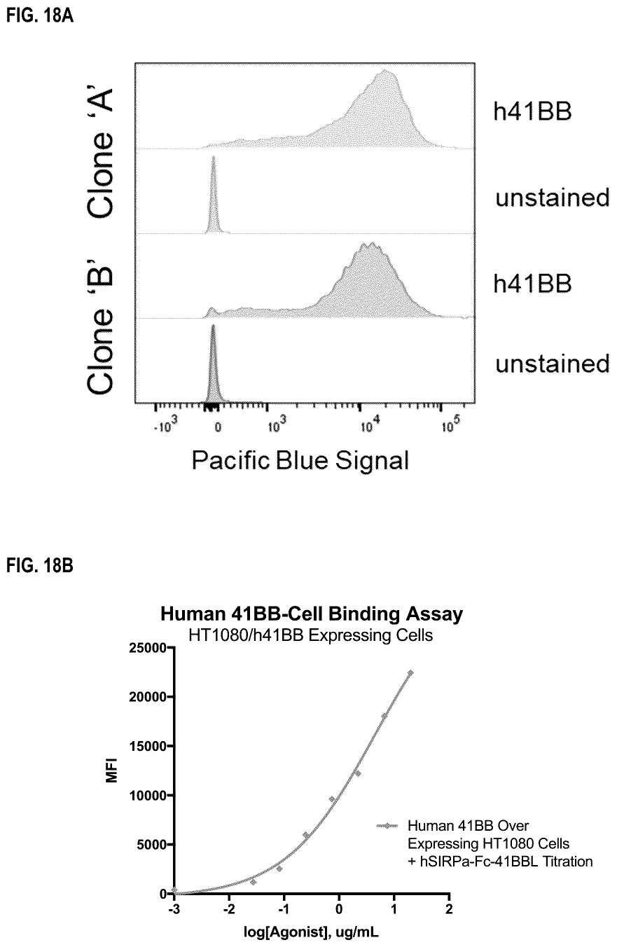

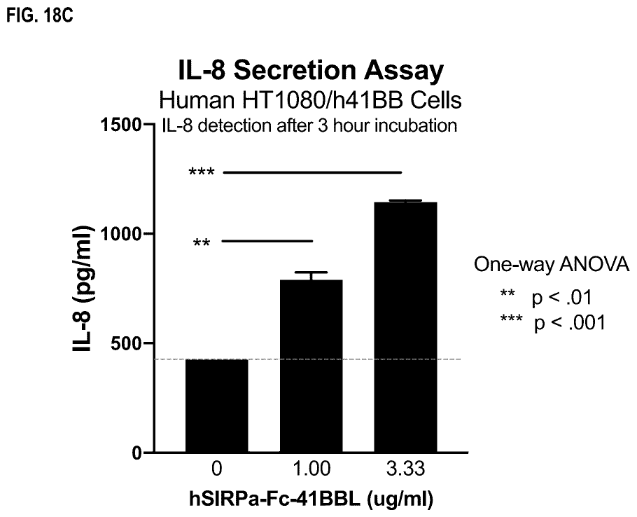

To further study binding of the SIRPα-Fc-4-1BBL chimeric protein to cells expressing 4-1BB, human fibrosarcoma HT1080 cells expressing 4-1BB (the HT1080/4-1BB cells) were generated using standard techniques. Two positive clones, HT1080/4-16B+ clones A and B were used for binding studies. To determine whether the SIRPα-Fc-4-1BBL chimeric protein can specifically bind the HT1080/4-1BB cells, a flow-cytometry-based assay was carried out. The HT1080/4-1BB cells were incubated with buffer alone or the SIRPα-Fc-4-1BBL chimeric protein. The bound cells were counterstained with an anti-mouse Fc antibody conjugated to Pacific Blue, and the binding of SIRPα-Fc-4-1BBL binding was detected by flow cytometry. As shown in FIG. 18A, the HT1080/4-1BB cells stained with the SIRPα-Fc-4-1BBL chimeric protein displayed more staining compared to the unstained HT1080/4-1BB cells. These data also demonstrated binding of the SIRPα-Fc-4-1BBL chimeric protein to both closes of the HT1080/4-1BB cells.

To quantitate binding, the HT1080/4-1BB cells were incubated with increasing concentrations of the SIRPα-Fc-4-1BBL chimeric protein. The bound cells were counterstained with an anti-mouse Fc antibody conjugated to Pacific Blue, and the binding of the SIRPα-Fc-4-1BBL chimeric protein binding was detected by flow cytometry. The mean fluorescent intensity (MFI) was plotted as a function of log concentration of the SIRPα-Fc-4-1BBL chimeric protein. As shown in FIG. 18B, the SIRPα-Fc-4-1BBL chimeric protein displayed a dose-dependent binding to the HT1080/4-1BB cells.

Whether the SIRPα-Fc-4-1BBL chimeric protein can activate 4-1BB/4-1BBL signaling was studied next. The HT1080/h4-1BB cells, which secrete IL-8 upon activation of 4-1BB/4-1BBL signaling, were used as a model. The HT1080/h4-1BB cells were grown overnight, and buffer alone, 1 μg/ml, or 3.33 μg/ml the SIRPα-Fc-4-1BBL chimeric protein was added. Incubation was continued for an additional 3 hours. After 3 hours, media were removed from the HT1080/h4-1BB cell culture and IL-8 was assessed by ELISA. IL-8 production was evaluated in the culture supernatant using Meso Scale Discovery (MSD) ELISA assays. As shown in FIG. 18C, compared to the buffer-only-treated HT1080/h4-1BB cells, significantly more IL-8 production was observed in HT1080/h4-1BB cells treated with 1 μg/ml (p<0.01), or 3.33 μg/ml (p<0.001) of the SIRPα-Fc-4-1BBL chimeric protein.

These results indicated, inter alia, that the SIRPα-Fc-4-1BBL chimeric protein activates 4-1BB/4-1BBL signaling.

### Example 15: Construction and Characterization of an Illustrative Mouse SIRPα-Fc-4-1BBL Chimeric Protein

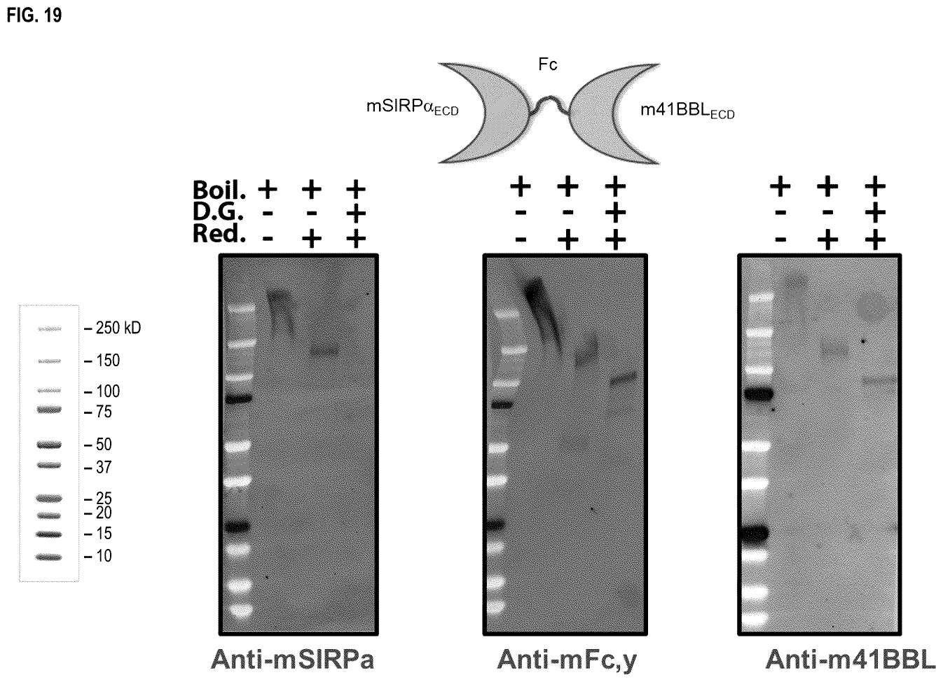

A construct encoding a mouse SIRPα- and 4-1BBL-based chimeric protein was generated for in vivo studies in mouse models. The “mSIRPα-Fc-4-1BBL” construct included an extracellular domain (ECD) of mouse SIRPα fused to an ECD of mouse 4-1BBL via a hinge-CH2-CH3 Fc domain. See, FIG. 19 (top panel).

The construct was codon optimized for expression in Chinese Hamster Ovary (CHO) cells, transfected into CHO cells and individual clones were selected for high expression. High expressing clones were then used for small-scale manufacturing in stirred bioreactors in serum-free media and the relevant chimeric fusion proteins were purified with Protein A binding resin columns.

The mSIRPα-Fc-4-1BBL construct was transiently expressed in 293 cells and purified using protein-A affinity chromatography. To understand the native structure of the SIRPα-Fc-4-1BBL chimeric protein, untreated denatured samples (i.e., boiled in the presence of SDS, without a treatment with a reducing agent or a deglycosylation agent) were compared with (i) reduced samples, which were not deglycosylated (i.e. treated only with β-mercaptoethanol, and boiled in the presence of SDS); and (ii) reduced and deglycosylated samples (i.e. treated both with β-mercaptoethanol and a deglycosylation agent, and boiled in the presence of SDS). In addition, to confirm the presence of each domain of the SIRPα-Fc-4-1BBL chimeric protein, the gels were run in triplicates and probed using an anti-SIRPα antibody (FIG. 19, bottom panel, left blot), an anti-mouse Fc antibody (FIG. 19, center blot), or an anti-4-1BBL antibody (FIG. 19, bottom panel, right blot). The western blots indicated the presence of a dominant dimer band in the non-reduced lanes (FIG. 19, bottom panel, second lane in each blot), which was reduced to a glycosylated monomeric band in the presence of the reducing agent, β-mercaptoethanol (FIG. 19, bottom panel, third lane in each blot). As shown in FIG. 19, bottom panel, fourth lane in each blot, the chimeric protein ran as a monomer in the presence of both a reducing agent (β-mercaptoethanol) and a deglycosylation agent. These results demonstrate that the mSIRPα-Fc-4-1BBL chimeric protein is glycosylated and has a tendency to form a multimeric the native state.

The binding of mSIRPα-Fc-4-1BBL to ligands of the three domains, mouse CD47, anti-Fc antibody, and mouse 4-1BB, was evaluated using the Meso Scale Discovery (MSD) ELISA assays.

An anti-mouse Fc-y antibody was coated on a plate and increasing amounts of the mSIRPα-Fc-4-1BBL chimeric protein was added to the plate for capture by the plate-bound mouse the anti-mouse Fc-y antibody. Binding of the chimeric proteins to the anti-mouse Fc-y antibody was detected using an anti-mouse-SULFO-TAG antibody. Binding was assayed using an electrochemiluminescence (ECL) readout. As shown in FIG. each of the mSIRPα-Fc-4-1BBL chimeric protein bound to mouse the anti-mouse Fc-y antibody in a dose-dependent. These data indicate that the mSIRPα-Fc-4-1BBL chimeric protein specifically binds to mouse the anti-mouse Fc-y antibody, the ligand of Fc domain.

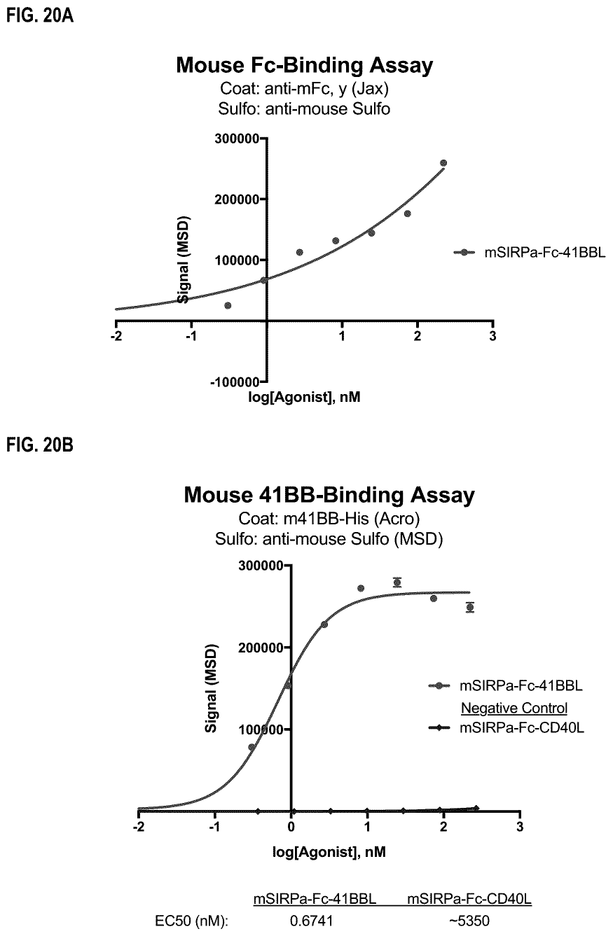

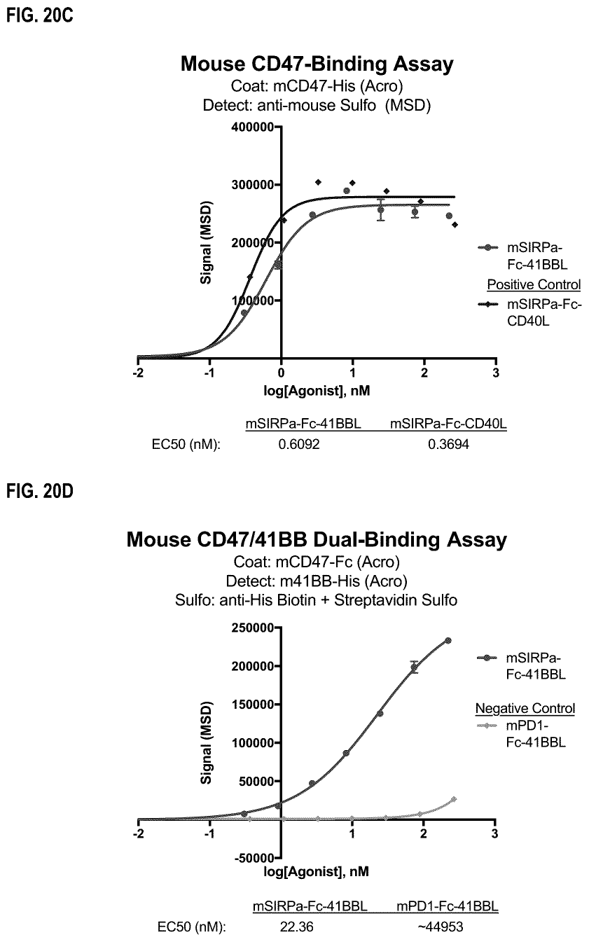

Mouse 4-1BB-His was coated on plates and increasing amounts of the mSIRPα-Fc-4-1BBL, or mSIRPα-Fc-CD40L chimeric proteins were added to the plates for capture by the plate-bound mouse 4-1BB-His. The mSIRPα-Fc-CD40L chimeric protein was used as a negative control for binding to mouse 4-1BB. Binding of the chimeric proteins to 4-1BB-His was detected using an anti-mouse-SULFO-TAG antibody. Binding was assayed using an electrochemiluminescence (ECL) readout. As shown in FIG. 20B, the mSIRPα-Fc-4-1BBL chimeric protein bound to mouse 4-1BB-His in a dose-dependent and saturable manner. In contrast, the mSIRPα-Fc-CD40L chimeric protein did not bind to mouse 4-1BB-His. These data indicate that the mSIRPα-Fc-CD40L chimeric protein specifically binds to mouse 4-1BB, the ligand of 4-1BBL.

Mouse CD47-His was coated on plates and increasing amounts of the mSIRPα-Fc-4-1BBL or mSIRPα-Fc-CD40L chimeric proteins were added to the plates for capture by the plate-bound mouse CD47-His. The mSIRPα-Fc-CD40L chimeric protein was used as a positive control for binding to mouse CD47. Binding of the chimeric proteins to CD47-His was detected using an anti-mouse SULFO-TAG antibody. Binding was assayed using an electrochemiluminescence (ECL) readout. As shown in FIG. 20C, each of the mSIRPα-Fc-4-1BBL and mSIRPα-Fc-CD40L chimeric proteins bound to mouse CD47-His in a dose-dependent and saturable manner, and with a comparable kinetics. These data indicate that the mSIRPα-Fc-4-1BBL chimeric protein specifically binds to mouse CD47, the ligand of SIRPα.

To understand whether the mouse SIRPα-Fc-4-1BBL chimeric protein can contemporaneously bind to mouse 4-1BB and CD47, mouse CD47-Fc was coated on plates and increasing amounts of the mSIRPα-Fc-4-1BBL, or PD-1-Fc-4-1BBL chimeric proteins were added to the plates for capture by the plate-bound mouse CD47-Fc. The PD-1-Fc-4-1BBL chimeric protein was used as a negative control. Then, mouse 4-1BB-His was added to the plate for capture by any chimeric protein, which was captured in turn by the mouse CD47-Fc protein. An anti-His-biotin and streptavidin-SULFO-TAG were used for detection. Binding was assayed using an electrochemiluminescence (ECL) readout. As shown in FIG. 20D, the mSIRPα-Fc-4-1BBL chimeric protein produced a signal consistent with contemporaneous, dose-dependent binding to mouse 4-1BB-His and CD47-Fc. In contrast, the PD-1-Fc-4-1BBL chimeric protein did not produce a signal. These data indicate that the mSIRPα-Fc-4-1BBL chimeric protein can contemporaneously bind to mouse 4-1BB and CD47.

### Example 16: Efficacy of the SIRPα-Fc-4-1BBL Chimeric Protein Against the Anti-PD-1 Sensitive, and Anti-PD-1 Resistant Tumors

Balb/c mice were inoculated in rear flank with 500,000 colorectal tumor CT26 cells or 4th generation anti-PD-1 resistant cells. When the mean starting tumor volume reached approximately 90 mm³ (day 0), the mice were randomly divided in the following treatment groups: (1) vehicle-alone (PBS, n=8), (2) 100 μg per dose of an anti-PD-1 antibody (clone RMP1-14, n=7), and (6) 200 μg per dose of the SIRPα-Fc-4-1BBL chimeric protein (n=8). Mice were dosed on days 0, 3, and 6 via intraperitoneal injections.

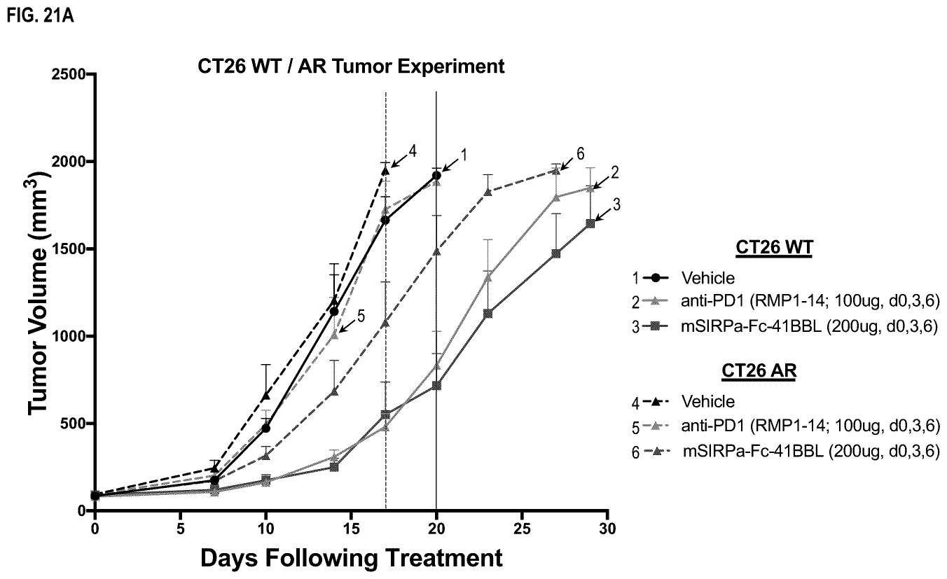

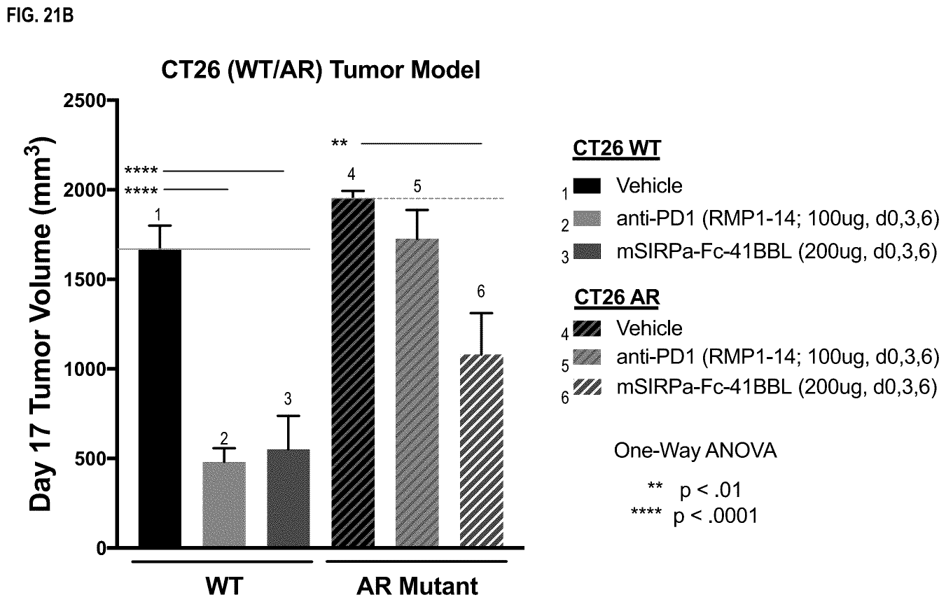

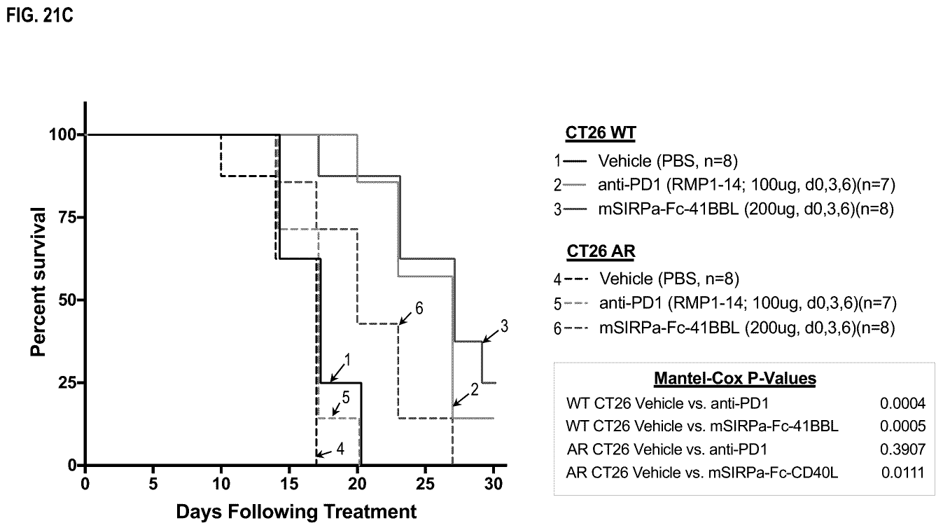

Tumor volumes for mice bearing wild type CT26 allografts (solid lines in FIG. 21A) were measured and plotted as a function of time after treatments were initiated. As shown in FIG. 21A, the treatment with either the anti-PD-1 antibody or the SIRPα-Fc-4-1BBL chimeric protein led to an inhibition of CT26 (wild type) tumor growth compared to vehicle-alone treated mice (compare curves 2 and 3 in FIG. 21A with curve 1). The efficacy of the SIRPα-Fc-4-1 BBL chimeric protein appeared to be similar to that of anti-PD-1 antibody. Tumor sizes were measured on day 17 post-tumor inoculation and plotted. As shown in FIG. 21B, mice bearing wild type CT26 tumors showed a statistically significant reduction in tumor sizes following the treatment with the anti-PD-1 antibody (p<0.0001) or the SIRPα-Fc-4-1BBL chimeric protein (p<0.0001) compared to vehicle-alone treated mice. A Kaplan-Meier survival curve was plotted for mice bearing wild type CT26 allografts (solid lines in FIG. 21C). As shown in FIG. 21C, all mice bearing wild type CT26 tumors died by day 21, but the mice bearing wild type CT26 allografts showed improved survival following the treatment with the anti-PD-1 antibody (Mantel-Cox p value 0.0004) or the SIRPα-Fc-4-1BBL chimeric protein (Mantel-Cox p value 0.0005).

Tumor volumes for mice bearing 4th generation anti-PD-1 resistant cell allografts (dotted lines in FIG. 21A) were measured and plotted as a function of time after treatments were initiated. As shown in FIG. 21A, the SIRPα-Fc-4-1BBL chimeric protein led to an inhibition of 4th generation anti-PD-1 resistant cell tumor growth compared to vehicle-alone treated mice (compare curve 6 in FIG. 21A with curve 4). On the other hand, the treatment with the anti-PD-1 antibody had a slight effect on 4th generation anti-PD-1 resistant cell allograft tumor sizes (compare curve 5 in FIG. 21A with curve 4). Tumor sizes were measured on day 17 post-tumor inoculation and plotted (see patterned bars in FIG. 21B). As shown in FIG. 21B, mice bearing wild type 4th generation anti-PD-1 resistant tumors showed a statistically significant reduction in tumor size following the treatment with the SIRPα-Fc-4-1BBL chimeric protein (p<0.01) compared to vehicle-alone treated mice. On the other hand, the treatment of the mice bearing 4th generation anti-PD-1 resistant cell allografts with the anti-PD-1 antibody had a slight effect on tumor size (FIG. 21B). A Kaplan-Meier survival curve was plotted for mice bearing 4th generation anti-PD-1 resistant cell allografts (dotted lines in FIG. 21C). As shown in FIG. 21C, all mice bearing wild type 4th generation anti-PD-1 resistant tumors died by day 18, but the mice bearing 4th generation anti-PD-1 resistant cell allografts showed improved survival following the treatment with the SIRPα-Fc-4-1BBL chimeric protein (Mantel-Cox p value 0.0111), unlike the treatment with the anti-PD-1 antibody (Mantel-Cox p value 0.3907).

These results demonstrate, inter alia, that the SIRPα-Fc-4-1BBL chimeric protein significantly inhibited tumor growth and improves survival in animals suffering from both anti-PD-1 sensitive and anti-PD-1 resistant tumors.

### Example 17: Safety and Pharmacodynamic Activity (PD) of the TIGIT-Fc-LIGHT Chimeric Protein in Cynomolgus Macaque

Cynomolgus macaques were determined to be an appropriate species to examine the potential toxicity and immune properties of the human TIGIT-Fc-LIGHT chimeric protein due to the cross-binding of human TIGIT and LIGHT to cynomolgus macaque targets (data not shown). Groups of treatment-naïve cynomolgus macaques were repeatedly treated with intravenous infusions of TIGIT-Fc(IgG4)-LIGHT at doses of 0.1, 1.0, 40 mg/kg or vehicle control administered on days 1, 8, 15 and 22 of the study. the TIGIT-Fc-LIGHT chimeric protein was well tolerated by all dose groups.

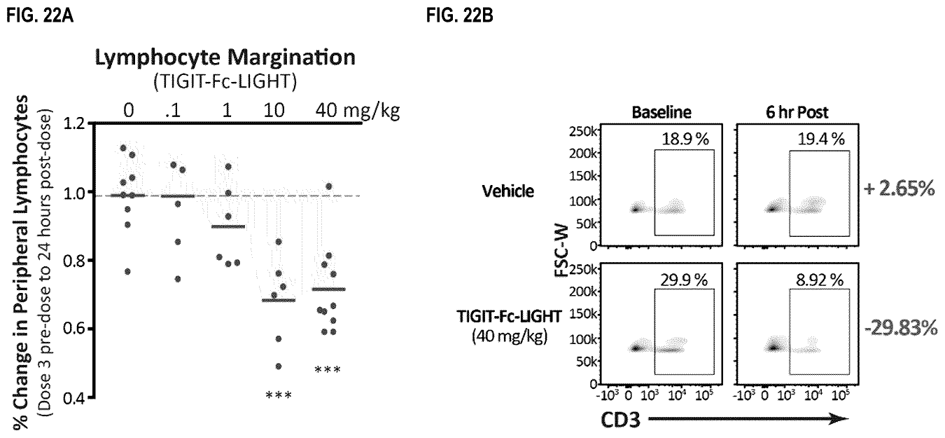

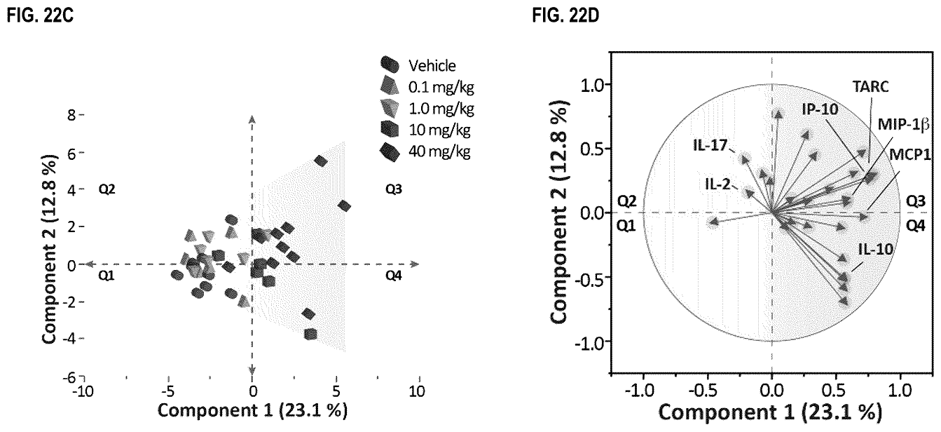

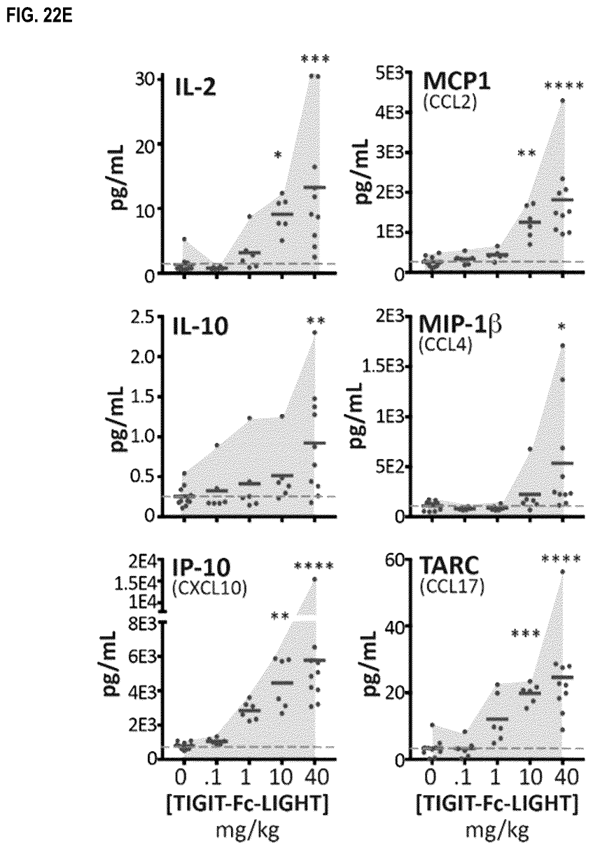

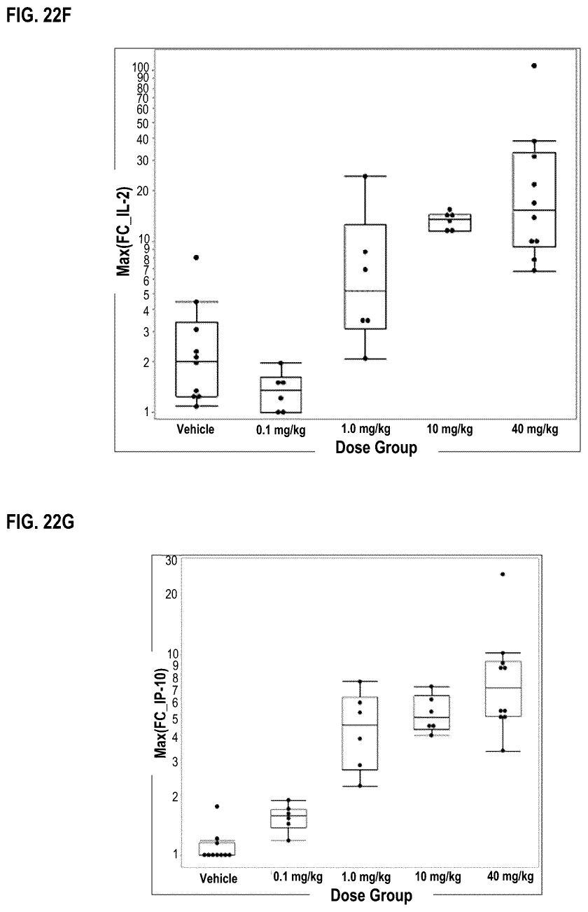

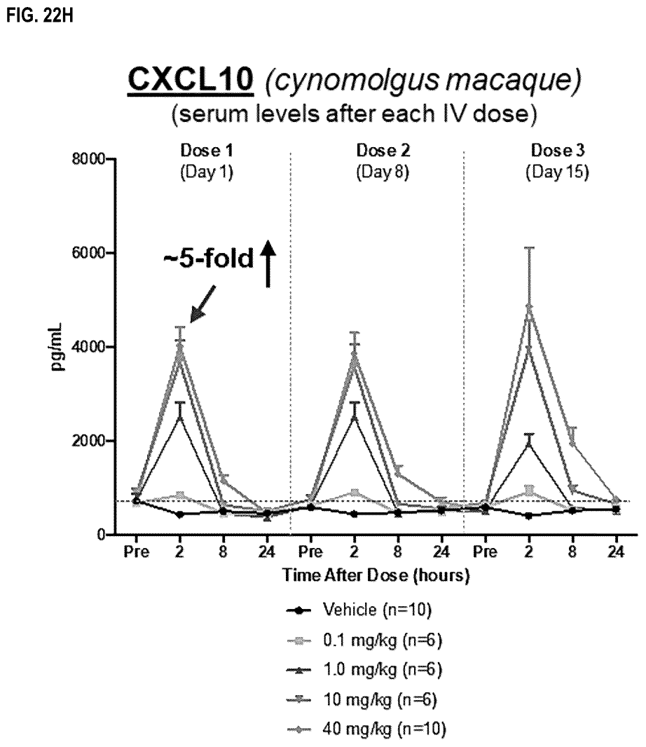

Pre- and post-dose lymphocyte counts were obtained on day 15 prior to the third dose, and on day 16 approximately 24 hours after the third dose. In this experiment, flow cytometry was performed to determine whether the observed changes in lymphocyte counts were driven by selective margination of HVEM expressing lymphocytes out of the peripheral circulation. Consistently, the TIGIT-Fc-LIGHT chimeric protein stimulated a dose-dependent margination of peripheral lymphocytes that was evident in the complete blood count analysis immediately post-dose (FIG. 22A). Therefore, the post-dose lymphocyte margination from day to day 16 was observed (FIG. 22A). The number of peripheral blood lymphocytes was observed to decrease in a dose-dependent manner following the Day 15 dose, and is plotted as the (100−((# of lymphocytes on Day 16)/(# of lymphocytes on Day 15)×100). Each data point indicates an individual animal. As shown in FIG. 22A, there was a dose-dependent decrease in the number of peripheral blood lymphocytes in the monkeys treated with the TIGIT-Fc-LIGHT chimeric protein compared to the control monkeys.

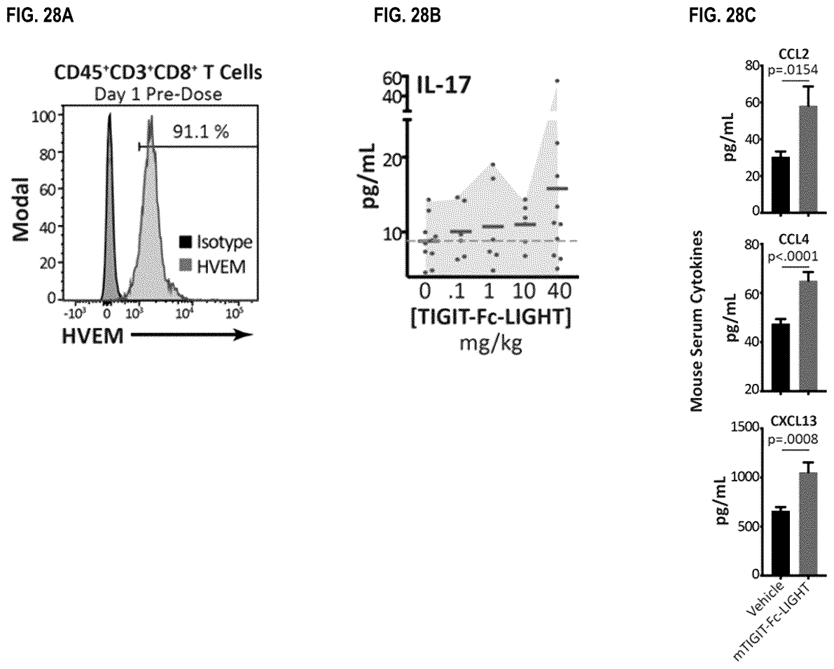

The effect of the TIGIT-Fc-LIGHT chimeric protein on peripheral CD3+ T cells was further explored in cynomolgus monkeys. Cynomolgus macaques were given IV infusion of vehicle or 40 mg/kg of the TIGIT-Fc-LIGHT chimeric protein. Pre- and post-dose lymphocyte counts were obtained on prior to the dose and on 6 hours after the dose. FIG. 22B shows the post-dose margination of CD3+ T cells from the six hours post dose samples. As shown in FIG. 22B, the number of peripheral blood CD3+ T cells decreased by about 30% in the monkeys treated with the TIGIT-Fc-LIGHT chimeric protein. In contrast, the number of peripheral blood CD3+ T cells increased in vehicle-treated monkeys by 2.65% (FIG. 22B). These data illustrate the post-dose margination of CD3+ T cells. These data illustrate the post-dose lymphocyte margination. More specifically, the TIGIT-Fc-LIGHT chimeric protein induced the margination of CD3⁺ T cells out of the periphery within 6 hours after the initial infusion (FIG. 22B), and these cells were demonstrated to express high levels of HVEM (FIG. 28A). The changes in peripheral lymphocytes were restricted to the lymphoid compartment, and no significant changes were observed in the number of peripheral neutrophils, basophils, eosinophils, platelets, nor differences in the levels of hemoglobin or hematocrit (data not shown).

Cynomolgus macaques were given 4 weekly IV infusions (days 1, 8, 15, 22) of vehicle or 0.1, 1.0, 10, or 40 mg/kg of the TIGIT-Fc-LIGHT chimeric protein. Various cytokines were measured 2-hour post-dose to determine a cytokine signature. A principal component analysis (PCA) was performed to visualize the distribution of animals based on the 2-hour post-dose cytokine signature. Dose-dependent increases in the serum concentration of multiple pro-inflammatory cytokines were noted, and unbiased principal component analysis (PCA) revealed a separation of dose treatment groups when all 30 cytokines from a multiplex array were included in the analysis (FIG. 22C). As shown in FIG. 22C, the cytokine profiles of the animals showed a trend of a dose-dependent change compared to vehicle-treated monkeys. Trends in various cytokines were plotted. JMP software was used to generate a vector plot that identified cytokines that dominated the migration of individual animals across PCA quadrants based on a specific cytokine signature (FIG. 22D). As shown in FIG. 22D, the pro-inflammatory cytokines dominated the clustering of samples in quadrants Q3 and Q4, and adaptive immune cytokines (e.g., IL-2 and IL-17) dominated Q2. The 2-hour-post-dose levels of various cytokines were assessed using Meso Scale Discovery (MSD) assays and plotted as a function of dose of the TIGIT-Fc-LIGHT chimeric protein. As shown in FIG. 22E, IL-2, IL-10, IP-10 (CXCL10), MCP-1, MIP-1β (CCL4) TARC (CCL17), IFNγ, IL-8, IL-12, and SDF1a (CXCL12) showed a dose-dependent increase. The signature that led to animal responses migrating to quadrants 3 and 4, included pro-inflammatory cytokines such as CXCL10, CCL2, CCL4, CCL17, and IL-10. Adaptive immune cytokines were also observed to follow a dose response, and clustered in quadrant 2, including IL-2 and IL-17 (FIG. 22E and FIG. 28B). Many of the serum cytokine responses were conserved with observations using the murine TIGIT-Fc-LIGHT chimeric protein (FIG. 28C). Together, these results indicated, inter alia, that the TIGIT-Fc-LIGHT chimeric protein exerted potent immunological effects based on the receptor/ligand interactions involved in the TIGIT and LIGHT pathways. Encouragingly, many of the serum cytokine changes observed in non-human primates overlapped with the cytokine changes observed in mouse in vivo and human in vitro assays (FIG. 24 and FIG. 26). FIG. 22E shows the cytokine response as assessed using Meso Scale Discovery (MSD) assays. The extent of induction of IL-2 and IP-10 was assayed as a function of dose of the TIGIT-Fc-LIGHT chimeric protein. As shown in FIG. 22F, an induction of IL-2 was observed in a dose-dependent manner. similarly, as shown in FIG. 22G, an induction of IP-10 was observed in a dose-dependent manner. The kinetics of induction was studied. FIG. 22G shows the fold induction of IP-10. FIG. 22H shows the kinetics of induction of CXCL-10 during and after first, second and third doses. As shown in FIG. 22H, the peak of induction was observed at 2 hr following the dose and a background level was achieved in about 8 hours.

These data show that a distinct profile of serum cytokines including IL-2 and IP-10 (above) was observed in a dose-dependent manner. Collectively, these results demonstrate that the TIGIT-Fc-LIGHT chimeric protein causes margination of T cells, and induction of cytokines such as IL-2, IL-10, IP-10 (CXCL10), MCP-1, MIP-1β (CCL4) TARC (CCL17), IFNγ, IL-8, IL-12, and SDF1a (CXCL12).

Overall, the TIGIT-Fc-LIGHT chimeric protein was well tolerated up to at least 40 mg/kg. Importantly, there was no evidence of cytokine release syndrome, despite the observed cytokine profile and relatively small increases in IL-6.

### INCORPORATION BY REFERENCE

All patents and publications referenced herein are hereby incorporated by reference in their entireties.

The publications discussed herein are provided solely for their disclosure prior to the filing date of the present application. Nothing herein is to be construed as an admission that the present disclosure is not entitled to antedate such publication by virtue of prior invention.

As used herein, all headings are simply for organization and are not intended to limit the disclosure in any manner. The content of any individual section may be equally applicable to all sections.

### EQUIVALENTS

While the invention has been disclosed in connection with specific embodiments thereof, it will be understood that it is capable of further modifications and this application is intended to cover any variations, uses, or adaptations of the invention following, in general, the principles of the invention and including such departures from the present disclosure as come within known or customary practice within the art to which the invention pertains and as may be applied to the essential features hereinbefore set forth and as follows in the scope of the appended claims.

Those skilled in the art will recognize, or be able to ascertain, using no more than routine experimentation, numerous equivalents to the specific embodiments disclosed specifically herein. Such equivalents are intended to be encompassed in the scope of the following claims.

What is claimed is:

1. A method for treating a cancer in a subject in need thereof the method comprising a step of administering to the subject an effective amount of a chimeric protein having a general structure of: N
terminus—(a)—(b)—(c)—C terminus,
wherein:
(a) is a first domain comprising an extracellular domain of human T cell immunoreceptor with Ig and ITIM domains (TIGIT),
(b) is a linker adjoining the first and second domains, wherein the linker comprises a hinge-CH2-CH3 Fc domain, and
(c) is a second domain comprising an extracellular domain of human LIGHT (lymphotoxin-like, exhibits inducible expression and competes with HSV glycoprotein D for herpesvirus entry mediator (HVEM), a receptor expressed by T lymphocytes),
wherein the dose of the chimeric protein administered is between about 0.0001 mg/kg and about mg/kg, optionally selected from about 1 mg/kg, about 3 mg/kg, about 6 mg/kg, or about 10 mg/kg, about 12 mg/kg, about 15 mg/kg, about 18 mg/kg, about 20 mg/kg, about 22 mg/kg, about 25 mg/kg, about 27 mg/kg, about 30 mg/kg, about 33 mg/kg, about 35 mg/kg, about 37 mg/kg, about 40 mg/kg, about 42 mg/kg, about mg/kg, about 48 mg/kg, and about 50 mg/kg.

2. The method of claim 1, wherein the subject is a human, optionally an adult human.

3. The method of claim 1 or claim 2, wherein the chimeric protein is administered at least about one time a week.

4. The method of claim 3, wherein the chimeric protein is administered at least about one time a month.

5. The method of claim 4, wherein the chimeric protein is administered at least about two times a month.

6. The method of claim 5, wherein the chimeric protein is administered at least about three times a month.

7. The method of any one of claims 1 to 6, wherein the cancer comprises a solid tumor (local and/or metastatic) or a lymphoma.

8. The method of claim 7, wherein the cancer is selected from Hodgkin's and non-Hodgkin's lymphoma, B-cell lymphoma (including low grade/follicular non-Hodgkin's lymphoma (NHL); small lymphocytic (SL) NHL; intermediate grade/follicular NHL; intermediate grade diffuse NHL; high grade immunoblastic NHL; high grade lymphoblastic NHL; high grade small non-cleaved cell NHL; bulky disease NHL; mantle cell lymphoma; AIDS-related lymphoma; and Waldenstrom's Macroglobulinemia; chronic lymphocytic leukemia (CLL); acute lymphoblastic leukemia (ALL); Hairy cell leukemia; or chronic myeloblastic leukemia, basal cell carcinoma, biliary tract cancer; bladder cancer; bone cancer; brain and central nervous system cancer; breast cancer; cancer of the peritoneum; cervical cancer; choriocarcinoma; colon and rectum cancer; connective tissue cancer; cancer of the digestive system; endometrial cancer; esophageal cancer; eye cancer; cancer of the head and neck; gastric cancer (including gastrointestinal cancer); glioblastoma; hepatic carcinoma; hepatoma; intra-epithelial neoplasm; kidney or renal cancer; larynx cancer; leukemia; liver cancer; lung cancer (e.g., small-cell lung cancer, non-small cell lung cancer, adenocarcinoma of the lung, and squamous carcinoma of the lung); melanoma; myeloma; neuroblastoma; oral cavity cancer (lip, tongue, mouth, and pharynx); ovarian cancer; pancreatic cancer; prostate cancer; retinoblastoma; rhabdomyosarcoma; rectal cancer; cancer of the respiratory system; salivary gland carcinoma; sarcoma; skin cancer; squamous cell cancer; stomach cancer; testicular cancer; thyroid cancer; uterine or endometrial cancer; cancer of the urinary system; vulval cancer; lymphoma including Hodgkin's and non-Hodgkin's lymphoma, as well as B-cell lymphoma (including low grade/follicular non-Hodgkin's lymphoma (NHL); small lymphocytic (SL) NHL; intermediate grade/follicular NHL; intermediate grade diffuse NHL; high grade immunoblastic NHL; high grade lymphoblastic NHL; high grade small non-cleaved cell NHL; bulky disease NHL; mantle cell lymphoma; AIDS-related lymphoma; and Waldenstrom's Macroglobulinemia; chronic lymphocytic leukemia (CLL); acute lymphoblastic leukemia (ALL); Hairy cell leukemia; chronic myeloblastic leukemia; as well as other carcinomas and sarcomas; and post-transplant lymphoproliferative disorder (PTLD), as well as abnormal vascular proliferation associated with phakomatoses, edema (e.g., that associated with brain tumors), Meigs' syndrome cancer; renal carcinoma; colorectal cancer; and adrenal cancer.

9. A method for inducing lymphocyte expansion in a subject in need thereof, the method comprising a step of administering to the subject an effective amount of a chimeric protein having a general structure of:
N terminus—(a)—(b)—(c)—C terminus,
wherein:
(a) is a first domain comprising an extracellular domain of human T cell immunoreceptor with Ig and ITIM domains (TIGIT),
(b) is a linker adjoining the first and second domains, wherein the linker comprises a hinge-CH2-CH3 Fc domain, and
(c) is a second domain comprising an extracellular domain of human LIGHT (lymphotoxin-like, exhibits inducible expression and competes with HSV glycoprotein D for herpesvirus entry mediator (HVEM), a receptor expressed by T lymphocytes).

10. A method for inducing lymphocyte margination in a subject in need thereof, the method comprising a step of administering to the subject an effective amount of a chimeric protein having a general structure of:
N terminus—(a)—(b)—(c)—C terminus,
wherein:
(a) is a first domain comprising an extracellular domain of human T cell immunoreceptor with Ig and ITIM domains (TIGIT),
(b) is a linker adjoining the first and second domains, wherein the linker comprises a hinge-CH2-CH3 Fc domain, and
(c) is a second domain comprising an extracellular domain of human LIGHT (lymphotoxin-like, exhibits inducible expression and competes with HSV glycoprotein D for herpesvirus entry mediator (HVEM), a receptor expressed by T lymphocytes).

11. The method of any one of claim 1, 9 or 10, wherein the subject is dosed with a dosing regimen selected from about every 3 days to about every 10 days, about every week to about every 2 weeks, about every 10 days to about every 3 weeks, about every 2 weeks to about every 4 weeks, about every 3 weeks to about every 5 weeks, about every 4 weeks to about every 6 weeks, about every 5 weeks to about every 7 weeks, about every 6 weeks to about every 8 weeks, and about every 6 weeks to about every 2 months.

12. The method of any one of claims 1 to 11, wherein the first domain is capable of binding a TIGIT ligand.

13. The method of any one of claims 1 to 12, wherein the first domain comprises substantially all of the extracellular domain of TIGIT.

14. The method of any one of claims 1 to 13, wherein the second domain is capable of binding a LIGHT receptor.

15. The method of any one of claims 1 to 14, wherein the second domain comprises substantially all of the extracellular domain of LIGHT.

16. The method of any one of claims 1 to 15, wherein the linker is a polypeptide selected from a flexible amino acid sequence, an IgG hinge region, and/or an antibody sequence.

17. The method of any one of claims 1 to 16, wherein the linker comprises hinge-CH2-CH3 Fc domain derived from IgG1 or IgG4.

18. The method of claim 17, wherein the hinge-CH2-CH3 Fc domain is derived from human IgG1 or human IgG4.

19. The method of claim 18, wherein the linker comprises an amino acid sequence that is at least 95%, or at least 96%, or at least 97%, or at least 98%, or at least 99% identical to the amino acid sequence of SEQ ID NO: 46, SEQ ID NO: 47, SEQ ID NO: 112, or SEQ ID NO: 113.

20. The method of any one of claims 1 to 19, wherein the linker comprises one or more joining linkers, such joining linkers independently selected from SEQ ID NOs: 49-95.

21. The method of claim 18, wherein the linker comprises two or more joining linkers each joining linker independently selected from SEQ ID NOs: 49-95; wherein one joining linker is N terminal to the hinge-CH2-CH3 Fc domain and another joining linker is C terminal to the hinge-CH2-CH3 Fc domain.

22. The method of any one of claims 1 to 21, wherein the first domain comprises an amino acid sequence that is at least 95%, or at least 96%, or at least 97%, or at least 98%, or at least 99% identical to the amino acid sequence of SEQ ID NO: 10.

23. The method of any one of claims 1 to 22, wherein the second domain comprises an amino acid sequence that is at least 95%, or at least 96%, or at least 97%, or at least 98%, or at least 99% identical to the amino acid sequence of SEQ ID NO: 2.

24. The method of any one of claims 1 to 23, wherein
(a) the first domain comprises an amino acid sequence that is at least 95% identical to the amino acid sequence of SEQ ID NO: 10,
(b) the second domain comprises the amino acid sequence that is at least 95% identical to the amino acid sequence of SEQ ID NO: 2, and
(c) the linker comprises an amino acid sequence that is at least 95% identical to the amino acid sequence of SEQ ID NO: 46, SEQ ID NO: 47, SEQ ID NO: 112, or SEQ ID NO: 113.

25. The method of any one of claims 1 to 24, wherein
(a) the first domain comprises the amino acid sequence of SEQ ID NO: 10,
(b) the second domain comprises the amino acid sequence of SEQ ID NO: 2, and
(c) the linker comprises an amino acid sequence that is at least 95% identical to the amino acid sequence of SEQ ID NO: 46, SEQ ID NO: 47, SEQ ID NO: 112, or SEQ ID NO: 113.

26. The method of claim 25, wherein the chimeric protein further comprises at least one joining linker comprising an amino acid sequence selected from SKYGPPCPSCP (SEQ ID NO: 49), SKYGPPCPPCP (SEQ ID NO: 50), IEGRMD (SEQ ID NO: 52).

27. The method of claim 26, wherein the chimeric protein comprises the joining linker comprising the amino acid sequence of IEGRMD (SEQ ID NO: 52).

28. The method of claim 27, wherein the amino acid sequence of IEGRMD is located at C-terminal end of the amino acid sequence of SEQ ID NO: 46, SEQ ID NO: 47, SEQ ID NO: 112, or SEQ ID NO: 113.

29. The method of any one of claims 1 to 28, wherein the chimeric protein comprises an amino acid sequence that is at least 95%, or at least 96%, or at least 97%, or at least 98%, or at least 99% identical to an amino acid sequence selected from SEQ ID NO: 11, SEQ ID NO: 109, and SEQ ID NO: 110.

30. The method of claim 29, wherein the chimeric protein comprises an amino acid sequence that is at least 98% identical to an amino acid sequence selected from SEQ ID NO: 11, SEQ ID NO: 109, and SEQ ID NO: 110.

31. The method of claim 30, wherein the chimeric protein comprises an amino acid sequence that is at least 99% identical to an amino acid sequence selected from SEQ ID NO: 11, SEQ ID NO: 109, and SEQ ID NO: 110.

32. The method of claim 31, wherein the chimeric protein comprises an amino acid sequence that is at least 99.2% identical to an amino acid sequence selected from SEQ ID NO: 11, SEQ ID NO: 109, and SEQ ID NO: 110.

33. The method of claim 32, wherein the chimeric protein comprises an amino acid sequence that is at least 99.4% identical to an amino acid sequence selected from SEQ ID NO: 11, SEQ ID NO: 109, and SEQ ID NO: 110.

34. The method of claim 33, wherein the chimeric protein comprises an amino acid sequence that is at least 99.6% identical to an amino acid sequence selected from SEQ ID NO: 11, SEQ ID NO: 109, and SEQ ID NO: 110.

35. The method of claim 34, wherein the chimeric protein comprises an amino acid sequence that is at least 99.8% identical to an amino acid sequence selected from SEQ ID NO: 11, SEQ ID NO: 109, and SEQ ID NO: 110.

36. The method of claim 35, wherein the chimeric protein comprises an amino acid sequence selected from SEQ ID NO: 11, SEQ ID NO: 109, and SEQ ID NO: 110.

37. The method of any one of claims 1 to 36, wherein the subject has received, been tolerant to, or is ineligible for standard therapy and/or the cancer has no approved therapy considered to be standard of care.

38. The method of any one of claims 1 to 37, wherein the subject is not receiving a concurrent chemotherapy, immunotherapy, biologic or hormonal therapy.

39. A method of evaluating the efficacy of a cancer treatment in a subject in need thereof, the method comprising the steps of:
(i) administering a dose of a chimeric protein having a general structure of:
N terminus—(a)—(b)—(c)—C terminus,
wherein:
(a) is a first domain comprising an extracellular domain of human T cell immunoreceptor with Ig and ITIM domains (TIGIT),
(b) is a linker adjoining the first and second domains, wherein the linker comprises a hinge-CH2-CH3 Fc domain, and
(c) is a second domain comprising an extracellular domain of human LIGHT (lymphotoxin-like, exhibits inducible expression and competes with HSV glycoprotein D for herpesvirus entry mediator (HVEM), a receptor expressed by T lymphocytes).
wherein the dose is from about 0.03 mg/kg to about 50 mg/kg;
(ii) obtaining a biological sample from the subject;
(iii) performing an assay on the biological sample to determine level and/or activity of a cytokine selected from IL-2, IL-10, IP-10 (CXCL10), MCP-1, MIP-1β (CCL4) TARC (CCL17), IFNγ, IL-8, IL-12, and SDF1a (CXCL12); and
(iv) continuing dosing if the subject has an increase in the level and/or activity of at least one cytokine selected from IL-2, IL-10, IP-10 (CXCL10), MCP-1, MIP-1β (CCL4) TARC (CCL17), IFNγ, IL-8, IL-12, and SDF1a (CXCL12).

40. A method of selecting a subject for treatment with a therapy for cancer, the method comprising the steps of:
(i) administering a dose of a chimeric protein having a general structure of:
N terminus—(a)—(b)—(c)—C terminus,
wherein:
(a) is a first domain comprising an extracellular domain of human T cell immunoreceptor with Ig and ITIM domains (TIGIT),
(b) is a linker adjoining the first and second domains, wherein the linker comprises a hinge-CH2-CH3 Fc domain, and
(c) is a second domain comprising an extracellular domain of human LIGHT (lymphotoxin-like, exhibits inducible expression and competes with HSV glycoprotein D for herpesvirus entry mediator (HVEM), a receptor expressed by T lymphocytes).
wherein the dose is from about 0.03 mg/kg to about 50 mg/kg;
(ii) obtaining a biological sample from the subject;
(iii) performing an assay on the biological sample to determine level and/or activity of a cytokine selected from IL-2, IL-10, IP-10 (CXCL10), MCP-1, MIP-1β (CCL4) TARC (CCL17), IFNγ, IL-8, IL-12, and SDF1a (CXCL12); and
(iv) selecting the subject for treatment with the therapy for cancer if the subject has an increase in the level and/or activity of at least one cytokine selected from IL-2, IL-10, IP-10 (CXCL10), MCP-1, MIP-1β (CCL4) TARC (CCL17), IFNγ, IL-8, IL-12, and SDF1a (CXCL12).

41. The method of any one of claims 39 to 40, wherein the biological sample is a fresh tissue sample, frozen tumor tissue specimen, cultured cells, circulating tumor cells, or a formalin-fixed paraffin-embedded tumor tissue specimen.

42. The method of any one of claims 39 to 41, wherein the biological sample is a biopsy sample.

43. The method of claim 42, wherein the biopsy sample is selected from endoscopic biopsy, bone marrow biopsy, endoscopic biopsy (e.g., cystoscopy, bronchoscopy and colonoscopy), needle biopsy (e.g., fine-needle aspiration, core needle biopsy, vacuum-assisted biopsy, X-ray-assisted biopsy, computerized tomography (CT)-assisted biopsy, magnetic resonance imaging (MRI)-assisted biopsy and ultrasound-assisted biopsy), skin biopsy (e.g., shave biopsy, punch biopsy, and incisional biopsy) and surgical biopsy.

44. The method of any one of claims 39 to 43, wherein the biological sample comprises a body fluid selected from blood, plasma, serum, lacrimal fluid, tears, bone marrow, blood, blood cells, ascites, tissue or fine needle biopsy sample, cell-containing body fluid, free floating nucleic acids, sputum, saliva, urine, cerebrospinal fluid, peritoneal fluid, pleural fluid, feces, lymph, gynecological fluid, skin swab, vaginal swab, oral swab, nasal swab, washing or lavage such as a ductal lavage or broncheoalveolar lavage, aspirate, scraping, bone marrow specimen, tissue biopsy specimen, surgical specimen, feces, other body fluids, secretions, and/or excretions, and/or cells therefrom.

45. The method of any one of claims 39 to 44, wherein the biological sample is obtained by a technique selected from scrapes, swabs, and biopsy.

46. The method of claim 45, wherein the biological sample is obtained by use of brushes, (cotton) swabs, spatula, rinse/wash fluids, punch biopsy devices, puncture of cavities with needles or surgical instrumentation.

47. The method of any one of claims 39 to 46, wherein the biological sample comprises at least one tumor cell.

48. The method of claim 47, wherein the tumor is selected from Hodgkin's and non-Hodgkin's lymphoma, B-cell lymphoma (including low grade/follicular non-Hodgkin's lymphoma (NHL); small lymphocytic (SL) NHL; intermediate grade/follicular NHL; intermediate grade diffuse NHL; high grade immunoblastic NHL; high grade lymphoblastic NHL; high grade small non-cleaved cell NHL; bulky disease NHL; mantle cell lymphoma; AIDS-related lymphoma; and Waldenstrom's Macroglobulinemia; chronic lymphocytic leukemia (CLL); acute lymphoblastic leukemia (ALL); Hairy cell leukemia; or chronic myeloblastic leukemia, basal cell carcinoma, biliary tract cancer; bladder cancer; bone cancer; brain and central nervous system cancer; breast cancer; cancer of the peritoneum; cervical cancer; choriocarcinoma; colon and rectum cancer; connective tissue cancer; cancer of the digestive system; endometrial cancer; esophageal cancer; eye cancer; cancer of the head and neck; gastric cancer (including gastrointestinal cancer); glioblastoma; hepatic carcinoma; hepatoma; intra-epithelial neoplasm; kidney or renal cancer; larynx cancer; leukemia; liver cancer; lung cancer (e.g., small-cell lung cancer, non-small cell lung cancer, adenocarcinoma of the lung, and squamous carcinoma of the lung); melanoma; myeloma; neuroblastoma; oral cavity cancer (lip, tongue, mouth, and pharynx); ovarian cancer; pancreatic cancer; prostate cancer; retinoblastoma; rhabdomyosarcoma; rectal cancer; cancer of the respiratory system; salivary gland carcinoma; sarcoma; skin cancer; squamous cell cancer; stomach cancer; testicular cancer; thyroid cancer; uterine or endometrial cancer; cancer of the urinary system; vulval cancer; lymphoma including Hodgkin's and non-Hodgkin's lymphoma, as well as B-cell lymphoma (including low grade/follicular non-Hodgkin's lymphoma (NHL); small lymphocytic (SL) NHL; intermediate grade/follicular NHL; intermediate grade diffuse NHL; high grade immunoblastic NHL; high grade lymphoblastic NHL; high grade small non-cleaved cell NHL; bulky disease NHL; mantle cell lymphoma; AIDS-related lymphoma; and Waldenstrom's Macroglobulinemia; chronic lymphocytic leukemia (CLL); acute lymphoblastic leukemia (ALL); Hairy cell leukemia; chronic myeloblastic leukemia; as well as other carcinomas and sarcomas; and post-transplant lymphoproliferative disorder (PTLD), as well as abnormal vascular proliferation associated with phakomatoses, edema (e.g., that associated with brain tumors), Meigs' syndrome cancer; renal carcinoma; colorectal cancer; and adrenal cancer.

49. The method of any one of claims 39 to 48, wherein the assay is performed by DNA sequencing, RNA sequencing, immunohistochemical staining, western blotting, in cell western, immunofluorescent staining, ELISA, and fluorescent activating cell sorting (FACS) or a combination thereof.

50. The method of claim 49, wherein the assay is performed by contacting the sample with one or more agents that specifically binds to at least one cytokine selected from IL-2, IL-10, IP-10 (CXCL10), MCP-1, MIP-1β (CCL4) TARC (CCL17), IFNγ, IL-8, IL-12, and SDF1a (CXCL12).

51. The method of claim 50, wherein the agents that specifically binds to at least one cytokine comprise one or more antibody, antibody-like molecule or binding a fragment thereof.

52. The method of claim 49, wherein the assay is performed by contacting the sample with one or more agents that specifically binds to at least one nucleic acid encoding a cytokine selected from IL-2, IL-10, IP-(CXCL10), MCP-1, MIP-1β (CCL4) TARC (CCL17), IFNγ, IL-8, IL-12, and SDF1a (CXCL12).

53. The method of claim 52, wherein the agent that specifically binds to at least one nucleic acid is a nucleic acid primer or probe.

54. A method of determining a cancer treatment for a patient, the method comprising:
(i) obtaining a biological sample from a subject;
(ii) evaluating the sample for
the upregulation of one or more genes associated with a gene ontology (GO) pathway selected from positive regulation of cell cycle process, regulation of G1/S transition, regulation of cell division, regulation of cell proliferation, positive regulation of IκB kinase/NFκB signaling, type I IFN signaling pathway, cellular response to IFNγ, positive regulation of IFNα production, positive regulation of defense response, positive regulation of IFNβ production, regulation of inflammatory response, regulation of innate immune response, negative regulation of antigen processing/presentation, and antigen processing/presentation of endogenous peptides via MHC class I; and/or
downregulation of one or more genes associated with a gene ontology (GO) pathway selected from phospholipid efflux, negative regulation of fibrinolysis, chylomicron assembly, plasma membrane repair, SRP-dependent co-translational protein targeting to membrane, ribosomal small subunit assembly, phospholipid efflux, regulation of translation, mitochondrial respiratory chain complex I, mitochondrial translational elongation, DNA-dependent DNA replication, and ATP biosynthetic process;
and
(iii) selecting the cancer therapy based on the evaluation of step (b), wherein the cancer therapy comprises a chimeric protein of a general structure of:
N terminus—(a)—(b)—(c)—C terminus,
wherein:
(A)
(a) is a first domain comprising an extracellular domain of a Type I transmembrane protein, the transmembrane protein being TIGIT,
(b) is a linker comprising at least one cysteine residue capable of forming a disulfide bond, and
(c) is a second domain comprising an extracellular domain of Type II transmembrane protein, the transmembrane protein being LIGHT, wherein the linker connects the first domain and the second domain and optionally comprises one or more joining linkers, such joining linkers being selected from SEQ ID NOs: 49-95; or
(B)
(a) is a first domain comprising an extracellular domain of a Type I transmembrane protein, the transmembrane protein being SIRPα,
(b) is a linker comprising at least one cysteine residue capable of forming a disulfide bond, and
(c) is a second domain comprising an extracellular domain of Type II transmembrane protein, the transmembrane protein being 4-1BBL, wherein the linker connects the first domain and the second domain and optionally comprises one or more joining linkers, such joining linkers being selected from SEQ ID NOs: 49-95.

55. A method for selecting a patient for a cancer treatment, the method comprising:
(i) obtaining a biological sample from a subject;
(ii) evaluating the sample for
the upregulation of one or more genes associated with a gene ontology (GO) pathway selected from positive regulation of cell cycle process, regulation of G1/S transition, regulation of cell division, regulation of cell proliferation, positive regulation of IκB kinase/NFκB signaling, type I IFN signaling pathway, cellular response to IFNγ, positive regulation of IFNα production, positive regulation of defense response, positive regulation of IFNβ production, regulation of inflammatory response, regulation of innate immune response, negative regulation of antigen processing/presentation, and antigen processing/presentation of endogenous peptides via MHC class I; and/or
downregulation of one or more genes associated with a gene ontology (GO) pathway selected from phospholipid efflux, negative regulation of fibrinolysis, chylomicron assembly, plasma membrane repair, SRP-dependent co-translational protein targeting to membrane, ribosomal small subunit assembly, phospholipid efflux, regulation of translation, mitochondrial respiratory chain complex I, mitochondrial translational elongation, DNA-dependent DNA replication, and ATP biosynthetic process;
and
(iii) selecting a cancer therapy, wherein the cancer therapy comprises a chimeric protein of a general structure of:
N terminus—(a)—(b)—(c)—C terminus,
wherein:
(A)
(a) is a first domain comprising an extracellular domain of a Type I transmembrane protein, the transmembrane protein being TIGIT,
(b) is a linker comprising at least one cysteine residue capable of forming a disulfide bond, and
(c) is a second domain comprising an extracellular domain of Type II transmembrane protein, the transmembrane protein being LIGHT, wherein the linker connects the first domain and the second domain and optionally comprises one or more joining linkers, such joining linkers being selected from SEQ ID NOs: 49-95; or
(B)
(a) is a first domain comprising an extracellular domain of a Type I transmembrane protein, the transmembrane protein being SIRPα,
(b) is a linker comprising at least one cysteine residue capable of forming a disulfide bond, and
(c) is a second domain comprising an extracellular domain of Type II transmembrane protein, the transmembrane protein being 4-1BBL, wherein the linker connects the first domain and the second domain and optionally comprises one or more joining linkers, such joining linkers being selected from SEQ ID NOs: 49-95.

56. A method of treating cancer, the method comprising:
(i) obtaining a biological sample from a subject;
(ii) evaluating the sample for
the upregulation of one or more genes associated with a gene ontology (GO) pathway selected from positive regulation of cell cycle process, regulation of G1/S transition, regulation of cell division, regulation of cell proliferation, positive regulation of IκB kinase/NFκB signaling, type I IFN signaling pathway, cellular response to IFNγ, positive regulation of IFNα production, positive regulation of defense response, positive regulation of IFNβ production, regulation of inflammatory response, regulation of innate immune response, negative regulation of antigen processing/presentation, and antigen processing/presentation of endogenous peptides via MHC class I; and/or
downregulation of one or more genes associated with a gene ontology (GO) pathway selected from phospholipid efflux, negative regulation of fibrinolysis, chylomicron assembly, plasma membrane repair, SRP-dependent co-translational protein targeting to membrane, ribosomal small subunit assembly, phospholipid efflux, regulation of translation, mitochondrial respiratory chain complex I, mitochondrial translational elongation, DNA-dependent DNA replication, and ATP biosynthetic process;
and
(iii) selecting the cancer therapy, wherein the cancer therapy comprises a chimeric protein of a general structure of:
N terminus—(a)—(b)—(c)—C terminus,
wherein:
(A)
(a) is a first domain comprising an extracellular domain of a Type I transmembrane protein, the transmembrane protein being TIGIT,
(b) is a linker comprising at least one cysteine residue capable of forming a disulfide bond, and
(c) is a second domain comprising an extracellular domain of Type II transmembrane protein, the transmembrane protein being LIGHT, wherein the linker connects the first domain and the second domain and optionally comprises one or more joining linkers, such joining linkers being selected from SEQ ID NOs: 49-95; or
(B)
(a) is a first domain comprising an extracellular domain of a Type I transmembrane protein, the transmembrane protein being SIRPα,
(b) is a linker comprising at least one cysteine residue capable of forming a disulfide bond, and
(c) is a second domain comprising an extracellular domain of Type II transmembrane protein, the transmembrane protein being 4-1BBL, wherein the linker connects the first domain and the second domain and optionally comprises one or more joining linkers, such joining linkers being selected from SEQ ID NOs: 49-95.

57. The method of any one of claims 54 to 56, wherein the upregulation is in comparison to a healthy tissue.

58. The method of any one of claims 54 to 56, wherein the upregulation is in comparison to another biological sample from patient that is known to be sensitive to anti-PD-1 therapy.

59. The method of any one of claims 54 to 56, wherein the upregulation is in comparison to a prior biological sample obtained from the subject.

60. The method of any one of claims 54 to 59, wherein the downregulation is in comparison to a healthy tissue.

61. The method of any one of claims 54 to 59, wherein the downregulation is in comparison to another biological sample from patient that is known to be sensitive to anti-PD-1 therapy.

62. The method of any one of claims 54 to 59, wherein the downregulation is in comparison to a prior biological sample obtained from the subject.

63. The method of any one of claims 54 to 62, wherein the biological sample is a fresh tissue sample, frozen tumor tissue specimen, cultured cells, circulating tumor cells, or a formalin-fixed paraffin-embedded tumor tissue specimen.

64. The method of claim 63, wherein the biological sample is a biopsy sample.

65. The method of claim 64, wherein the biopsy sample is selected from endoscopic biopsy, bone marrow biopsy, endoscopic biopsy (e.g., cystoscopy, bronchoscopy and colonoscopy), needle biopsy (e.g., fine-needle aspiration, core needle biopsy, vacuum-assisted biopsy, X-ray-assisted biopsy, computerized tomography (CT)-assisted biopsy, magnetic resonance imaging (MRI)-assisted biopsy and ultrasound-assisted biopsy), skin biopsy (e.g., shave biopsy, punch biopsy, and incisional biopsy) and surgical biopsy.

66. The method of any one of claims 54 to 65, wherein the biological sample comprises a body fluid selected from blood, plasma, serum, lacrimal fluid, tears, bone marrow, blood, blood cells, ascites, tissue or fine needle biopsy sample, cell-containing body fluid, free floating nucleic acids, sputum, saliva, urine, cerebrospinal fluid, peritoneal fluid, pleural fluid, feces, lymph, gynecological fluid, skin swab, vaginal swab, oral swab, nasal swab, washing or lavage such as a ductal lavage or broncheoalveolar lavage, aspirate, scraping, bone marrow specimen, tissue biopsy specimen, surgical specimen, feces, other body fluids, secretions, and/or excretions, and/or cells therefrom.

67. The method of any one of claims 54 to 66, wherein the biological sample is obtained by a technique selected from scrapes, swabs, and biopsy.

68. The method of claim 67, wherein the biological sample is obtained by use of brushes, (cotton) swabs, spatula, rinse/wash fluids, punch biopsy devices, puncture of cavities with needles or surgical instrumentation.

69. The method of any one of claims 64 to 68, wherein the biological sample comprises at least one tumor cell.

70. The method of claim 69, wherein the tumor is selected from Hodgkin's and non-Hodgkin's lymphoma, B-cell lymphoma (including low grade/follicular non-Hodgkin's lymphoma (NHL); small lymphocytic (SL) NHL; intermediate grade/follicular NHL; intermediate grade diffuse NHL; high grade immunoblastic NHL; high grade lymphoblastic NHL; high grade small non-cleaved cell NHL; bulky disease NHL; mantle cell lymphoma; AIDS-related lymphoma; and Waldenstrom's Macroglobulinemia; chronic lymphocytic leukemia (CLL); acute lymphoblastic leukemia (ALL); Hairy cell leukemia; or chronic myeloblastic leukemia, basal cell carcinoma, biliary tract cancer; bladder cancer; bone cancer; brain and central nervous system cancer; breast cancer; cancer of the peritoneum; cervical cancer; choriocarcinoma; colon and rectum cancer; connective tissue cancer; cancer of the digestive system; endometrial cancer; esophageal cancer; eye cancer; cancer of the head and neck; gastric cancer (including gastrointestinal cancer); glioblastoma; hepatic carcinoma; hepatoma; intra-epithelial neoplasm; kidney or renal cancer; larynx cancer; leukemia; liver cancer; lung cancer (e.g., small-cell lung cancer, non-small cell lung cancer, adenocarcinoma of the lung, and squamous carcinoma of the lung); melanoma; myeloma; neuroblastoma; oral cavity cancer (lip, tongue, mouth, and pharynx); ovarian cancer; pancreatic cancer; prostate cancer; retinoblastoma; rhabdomyosarcoma; rectal cancer; cancer of the respiratory system; salivary gland carcinoma; sarcoma; skin cancer; squamous cell cancer; stomach cancer; testicular cancer; thyroid cancer; uterine or endometrial cancer; cancer of the urinary system; vulval cancer; lymphoma including Hodgkin's and non-Hodgkin's lymphoma, as well as B-cell lymphoma (including low grade/follicular non-Hodgkin's lymphoma (NHL); small lymphocytic (SL) NHL; intermediate grade/follicular NHL; intermediate grade diffuse NHL; high grade immunoblastic NHL; high grade lymphoblastic NHL; high grade small non-cleaved cell NHL; bulky disease NHL; mantle cell lymphoma; AIDS-related lymphoma; and Waldenstrom's Macroglobulinemia; chronic lymphocytic leukemia (CLL); acute lymphoblastic leukemia (ALL); Hairy cell leukemia; chronic myeloblastic leukemia; as well as other carcinomas and sarcomas; and post-transplant lymphoproliferative disorder (PTLD), as well as abnormal vascular proliferation associated with phakomatoses, edema (e.g., that associated with brain tumors), Meigs' syndrome cancer; renal carcinoma; colorectal cancer; and adrenal cancer.

71. The method of any one of claims 54 to 70, wherein the evaluating is performed by DNA sequencing, RNA sequencing, immunohistochemical staining, western blotting, in cell western, immunofluorescent staining, ELISA, and fluorescent activating cell sorting (FACS) or a combination thereof.

72. The method of any one of claims 54 to 71, wherein the evaluating is performed by contacting the sample with an agent that specifically binds to one or more proteins encoded by one or more genes associated with a gene ontology (GO) pathway selected from:
(i) positive regulation of cell cycle process, regulation of G1/S transition, regulation of cell division, regulation of cell proliferation, positive regulation of IκB kinase/NFκB signaling, type I IFN signaling pathway, cellular response to IFNγ, positive regulation of IFNα production, positive regulation of defense response, positive regulation of IFNβ production, regulation of inflammatory response, regulation of innate immune response, negative regulation of antigen processing/presentation, and antigen processing/presentation of endogenous peptides via MHC class I; and/or
(ii) phospholipid efflux, negative regulation of fibrinolysis, chylomicron assembly, plasma membrane repair, SRP-dependent co-translational protein targeting to membrane, ribosomal small subunit assembly, phospholipid efflux, regulation of translation, mitochondrial respiratory chain complex I, mitochondrial translational elongation, DNA-dependent DNA replication, and ATP biosynthetic process.

73. The method of claim 72, wherein the evaluating is performed by contacting the sample with an agent that specifically binds to one or more proteins encoded by one or more genes associated with a gene ontology (GO) pathway selected from (a) cellular response to IFNγ, (b) negative regulation of antigen processing/presentation, (c) type I IFN signaling pathway, (d) positive regulation of IκB kinase/NFκB signaling, and antigen processing, and (e) presentation of endogenous peptides via MHC class I.

74. The method of claim 72 or claim 73, wherein the evaluating is performed by contacting the sample with an agent that specifically binds to one or more proteins encoded by one or more genes associated with cellular response to IFNγ.

75. The method of any one of claims 72 to 74, wherein the evaluating is performed by contacting the sample with an agent that specifically binds to one or more proteins encoded by one or more genes associated with type I IFN signaling pathway.

76. The method of any one of claims 54 to 71, wherein the evaluating is performed by contacting the sample with an agent that specifically binds to one or more of nucleic acids of one or more genes associated with a gene ontology (GO) pathway selected from:
(i) positive regulation of cell cycle process, regulation of G1/S transition, regulation of cell division, regulation of cell proliferation, positive regulation of IκB kinase/NFκB signaling, type I IFN signaling pathway, cellular response to IFNγ, positive regulation of IFNα production, positive regulation of defense response, positive regulation of IFNβ production, regulation of inflammatory response, regulation of innate immune response, negative regulation of antigen processing/presentation, and antigen processing/presentation of endogenous peptides via MHC class I; and/or
(ii) phospholipid efflux, negative regulation of fibrinolysis, chylomicron assembly, plasma membrane repair, SRP-dependent co-translational protein targeting to membrane, ribosomal small subunit assembly, phospholipid efflux, regulation of translation, mitochondrial respiratory chain complex I, mitochondrial translational elongation, DNA-dependent DNA replication, and ATP biosynthetic process.

77. The method of claim 76, wherein the evaluating is performed by contacting the sample with an agent that specifically binds to one or more nucleic acid of one or more genes associated with a gene ontology (GO) pathway selected from (a) cellular response to IFNγ, (b) negative regulation of antigen processing/presentation, (c) type I IFN signaling pathway, (d) positive regulation of IκB kinase/NFκB signaling, and antigen processing, and (e) presentation of endogenous peptides via MHC class I.

78. The method of claim 76 or claim 77, wherein the evaluating is performed by contacting the sample with an agent that specifically binds to one or more nucleic acid of one or more genes associated with cellular response to IFNγ.

79. The method of any one of claims 76 to 78, wherein the evaluating is performed by contacting the sample with an agent that specifically binds to one or more nucleic acid of one or more genes associated with type I IFN signaling pathway.

80. The method of any one of claims 76 to 79, wherein the agent that specifically binds to one or more of the nucleic acids is a nucleic acid primer or probe.

81. The method of any one of claims 54 to 80, wherein the evaluating informs classifying the patient into a high or low risk group.

82. The method of claim 81, wherein the high risk classification comprises a high level of tumor cells having resistance to the cancer therapy with an ability to inhibit function and/or activity of PD-1, PD-L1 and/or PD-L2.

83. The method of claim 81, wherein the low risk classification comprises a low level of tumor cells having resistance to the cancer therapy with an ability to inhibit function and/or activity of PD-1, PD-L1 and/or PD-L2.

84. The method of any one of claims 81 to 83, wherein the low risk classification is indicative of withholding of the cancer therapy.

85. The method of any one of claims 81 to 83, wherein the high risk classification is indicative of administering the cancer therapy.

86. A method for treating a cancer in a subject in need thereof comprising administering an effective amount of a pharmaceutical composition to a subject in need thereof, the pharmaceutical composition comprising a chimeric protein comprising:
N terminus—(a)—(b)—(c)—C terminus,
wherein:
(A)
(a) is a first domain comprising an extracellular domain of a Type I transmembrane protein, the transmembrane protein being TIGIT,
(b) is a linker comprising at least one cysteine residue capable of forming a disulfide bond, and
(c) is a second domain comprising an extracellular domain of Type II transmembrane protein, the transmembrane protein being LIGHT, wherein the linker connects the first domain and the second domain and optionally comprises one or more joining linkers, such joining linkers being selected from SEQ ID NOs: 49-95; or
(B)
(a) is a first domain comprising an extracellular domain of a Type I transmembrane protein, the transmembrane protein being SIRPα,
(b) is a linker comprising at least one cysteine residue capable of forming a disulfide bond, and
(c) is a second domain comprising an extracellular domain of Type II transmembrane protein, the transmembrane protein being 4-1BBL, wherein the linker connects the first domain and the second domain and optionally comprises one or more joining linkers, such joining linkers being selected from SEQ ID NOs: 49-95,
wherein the cancer is or is believed to be resistant to an anti-checkpoint agent having an ability to inhibit function and/or activity of PD-1, PD-L1 and/or PD-L2.

87. The method of any one of claims 54 to 86, wherein the linker is a polypeptide selected from a flexible amino acid sequence, an IgG hinge region, or an antibody sequence.

88. The method of any one of claims 54 to 87, wherein the linker comprises hinge-CH2-CH3 Fc domain derived from IgG1 or IgG4.

89. The method of any one of claims 54 to 88, wherein the hinge-CH2-CH3 Fc domain is derived from human IgG1 or human IgG4.

90. The method of claim 89, wherein the linker comprises an amino acid sequence that is at least 95%, or at least 96%, or at least 97%, or at least 98%, or at least 99% identical to the amino acid sequence of SEQ ID NO: 46, SEQ ID NO: 47, SEQ ID NO: 112, or SEQ ID NO: 113.

91. The method of any one of claims 54 to 90, wherein the linker comprises two or more joining linkers each joining linker independently selected from SEQ ID NOs: 49-95; wherein one joining linker is N terminal to the hinge-CH2-CH3 Fc domain and another joining linker is C terminal to the hinge-CH2-CH3 Fc domain.

92. The method of any one of claims 54 to 91, wherein the first domain comprises an amino acid sequence that is at least 95%, or at least 96%, or at least 97%, or at least 98%, or at least 99% identical to the amino acid sequence of SEQ ID NO: 10.

93. The method of any one of claims 54 to 92, wherein the first domain comprises an amino acid sequence that is at least 95%, or at least 96%, or at least 97%, or at least 98%, or at least 99% identical to the amino acid sequence of SEQ ID NO: 2.

94. The method of any one of claims 54 to 93, wherein the first domain comprises an amino acid sequence of the amino acid sequence of SEQ ID NO: 2.

95. The method of any one of claims 54 to 93, wherein the chimeric protein comprises an amino acid sequence that is at least 95%, or at least 96%, or at least 97%, or at least 98%, or at least 99% identical to the amino acid sequence selected from SEQ ID NO: 11, SEQ ID NO: 109, and SEQ ID NO: 110.

96. The method of any one of claims 54 to 95, wherein the chimeric protein is a recombinant fusion protein.

97. The method of any one of claims 54 to 96, wherein the anti-checkpoint agent an antibody selected from nivolumab (OPDIVO), pembrolizumab (KEYTRUDA), pidilizumab (CT-011, CURE TECH), MK-3475 (MERCK), BMS 936559, MPDL328OA (ROCHE), Cemiplimab (LIBTAYO), Atezolizumab (TECENTRIQ), Avelumab (BAVENCIO), and Durvalumab (imfinzi).

98. The method of any one of claims 86 to 97, further comprising administration of an anti-checkpoint agent.

99. The method of claim 98, wherein the anti-checkpoint agent an antibody selected from nivolumab (OPDIVO), pembrolizumab (KEYTRUDA), pidilizumab (CT-011, CURE TECH), MK-3475 (MERCK), BMS 936559, MPDL328OA (ROCHE), Cemiplimab (LIBTAYO), Atezolizumab (TECENTRIQ), Avelumab (BAVENCIO), and Durvalumab (imfinzi).

100. The method of claim 98 or claim 99, wherein the pharmaceutical composition comprising the chimeric protein and the anti-checkpoint agent are administered simultaneously or contemporaneously.

101. The method of any one of claims 98 to 100, wherein the pharmaceutical composition comprising the chimeric protein is administered after the anti-checkpoint agent is administered.

102. The method of any one of claims 98 to 100, wherein the pharmaceutical composition comprising the chimeric protein is administered before the anti-checkpoint agent is administered.

103. The method of any one of claims 98 to 102, wherein the dose of the pharmaceutical composition comprising the chimeric protein is less than the dose of the pharmaceutical composition comprising the chimeric protein administered to a subject who has not undergone or is not undergoing treatment with the anti-checkpoint agent.

104. The method of any one of claims 98 to 103, wherein the dose of the anti-checkpoint agent administered is less than the dose of the anti-checkpoint agent administered to a subject who has not undergone or is not undergoing treatment with the pharmaceutical composition comprising the chimeric protein.

105. The method of any one of claims 98 to 104, wherein the subject has an increased chance of survival, without gastrointestinal inflammation and weight loss, and/or a reduction in tumor size or cancer prevalence when compared to a subject who has only undergone or is only undergoing treatment with the pharmaceutical composition comprising the chimeric protein.

106. The method of any one of claims 98 to 105, wherein the subject has an increased chance of survival, without gastrointestinal inflammation and weight loss, and/or a reduction in tumor size or cancer prevalence when compared to a subject who has only undergone or is only undergoing treatment with the anti-checkpoint agent.

107. A method of determining a cancer treatment for a patient, the method comprising:
(I) obtaining a biological sample from a subject;
(II) evaluating the biological sample for the expression of:
(i) a gene selected from CD274, B2M, STAT1, STAT2, TRIM7, IRF1, TAP1, TAP2, CASP1, IRF, LTBR, PVR, GASTA3, LRG1, SPRY2, ARG1, TRIMS, TRIM2, MAPK81P1, TRIM6, and KRT1; and/or
(ii) a gene selected from RPL41, RPS15, RPS8, TRIM7 and LRG1; and
(III) selecting the cancer therapy based on the evaluation of step (II), wherein the cancer therapy comprises a chimeric protein of a general structure of:
N terminus—(a)—(b)—(c)—C terminus,
wherein:
(A)
(a) is a first domain comprising an extracellular domain of a Type I transmembrane protein, the transmembrane protein being TIGIT,
(b) is a linker comprising at least one cysteine residue capable of forming a disulfide bond, and
(c) is a second domain comprising an extracellular domain of Type II transmembrane protein, the transmembrane protein being LIGHT, wherein the linker connects the first domain and the second domain and optionally comprises one or more joining linkers, such joining linkers being selected from SEQ ID NOs: 49-95; or
(B)
(a) is a first domain comprising an extracellular domain of a Type I transmembrane protein, the transmembrane protein being SIRPα,
(b) is a linker comprising at least one cysteine residue capable of forming a disulfide bond, and
(c) is a second domain comprising an extracellular domain of Type II transmembrane protein, the transmembrane protein being 4-1BBL, wherein the linker connects the first domain and the second domain and optionally comprises one or more joining linkers, such joining linkers being selected from SEQ ID NOs:
49-95.

108. A method for selecting a patient for a cancer treatment, the method comprising:
(I) obtaining a biological sample from a subject;
(II) evaluating the biological sample for the expression of:
(i) a gene selected from CD274, B2M, STAT1, STAT2, TRIM7, IRF1, TAP1, TAP2, CASP1, IRF, LTBR, PVR, GASTA3, LRG1, SPRY2, ARG1, TRIM8, TRIM2, MAPK81P1, TRIM6, and KRT1; and/or
(ii) a gene selected from RPL41, RPS15, RPS8, TRIM7 and LRG1; and
and
(III) selecting the cancer therapy based on the evaluation of step (II), wherein the cancer therapy comprises a chimeric protein of a general structure of:
N terminus—(a)—(b)—(c)—C terminus,
wherein:
(A)
(a) is a first domain comprising an extracellular domain of a Type I transmembrane protein, the transmembrane protein being TIGIT,
(b) is a linker comprising at least one cysteine residue capable of forming a disulfide bond, and
(c) is a second domain comprising an extracellular domain of Type II transmembrane protein, the transmembrane protein being LIGHT, wherein the linker connects the first domain and the second domain and optionally comprises one or more joining linkers, such joining linkers being selected from SEQ ID NOs: 49-95; or
(B)
(a) is a first domain comprising an extracellular domain of a Type I transmembrane protein, the transmembrane protein being SIRPα,
(b) is a linker comprising at least one cysteine residue capable of forming a disulfide bond, and
(c) is a second domain comprising an extracellular domain of Type II transmembrane protein, the transmembrane protein being 4-1BBL, wherein the linker connects the first domain and the second domain and optionally comprises one or more joining linkers, such joining linkers being selected from SEQ ID NOs:
49-95.

109. A method of treating cancer, the method comprising:
(I) obtaining a biological sample from a subject;
(II) evaluating the biological sample for the expression of:
(i) a gene selected from CD274, B2M, STAT1, STAT2, TRIM7, IRF1, TAP1, TAP2, CASP1, IRF, LTBR, PVR, GASTA3, LRG1, SPRY2, ARG1, TRIM8, TRIM2, MAPK81P1, TRIM6, and KRT1; and/or
(ii) a gene selected from RPL41, RPS15, RPS8, TRIM7 and LRG1; and
(III) wherein the cancer therapy comprises a chimeric protein of a general structure of:
N terminus—(a)—(b)—(c)—C terminus,
wherein:
(A)
(a) is a first domain comprising an extracellular domain of a Type I transmembrane protein, the transmembrane protein being TIGIT,
(b) is a linker comprising at least one cysteine residue capable of forming a disulfide bond, and
(c) is a second domain comprising an extracellular domain of Type II transmembrane protein, the transmembrane protein being LIGHT, wherein the linker connects the first domain and the second domain and optionally comprises one or more joining linkers, such joining linkers being selected from SEQ ID NOs: 49-95; or
(B)
(a) is a first domain comprising an extracellular domain of a Type I transmembrane protein, the transmembrane protein being SIRPα,
(b) is a linker comprising at least one cysteine residue capable of forming a disulfide bond, and
(c) is a second domain comprising an extracellular domain of Type II transmembrane protein, the transmembrane protein being 4-1BBL, wherein the linker connects the first domain and the second domain and optionally comprises one or more joining linkers, such joining linkers being selected from SEQ ID NOs: 49-95; and
(IV) optionally administering the cancer therapy with an ability to inhibit function and/or activity of PD-1, PD-L1 and/or PD-L2.

110. The method of any one of claims 107 to 109, wherein the cancer therapy with an ability to inhibit function and/or activity of PD-1, PD-L1 and/or PD-L2 is selected when the biological sample comprises at least one tumor cell, and
a gene selected from CD274, B2M, STAT1, STAT2, TRIM7, IRF1, TAP1, TAP2, CASP1, IRF, LTBR, PVR, GASTA3, LRG1, SPRY2, ARG1, TRIM8, TRIM2, MAPK8IP1, TRIM6, and KRT1 is not upregulated in the at least one tumor cell compared to a healthy tissue, a prior biological sample obtained from the subject, or another biological sample from patient that is known to be sensitive to anti-PD-1 therapy; and/or
a gene selected from RPL41, RPS15, RPS8, TRIM7 and LRG1 is not downregulated in the at least one tumor cell compared to a healthy tissue, a prior biological sample obtained from the subject, or another biological sample from patient that is known to be sensitive to anti-PD-1 therapy.

111. The method of any one of claims 107 to 109, wherein, when the biological sample comprises at least one tumor cell, and
a gene selected from CD274, B2M, STAT1, STAT2, TRIM7, IRF1, TAP1, TAP2, CASP1, IRF, LTBR, PVR, GASTA3, LRG1, SPRY2, ARG1, TRIMS, TRIM2, MAPK8IP1, TRIM6, and KRT1 is upregulated in the at least one tumor cell compared to a healthy tissue, a prior biological sample obtained from the subject, or another biological sample from patient that is known to be sensitive to anti-PD-1 therapy; and/or
a gene selected from RPL41, RPS15, RPS8, TRIM7 and LRG1 is downregulated in the at least one tumor cell compared to a healthy tissue, a prior biological sample obtained from the subject, or another biological sample from patient that is known to be sensitive to anti-PD-1 therapy, and the cancer therapy comprises a chimeric protein of a general structure of:
N terminus—(a)—(b)—(c)—C terminus,
wherein:
(A)
(a) is a first domain comprising an extracellular domain of a Type I transmembrane protein, the transmembrane protein being TIGIT,
(b) is a linker comprising at least one cysteine residue capable of forming a disulfide bond, and
(c) is a second domain comprising an extracellular domain of Type II transmembrane protein, the transmembrane protein being LIGHT, wherein the linker connects the first domain and the second domain and optionally comprises one or more joining linkers, such joining linkers being selected from SEQ ID NOs: 49-95; or
(B)
(a) is a first domain comprising an extracellular domain of a Type I transmembrane protein, the transmembrane protein being SIRPα,
(b) is a linker comprising at least one cysteine residue capable of forming a disulfide bond, and
(c) is a second domain comprising an extracellular domain of Type II transmembrane protein, the transmembrane protein being 4-1BBL, wherein the linker connects the first domain and the second domain and optionally comprises one or more joining linkers, such joining linkers being selected from SEQ ID NOs: 49-95.

112. The method of any one of claims 107 to 111, wherein an upregulation of one or more genes listed in (b)(i) compared to a healthy tissue indicates a lack of response, resistance or recalcitrance to the cancer therapy with an ability to inhibit function and/or activity of PD-1, PD-L1 and/or PD-L2.

113. The method of any one of claims 107 to 112, wherein an downregulation of one or more genes listed in (b)(ii) compared to a healthy tissue indicates a lack of response, resistance or recalcitrance to the cancer therapy with an ability to inhibit function and/or activity of PD-1, PD-L1 and/or PD-L2.

114. The method of any one of claims 107 to 113, wherein an upregulation of one or more genes listed in (b)(i) compared to another biological sample from patient that is known to be sensitive to anti-PD-1 therapy indicates a lack of response, resistance or recalcitrance to the cancer therapy with an ability to inhibit function and/or activity of PD-1, PD-L1 and/or PD-L2.

115. The method of any one of claims 107 to 113, wherein a downregulation of one or more genes listed in (b)(ii) compared to another biological sample from patient that is known to be sensitive to anti-PD-1 therapy indicates a lack of response, resistance or recalcitrance to the cancer therapy with an ability to inhibit function and/or activity of PD-1, PD-L1 and/or PD-L2.

116. The method of any one of claims 107 to 115, wherein an upregulation of one or more genes listed in (b)(i) compared to a prior biological sample obtained from the subject indicates a development of a lack of response, resistance or recalcitrance to the cancer therapy with an ability to inhibit function and/or activity of PD-1, PD-L1 and/or PD-L2.

117. The method of any one of claims 107 to 116, wherein a downregulation of one or more genes listed in (b)(ii) compared to a prior biological sample obtained from the subject indicates a development of a lack of response, resistance or recalcitrance to the cancer therapy with an ability to inhibit function and/or activity of PD-1, PD-L1 and/or PD-L2.

118. The method of any one of claims 107 to 117, wherein a lack of upregulation of one or more genes listed in (b)(i) compared to a healthy tissue indicates a response to the cancer therapy with an ability to inhibit function and/or activity of PD-1, PD-L1 and/or PD-L2.

119. The method of any one of claims 107 to 118, wherein a lack of downregulation of one or more genes listed in (b)(ii) compared to a healthy tissue indicates a response to the cancer therapy with an ability to inhibit function and/or activity of PD-1, PD-L1 and/or PD-L2.

120. The method of any one of claims 107 to 119, wherein a lack of upregulation of one or more genes listed in (b)(i) compared to another biological sample from patient that is known to be sensitive to anti-PD-1 therapy indicates a response to the cancer therapy with an ability to inhibit function and/or activity of PD-1, PD-L1 and/or PD-L2.

121. The method of any one of claims 107 to 120, wherein a lack of downregulation of one or more genes listed in (b)(ii) compared to another biological sample from patient that is known to be sensitive to anti-PD-1 therapy indicates a response to the cancer therapy with an ability to inhibit function and/or activity of PD-1, PD-L1 and/or PD-L2.

122. The method of any one of claims 107 to 121, wherein a lack of upregulation of one or more genes listed in (b)(i) compared to a prior biological sample obtained from the subject indicates a development of lack of a response to the cancer therapy with an ability to inhibit function and/or activity of PD-1, PD-L1 and/or PD-L2.

123. The method of any one of claims 107 to 122, wherein a lack of downregulation of one or more genes listed in (b)(ii) compared to a prior biological sample obtained from the subject indicates a development of lack of a response to the cancer therapy with an ability to inhibit function and/or activity of PD-1, PD-L1 and/or PD-L2.

124. The method of any one of claims 107 to 123, wherein the biological sample is a fresh tissue sample, frozen tumor tissue specimen, cultured cells, circulating tumor cells, or a formalin-fixed paraffin-embedded tumor tissue specimen.

125. The method of any one of claims 107 to 124, wherein the biological sample is a biopsy sample, optionally wherein the biopsy sample is selected from endoscopic biopsy, bone marrow biopsy, endoscopic biopsy (e.g., cystoscopy, bronchoscopy and colonoscopy), needle biopsy (e.g., fine-needle aspiration, core needle biopsy, vacuum-assisted biopsy, X-ray-assisted biopsy, computerized tomography (CT)-assisted biopsy, magnetic resonance imaging (MRI)-assisted biopsy and ultrasound-assisted biopsy), skin biopsy (e.g., shave biopsy, punch biopsy, and incisional biopsy) and surgical biopsy.

126. The method of any one of claims 107 to 125, wherein the biological sample comprises a body fluid selected from blood, plasma, serum, lacrimal fluid, tears, bone marrow, blood, blood cells, ascites, tissue or fine needle biopsy sample, cell-containing body fluid, free floating nucleic acids, sputum, saliva, urine, cerebrospinal fluid, peritoneal fluid, pleural fluid, feces, lymph, gynecological fluid, skin swab, vaginal swab, oral swab, nasal swab, washing or lavage such as a ductal lavage or broncheoalveolar lavage, aspirate, scraping, bone marrow specimen, tissue biopsy specimen, surgical specimen, feces, other body fluids, secretions, and/or excretions, and/or cells therefrom.

127. The method of any one of claims 107 to 126, wherein the biological sample is obtained by a technique selected from scrapes, swabs, and biopsy, optionally wherein the biological sample is obtained by use of brushes, (cotton) swabs, spatula, rinse/wash fluids, punch biopsy devices, puncture of cavities with needles or surgical instrumentation.

128. The method of any one of claims 121 to 127, wherein the biological sample comprises at least one tumor cell.

129. The method of claim 108, 109 or 128, wherein the tumor is selected from Hodgkin's and non-Hodgkin's lymphoma, B-cell lymphoma (including low grade/follicular non-Hodgkin's lymphoma (NHL); small lymphocytic (SL) NHL; intermediate grade/follicular NHL; intermediate grade diffuse NHL; high grade immunoblastic NHL; high grade lymphoblastic NHL; high grade small non-cleaved cell NHL; bulky disease NHL; mantle cell lymphoma; AIDS-related lymphoma; and Waldenstrom's Macroglobulinemia; chronic lymphocytic leukemia (CLL); acute lymphoblastic leukemia (ALL); Hairy cell leukemia; or chronic myeloblastic leukemia, basal cell carcinoma, biliary tract cancer; bladder cancer; bone cancer; brain and central nervous system cancer; breast cancer; cancer of the peritoneum; cervical cancer; choriocarcinoma; colon and rectum cancer; connective tissue cancer; cancer of the digestive system; endometrial cancer; esophageal cancer; eye cancer; cancer of the head and neck; gastric cancer (including gastrointestinal cancer); glioblastoma; hepatic carcinoma; hepatoma; intra-epithelial neoplasm; kidney or renal cancer; larynx cancer; leukemia; liver cancer; lung cancer (e.g., small-cell lung cancer, non-small cell lung cancer, adenocarcinoma of the lung, and squamous carcinoma of the lung); melanoma; myeloma; neuroblastoma; oral cavity cancer (lip, tongue, mouth, and pharynx); ovarian cancer; pancreatic cancer; prostate cancer; retinoblastoma; rhabdomyosarcoma; rectal cancer; cancer of the respiratory system; salivary gland carcinoma; sarcoma; skin cancer; squamous cell cancer; stomach cancer; testicular cancer; thyroid cancer; uterine or endometrial cancer; cancer of the urinary system; vulval cancer; lymphoma including Hodgkin's and non-Hodgkin's lymphoma, as well as B-cell lymphoma (including low grade/follicular non-Hodgkin's lymphoma (NHL); small lymphocytic (SL) NHL; intermediate grade/follicular NHL; intermediate grade diffuse NHL; high grade immunoblastic NHL; high grade lymphoblastic NHL; high grade small non-cleaved cell NHL; bulky disease NHL; mantle cell lymphoma; AIDS-related lymphoma; and Waldenstrom's Macroglobulinemia; chronic lymphocytic leukemia (CLL); acute lymphoblastic leukemia (ALL); Hairy cell leukemia; chronic myeloblastic leukemia; as well as other carcinomas and sarcomas; and post-transplant lymphoproliferative disorder (PTLD), as well as abnormal vascular proliferation associated with phakomatoses, edema (e.g., that associated with brain tumors), Meigs' syndrome cancer; renal carcinoma; colorectal cancer; and adrenal cancer.

130. The method of any one of claims 107 to 129, wherein the evaluating is performed by DNA sequencing, RNA sequencing, immunohistochemical staining, western blotting, in cell western, immunofluorescent staining, ELISA, and fluorescent activating cell sorting (FACS) or a combination thereof.

131. The method of any one of claims 107 to 130, wherein the evaluating is performed by contacting the sample with an agent that specifically binds to one or more proteins encoded by one or more genes listed in (b)(i) and/or (b)(ii), optionally wherein the agent that specifically binds to one or proteins comprises an antibody, antibody-like molecule or binding a fragment thereof.

132. The method of any one of claims 107 to 131, wherein the evaluating is performed by contacting the sample with an agent that specifically binds to one or more of nucleic acids of one or more genes associated with a gene listed in (b)(i) and/or (b)(ii).

133. The method of claim 132, wherein the agent that specifically binds to one or more of the nucleic acids is a nucleic acid primer or probe.

134. A method of determining a cancer treatment for a patient, the method comprising:
(I) obtaining a biological sample from a subject;
(II) evaluating the biological sample for the activation of a pathway selected from Mapk8ip1, Trim7, Elk1, Lrg1, Arg1, Rap1, and Ras; and
(III) selecting the cancer therapy based on the evaluation of step (II), wherein the cancer therapy comprises a chimeric protein of a general structure of:
N terminus—(a)—(b)—(c)—C terminus,
wherein:
(A)
(a) is a first domain comprising an extracellular domain of a Type I transmembrane protein, the transmembrane protein being TIGIT,
(b) is a linker comprising at least one cysteine residue capable of forming a disulfide bond, and
(c) is a second domain comprising an extracellular domain of Type II transmembrane protein, the transmembrane protein being LIGHT, wherein the linker connects the first domain and the second domain and optionally comprises one or more joining linkers, such joining linkers being selected from SEQ ID NOs: 49-95; or
(B)
(a) is a first domain comprising an extracellular domain of a Type I transmembrane protein, the transmembrane protein being SIRPα,
(b) is a linker comprising at least one cysteine residue capable of forming a disulfide bond, and
(c) is a second domain comprising an extracellular domain of Type II transmembrane protein, the transmembrane protein being 4-1BBL, wherein the linker connects the first domain and the second domain and optionally comprises one or more joining linkers, such joining linkers being selected from SEQ ID NOs:
49-95.

135. A method for selecting a patient for a cancer treatment, the method comprising:
(I) obtaining a biological sample from a subject;
(II) evaluating the biological sample for the activation of a pathway selected from Mapk8ip1, Trim7, Elk1, Lrg1, Arg1, Rap1, and Ras; and
(III) selecting the cancer therapy based on the evaluation of step (II), wherein the cancer therapy comprises a chimeric protein of a general structure of:
N terminus—(a)—(b)—(c)—C terminus,
wherein:
(A)
(a) is a first domain comprising an extracellular domain of a Type I transmembrane protein, the transmembrane protein being TIGIT,
(b) is a linker comprising at least one cysteine residue capable of forming a disulfide bond, and
(c) is a second domain comprising an extracellular domain of Type II transmembrane protein, the transmembrane protein being LIGHT, wherein the linker connects the first domain and the second domain and optionally comprises one or more joining linkers, such joining linkers being selected from SEQ ID NOs: 49-95; or
(B)
(a) is a first domain comprising an extracellular domain of a Type I transmembrane protein, the transmembrane protein being SIRPα,
(b) is a linker comprising at least one cysteine residue capable of forming a disulfide bond, and
(c) is a second domain comprising an extracellular domain of Type II transmembrane protein, the transmembrane protein being 4-1BBL, wherein the linker connects the first domain and the second domain and optionally comprises one or more joining linkers, such joining linkers being selected from SEQ ID NOs: 49-95.

136. A method of treating cancer, the method comprising:
(I) obtaining a biological sample from a subject;
(II) evaluating the biological sample for the activation of a pathway selected from Mapk8ip1, Trim7, Elk1, Lrg1, Arg1, Rap1, and Ras; and
(II) wherein the cancer therapy comprises a chimeric protein of a general structure of:
N terminus—(a)—(b)—(c)—C terminus,
wherein:
(A)
(a) is a first domain comprising an extracellular domain of a Type I transmembrane protein, the transmembrane protein being TIGIT,
(b) is a linker comprising at least one cysteine residue capable of forming a disulfide bond, and
(c) is a second domain comprising an extracellular domain of Type II transmembrane protein, the transmembrane protein being LIGHT, wherein the linker connects the first domain and the second domain and optionally comprises one or more joining linkers, such joining linkers being selected from SEQ ID NOs: 49-95; or
(B)
(a) is a first domain comprising an extracellular domain of a Type I transmembrane protein, the transmembrane protein being SIRPα,
(b) is a linker comprising at least one cysteine residue capable of forming a disulfide bond, and
(c) is a second domain comprising an extracellular domain of Type II transmembrane protein, the transmembrane protein being 4-1BBL, wherein the linker connects the first domain and the second domain and optionally comprises one or more joining linkers, such joining linkers being selected from SEQ ID NOs: 49-95; and
(IVI) optionally administering the cancer therapy with an ability to inhibit function and/or activity of PD-1, PD-L1 and/or PD-L2.

137. The method of any one of claims 134 to 136, wherein the cancer therapy with an ability to inhibit function and/or activity of PD-1, PD-L1 and/or PD-L2 is selected when the biological sample comprises at least one tumor cell, and
pathway selected from Mapk8ip1, Trim7, Elk1, Lrg1, Arg1, Rap1, and Ras is not upregulated in the at least one tumor cell compared to a healthy tissue, a prior biological sample obtained from the subject, or another biological sample from patient that is known to be sensitive to anti-PD-1 therapy.

138. The method of any one of claims 134 to 137, wherein, when the biological sample comprises at least one tumor cell, and
pathway selected from Mapk8ip1, Trim7, Elk1, Lrg1, Arg1, Rap1, and Ras is upregulated in the at least one tumor cell compared to a healthy tissue, a prior biological sample obtained from the subject, or another biological sample from patient that is known to be sensitive to anti-PD-1 therapy, and the cancer therapy comprises a chimeric protein of a general structure of:
N terminus—(a)—(b)—(c)—C terminus,
wherein:
(A)
(a) is a first domain comprising an extracellular domain of a Type I transmembrane protein, the transmembrane protein being TIGIT,
(b) is a linker comprising at least one cysteine residue capable of forming a disulfide bond, and
(c) is a second domain comprising an extracellular domain of Type II transmembrane protein, the transmembrane protein being LIGHT, wherein the linker connects the first domain and the second domain and optionally comprises one or more joining linkers, such joining linkers being selected from SEQ ID NOs: 49-95; or
(B)
(a) is a first domain comprising an extracellular domain of a Type I transmembrane protein, the transmembrane protein being SIRPα,
(b) is a linker comprising at least one cysteine residue capable of forming a disulfide bond, and
(c) is a second domain comprising an extracellular domain of Type II transmembrane protein, the transmembrane protein being 4-1BBL, wherein the linker connects the first domain and the second domain and optionally comprises one or more joining linkers, such joining linkers being selected from SEQ ID NOs: 49-95.

139. The method of any one of claims 134 to 138, wherein an upregulation of pathway selected from Mapk8ip1, Trim7, Elk1, Lrg1, Arg1, Rap1, and Ras compared to a healthy tissue indicates a lack of response, resistance or recalcitrance to the cancer therapy with an ability to inhibit function and/or activity of PD-1, PD-L1 and/or PD-L2.

140. The method of any one of claims 134 to 139, wherein an upregulation of pathway selected from Mapk8ip1, Trim7, Elk1, Lrg1, Arg1, Rap1, and Ras compared to another biological sample from patient that is known to be sensitive to anti-PD-1 therapy indicates a lack of response, resistance or recalcitrance to the cancer therapy with an ability to inhibit function and/or activity of PD-1, PD-L1 and/or PD-L2.

141. The method of any one of claims 134 to 140, wherein an upregulation of pathway selected from Mapk8ip1, Trim7, Elk1, Lrg1, Arg1, Rap1, and Ras compared to a prior biological sample obtained from the subject indicates a development of a lack of response, resistance or recalcitrance to the cancer therapy with an ability to inhibit function and/or activity of PD-1, PD-L1 and/or PD-L2.

142. The method of any one of claims 134 to 141, wherein a lack of upregulation of pathway selected from Mapk8ip1, Trim7, Elk1, Lrg1, Arg1, Rap1, and Ras compared to a healthy tissue indicates a response to the cancer therapy with an ability to inhibit function and/or activity of PD-1, PD-L1 and/or PD-L2.

143. The method of any one of claims 134 to 142, wherein a lack of upregulation of pathway selected from Mapk8ip1, Trim7, Elk1, Lrg1, Arg1, Rap1, and Ras compared to another biological sample from patient that is known to be sensitive to anti-PD-1 therapy indicates a response to the cancer therapy with an ability to inhibit function and/or activity of PD-1, PD-L1 and/or PD-L2.

144. The method of any one of claims 134 to 143, wherein a lack of upregulation of pathway selected from Mapk8ip1, Trim7, Elk1, Lrg1, Arg1, Rap1, and Ras compared to a prior biological sample obtained from the subject indicates a development of lack of a response to the cancer therapy with an ability to inhibit function and/or activity of PD-1, PD-L1 and/or PD-L2.

145. The method of any one of claims 134 to 144, wherein the biological sample is a fresh tissue sample, frozen tumor tissue specimen, cultured cells, circulating tumor cells, or a formalin-fixed paraffin-embedded tumor tissue specimen.

146. The method of any one of claims 134 to 145, wherein the biological sample is a biopsy sample.

147. The method of claim 146, wherein the biopsy sample is selected from endoscopic biopsy, bone marrow biopsy, endoscopic biopsy (e.g., cystoscopy, bronchoscopy and colonoscopy), needle biopsy (e.g., fine-needle aspiration, core needle biopsy, vacuum-assisted biopsy, X-ray-assisted biopsy, computerized tomography (CT)-assisted biopsy, magnetic resonance imaging (MRI)-assisted biopsy and ultrasound-assisted biopsy), skin biopsy (e.g., shave biopsy, punch biopsy, and incisional biopsy) and surgical biopsy.

148. The method of any one of claims 134 to 147, wherein the biological sample comprises a body fluid selected from blood, plasma, serum, lacrimal fluid, tears, bone marrow, blood, blood cells, ascites, tissue or fine needle biopsy sample, cell-containing body fluid, free floating nucleic acids, sputum, saliva, urine, cerebrospinal fluid, peritoneal fluid, pleural fluid, feces, lymph, gynecological fluid, skin swab, vaginal swab, oral swab, nasal swab, washing or lavage such as a ductal lavage or broncheoalveolar lavage, aspirate, scraping, bone marrow specimen, tissue biopsy specimen, surgical specimen, feces, other body fluids, secretions, and/or excretions, and/or cells therefrom.

149. The method of any one of claims 134 to 148, wherein the biological sample is obtained by a technique selected from scrapes, swabs, and biopsy.

150. The method of claim 149, wherein the biological sample is obtained by use of brushes, (cotton) swabs, spatula, rinse/wash fluids, punch biopsy devices, puncture of cavities with needles or surgical instrumentation.

151. The method of any one of claims 134 to 150, wherein the biological sample comprises at least one tumor cell.

152. The method of claim 135, 136 or 151, wherein the tumor is selected from Hodgkin's and non-Hodgkin's lymphoma, B-cell lymphoma (including low grade/follicular non-Hodgkin's lymphoma (NHL); small lymphocytic (SL) NHL; intermediate grade/follicular NHL; intermediate grade diffuse NHL; high grade immunoblastic NHL; high grade lymphoblastic NHL; high grade small non-cleaved cell NHL; bulky disease NHL; mantle cell lymphoma; AIDS-related lymphoma; and Waldenstrom's Macroglobulinemia; chronic lymphocytic leukemia (CLL); acute lymphoblastic leukemia (ALL); Hairy cell leukemia; or chronic myeloblastic leukemia, basal cell carcinoma, biliary tract cancer; bladder cancer; bone cancer; brain and central nervous system cancer; breast cancer; cancer of the peritoneum; cervical cancer; choriocarcinoma; colon and rectum cancer; connective tissue cancer; cancer of the digestive system; endometrial cancer; esophageal cancer; eye cancer; cancer of the head and neck; gastric cancer (including gastrointestinal cancer); glioblastoma; hepatic carcinoma; hepatoma; intra-epithelial neoplasm; kidney or renal cancer; larynx cancer; leukemia; liver cancer; lung cancer (e.g., small-cell lung cancer, non-small cell lung cancer, adenocarcinoma of the lung, and squamous carcinoma of the lung); melanoma; myeloma; neuroblastoma; oral cavity cancer (lip, tongue, mouth, and pharynx); ovarian cancer; pancreatic cancer; prostate cancer; retinoblastoma; rhabdomyosarcoma; rectal cancer; cancer of the respiratory system; salivary gland carcinoma; sarcoma; skin cancer; squamous cell cancer; stomach cancer; testicular cancer; thyroid cancer; uterine or endometrial cancer; cancer of the urinary system; vulval cancer; lymphoma including Hodgkin's and non-Hodgkin's lymphoma, as well as B-cell lymphoma (including low grade/follicular non-Hodgkin's lymphoma (NHL); small lymphocytic (SL) NHL; intermediate grade/follicular NHL; intermediate grade diffuse NHL; high grade immunoblastic NHL; high grade lymphoblastic NHL; high grade small non-cleaved cell NHL; bulky disease NHL; mantle cell lymphoma; AIDS-related lymphoma; and Waldenstrom's Macroglobulinemia; chronic lymphocytic leukemia (CLL); acute lymphoblastic leukemia (ALL); Hairy cell leukemia; chronic myeloblastic leukemia; as well as other carcinomas and sarcomas; and post-transplant lymphoproliferative disorder (PTLD), as well as abnormal vascular proliferation associated with phakomatoses, edema (e.g., that associated with brain tumors), Meigs' syndrome cancer; renal carcinoma; colorectal cancer; and adrenal cancer.

153. The method of any one of claims 134 to 152, wherein the evaluating is performed by DNA sequencing, RNA sequencing, immunohistochemical staining, western blotting, in cell western, immunofluorescent staining, ELISA, and fluorescent activating cell sorting (FACS) or a combination thereof.

154. The method of any one of claims 134 to 153, wherein the evaluating is performed by contacting the sample with an agent that specifically binds to one or more proteins encoded by a pathway selected from Mapk8ip1, Trim7, Elk1, Lrg1, Arg1, Rap1, and Ras.

155. The method of claim 154, wherein the agent that specifically binds to one or proteins comprises an antibody, antibody-like molecule or binding a fragment thereof.

156. The method of any one of claims 134 to 154, wherein the evaluating is performed by contacting the sample with an agent that specifically binds to one or more of nucleic acids of one or more genes associated with a pathway selected from Mapk8ip1, Trim7, Elk1, Lrg1, Arg1, Rap1, and Ras.

157. The method of claim 156, wherein the agent that specifically binds to one or more of the nucleic acids is a nucleic acid primer or probe.
